(19) United States
(12) Patent Application Publication
(10) Pub. No.: US 2023/0355595 A1
(43) Pub. Date: Nov. 9, 2023

(54) USE OF PRIDOPIDINE FOR TREATING FUNCTIONAL DECLINE

(71) Applicant: Prilenia Neurotherapeutics Ltd., Yakum (IL)

(72) Inventors: Michael HAYDEN, Yakum (IL); Michal GEVA, Even-Yehuda (IL)

(73) Assignee: Prilenia Neurotherapeutics Ltd., Yakum (IL)

(21) Appl. No.: 18/324,980

(22) Filed: May 28, 2023

Related U.S. Application Data
(63) Continuation-in-part of application No. 18/306,257, filed on Apr. 25, 2023.
Application No. 18/306,257 is a continuation-in-part of application No. 18/164,587, filed on Feb. 5, 2023.
Application No. 18/164,587 is a continuation of application No. 17/019,346, filed on Sep. 13, 2020, now abandoned.
Application No. 17/019,346 is a continuation-in-part of application No. 15/685,993, filed on Aug. 24, 2017, now Pat. No. 11,207,310.
(60) Provisional application No. 62/416,685, filed on Nov. 2, 2016.
(60) Provisional application No. 62/411,511, filed on Oct. 21, 2016.
(60) Provisional application No. 62/395,263, filed on Sep. 15, 2016.
(60) Provisional application No. 62/379,175, filed on Aug. 24, 2016.

(51) Int. Cl. A61K 31/44; A61P 25/14; A61K 9/48

(52) U.S. Cl. CPC A61K 31/44; A61P 25/14; A61K 9/48

(57) ABSTRACT

Provided herein a method of maintaining, improving, or lessening the decline of functional capacity, cognition, motor function and quality of life of a subject afflicted with Huntington disease, including those afflicted with early-stage Huntington disease (HD1 and HD2, TFC 7-13) by orally administering to the subject a pharmaceutical composition comprising pridopidine or a pharmaceutically acceptable salt thereof.

### CROSS-REFERENCE TO RELATED APPLICATIONS

This application is a Continuation in Part from United-States application Ser. No. 18/306,257 filed Apr. 25, 2023, which is a Continuation in Part from United-States application Ser. No. 18/164,587 filed Feb. 5, 2023, which is a Continuation from United-States application Ser. No. 17/019,346 filed Sep. 13, 2020, which is a Continuation in Part from United-States application Ser. No. 15/685,993 filed Aug. 24, 2017, now U.S. Pat. No. 11,207,310 issued Dec. 28, 2021, which claims the benefit of United-States Provisional Application No. 62/416,685, filed Nov. 2, 2016, U.S. Provisional Application No. 62/411,511, filed Oct. 21, 2016, United-States Provisional Application No. 62/395,263, filed Sep. 15, 2016, and United-States Provisional Application No. 62/379,175, filed Aug. 24, 2016, which are all hereby incorporated by reference in their entirety.

Throughout this application, various publications are referred to by the first author and year of publication. Full citations for these publications are presented in a References section immediately before the claims. Disclosures of the publications cited in the References section are hereby incorporated by reference in their entireties into this application to describe the state of the art more fully as of the date of the invention described herein.

### FIELD OF THE INVENTION

Disclosed herein are methods of use of pridopidine or a pharmaceutically acceptable salt thereof, for maintaining, improving, or lessening the decline of functional capacity, cognition, motor function and quality of life in a subject afflicted with Huntington disease, including those afflicted with early-stage Huntington disease (HD1 and HD2, TFC 7-13).

### BACKGROUND OF INVENTION

### Huntington Disease

Huntington disease (HD) is a fatal neurodegenerative disorder with an autosomal dominant mode of inheritance. The disease is associated with a triad of motor, behavioral, and cognitive symptoms. Motor disturbances are the defining feature of the disease, with chorea the most evident motor symptom. Although useful for diagnosis, chorea is a poor marker of disease severity. Rather, disability and disease severity best correlate with negative functional, cognitive, and motor features such as decline in functional capacity, HD-Quality of Life scale (HD-QoL), Stroop word test (SWR), symbol digit modality test (SDMT) and impairment in fine motor skills, bradykinesia, and gross motor coordination skills, including speech difficulties, gait, and postural dysfunction (Mahant 2003).

Disease progression in HD is defined by clinical rating scales, i.e. The Unified Huntington's Disease Rating Scale Total Functional Capacity (UHDRS-TFC), cUHDRS as a global measure of disease progression.

Several medications are prescribed to ameliorate the motor and emotional problems associated with HD. However, the scientific evidence for the usefulness of various drugs in HD is poor (Mestre 2009). Only tetrabenazine and deutetrabenazine, which reduce dopamine availability and transmission, are registered specifically for the treatment of patients with HD for the management of chorea only. No registered drugs are available for the management of the multifaceted symptoms of HD, resulting in inexorable functional capacity decline throughout the course of the disease. As such, there is a significant unmet medical need to develop medications to retard or ameliorate functional deficits in HD.

### Pridopidine

Pridopidine (4-[3-(methyl sulfonyl)phenyl]-1-propyl-piperidine) (formerly known as ACR16) is a drug under development for the treatment of Huntington disease. Pridopidine has a selective and high affinity for the sigma-1 receptor (S1R, binding IC50˜100 nM), with low-affinity binding to additional receptors, including the dopamine D2/D3 receptors (in the micromolar range).

The S1R is an endoplasmic reticulum (ER) chaperone protein implicated in cellular differentiation, neuroplasticity and neuroprotection. Activation of the S1R by pridopidine leads to upregulation of pathways known to promote neuronal plasticity and survival, including the AKT/Phosphoinositide kinase (PI3K) pathway and the dopamine receptor 1 (D1R). Upregulation of these pathways demonstrates therapeutic benefit in HD preclinical models (Geva et al., 2016). In primary mouse neurons transfected with mHtt as well as in HD patient-derived iPSCs (induced pluripotent stem cells), pridopidine shows a robust and dose dependent rescue of mHtt-induced cell death (Eddings et al., 2019). These neuroprotective effects are mediated by activation of the S1R as pharmacological inhibition of the S1R and genetic deletion of the S1R completely abolishes the effects (Eddings et al, 2019).

Pridopidine upregulates the secretion and downstream signaling of the neuroprotective brain-derived neuroptrophic factor (BDNF) (Geva et al., 2016). A decrease in BDNF is associated with HD pathogenesis. Preclinical studies consistently show that BDNF is highly protective against the toxic effects of mutant Huntingtin (mHtt). Homeostatic synaptic plasticity (HSP), the processes that maintain the stability of neuronal networks and underlie learning and cognitive capabilities, are disrupted in HD and regulated by BDNF (Smith-Dijak et al., 2019). Treatment of cultured cortical neurons from the HD YAC128 mouse model with pridopidine rescues the impaired HSP (Smith-Dijak et al., 2019). Modulation of the BDNF pathway is a major component of pridopidine's S1R-mediate neuroprotective effects.

cUHDRS: Composite Unified Huntington Disease Rating Scale

The cUHDRS scoring system combines four measurement scales for assessing functional, motor, and cognitive function to provide a quantitative holistic measure of patient experience. The scales included in cUHDRS are the Total Motor Scale (TMS, a motor assessment), Total Functional Capacity (TFC, a functional assessment), the Symbol Digit Modality Test (SDMT, a cognitive assessment), and the Stroop Word Reading Test (SWR, a cognitive assessment). This composite measure characterizes the clinical progression of HD and is strongly associated with brain measures of progressive atrophy in the corticostriatal tract relevant to the disease. The cUHDRS increases the low signal-to-noise ratio (SN) that afflicts the individual measures and provides a good coverage of key features including functional, motor, and cognitive assessments in the early HD population (Schobel et al., 2017).

cUHDRS shows increased sensitivity to clinical changes especially in early symptomatic HD patients. In addition, cUHDRS has greater statistical power to detect success in clinical trials aiming to slow clinical progression compared to TFC and TMS alone. Therefore, the cUHDRS may be beneficial as an endpoint in interventional clinical trials in the early HD population, where it can assist in maximizing sensitivity while lowering patient burden and reducing sample size for a potentially more efficient trial (Schobel et al., 2017).

### BRIEF SUMMARY OF THE INVENTION

In one aspect, provided herein is a method of maintaining, improving, or lessening the decline of motor function and functional capacity in a human patient afflicted with early-stage Huntington disease (HD1 and HD2, TFC 7-13). Said method comprises orally administering to the patient a pharmaceutical composition comprising pridopidine or a pharmaceutically acceptable salt thereof, wherein assessment of said maintaining, improving, or lessening the decline of motor and functional capacity comprises using a composite Unified Huntington Disease rating scale (cUHDRS). Said cUHDRS comprises measurement of the total functional capacity (TFC), total motor score (TMS), symbol digital modalities test (SDMT), and Stroop Word Reading Test (SWR) of the patient according to the following equation:

$$cUHDRS = [ ( \frac{T F C - 10.4}{1.9} ) - ( \frac{TMS - 29.7}{14.9} ) + ( \frac{S D M T - 28.4}{11.3} ) + ( \frac{SWR - 66.1}{20.1} ) ] + 10 .$$

In a related aspect, pridopidine or pharmaceutically acceptable salt thereof is administered at a dose of 90 mg/day (e.g. 45 mg bid per day).

In another related aspect, the composition is administered twice per day, wherein pridopidine or pharmaceutically acceptable salt thereof is administered at a dose of 45 mg bid per day.

In another related aspect, the administration is for at least 26 weeks, at least 39 weeks, at least 52 weeks, at least 65 weeks, at least 78 weeks, at least 24 months, at least 36 months, at least 48 months, or at least 60 months.

In a further related aspect, pridopidine or a pharmaceutically acceptable salt thereof is administered at a dose of 90 mg per day for a period of at least 26 weeks, at least 39 weeks, at least 52 weeks, at least 65 weeks, at least 78 weeks, at least 24 months, at least 36 months, at least 48 months, or at least 60 months.

In a further related aspect, pridopidine or a pharmaceutically acceptable salt thereof is administered at a dose of 45 mg bid (per day) for a period of at least 26 weeks, at least 39 weeks, at least 52 weeks, at least 65 weeks, at least 78 weeks, at least 24 months, at least 36 months, at least 48 months, or at least 60 months.

In another related aspect, a method comprises maintaining, improving, or lessening the decline of total functional capacity (TFC) of said patient. In another related aspect, the method comprises maintaining, improving, or lessening the decline of Stroop Word Reading (SWR) test of said patient. In still another related aspect, a method comprises maintaining, improving, or lessening the decline of motor function is said to be patient. In yet another related aspect, the composite Unified Huntington Disease rating scale (cUHDRS) produces an improved longitudinal Signal to Noise (S/N) ratio compared with a longitudinal S/N ratio of at least one of the independent UHDRS clinical measures of TFC, TMS, SDMT, SWR, HD-QoL, gait and balance score. In another related aspect, composite Unified Huntington Disease rating scale (cUHDRS) produces an improved measurement values compared with any one of the independent UHDRS clinical measures of TFC, TMS, SDMT, SWR, HD-QoL, gait and balance score.

In another related aspect, a patient has greater than or equal to 36 CAG repeats in the Huntingtin gene and wherein said early-stage Huntington disease (HD1 and HD2, TFC 7-13) comprises a baseline TFC score greater than or equal to 7.

In another related aspect, oral administration comprises administration of a capsule.

This invention provides a method of maintaining functional capacity, improving functional capacity, or lessening the decline of functional capacity in a human patient comprising periodically orally administering to the patient a pharmaceutical composition comprising pridopidine such that a dose of 90 mg (e.g. 45 mg bid) of pridopidine is administered to the patient per day, so as to thereby maintain functional capacity, improve functional capacity, or lessen the decline of functional capacity in the human patient. In some embodiments the patient is a Huntington disease (HD) patient.

This invention provides a method of maintaining functional capacity, improving functional capacity, or reducing the rate of decline of functional capacity in a human patient comprising periodically orally administering to the patient a pharmaceutical composition comprising pridopidine or pharmaceutically acceptable salt thereof such that a dose of 90 mg of pridopidine is administered to the patient per day, so as to thereby maintain functional capacity, improve functional capacity, or reduce the rate of decline of functional capacity in the human patient. In some embodiments the method includes a dose of 45 mg bid of pridopidine administered to the patient per day. In some embodiments the patient is an HD patient.

The invention additionally provides a method of maintaining functional capacity, improving functional capacity, or slowing the clinical progression of HD as measured by total functional capacity in a human patient comprising periodically orally administering to the patient afflicted with HD a pharmaceutical composition comprising pridopidine such that a dose of 90 mg of pridopidine is administered to the patient per day, so as to thereby slow the clinical progression of HD in the patient as measured by total functional capacity. In some embodiments the method includes a dose of 45 mg bid of pridopidine administered to the patient per day.

Further provided is a method of decreasing functional decline in a human HD patient comprising periodically orally administering to the patient a pharmaceutical composition comprising pridopidine such that a dose of 90 mg/day of pridopidine or pharmaceutically acceptable salt thereof, is administered to the patient, to thereby decrease the functional decline in the patient. In some embodiments, functional decline from baseline in comparison to placebo (a HD subject not receiving pridopidine) is decreased by at least 5%, by at least 10%, by at least 15%, by at least 20%, by at least 25%, by at least 30%, by at least 35% or by at least 40%. In some embodiments the method includes a dose of about 45 mg bid of pridopidine administered to the patient per day. In some embodiments of the method, pridopidine is administered orally. In some embodiments of the method, the administration continues for at least 26 weeks, at least 39 weeks, at least 52 weeks, at least 65 weeks, about 78 weeks or at least 78 weeks. In some embodiments of the method, the HD patient is an adult patient. HD patient is classified as an early-stage patient, for example, as a stage 1 or stage 2 HD (HD1, TFC 11-13 or HD2, TFC 7-10) patient. In some embodiments, the patient has a baseline TFC score of 7-13 or at least 7, at least 8, at least 9, at least 10, at least 11, at least 12, 13 or 7-10 or 11-13. In some embodiments, functional capacity of a patient is measured using the Total Functional Capacity (TFC) scale of the Unified Huntington's Disease Rating Scale (UHDRS), UHDRS-TFC. In some embodiments of the method, the patient's baseline functional capacity and one or more subsequent functional capacity assessments are performed to determine any change in functional decline.

Further provided is a method of achieving a reduced change from baseline in the UHDRS-TFC score in a human HD patient comprising periodically orally administering to the patient a pharmaceutical composition comprising pridopidine such that a dose of 90 mg of pridopidine is administered to the patient per day, so as to thereby affect a change in the UHDRS-TFC score in the patient when compared to a HD subject not receiving pridopidine. In some embodiments the method includes a dose of about 45 mg bid of pridopidine administered to the patient per day. In some embodiments of the method, the administration continues for at least 26 weeks, or at least 39 weeks, or at least 52 weeks, or at least 65 weeks, or about 78 weeks or at least 78 weeks. In some embodiments of the method, the HD patient is classified as a stage 1 or stage 2 HD patient based on the patient's UHDRS-TFC score. In some embodiments, the patient has a baseline TFC score of 7-13 or at least 7, at least 8, at least 9, at least 10, at least 11, at least 12, 13 or 7-10 or 11-13. In some embodiments of the method, the difference in change from baseline in the UHDRS-TFC score, when compared to a HD subject not receiving pridopidine is reduced by at least 0.2 points over a period of 26 weeks or by at least 0.3 points over 52 weeks or by at least 0.3 points over 65 weeks or by 0.5 points over 78 weeks. In some embodiments of the method, the difference in change from baseline in the UHDRS-TFC score, when compared to a HD subject not receiving pridopidine, is a decrease in the rate of TFC decline by at least 5%, by at least 10%, by at least 20%, by at least 30% by at least 40% or by at least 50% at 65 weeks or 78 weeks.

In some embodiments of the methods disclosed herein, TFC includes one or more of maintaining occupation, taking care of finances, domestic chores, requiring low level of care and activities of daily living (ADL).

The invention additionally provides a method of achieving a reduced change from baseline in the Timed Up and Go (TUG) test in a human HD patient comprising periodically orally administering to the patient a pharmaceutical composition comprising pridopidine such that a dose of 90 mg of pridopidine is administered to the patient per day, so as to thereby reduce the change in the TUG test in the patient compared to a HD subject not receiving pridopidine.

The invention additionally provides a method of achieving a reduced change from baseline in the TUG test in a human HD patient comprising periodically orally administering to the patient a pharmaceutical composition comprising pridopidine such that a dose of 45 mg bid of pridopidine is administered to the patient per day, so as to thereby reduce the change in the TUG test in the patient compared to a HD subject not receiving pridopidine.

The invention additionally provides a method of achieving a reduced change from baseline in the Symbol Digit Modalities test (SDMT) test in a human HD patient comprising periodically orally administering to the patient a pharmaceutical composition comprising pridopidine such that a dose of 90 mg of pridopidine is administered to the patient per day (e.g. 45 mg twice a day), so as to thereby reduce the change in the SDMT test in the patient compared to a HD subject not receiving pridopidine.

The invention additionally provides a method of achieving a reduced change from baseline in the Stroop Word test (SWR) in a human HD patient comprising periodically orally administering to the patient a pharmaceutical composition comprising pridopidine or pharmaceutically acceptable salt thereof such that a dose of 90 mg of pridopidine is administered to the patient per day, so as to thereby reduce the change in the Stroop Word test in the patient compared to a HD subject not receiving pridopidine.

The invention additionally provides a method of achieving a reduced change from baseline in the UHDRS-Independence Scale (UHDRS-IS) in a human HD patient comprising periodically orally administering to the patient a pharmaceutical composition comprising pridopidine or pharmaceutically acceptable salt thereof such that a dose of 90 mg of pridopidine is administered to the patient per day (e.g. 45 mg bid per day), so as to thereby reduce the change in the UHDRS-IS in the patient compared to a HD subject not receiving pridopidine.

The invention additionally provides a method of achieving a reduced change from baseline in the gait and balance score as defined by the sum of the UHDRS-Total Motor Score (UHDRS-TMS) domains gait, tandem walking and retropulsion pull test in a human HD patient comprising periodically orally administering to the patient a pharmaceutical composition comprising pridopidine or pharmaceutically acceptable salt thereof such that a dose of 90 mg of pridopidine is administered to the patient per day (e.g. 45 mg bid per day), so as to thereby reduce the change in the gait and balance score in the patient compared to a HD subject not receiving pridopidine.

The invention additionally provides a method of achieving a reduced change from baseline in the UHDRS-TMS chorea subscore in a human HD patient comprising periodically orally administering to the patient a pharmaceutical composition comprising pridopidine such that a dose of 90-180 mg of pridopidine is administered to the patient per day, so as to thereby reduce the change in the UHDRS-TMS chorea subscore in the patient compared to a HD subject not receiving pridopidine.

This invention also provides a method of maintaining or improving a human patient's ability to perform activities of daily living comprising periodically orally administering to the patient a pharmaceutical composition comprising pridopidine such that a dose of 90 mg of pridopidine or pharmaceutically acceptable salt thereof is administered to the patient per day (e.g. 45 mg bid), so as to thereby maintain or improve the human patient's ability to perform activities of daily living.

The invention further provides a method of improving or maintaining, a human patient's gait and balance comprising periodically orally administering to the patient a pharmaceutical composition comprising pridopidine such that a dose of 90-225 mg of pridopidine is administered to the patient per day, so as to thereby improve or maintain, a human patient's gait and balance.

Additionally provided is a method of improving, maintaining, or slowing the decline of, a human patient's gait and balance comprising periodically orally administering to the patient a pharmaceutical composition comprising pridopidine such that a dose of 90 mg of pridopidine or pharmaceutically acceptable salt thereof is administered to the patient per day (e.g. 45 mg bid), so as to thereby improve, maintain, or slow the decline of, a human patient's gait and balance.

The invention also provides a method of improving or maintaining a human patient's independence comprising periodically orally administering to the patient a pharmaceutical composition comprising pridopidine such that a dose of 90-225 mg of pridopidine is administered to the patient per day, so as to thereby improve or maintain a human patient's independence.

The invention also provides a method of improving, maintaining, or slowing the decline of, a human patient's independence comprising periodically orally administering to the patient a pharmaceutical composition comprising pridopidine or pharmaceutically acceptable salt thereof such that a dose of 90 mg of pridopidine is administered to the patient per day (e.g. 45 mg bid), so as to thereby improve, maintain, or slow the decline of, a human patient's independence.

The invention also provides a method of improving or maintaining or slowing the decline of a human patient's Motor domains comprising periodically orally administering to the patient a pharmaceutical composition comprising pridopidine or pharmaceutically acceptable salt thereof, such that a dose of 90 mg of pridopidine is administered to the patient per day (e.g. 45 mg bid), so as to thereby improve or maintain or slow the decline of the human patient's Motor domains.

Further provided is a method of improving, maintaining, or slowing the decline of, a human patient's Cognitive domains comprising periodically orally administering to the patient a pharmaceutical composition comprising pridopidine or pharmaceutically acceptable salt thereof, such that a dose of 90 mg of pridopidine is administered to the patient per day (e.g. 45 mg bid), so as to thereby improve, maintain, or slow the decline of, a human patient's cognitive function.

The invention also provides a method of improving or maintaining or slow the decline of a human patient's Quality of Life comprising periodically orally administering to the patient a pharmaceutical composition comprising pridopidine or pharmaceutically acceptable salt thereof, such that a dose of 90 mg of pridopidine is administered to the patient per day (e.g. 45 mg bid), so as to thereby improve or maintain the human patient's Quality of Life.

Further provided is a method of improving, maintaining, or slowing the decline of, a human patient's Quality of Life comprising periodically orally administering to the patient a pharmaceutical composition comprising pridopidine such that a dose of 45 mg bid of pridopidine is administered to the patient per day, so as to thereby improve, maintain, or slow the decline of, a human patient's Quality of Life.

The invention also provides a method of improving or maintaining or slow the decline of a human patient's cognitive domains comprising periodically orally administering to the patient a pharmaceutical composition comprising pridopidine or pharmaceutically acceptable salt thereof, such that a dose of 90 mg of pridopidine is administered to the patient per day (e.g. 45 mg bid), so as to thereby improve or maintain the human patient's cognitive domains.

Further provided is a method of improving, maintaining, or slowing the decline of, a human patient's cognitive domains comprising periodically orally administering to the patient a pharmaceutical composition comprising pridopidine or pharmaceutically acceptable salt thereof, such that a dose of 90 mg of pridopidine is administered to the patient per day (e.g. 45 mg bid), so as to thereby improve, maintain, or slow the decline of, a human patient's cognitive domains. A patient's cognitive domains may also be the patient's cognitive performance across a variety of domains.

The human patient's cognitive domains may be measured, for example, by the cognitive assessment battery (CAB) and/or the Hopkins Verbal Learning Test—Revised (HVLT-R). The cognitive domains may also be measured by the trail making test B (TMT-B). The cognitive domains may also be measured by the HD Cognitive Assessment Battery (HD-CAB), which includes 6 tests.

Further provided is a method of improving or maintaining motor ability in a human patient comprising periodically orally administering to the patient a pharmaceutical composition comprising pridopidine such that a dose of 90-225 mg of pridopidine is administered to the patient per day, so as to thereby improve motor ability in the human patient.

Motor ability may be measured, for example, by the UHDRS Total Motor Score (TMS) score, the UHDRS TMS score excluding chorea or UHDRS TMS score excluding dystonia.

The invention also provides a method of reducing or maintaining the level of chorea in a human patient comprising periodically orally administering to the patient a pharmaceutical composition comprising pridopidine or pharmaceutically acceptable salt thereof, such that a dose of 90 mg of pridopidine is administered to the patient per day (e.g. 45 mg bid), so as to thereby reduce or maintain the level of chorea in a human patient.

The level of chorea may also be slowed. Accordingly, the invention provides a method of reducing, maintaining, or slowing the increase of, chorea in a human patient comprising periodically orally administering to the patient a pharmaceutical composition comprising pridopidine or pharmaceutically acceptable salt thereof, such that a dose of 90 mg of pridopidine is administered to the patient per day (e.g. 45 mg bid), to thereby reduce, maintain, or slow the increase of, chorea in a human patient.

The human patient's chorea may be measured by the UHDRS TMS chorea score.

The invention further provides a method of improving or maintaining a human patient's behavior and/or psychiatric state comprising periodically orally administering to the patient a pharmaceutical composition comprising pridopidine or pharmaceutically acceptable salt thereof, such that a dose of 90 mg of pridopidine is administered to the patient per day (e.g. 45 mg bid), so as to thereby improve or maintain the human patient's behavior and/or psychiatric state.

The human patient's behavior and/or psychiatric state may be measured, for example, by the Problem Behaviors Assessment total score. The human patient's behavior and/or psychiatric state may also be measured by the Problem Behaviors Assessment for depressed mood. The human patient's behavior and/or psychiatric state may also be measured by the Problem Behaviors Assessment for irritability. The human patient's behavior and/or psychiatric state may also be measured by the Problem Behaviors Assessment for lack of initiative or apathy. The human patient's behavior and/or psychiatric state may be measured, for example, by the Problem Behaviors Assessment for obsessive-compulsiveness. The human patient's behavior and/or psychiatric state may also be measured by the Problem Behaviors Assessment for disoriented behavior.

Further provided is a method of improving or lessening decline of lack of initiative or apathy in a human HD patient comprising periodically orally administering to the patient a pharmaceutical composition comprising pridopidine or pharmaceutically acceptable salt thereof thereof, such that a dose of 90 mg of pridopidine is administered to the patient per day (e.g. 45 mg bid), so as to thereby improve or lessen decline of lack of initiative or apathy in the patient.

The invention also provides a method of reducing or maintaining a human patient's involuntary movements comprising periodically orally administering to the patient a pharmaceutical composition comprising pridopidine such that a dose of 90-225 mg of pridopidine is administered to the patient per day, to thereby reduce or maintain a human patient's involuntary movements.

The invention further provides method of improving or maintaining a human patient's mobility comprising periodically orally administering to the patient a pharmaceutical composition comprising pridopidine such that a dose of 90-225 mg of pridopidine is administered to the patient per day, to thereby improve or maintain the human patient's mobility.

In some embodiments of the methods disclosed above, a dose of 90 mg or 180 mg of pridopidine or pharmaceutically acceptable salt thereof is administered to the patient per day. In some embodiments of the methods disclosed above, a dose of 45 mg of pridopidine is administered to the patient twice a day. In preferred embodiments of the methods disclosed above, the dose of 90 mg of pridopidine is administered to the patient as 45 mg bid per day.

In some embodiments of the methods disclosed above, the administration continues for at least 12 weeks, at least 26 weeks, more than 26 weeks, at least 39 weeks, at least 52 weeks, at least 65 weeks, or at least 78 weeks. In some embodiments of the methods disclosed above, the administration continues for 52 weeks or 65 weeks or 78 weeks. In some embodiments of the methods disclosed above, the HD patient is an early-stage HD patient and has a baseline TFC score of at least 7, at least 8, at least 9, at least 10, at least 11, at least 12, 13, or 7-13, or 7-10 or 11-13. In some embodiments of the methods disclosed above, the HD patient has been diagnosed as having at least 36 CAG repeats in the huntingtin gene. In some embodiments, the HD patient has been diagnosed as having at least 40 repeats in the huntingtin gene. In some embodiments, the HD patient has been diagnosed as having at least 44 repeats in the huntingtin gene. In some embodiments of the methods disclosed above the HD patient is an adult patient and is at least 18 years old or is at least 21 years old. In some embodiments of the methods disclosed above, the HD patient is an early stage HD patient. In some embodiments the patient is a stage 1 HD (HD1, TFC 11-13) patient or stage 2 HD (HD2, TFC 7-10) patient. In some embodiments, the patient is HD1 patient and is experiencing one or more symptom of HD. In some embodiments, the HD patient is not a pre-manifest HD patient. In some embodiments the patient is an Integrated Staging System (ISS-HD) stage 0, ISS-HD stage 1, ISS-HD stage 2, ISS-HD stage 3 or ISS-HD stage 4.

Provided herein is a pharmaceutical composition comprising pridopidine for use in lessening the decline of functional capacity in a human patient wherein the pharmaceutical composition is to be periodically orally administered to the patient such that a dose of 90 mg of pridopidine is to be administered to the patient per day. In some embodiments functional capacity is total functional capacity. In some embodiments the daily dose is 45 mg bid.

Provided herein is a pharmaceutical composition comprising pridopidine for use in maintaining functional capacity in a human patient wherein the pharmaceutical composition is to be periodically orally administered to the patient such that a dose of 90 mg of pridopidine is to be administered to the patient per day (e.g. 45 mg bid). In some embodiments functional capacity includes activities of daily living (ADL).

Provided herein is use of an amount of pridopidine in the manufacture of a medicament maintaining functional capacity in a human patient wherein the medicament is formulated for periodic oral administration to the patient such that a dose of 90 mg of pridopidine is to be administered to the patient per day (e.g. 45 mg bid). In some embodiments functional capacity includes ADL.

Provided herein is a pharmaceutical composition comprising pridopidine for use in slowing the clinical progression of HD as measured by total functional capacity in a human patient wherein the pharmaceutical composition is to be periodically orally administered to the patient such that a dose of 90 mg of pridopidine is to be administered to the patient per day (e.g. 45 mg bid). In many embodiments, (a) the pharmaceutical composition is administered for more than 26 weeks or (b) the human patient is afflicted with early stage HD.

In some embodiments of the pharmaceutical compositions and uses, TFC includes one or more of maintaining occupation, taking care of finances, domestic chores, requiring low level of care and activities of daily living (ADL).

Provided herein is a use of an amount of pridopidine in the manufacture of a medicament for slowing the clinical progression of HD as measured by total functional capacity in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in maintaining a human patient's ability to perform activities of daily living in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in maintaining a human patient's ability to perform activities of daily living in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining gait and balance in a human patient wherein the pharmaceutical composition is to be periodically orally administered to the patient such that a dose of 90-225 mg of pridopidine is to be administered to the patient per day. In some embodiments the administration slows the decline of a patient's gait and balance.

Provided herein is use of an amount of pridopidine in the manufacture of a medicament for use in improving or maintaining, a human patient's gait and balance in a human patient wherein the medicament is formulated for periodic oral administration to the patient such that a dose of 90-225 mg of pridopidine is to be administered to the patient per day. In some embodiments the administration slows the decline of a patient's gait and balance.

Provided herein is a pharmaceutical composition comprising pridopidine for use in improving, maintaining, or slowing the decline of gait and balance in a human patient wherein the pharmaceutical composition is to be periodically orally administered to the patient such that a dose of 90 mg of pridopidine is to be administered to the patient per day (e.g. 45 mg bid). In some embodiments the administration slows the decline of a patient's gait and balance.

Provided herein is use of an amount of pridopidine in the manufacture of a medicament for use in improving, maintaining, or slowing the decline of, a human patient's gait and balance in a human patient wherein the medicament is formulated for periodic oral administration to the patient such that a dose of 90 mg of pridopidine is to be administered to the patient per day (e.g. 45 mg bid). In some embodiments the administration slows the decline of a patient's gait and balance.

Provided herein is a pharmaceutical composition comprising pridopidine for use in improving or maintaining independence in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining, a human patient's independence wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining or slowing the decline of a human patient's independence wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining, or slowing the decline of a human patient's independence wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining a human patient's cognitive domains wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining a human patient's cognitive domains wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining or slowing the decline of a human patient's cognitive domains wherein the pharmaceutical composition is to be periodically orally administered to the patient such that a dose of 90 mg of pridopidine is to be administered to the patient per day.

Provided herein is use of an amount of pridopidine in the manufacture of a medicament for use in improving or maintaining or slowing the decline of a human patient's cognitive domains wherein the medicament is formulated for periodic oral administration to the patient such that a dose of 90 mg of pridopidine is to be administered to the patient per day.

Provided herein is a pharmaceutical composition comprising pridopidine for use in reducing the severity of the sustained or intermittent muscle contractions associated with dystonia in a human patient wherein the pharmaceutical composition is to be periodically orally administered to the patient such that a dose of 90 mg of pridopidine is to be administered to the patient per day.

Provided herein is use of an amount of pridopidine in the manufacture of a medicament for use in reducing the severity of the sustained or intermittent muscle contractions associated with dystonia in a human patient wherein the medicament is formulated for periodic oral administration to the patient such that a dose of 90 mg of pridopidine is to be administered to the patient per day.

Provided herein is a pharmaceutical composition comprising pridopidine for use in improving or maintaining motor ability in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining motor ability in a human patient wherein the medicament is formulated for periodic oral administration to the patient such that a dose of 90 mg of pridopidine is to be administered to the patient per day (e.g. 45 mg bid).

Provided herein is use of an amount of pridopidine in the manufacture of a medicament for use in reducing or maintaining the level of chorea in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in reducing or maintaining or slowing the increase of chorea in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in reducing or maintaining or slowing the increase of chorea in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining a human patient's behavior and/or psychiatric state wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining a human patient's behavior and/or psychiatric state wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in reducing or maintaining a human patient's involuntary movements wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in reducing or maintaining a human patient's involuntary movements wherein the medicament is formulated for periodic oral administration to the patient such that a dose of 90-225 mg of pridopidine is to be administered to the patient per day.

Provided herein is a pharmaceutical composition comprising pridopidine for use in improving or maintaining a human patient's mobility wherein the pharmaceutical composition is to be periodically orally administered to the patient such that a dose of 90-225 mg of pridopidine is to be administered to the patient per day.

Provided herein is use of an amount of pridopidine in the manufacture of a medicament for use in improving or maintaining a human patient's mobility wherein the medicament is formulated for periodic oral administration to the patient such that a dose of 90-225 mg of pridopidine is to be administered to the patient per day.

Provided herein is a pharmaceutical composition comprising pridopidine for use in improving or maintaining a human patient's ability to perform physical tasks wherein the pharmaceutical composition is to be periodically orally administered to the patient such that a dose of 90 mg of pridopidine is to be administered to the patient per day (e.g. 45 mg bid).

Provided herein is a use of an amount of pridopidine in the manufacture of a medicament for use in improving or maintaining a human patient's ability to perform physical tasks wherein the medicament is formulated for periodic oral administration to the patient such that a dose of 90 mg of pridopidine is to be administered to the patient per day (e.g. 45 mg bid).

The methods, compositions and uses disclosed herein are applicable, for example, to a human patient afflicted with HD. In some embodiments of the methods compositions and uses, the human patient is afflicted with HD and has a baseline TMS score which is in the least severe quarter of the overall population of patients afflicted with Huntington disease; or the human patient is afflicted with HD and has a baseline TMS score which is in the two least severe quarters of the overall population of patients afflicted with Huntington disease; or the human patient is afflicted with HD and has a baseline TMS score which is in the three least severe quarters of the overall population of patients afflicted with Huntington disease; or the human patient is afflicted with HD and has a baseline TMS score which is in the three least severe quarters of the overall population of patients afflicted with HD or a baseline TFC score which is greater than or equal to 9; or the human patient is afflicted with HD and has a baseline TMS score which is in the three least severe quarters of the overall population of patients afflicted with HD or a baseline TFC score which is greater than or equal to 9 or less than 44 CAG repeats in the Huntingtin gene; or the human patient is afflicted with HD and has a baseline TMS score which is in the two least severe quarters of the overall population of patients afflicted with HD; or the human patient is afflicted with HD and has a baseline TFC score which is greater than or equal to 7; or the human patient is afflicted with HD and has a baseline TFC score of 11-13; or the human patient is afflicted with HD and has a baseline TFC score which is greater than or equal to 9 or greater than 44 CAG repeats in the huntingtin gene; or the human patient is afflicted with HD and has a baseline TMS score which is in the three least severe quarters of the overall population of patients afflicted with HD or less than 44 CAG repeats in the huntingtin gene; or the human patient is afflicted with HD and has a baseline TFC score which is greater than or equal to 9 or a baseline TMS score which is in the three least severe quarters of the overall population of patients afflicted with HD.

In some embodiments of the methods, compositions and uses disclosed herein the pridopidine or a pharmaceutically acceptable salt thereof is pridopidine hydrochloride.

A pharmaceutical composition comprising pridopidine or a pharmaceutically acceptable salt thereof, for example pridopidine hydrochloride, is to be periodically orally administered to the patient such that a dose of 90 mg of pridopidine is to be administered to the patient per day (e.g. 45 mg bid).

In some embodiments of the compositions and uses disclosed above, a dose of 90 mg of pridopidine is to be administered to the patient per day. In some embodiments of the methods disclosed above, a dose of 90 mg of pridopidine is to be administered to the patient per day. In preferred embodiments of the methods disclosed above, the dose of 90 mg of pridopidine to be administered to the patient per day is to be administered to the patient as 45 mg bid.

In some embodiments, the patient is to be administered 45 mg pridopidine once daily (qd) for about one to two weeks and 45 mg pridopidine bid thereafter. In some embodiments of the methods disclosed above, the administration continues for at least 12 weeks, at least 26 weeks, at least 39 weeks, at least 52 weeks, at least 65 weeks, or at least 78 weeks. In some embodiments of the methods disclosed above, the administration continues for 52 weeks or 78 weeks. In some embodiments of the methods disclosed above, the HD patient is a stage 1 or stage 2 HD patient and has a baseline TFC score of at least 7, at least 8, at least 9, at least 10, at least 11, at least 12, 13, or 7-10 or 11-13. In some embodiments of the methods disclosed above, the HD patient has been diagnosed as having at least 36 CAG repeats in the huntingtin gene. In some embodiments of the methods disclosed above, the HD patient has been diagnosed as having at least 40 CAG repeats in the huntingtin gene. In some embodiments of the methods disclosed above, the HD patient has been diagnosed as having at least 44 CAG repeats in the huntingtin gene. In some embodiments of the methods disclosed above the HD patient is 21 years old or older.

In some embodiments of the methods, compositions and uses disclosed above, the HD patient is a HD1 or HD2 patient and is not a pre-manifest HD patient.

### BRIEF DESCRIPTION OF THE SEVERAL VIEWS OF THE DRAWINGS

In the following brief descriptions of the figures and the corresponding figures, efficacy was assessed at different time points in different studies. For example, throughout the 78-week period using the Mixed Models Repeated Measures (MMRM) analyses of change from baseline (prior to administration of pridopidine at week 0) were assessed. These included the evaluation of UHDRS-TMS, UHDRS-Behavioral, UHDRS-Cognitive, UHDRS-TFC, UHDRS-Functional Assessment, UHDRS-Independence Scale, individual TMS subscales, HD-Cognitive Assessment Battery (HD-CAB), Problem Behavior Assessment Short-Form (PBA-s), HD-QoL, Q-Motor and other outcomes. Other time points for assessments as described and exemplified herein include 26 weeks, 39 weeks, 52 weeks, 65 weeks, 78 weeks, 24 months, 36 months, 48 months, and 60 months. Assessments also included TFC, cUHDRS, HD-Quality of Life scale (HD-QoL), stroop word test (SWR), symbol digit modality test (SDMT) and motor skills, bradykinesia, and gross motor coordination skills, including speech difficulties, gait, and postural dysfunction.

FIG. 1: Pridopidine concentration in patients' blood (ng/mL; Mean (+/−sd) measured values). “Pre” means pre-dose and “post” mean post dose. V2 means visit 2, V3 means visit 3, etc. Wk2 means second week, Wk3 means third week, etc.

FIG. 2: Pridopidine concentration in patients' blood (ng/mL). Post-dose (“Cmax”) (+/−sd) at Steady State.

FIG. 3: Total Motor Score (TMS) Change from Baseline (BL) with pridopidine administration. All doses of pridopidine demonstrated an improvement in TMS from baseline. A decrease in TMS indicates an improvement. Table 1 below shows the P-Values corresponding to FIG. 3.

TABLE 1
p-values for TMS change from baseline
per treatment group per visit

| Week | 45 mg bid | 67.5 mg bid | 90 mg bid | 112.5 mg bid |
|---|---|---|---|---|
| 4 | 0.0304 | 0.0004 | <.0001 | <.0001 |
| 8 | <.0001 | <.0001 | <.0001 | <.0001 |
| 12 | 0.0002 | 0.0003 | <.0001 | 0.0002 |
| 16 | <.0001 | <.0001 | <.0001 | <.0001 |
| 20 | <.0001 | <.0001 | <.0001 | <.0001 |
| 26 | 0.0013 | 0.0024 | <.0001 | 0.0063 |

FIG. 4: Total Motor Score (TMS)— Change from Baseline PRIDE-HD placebo vs historical placebo in HART and MermaiHD clinical trials. A lower number indicates improvement. At week 12 placebo effect was observed in HART MermaiHD and PRIDE-HD (i.e placebo group showed improvement). At 26 weeks there was no placebo effect in MermaiHD (positive change=worsening). However, in PRIDE-HD placebo effect was maintained (negative change=improvement) at week 26. There is about a 6.5 TMS point difference at week 26 between the placebo group in MermaiHD and placebo group in PRIDE-HD (indicating improvement in PRIDE).

FIGS. 5A and 5B: Change from baseline in TMS. FIG. 5A: Using historical placebo in HART and MermaiHD clinical trials, TMS (change from baseline) results are significant for both pridopidine 45 mg bid and 90 mg bid. A lower number indicates improvement. FIG. 5B: Change from baseline UHDRS-TMS full analysis set plotted over time. PRIDE-HD replicates previous data in TMS changes from baseline as the change from baseline values were similar to those in HART and MermaiHD. In this graph, a decrease in TMS change from baseline indicates improvement. Dark line with diamonds represents placebo, line with open circles represents 45 mg bid, gray line with triangles represents 67.5 mg bid, gray line with diamonds represents 90 mg bid, line with squares represents 112.5 mg bid. The 90 mg bid dose demonstrated the largest treatment effect.

FIG. 6A. Comparison of patients with baseline (BL) dystonia score of ≥4 at 52 weeks after dosage with either placebo or 45 mg pridopidine bid. Within the full analysis set, no clinically meaningful changes from baseline were noted for patients at Week 26 or Week 52 in the dystonia score across the placebo and active treatment group (not shown). In patients with a baseline total dystonia score greater than or equal to 4 assessed at Week 52, a directional clinical improvement in dystonia was noted for the 45 mg bid treatment group (change vs placebo is −1.54, p=0.0571).

Table 2 below shows change from baseline in UHDRS dystonia score over time for pridopidine 45 mg bid in the integrated data set from HART MermaiHD and PRIDE-HD.

TABLE 2
Change from baseline in UHDRS dystonia score over time in
an integrated dataset from HART, MermaiHD and PRIDE

| Week | 12 | 26 | 52 |
|---|---|---|---|
| Placebo n = | 111 | 83 | 33 |
| Pridopidine | 102 | 81 | 21 |
| 45 mg bid n= | | | |
| Δ to placebo | −0.96 | −1.01 | −1.54 |
| p value | 0.0232 | 0.0326 | 0.0571 |

Week 12 is integrated data from HART, MermaiHD and PRIDE-HD, Week 26 in integrated data from HART, MermaiHD and PRIDE-HD (because HART was a 12-week study) and week 52 is PRIDE-HD data (MermaiHD was a 26-week study)

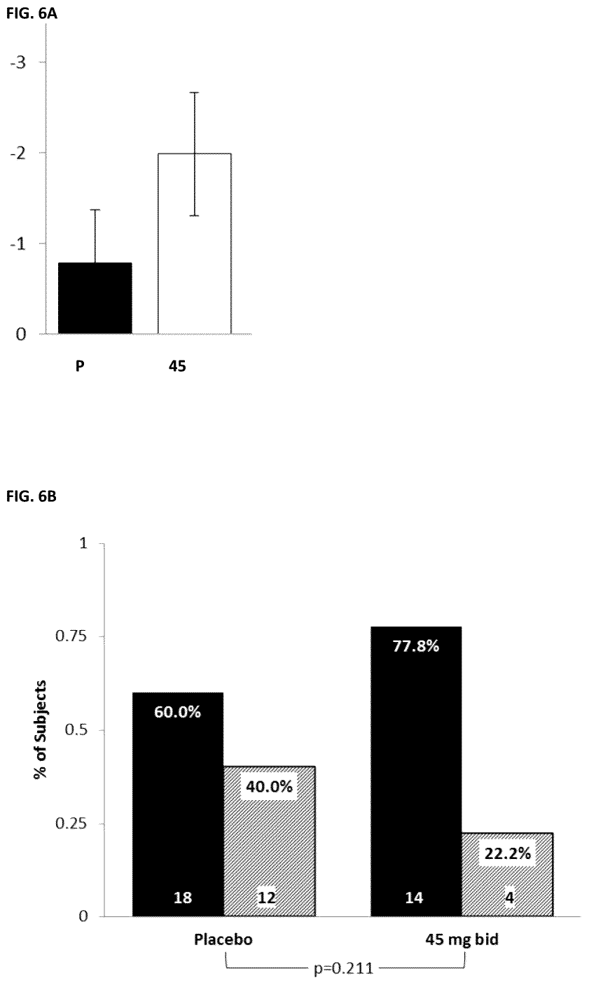

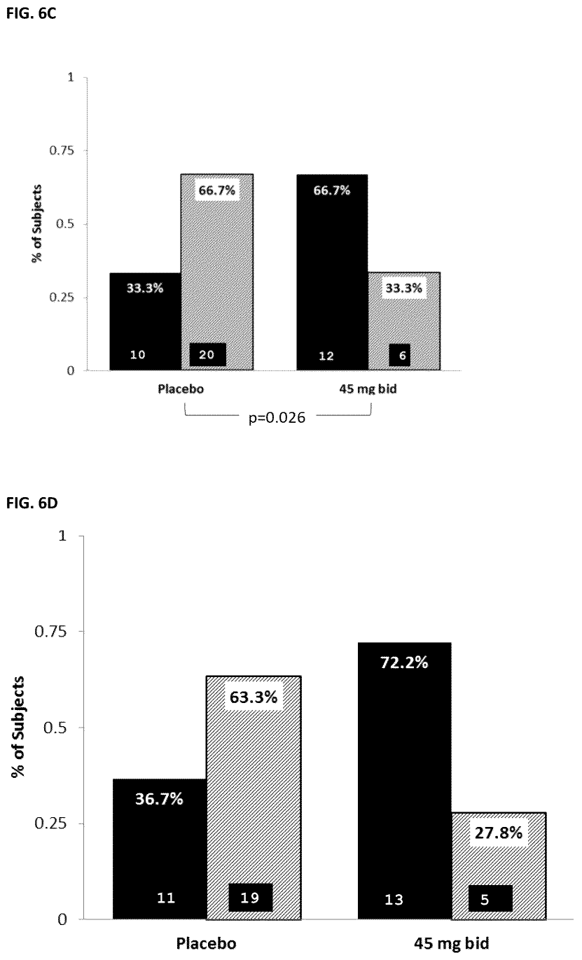

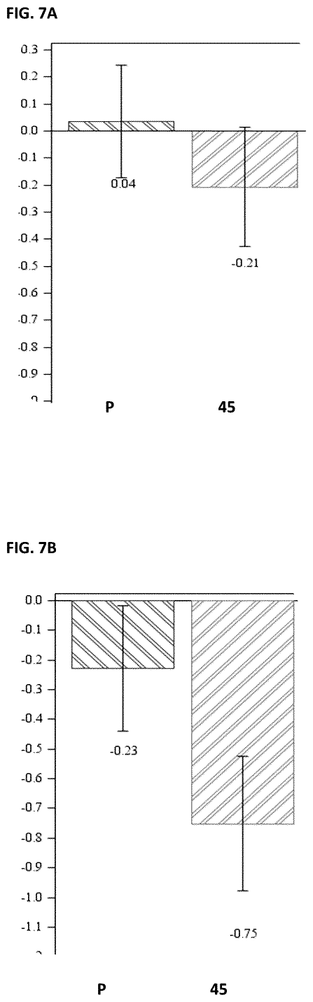

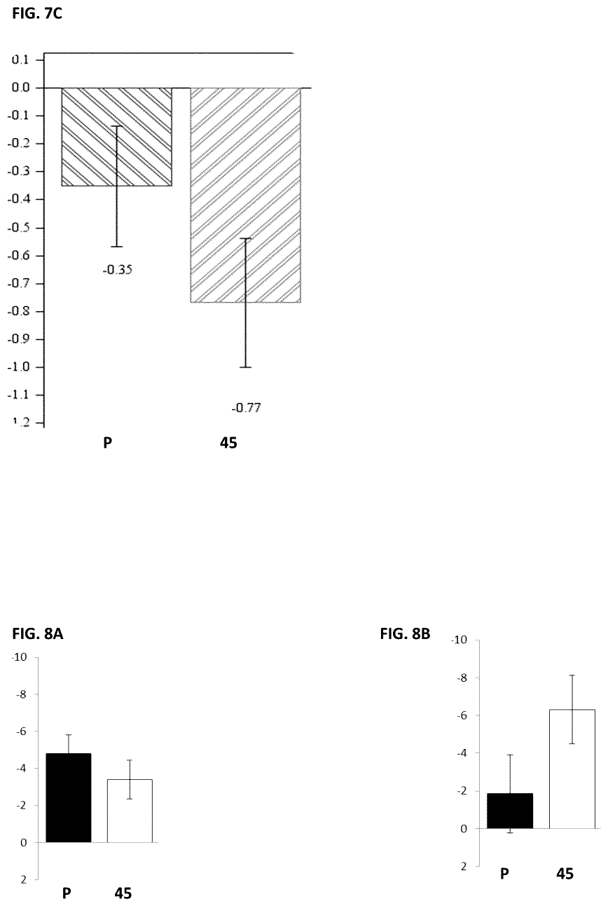

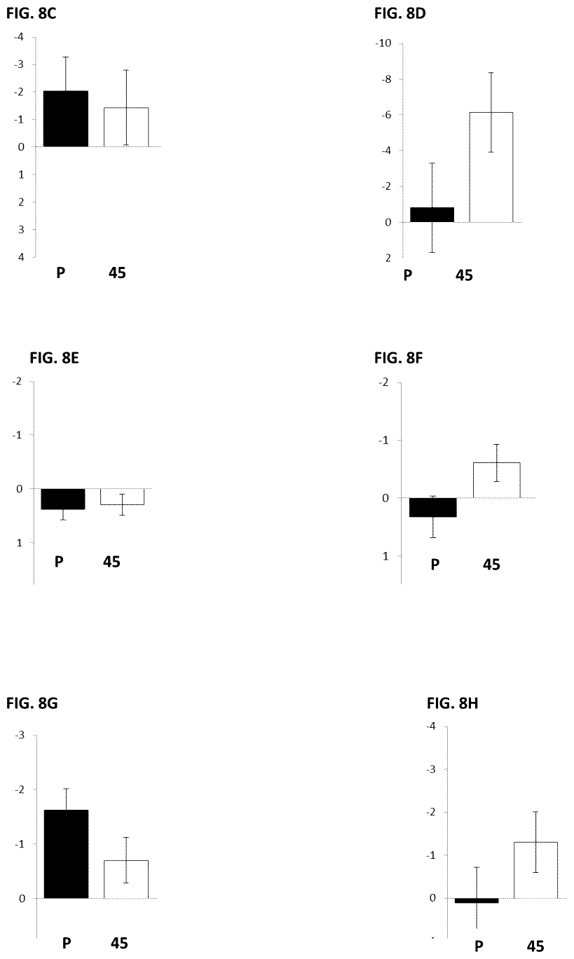

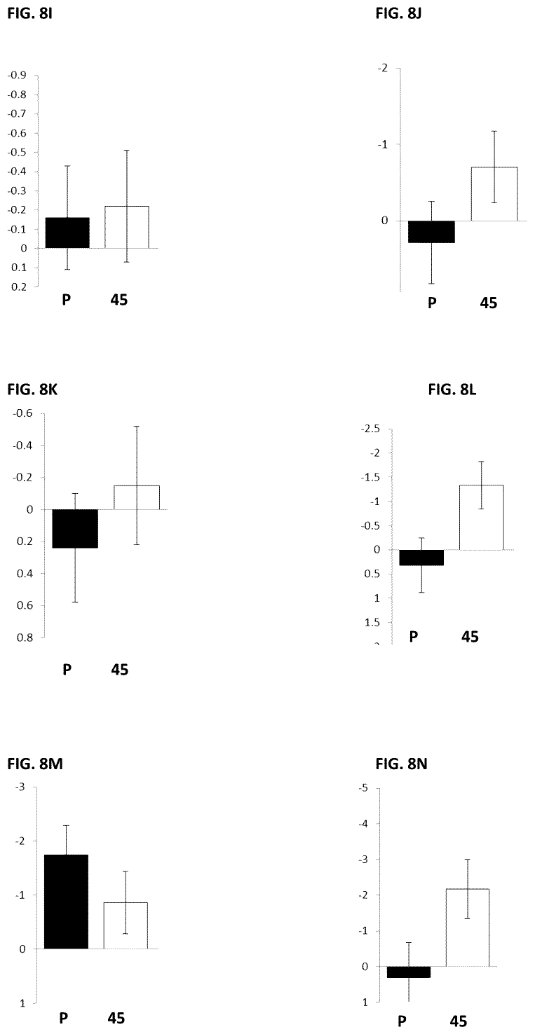

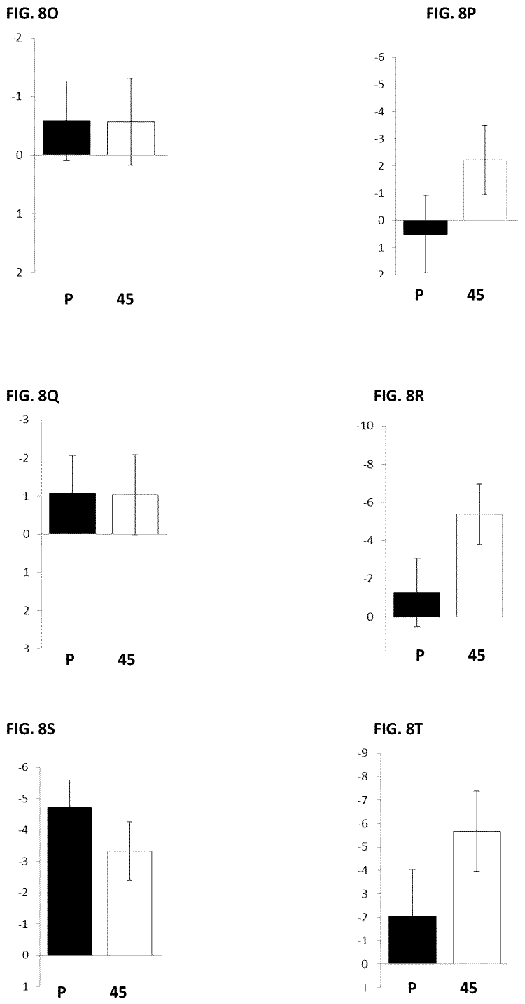

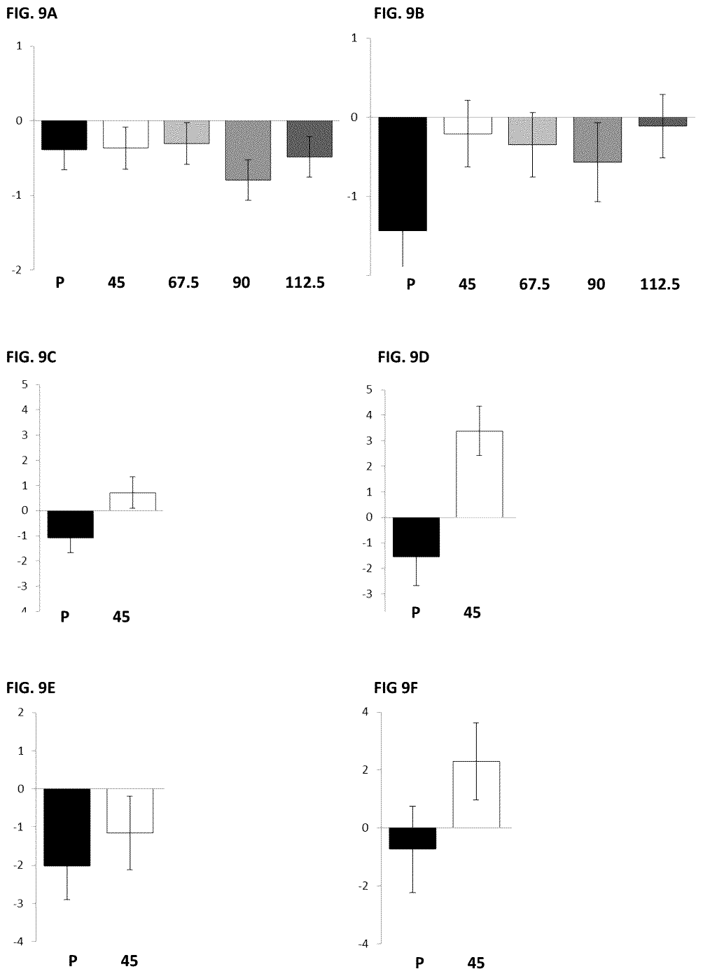

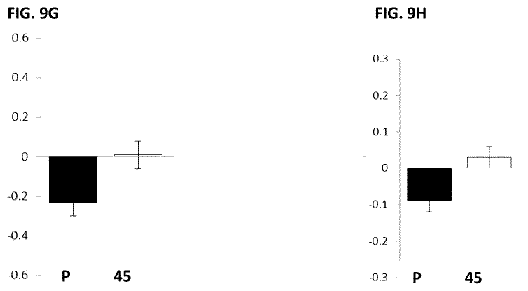

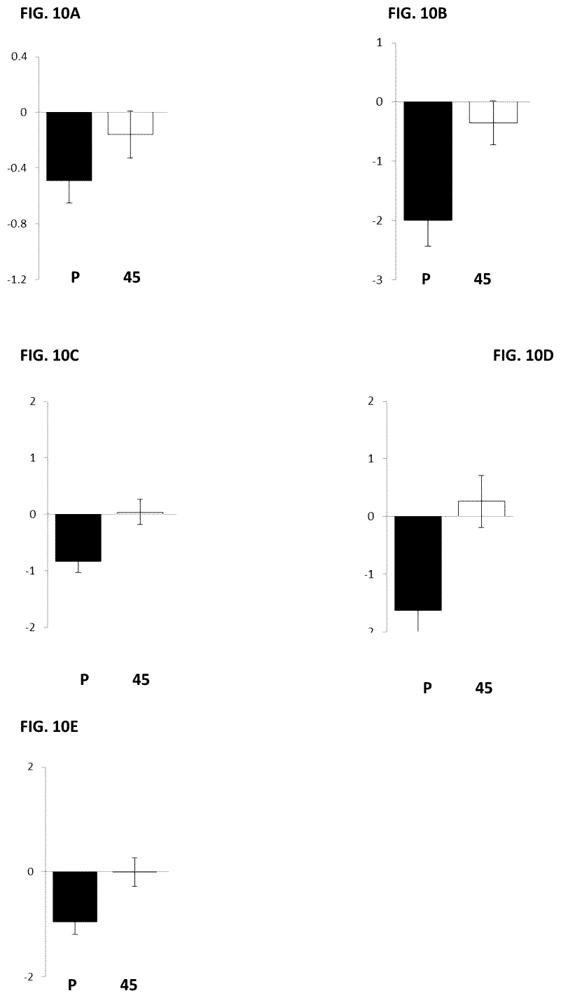

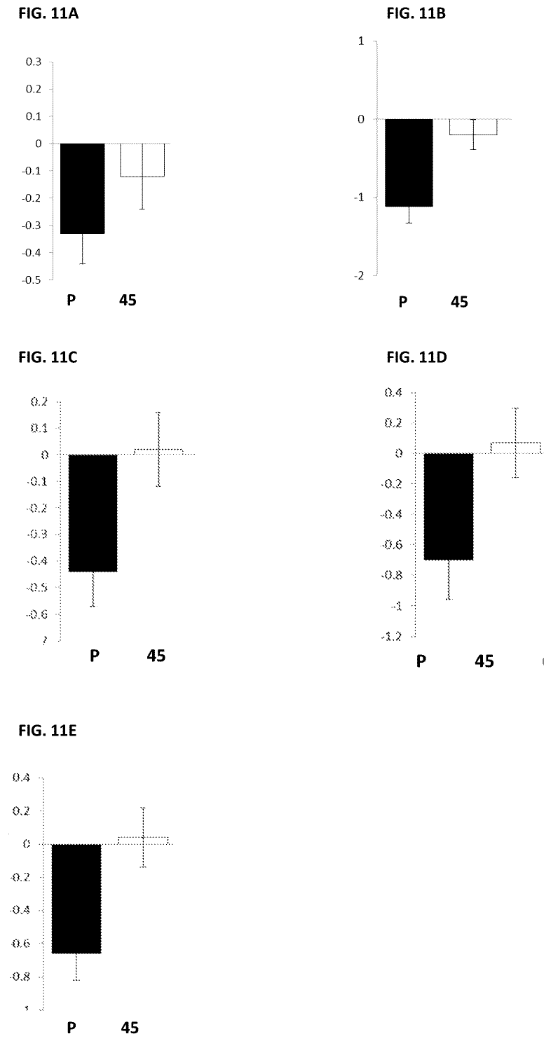

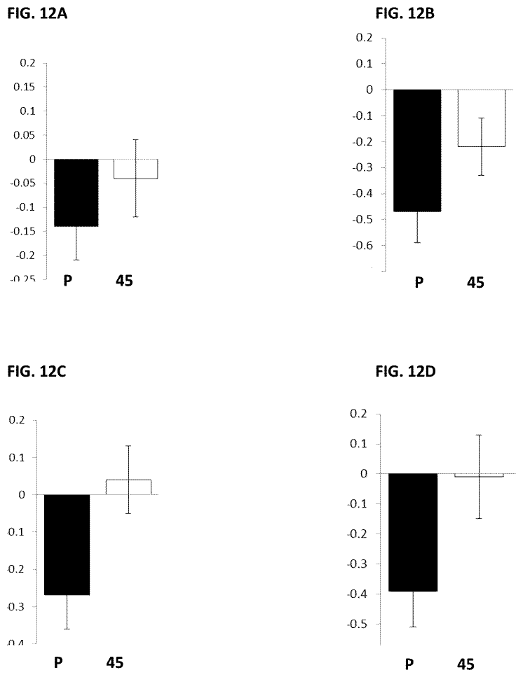

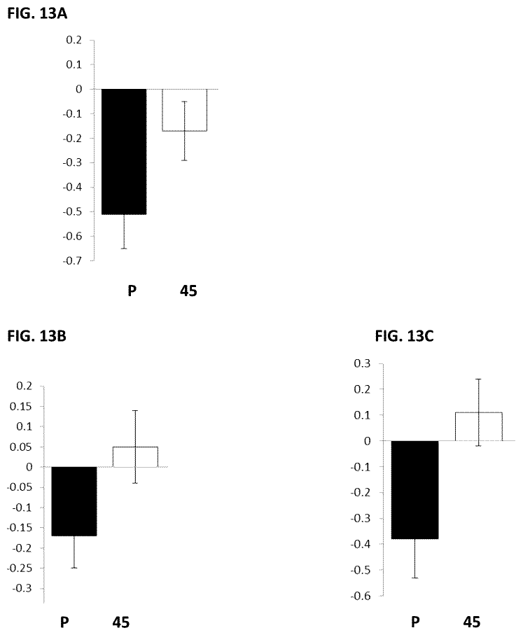

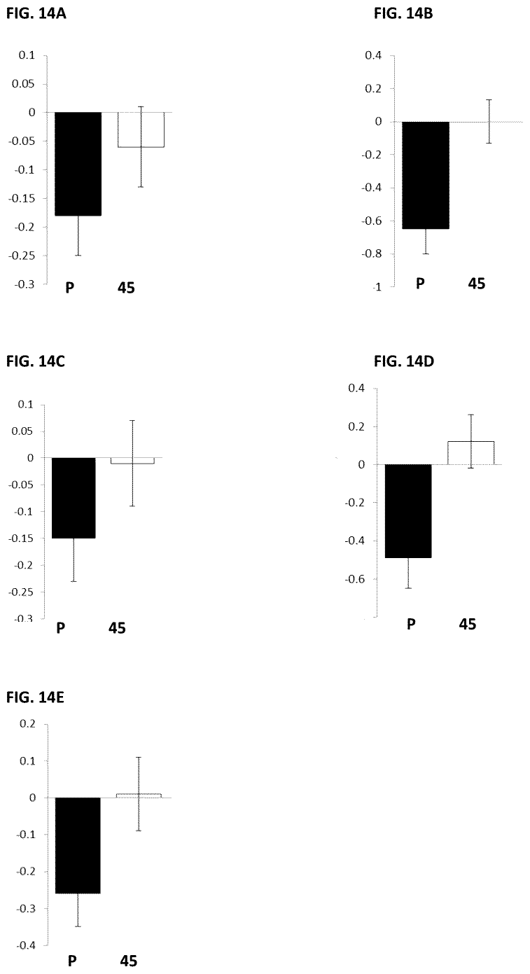

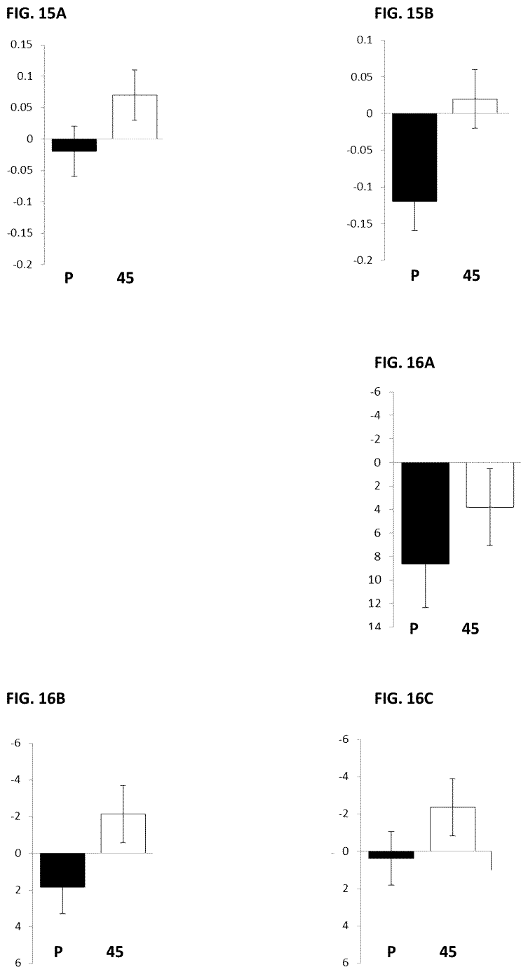

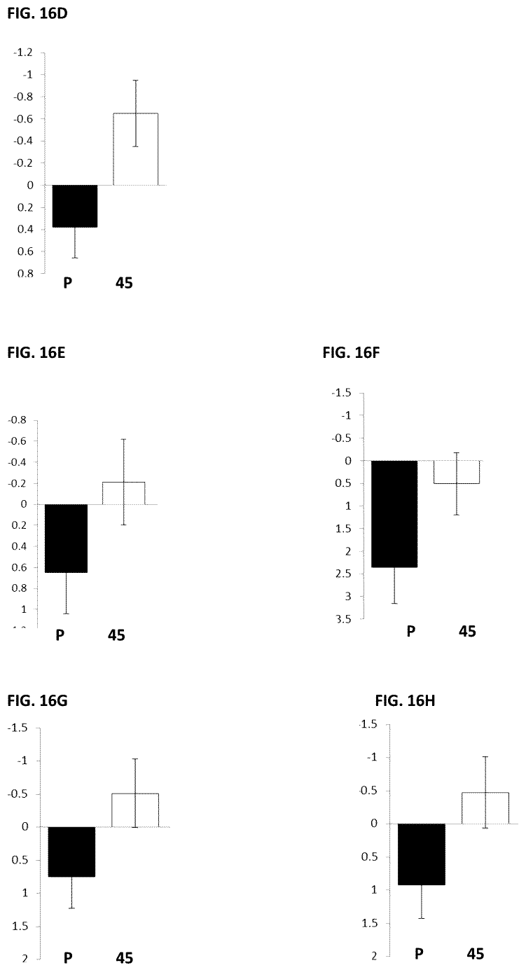

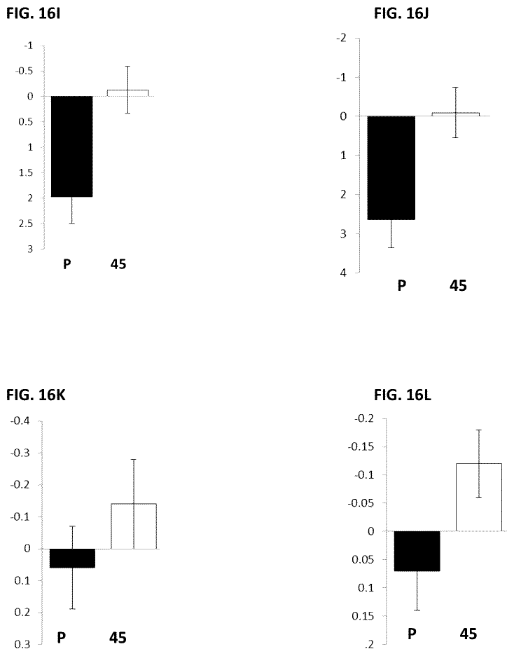

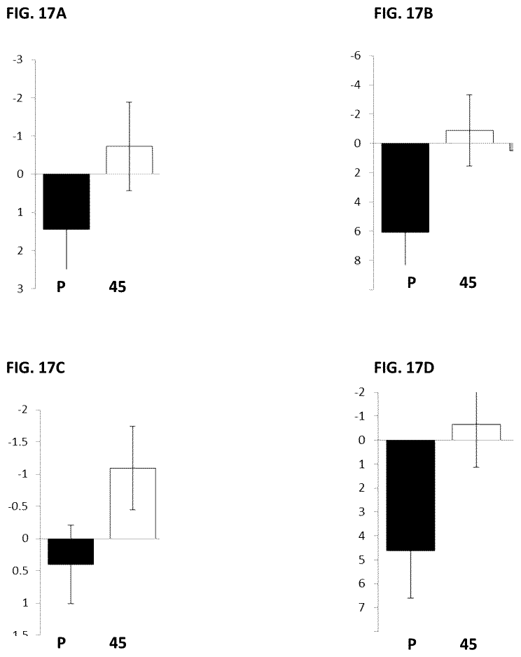

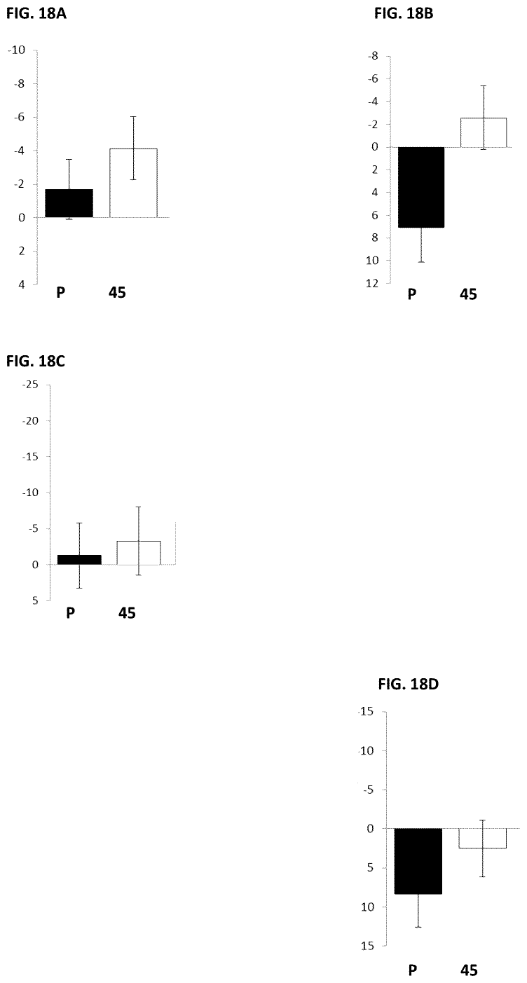

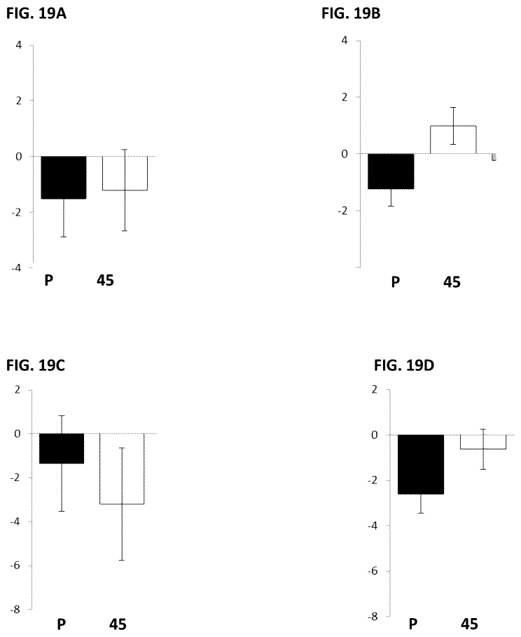

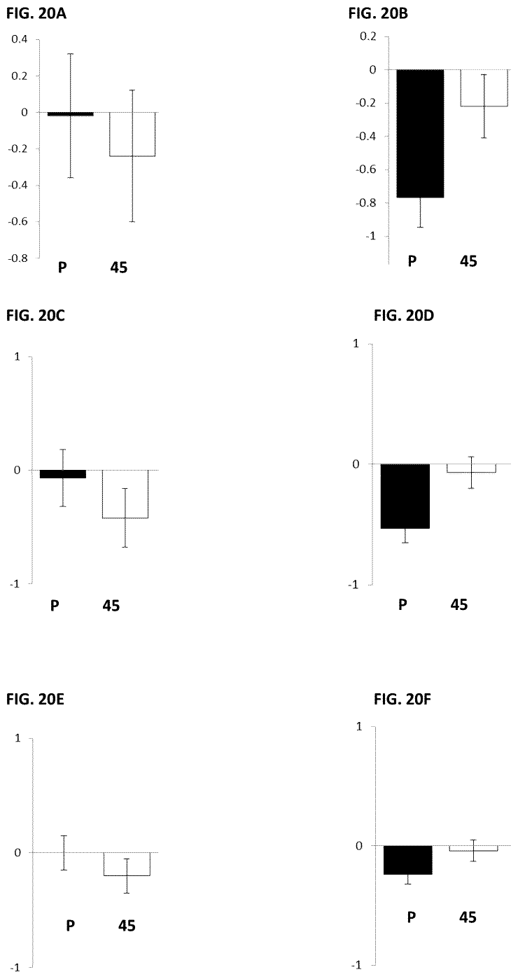

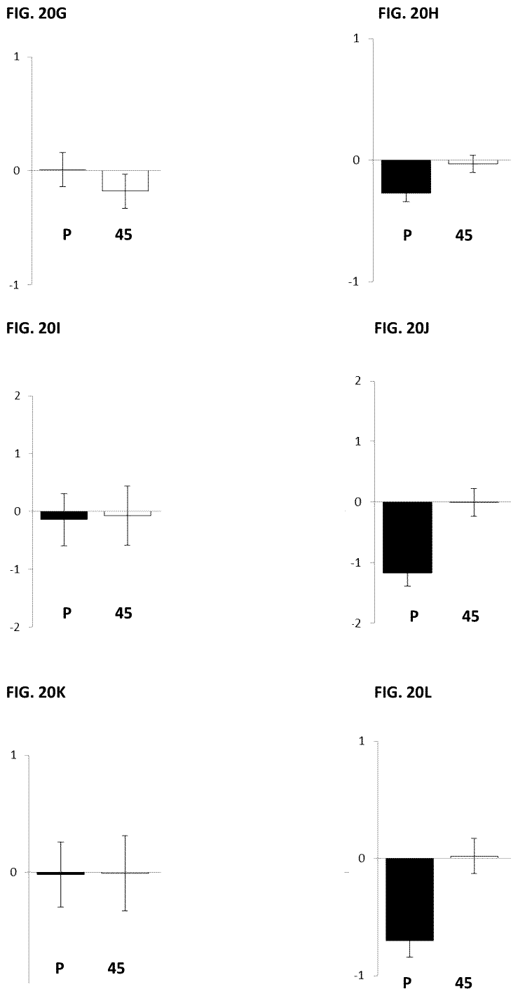

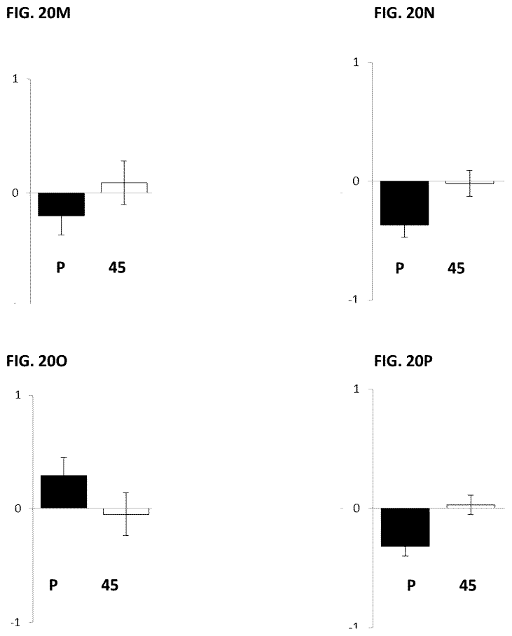

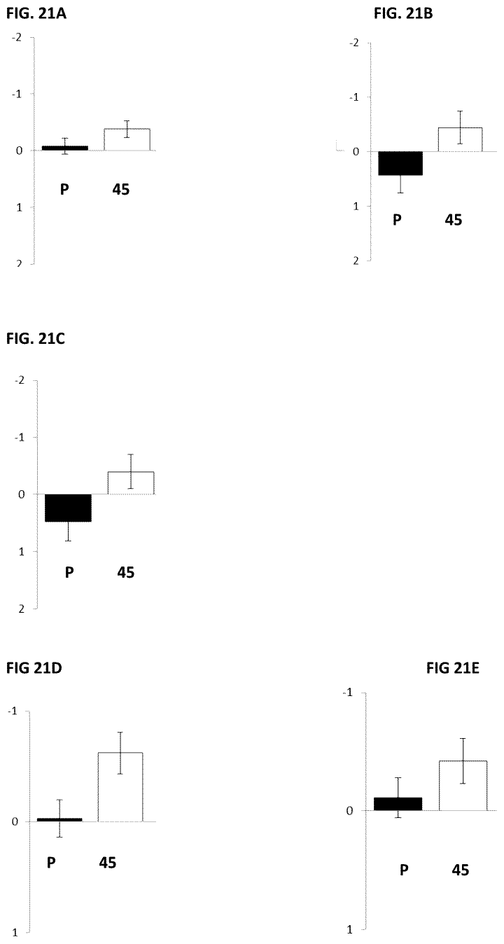

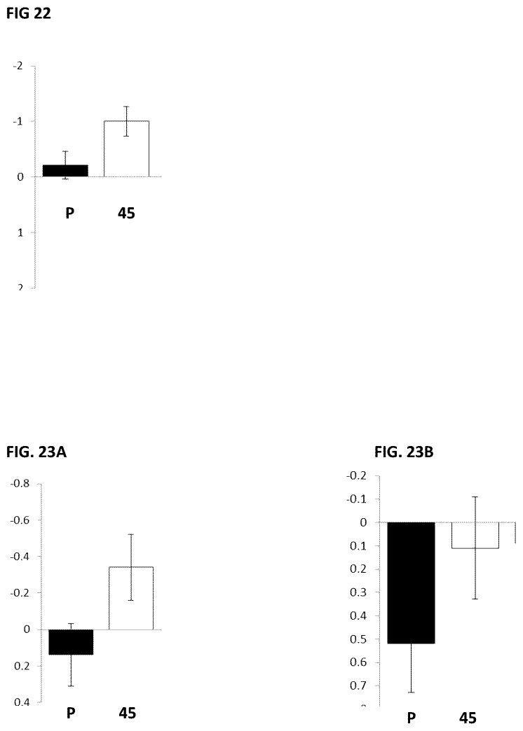

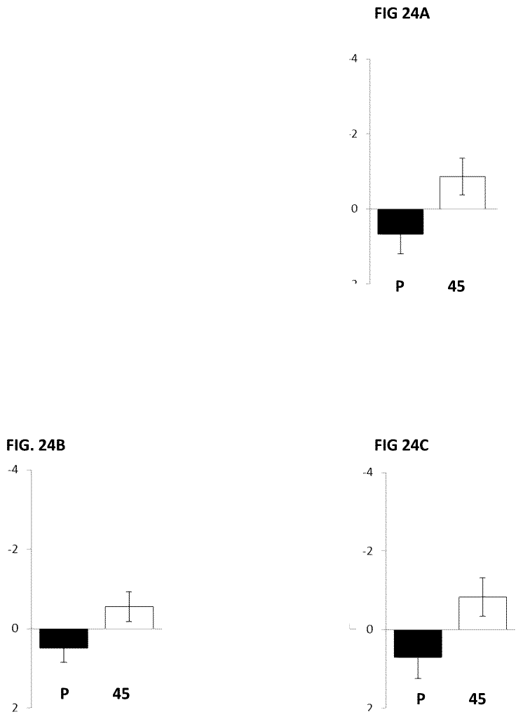

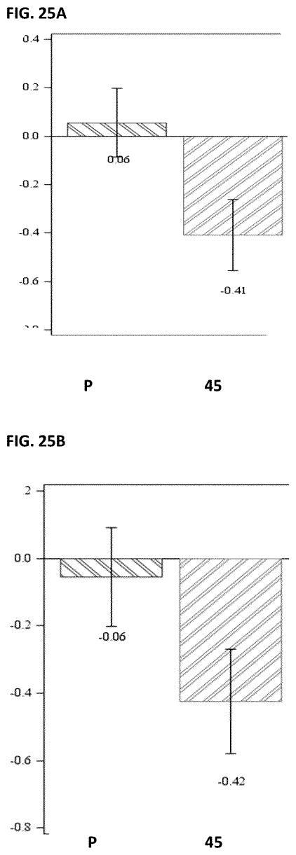

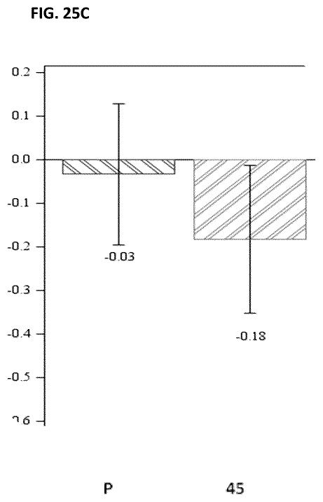

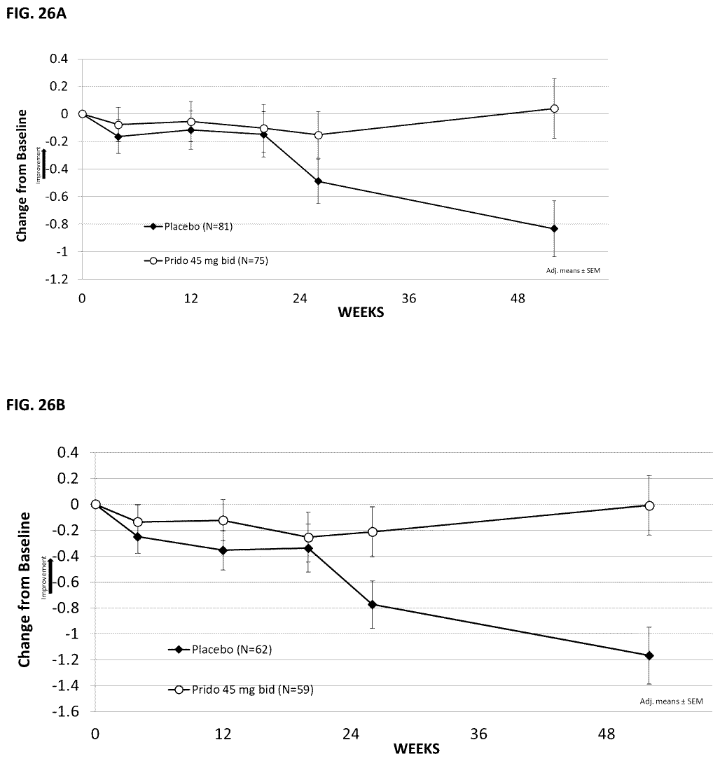

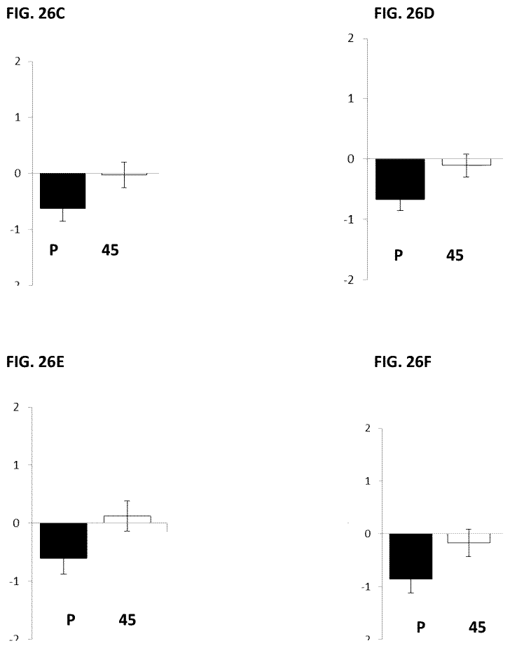

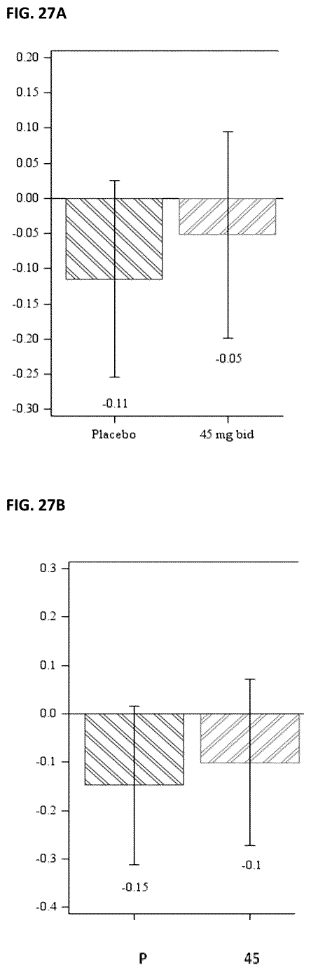

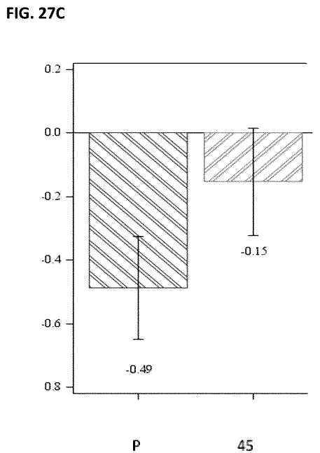

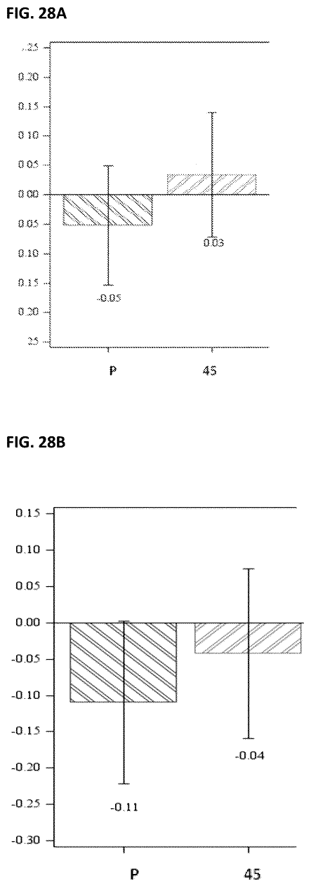

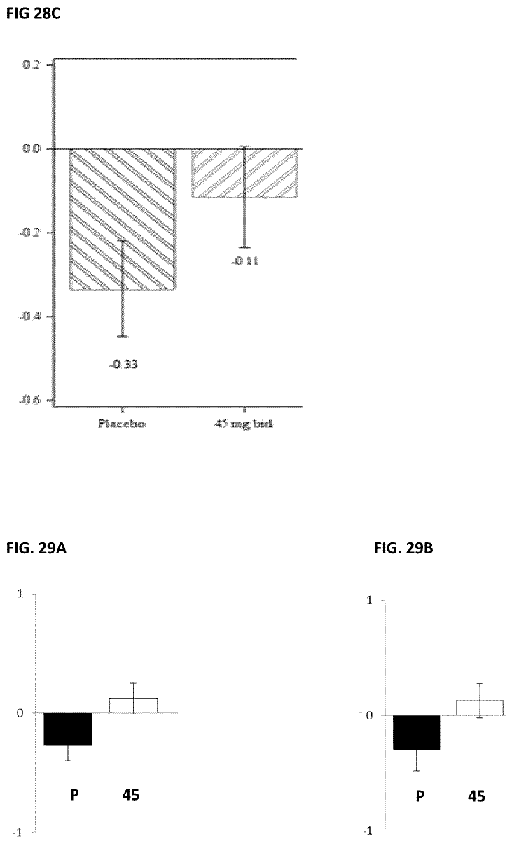

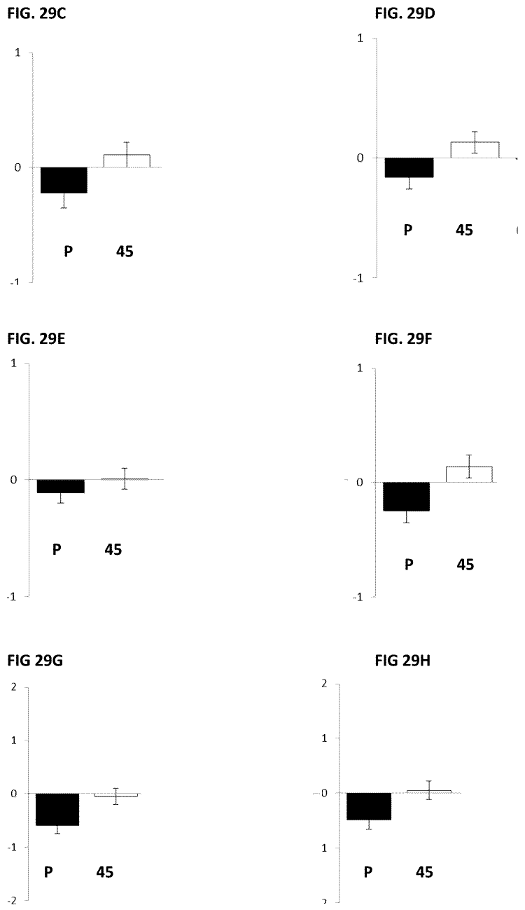

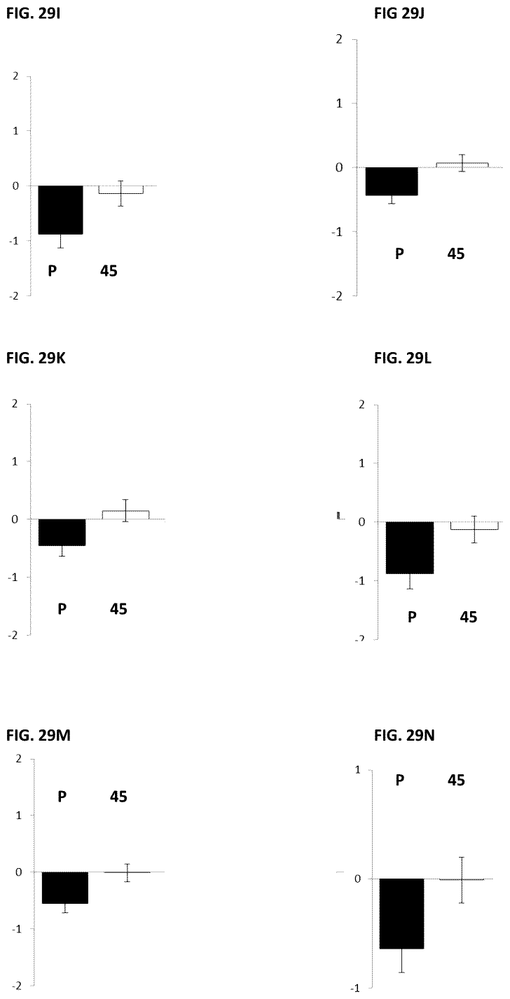

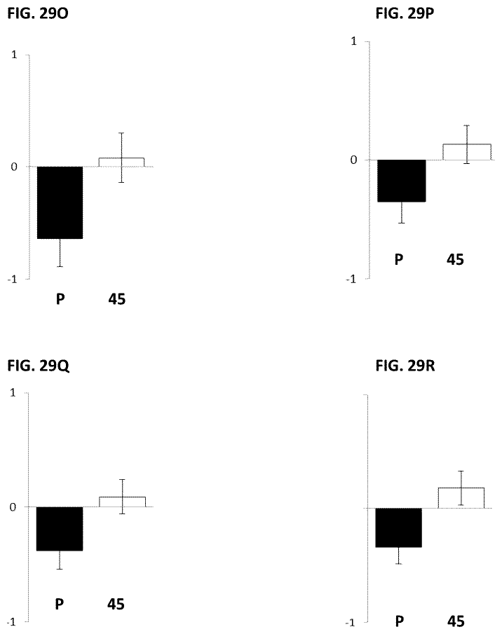

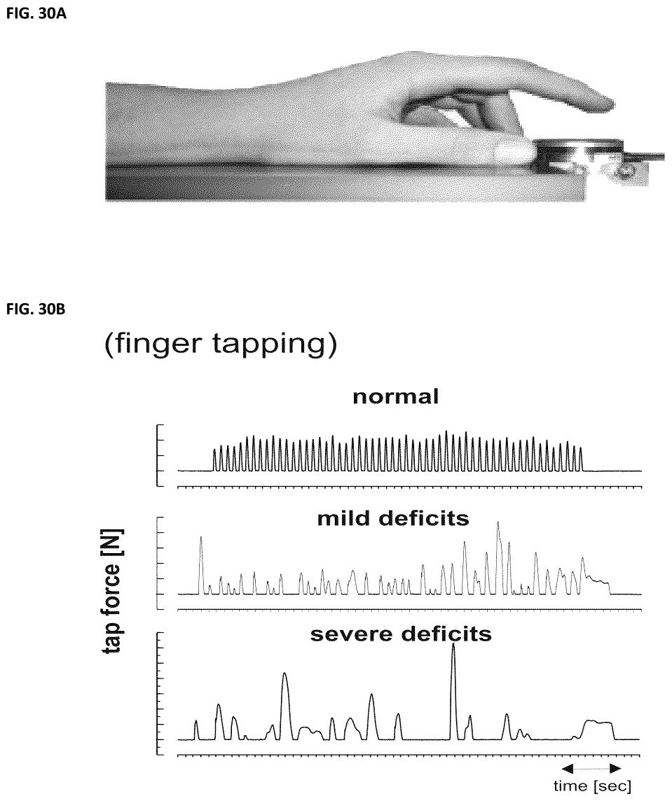

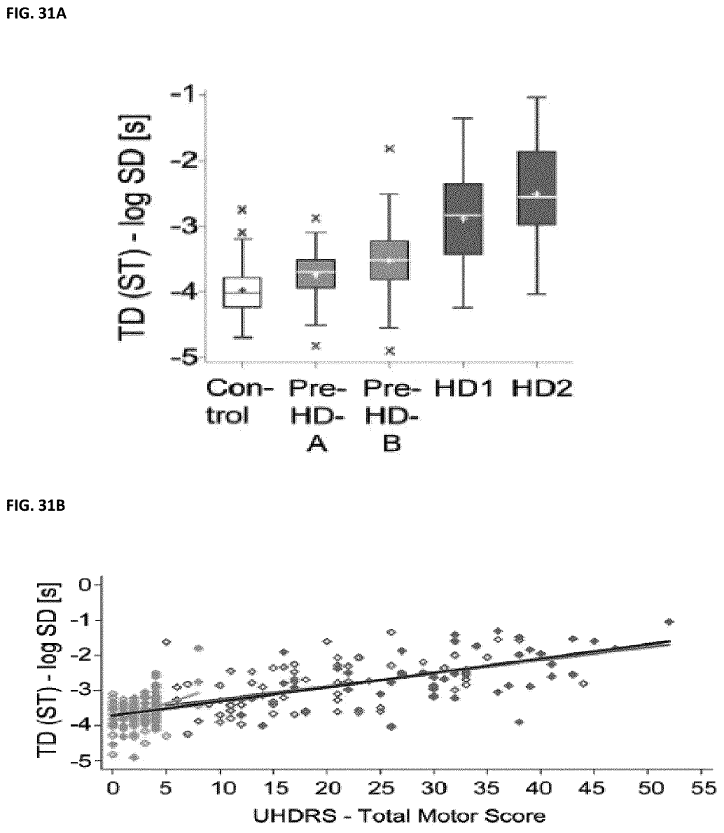

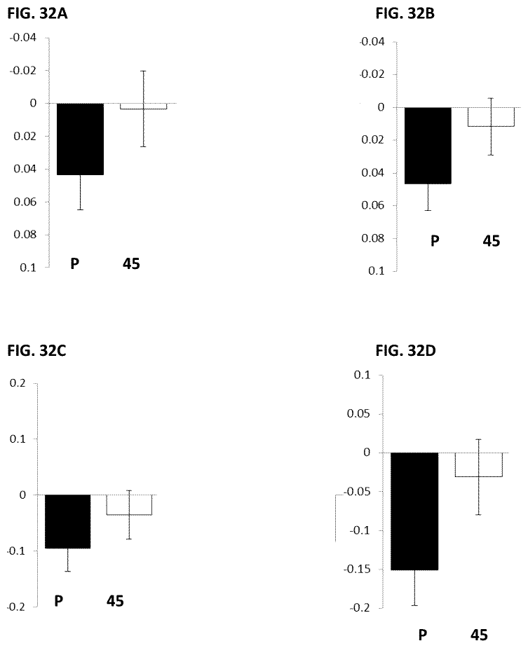

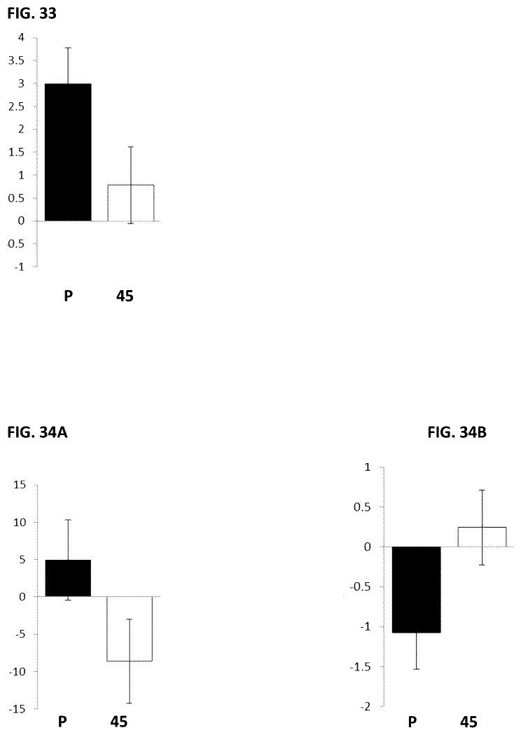

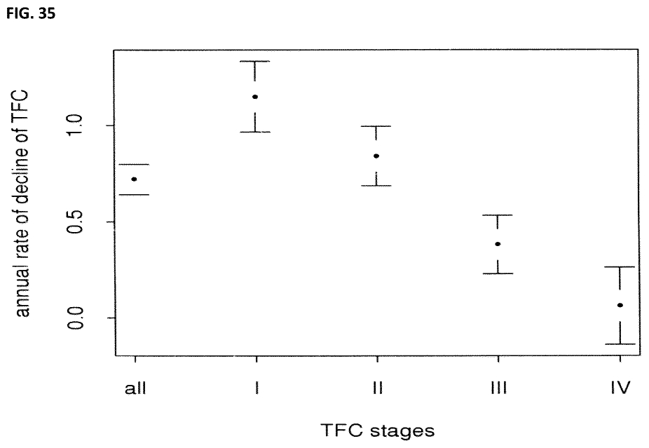

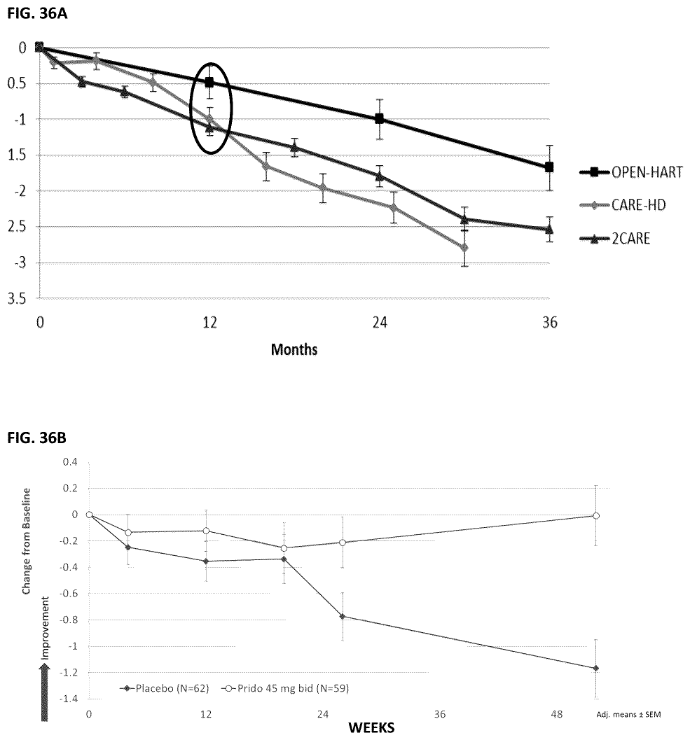

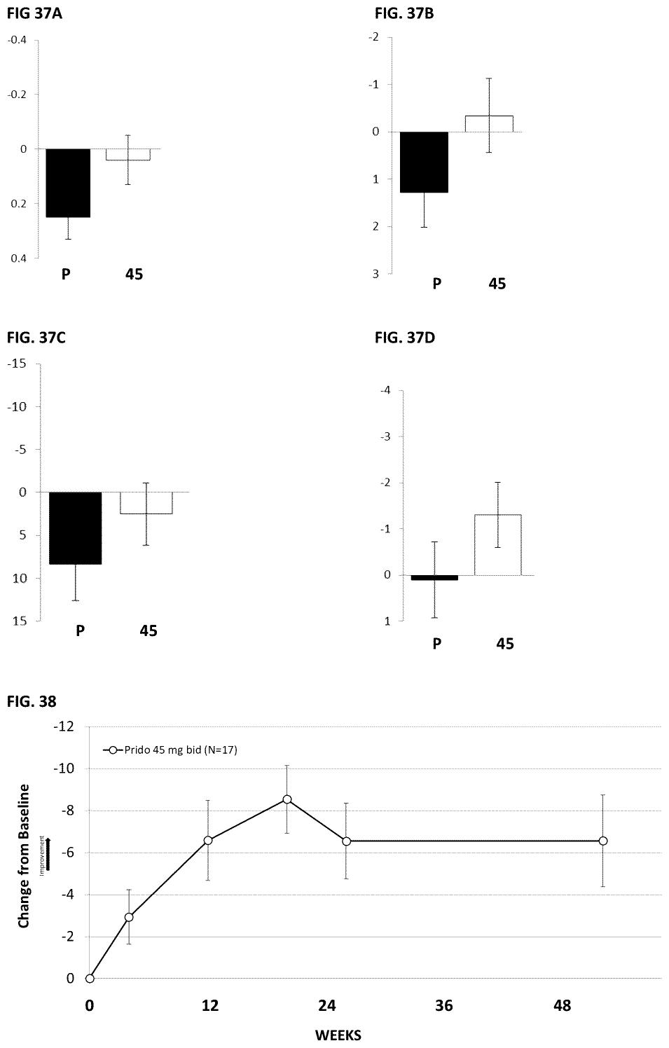

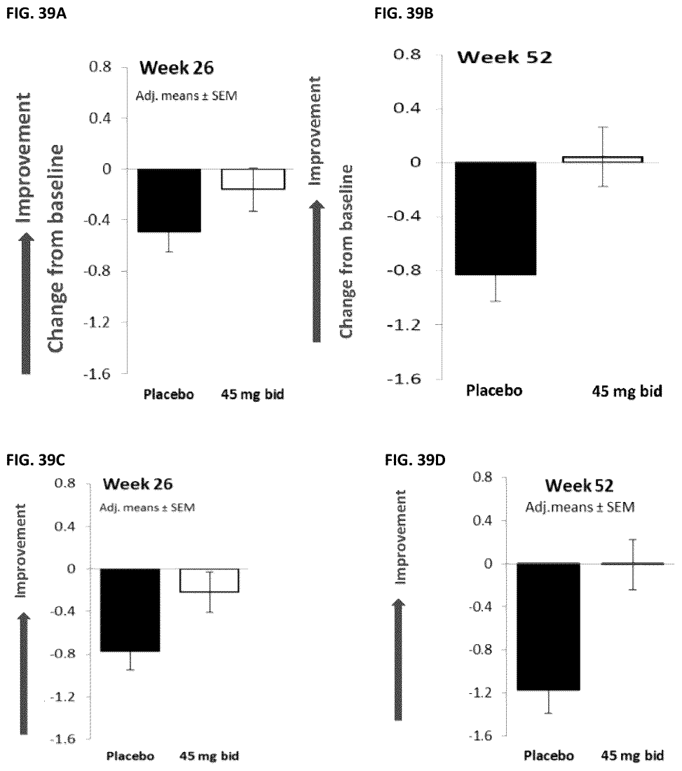

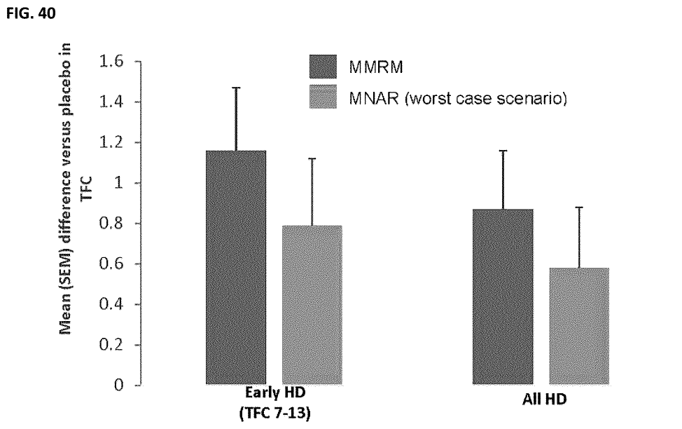

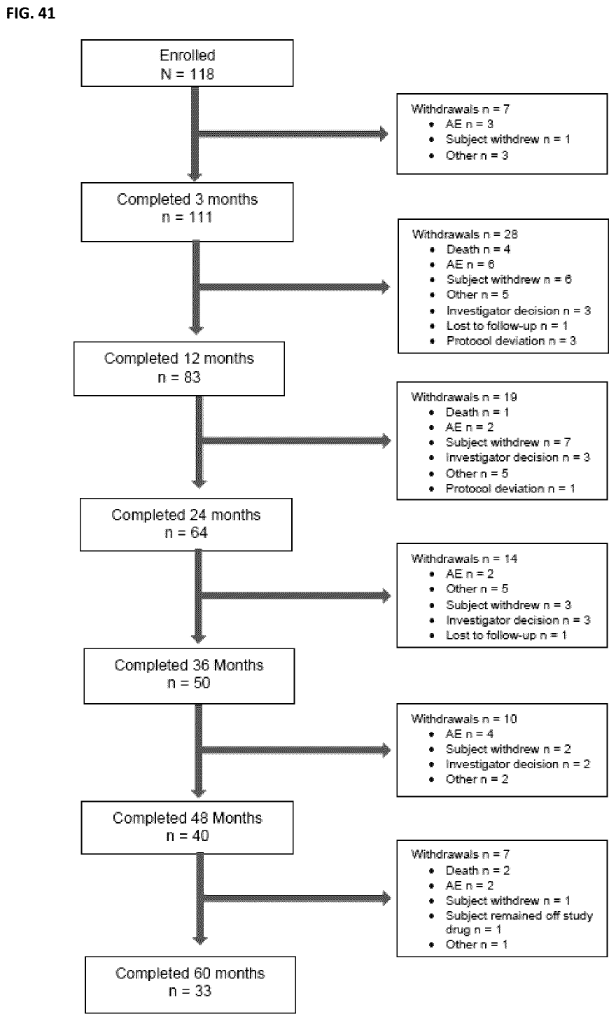

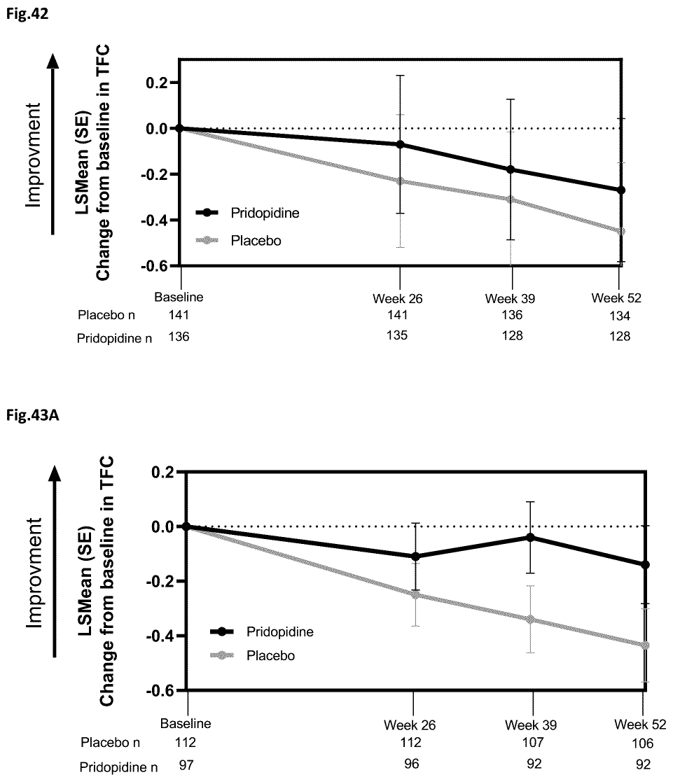

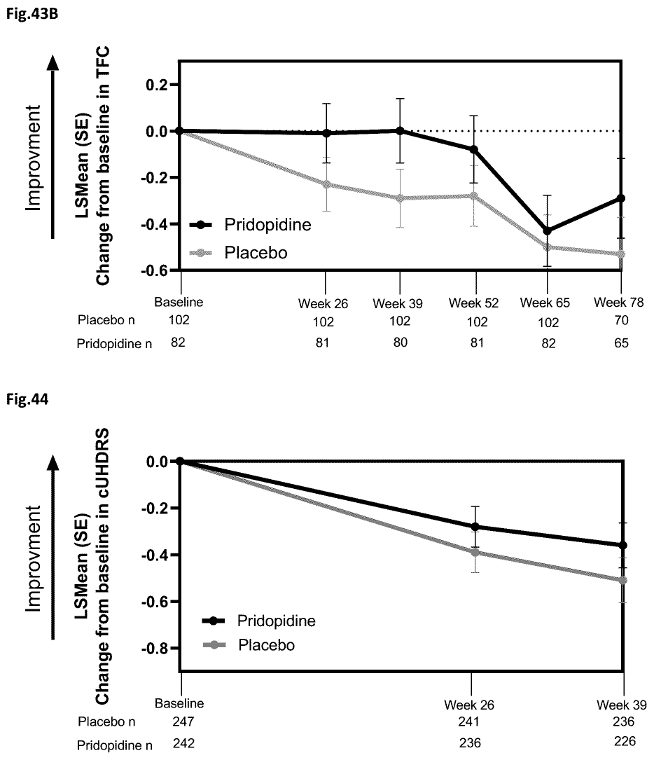

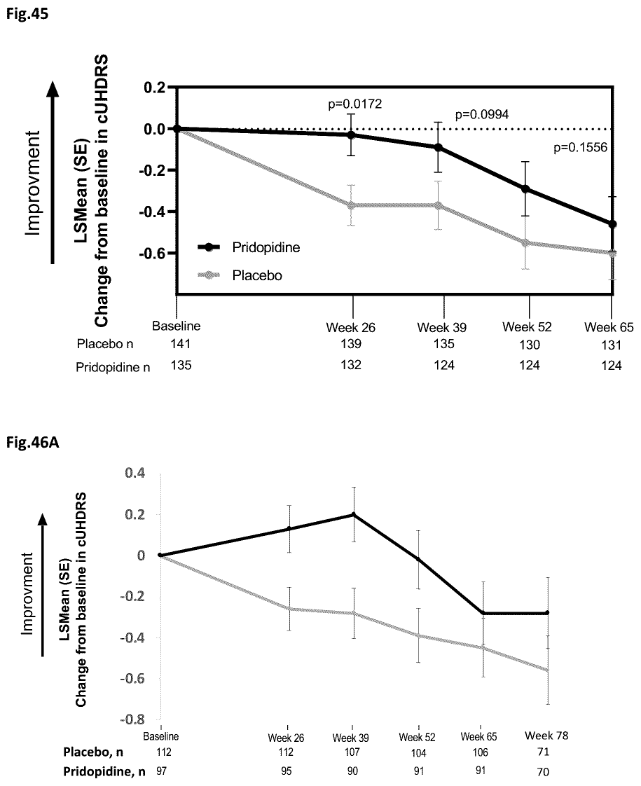

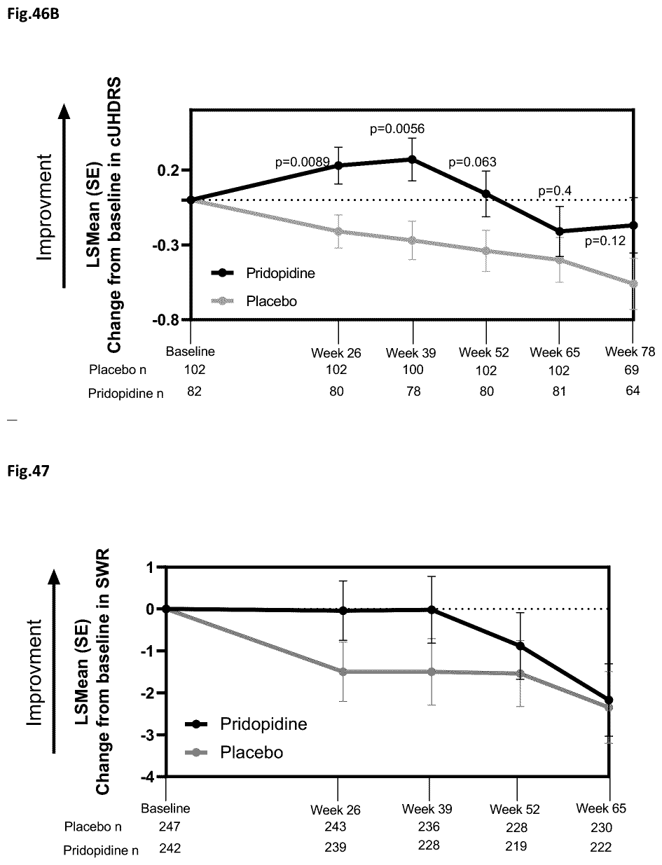

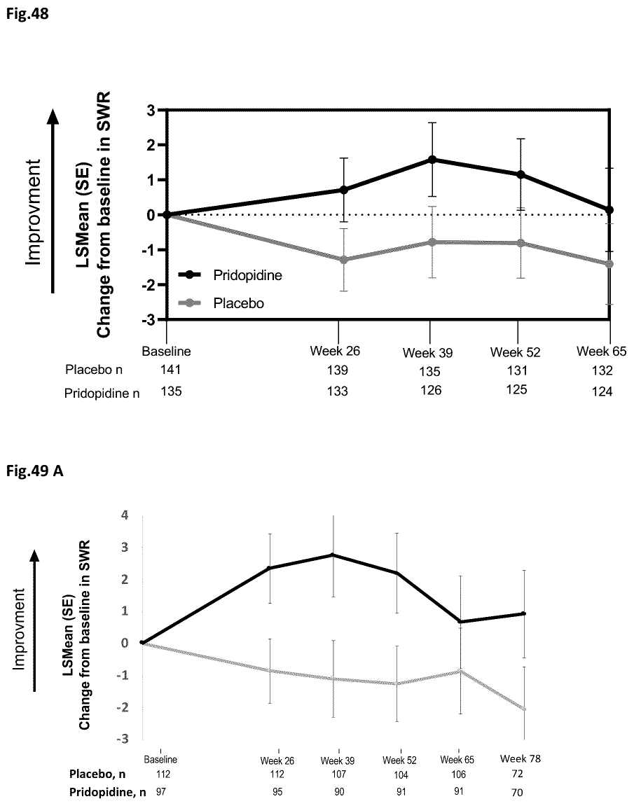

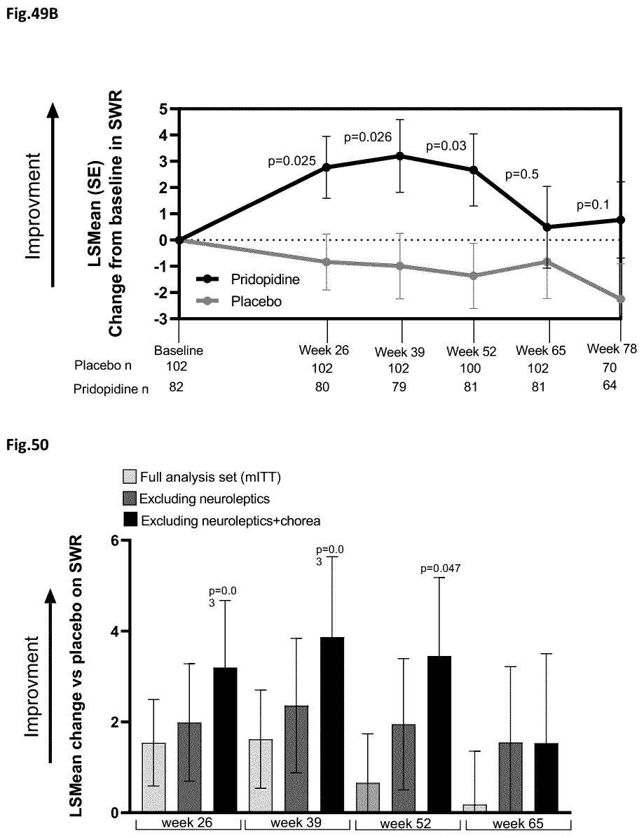

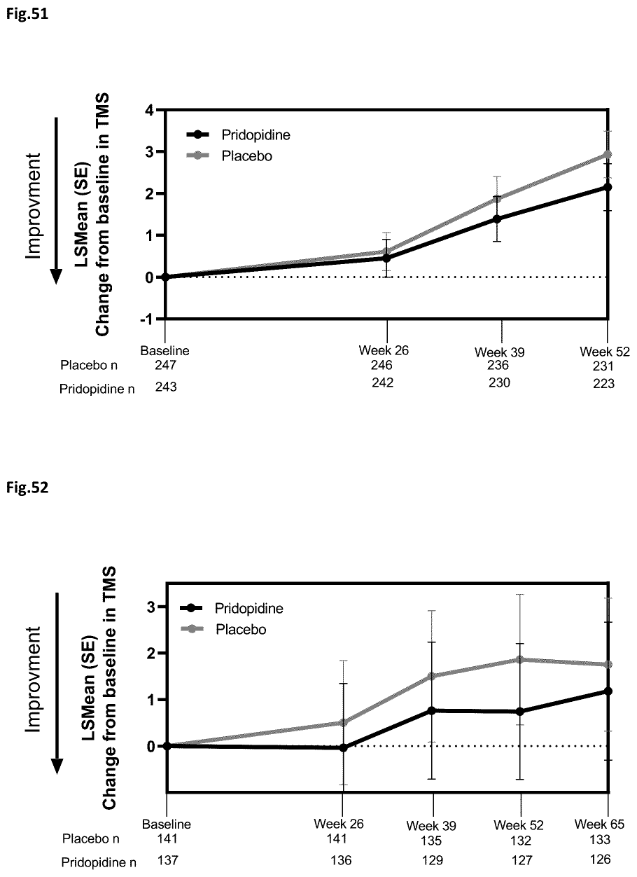

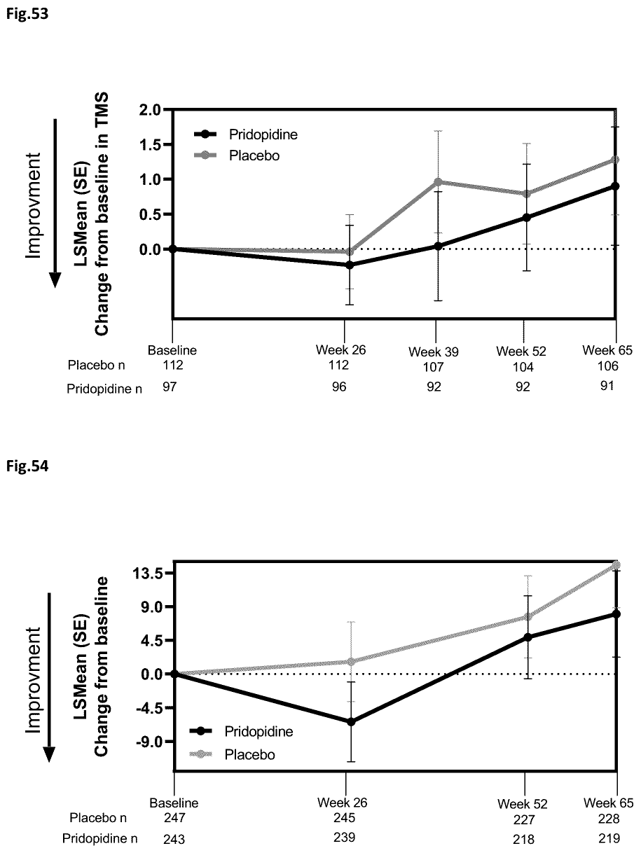

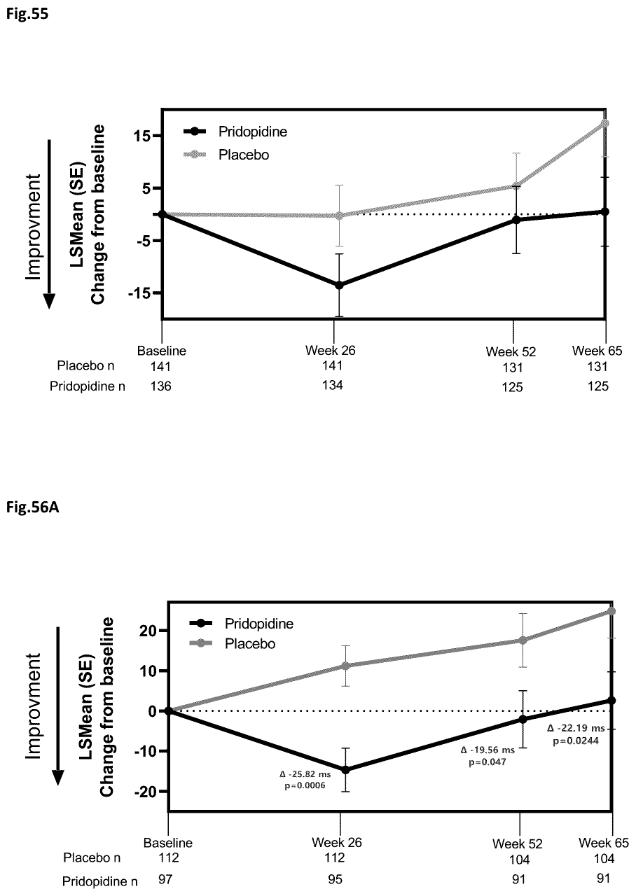

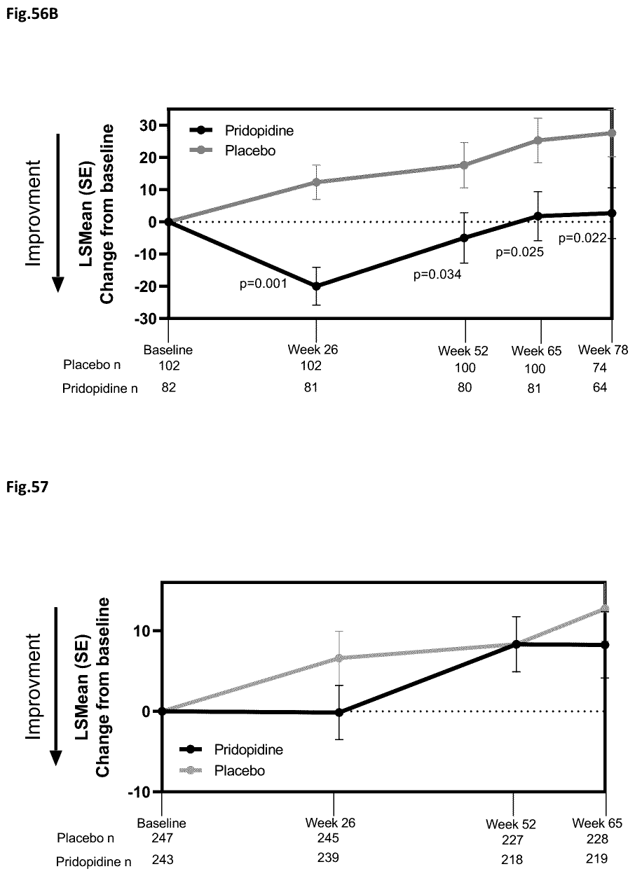

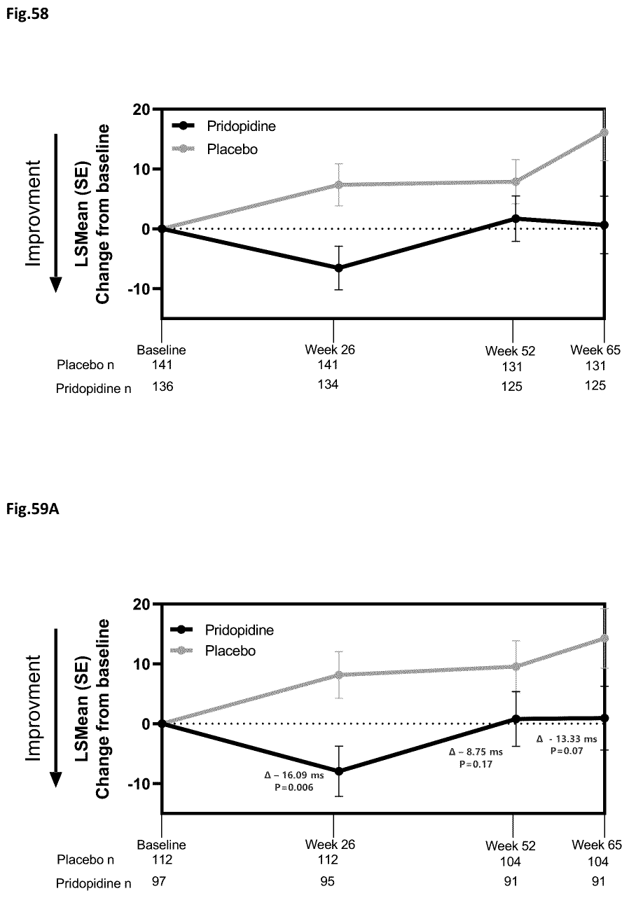

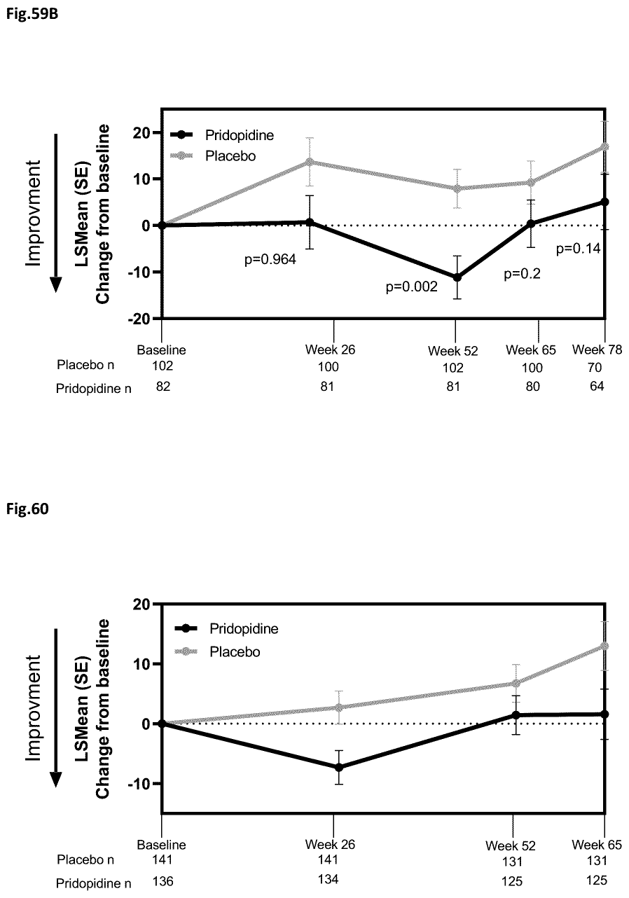

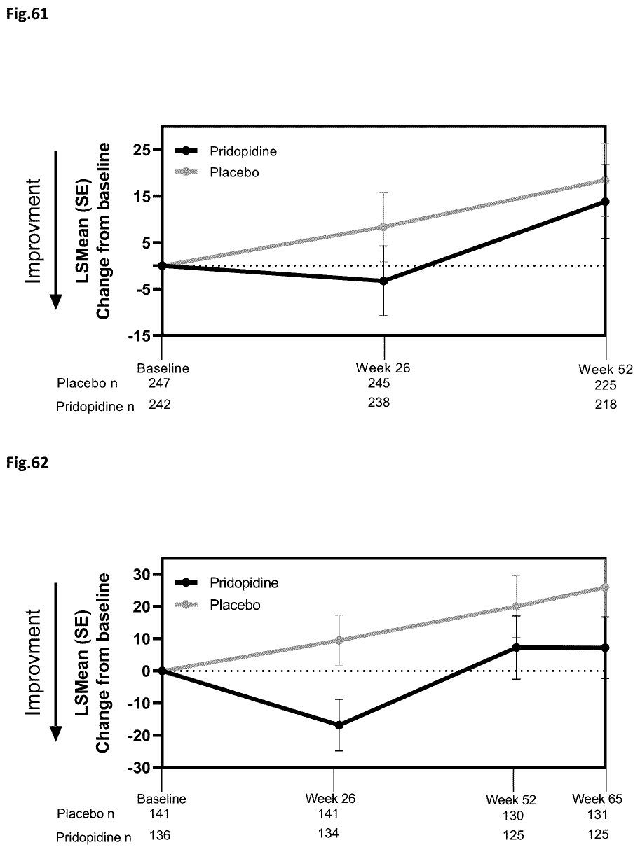

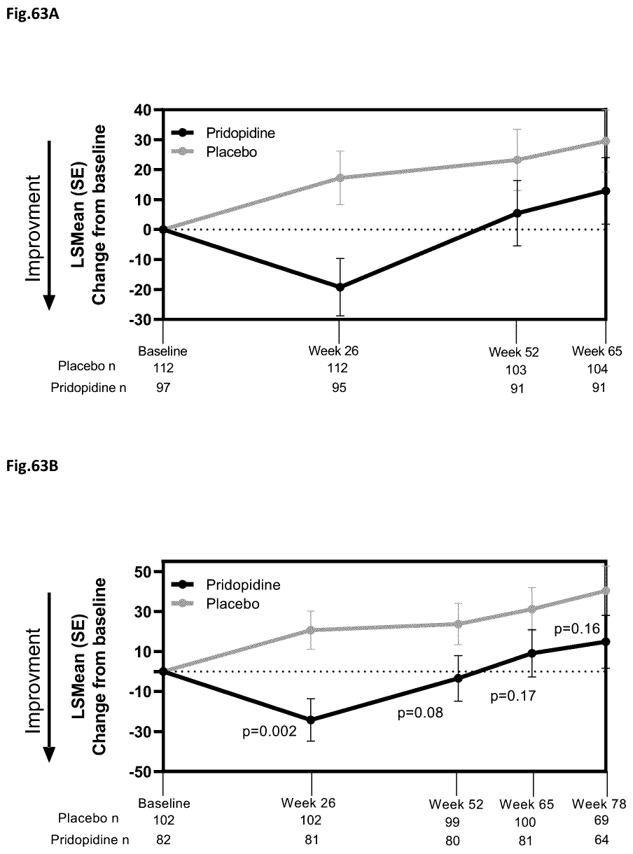

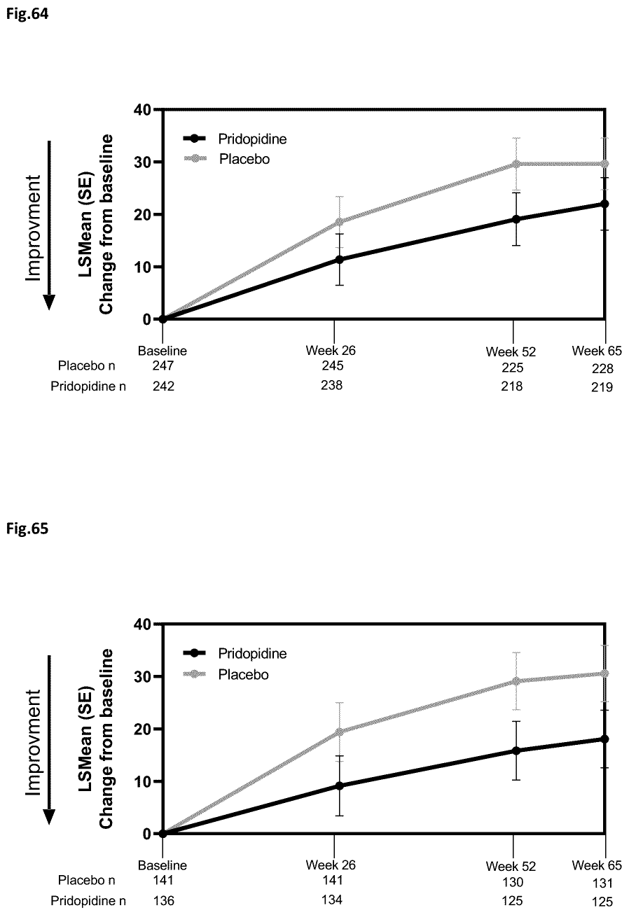

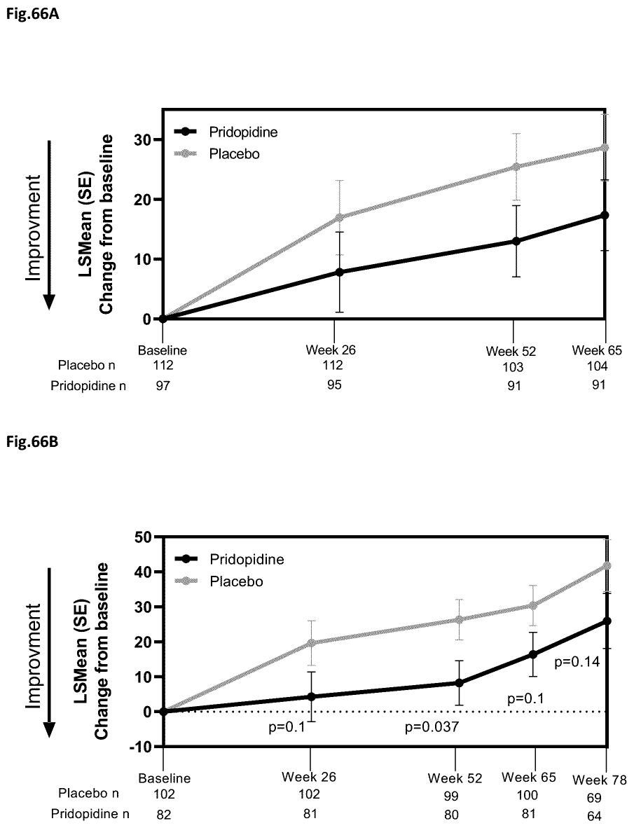

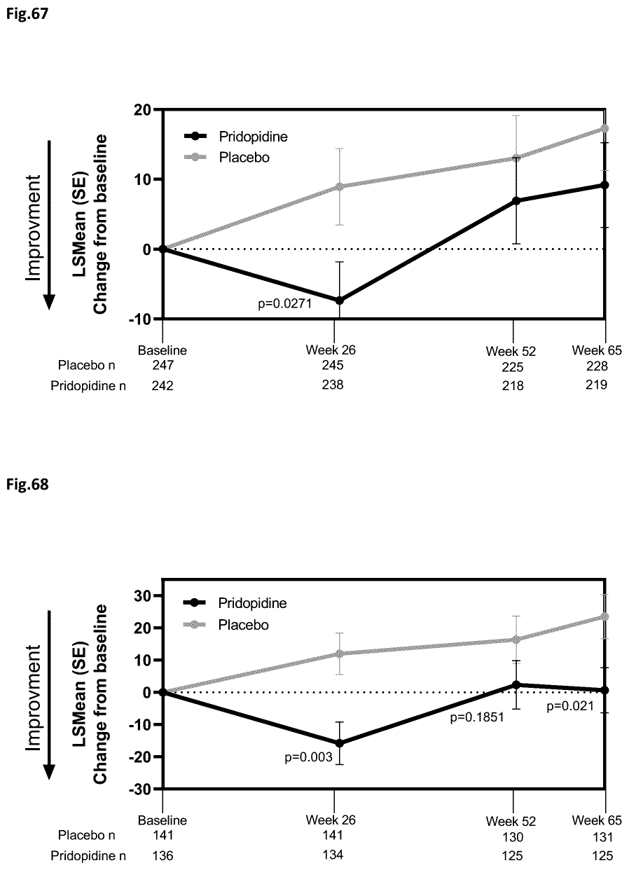

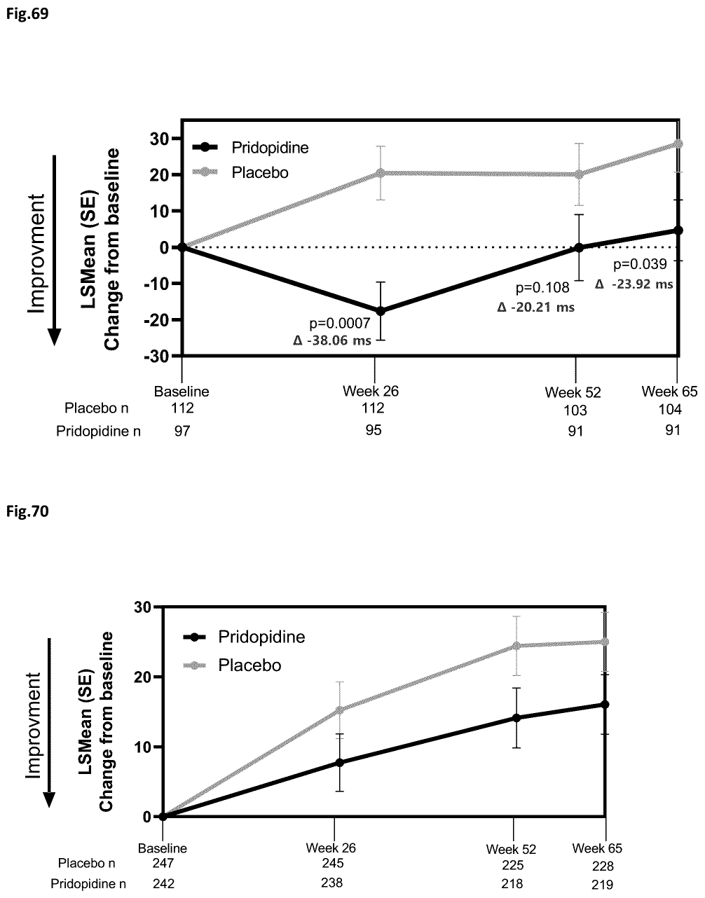

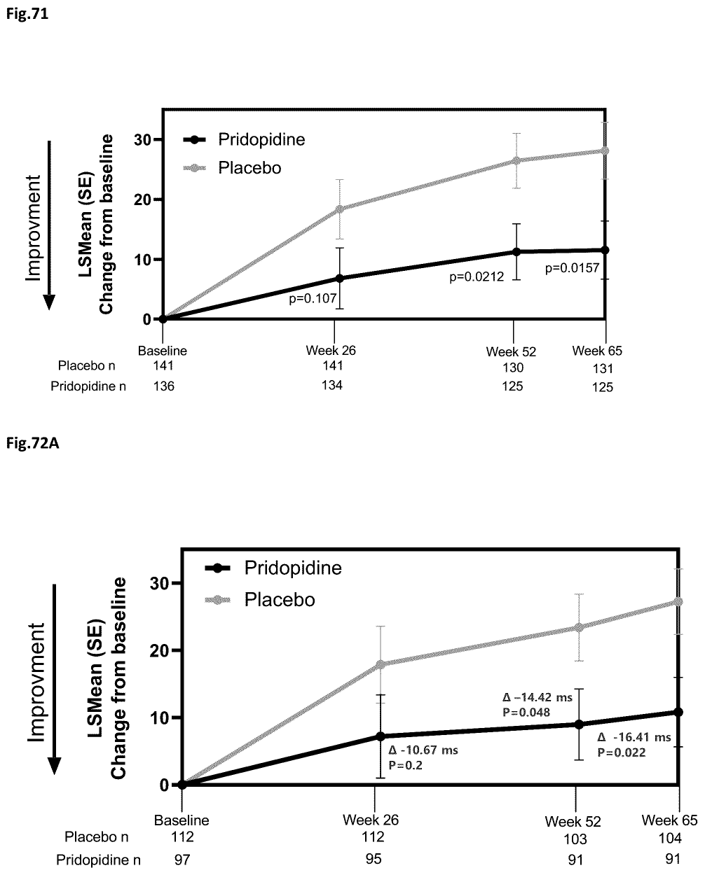

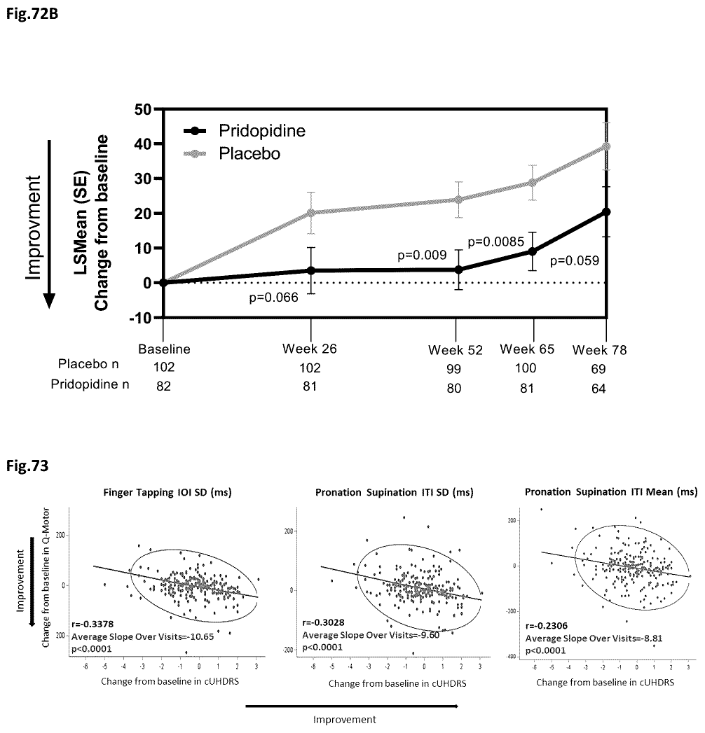

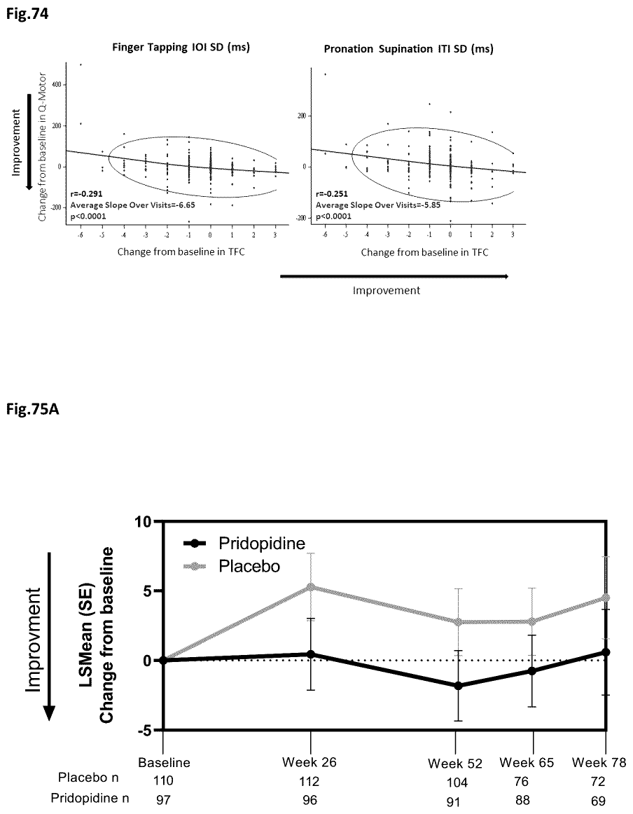

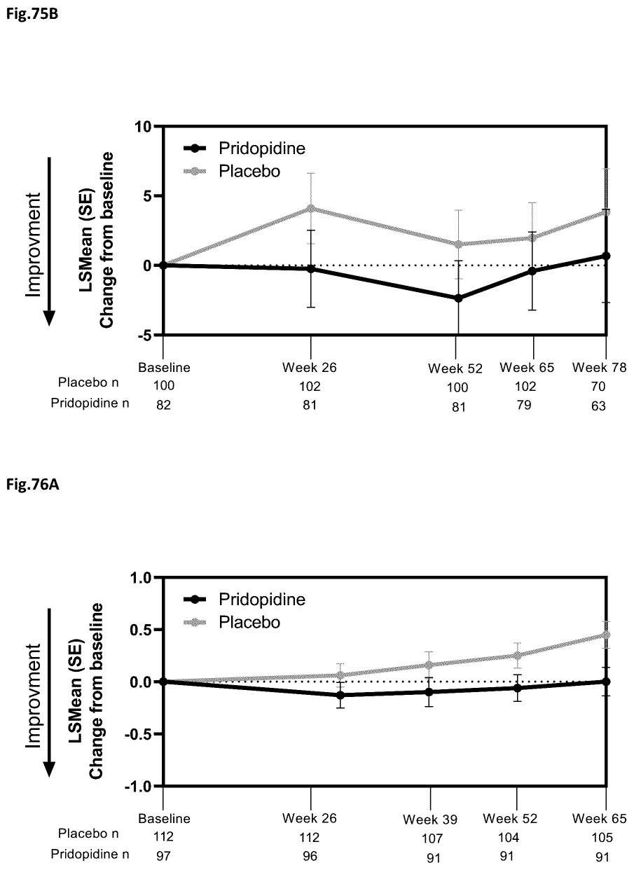

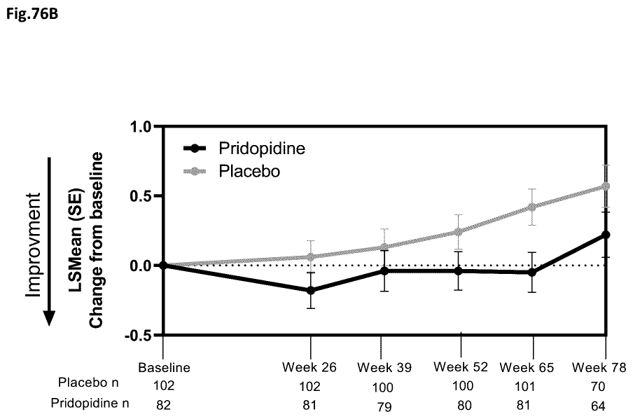

FIGS. 6B-6C: Black columns refer to responders: subjects with improvement or no change in UHDRS dystonia score. Gray columns refer to non-responders: FIG. 6B: Percentage of subjects with UHDRS TMS dystonia (≥0) receiving either placebo or 45 mg pridopidine bid that were either responders or non-responders. Of those patients with baseline (BL) dystonia score of ≥4 who completed 52 weeks of treatment with either placebo or 45 mg pridopidine bid, the percentage who were categorized based on the change in UHDRS TMS dystonia from BL to 52 weeks as responders (improved or no change, e.g. change ≥0) or non-responders (worsened, change <0). Responder Analysis for dystonia items support a trend toward improvement by showing that a higher percentage of patients were categorized as Responders within the dystonia items in the 45 mg bid treatment group compared to the placebo group (14 patients [77.8%] vs 18 patients [60.0%], respectively). FIG. 6C: Of those patients with baseline (BL) dystonia score of ≥4 who completed 52 weeks of treatment with either placebo or 45 mg pridopidine bid, the percentage who were categorized based on the change in UHDRS TMS dystonia from BL to 52 weeks as responders (improved, e.g. change ≥1) or non-responders (worsened or no change <1). There were statistically significantly more patients showing improvement in dystonia in the 45 mg bid group (66.7% responders) compared to patients receiving placebo (33.3% responders) (p=0.026).

FIG. 6D: PRIDE-HD patients with baseline (BL) dystonia score of ≥4 who completed 52 weeks of treatment with either placebo or 45 mg pridopidine bid were categorized based on the change in UHDRS limb dystonia from BL to 52 weeks as responders (improved, e.g. change ≥1) or non-responders (worsened or no change <1). A greater percentage of patients were categorized as Responders for the UHDRS-Limb Dystonia item in the pridopidine 45 mg bid treatment group compared to the placebo group (72.2% and 36.7%, respectively).

FIG. 7A: Beneficial effect of pridopidine 45 mg bid vs placebo in change in dystonia in limbs (UHDRS-dystonia limbs) at week 12; FIG. 7B: Finger Taps and Pronate-Supinate (P/S) hands at week 20; FIG. 7C: Finger Taps and P/S hands at week 26. Finger Taps and Pronate-Supinate (P/S) hands is a combination of finger tapping (the ability to tap the fingers of both hands where 15 repetitions in 5 seconds is considered normal) with pronation/supination (the ability to rotate the forearm and hand such that the palm is down (pronation) and to rotate the forearm and hand such that the palm is up (supination) on both sides of the body). Pronate-Supinate Hands is also known as the “Q-Motor: Pro-Sup-Frequency-MN-Hand (Hz)”. All data show to adjusted means+SE of change in full analysis set for FIGS. 7A-7C.

In the tables below, data and the P-values corresponding to the Figs. are provided. N refers to number of patients. Wk 26 refers to relevant score at week 26. Wk 52 refers to relevant score at week 52. “A to placebo” refers to the difference in score compared to placebo, specifically, the average change from baseline in the placebo group compared to the average change from baseline of the relevant group. “ALL” refers to pridopidine treated patients irrespective of disease stage. Y-axes are change from baseline for characteristic listed above the table. X-axes are dose whereby P means “placebo”, 45 means “45 mg bid”. In the Figs., improvement is in the direction from bottom of the graph to top of the graph.

For example, FIG. 8B shows the average difference in the UHDRS TMS score of the indicated group of patients (i.e. patients having a TFC score of 11-13 at baseline, i.e. HD1) between the score at baseline and the score after 26 weeks of administration of pridopidine (at week 26). Pridopidine 45 mg bid dose shows an improvement compared to placebo, with an approximately 6 point improvement compared to baseline (i.e. ˜−6 UHDRS TMS score at week 26 compared to baseline). The table below the description of FIG. 8B (table 4) shows that the 45 mg bid group had 17 patients (“N” row) and an average UHDRS TMS score of 35.4 at baseline (“Baseline” row). The table below the description of FIG. 8B also shows that the 45 mg bid group's change from baseline (about −6, shown in Fig., not shown in table) is 4.47 points better (−4.47) than the placebo group's change from placebo (about −2, shown in Fig., not shown in table) (“Δ to placebo” row). HD1 refers to an early stage HD patient with a baseline TFC score of 11-13. HD2 refers to an early stage HD patient with a baseline TFC score of 7-10.

FIG. 8A: Change from baseline in UHDRS TMS Week 26 ALL Table 3 below and FIG. 8A show no significant improvement in UHDRS TMS in the pridopidine treated patients at 26 weeks compared to placebo. Improvement is evidenced by a more negative value in the UHDRS TMS score. P means “placebo”, 45 means “45 mg bid”.

TABLE 3
Change from baseline in UHDRS
TMS Week 26, all HD stages

| | Placebo | 45 mg bid |
|---|---|---|
| N | 81 | 75 |
| Baseline | 46.9 | 44.5 |
| Δ to placebo | | 1.42 |
| p value | | 0.3199 |

FIG. 8B: Change from baseline in UHDRS TMS Week 26 Stage 1 BL TFC 11-13. (The UHDRS TMS score at week 26 of pridopidine treated patients with a baseline Total Functional Capacity (BL TFC) score of 11 to 13). HD patients with a baseline TFC score of 11-13 are generally considered to be first stage (stage 1) HD patients. Table 4 below and FIG. 8B show a trend towards improvement in UHDRS TMS in HD1 pridopidine treated patients at 26 weeks compared to placebo. P means “placebo”, 45 means “45 mg bid”.

TABLE 4
Change from baseline in UHDRS
TMS Week 26, Stage 1 BL TFC 11-13

| | Placebo | 45 mg bid |
|---|---|---|
| N | 12 | 17 |
| Baseline | 37.3 | 35.4 |
| Δ to placebo | | −4.47 |
| p value | | 0.0976 |

FIG. 8C: Change from baseline in UHDRS TMS Week 52 ALL. Table 5 below and FIG. 8C show no significant improvement in UHDRS TMS in all pridopidine treated patients at 52 weeks, compared to placebo. P means “placebo”, 45 means “45 mg bid”.

TABLE 5
Change from baseline in UHDRS
TMS Week 52, all HD stages

| | Placebo | 45 mg bid |
|---|---|---|
| N | 81 | 75 |
| Baseline | 46.9 | 44.5 |
| Δ to placebo | | 0.59 |
| p value | | 0.7468 |

FIG. 8D: Change from baseline in UHDRS TMS Week 52 Stage 1 BL TFC 11-13. Table 6 below and FIG. 8D show a trend towards improvement in UHDRS TMS in HD1 pridopidine treated patients at 52 weeks. P means “placebo”, 45 means “45 mg bid”.

TABLE 6
Change from baseline in UHDRS
TMS Week 52 Stage 1 BL TFC 11-13

| | Placebo | 45 mg bid |
|---|---|---|
| N | 12 | 17 |
| Baseline | 37.3 | 35.4 |
| Wk52 Δ to placebo | | −5.32 |
| p value | | 0.1065 |

FIG. 8E: Change from baseline in UHDRS TMS Gait and Balances Week 52. Table 7 below and FIG. 8E show no significant improvement in UHDRS TMS gait and balances in all pridopidine treated patients at 52 weeks. P means “placebo”, 45 means “45 mg bid”.

TABLE 7
Change from baseline in UHDRS TMS
gait and balance Week 52, all HD stages

| | Placebo | 45 mg bid |
|---|---|---|
| N | 81 | 75 |
| Baseline | 3.8 | 4.1 |
| Δ to placebo | | −0.09 |
| p value | | 0.7404 |

FIG. 8F: Change from baseline in UHDRS TMS Gait and Balances Week 52 Stage 1 BL TFC 11-13. Table 8 below and FIG. 8F show a significant improvement in UHDRS TMS gait and balance in HD1 pridopidine treated patients at 52 weeks. P means “placebo”, 45 means “45 mg bid”.

TABLE 8
Change from baseline in UHDRS TMS gait
and balance Week 52, Stage 1 BL TFC 11-13

| | Placebo | 45 mg bid |
|---|---|---|
| N | 12 | 17 |
| Baseline | 7.3 | 2.8 |
| Δ to placebo | | −0.94 |
| p value | | 0.0445 |

FIG. 8G: Change from baseline in UHDRS TMS Chorea Week 26 ALL. Table 9 below and FIG. 8G show no significant improvement in UHDRS TMS chorea in pridopidine 45 mg bid treated patients at all stages at 26 weeks. P means “placebo”, 45 means “45 mg bid”.

TABLE 9
Change from baseline in UHDRS
TMS Chorea Week 26 ALL

| | Placebo | 45 mg, bid |
|---|---|---|
| N | 81 | 75 |
| Baseline | 11.4 | 10.9 |
| Δ to placebo | | 0.92 |
| p value | | 0.1083 |

FIG. 8H: Change from baseline in UHDRS TMS Chorea Week 26 Stage 1 BL TFC 11-13. The table below and FIG. 8H show a trend towards improvement in UHDRS TMS chorea in HD1 pridopidine treated patients at 26 weeks. P means “placebo”, 45 means “45 mg bid”.

TABLE 10
Change from baseline in UHDRS TMS
Chorea Week 26, Stage 1 HD BL TFC 11-13

| | Placebo | 45 mg bid |
|---|---|---|
| N | 12 | 17 |
| Baseline | 8.8 | 9.9 |
| Wk26 Δ to placebo | | −1.4 |
| p value | | 0.1805 |

FIG. 8L Change from baseline in UHDRS TMS Dystonia Week 26 ALL. Table 11 below and FIG. 8I show no effect in UHDRS TMS dystonia in all pridopidine 45 mg bid treated patients at 26 weeks. P means “placebo”, 45 means “45 mg bid”.

TABLE 11
Change from baseline in UHDRS
dystonia at week 26 in all HD stages

| | Placebo | 45 mg bid |
|---|---|---|
| N | 81 | 75 |
| Baseline | 4.1 | 3.6 |
| Δ to placebo | | −0.06 |
| p value | | 0.8711 |

FIG. 8J Change from baseline in UHDRS TMS Dystonia Week 26 Stage 1 BL TFC 11-13. Table 12 below and FIG. 8J show a trend towards improvement in UHDRS TMS dystonia in HD1 pridopidine 45 mg bid treated patients at 26 weeks. P means “placebo”, 45 means “45 mg bid”.

TABLE 12
Change from baseline in UHDRS dystonia
at week 26 in Stage 1 HD BL TFC 11-13

| | Placebo | 45 mg bid |
|---|---|---|
| N | 12 | 17 |
| Baseline | 2.8 | 2.1 |
| Δ to placebo | | −0.99 |
| p value | | 0.1569 |

FIG. 8K: Change from baseline in UHDRS TMS Dystonia Week 52. Table 13 below and FIG. 8K show a trend toward improvement in UHDRS TMS dystonia in all pridopidine treated patients at 52 weeks. P means “placebo”, 45 means “45 mg bid”.

TABLE 13
Change from baseline in UHDRS TMS
Dystonia Week 52, all HD stages

| | Placebo | 45 mg bid |
|---|---|---|
| N | 81 | 75 |
| Baseline | 4.1 | 3.6 |
| Δ to placebo | | −0.39 |
| p value | | 0.4358 |

FIG. 8L: Change from baseline in UHDRS TMS Dystonia Week 52 Stage 1 BL TFC 11-13. Table 14 below and FIG. 8L show a significant improvement in UHDRS TMS dystonia in HD1 pridopidine 45 mg bid treated patients at 52 weeks. P means “placebo”, 45 means “45 mg bid”.

TABLE 14
Change from baseline in UHDRS TMS
Dystonia Week 52 Stage 1 HD BL TFC 11-13

| | Placebo | 45 mg bid |
|---|---|---|
| N | 12 | 17 |
| Baseline | 2.8 | 2.1 |
| Δ to placebo | | −1.65 |
| p value | | 0.0243 |

FIG. 8M: Change from baseline in UHDRS TMS Involuntary Movements Week 26 ALL. Table 15 below and FIG. 8M show no significant improvement in UHDRS TMS Involuntary Movements in pridopidine 45 mg bid treated patients at 26 weeks. P means “placebo”, 45 means “45 mg bid”.

TABLE 15
Change from baseline in UHDRS TMS
Involuntary Movements Week 26 all HD stages

| | Placebo | 45 mg bid |
|---|---|---|
| N | 81 | 75 |
| Baseline | 15.6 | 14.4 |
| Δ to placebo | | 0.89 |
| p value | | 0.2594 |

FIG. 8N: Change from baseline in UHDRS TMS Involuntary Movements Week 26 Stage 1 BL TFC 11-13. Table 16 below and FIG. 8N show significant improvement in UHDRS TMS Involuntary Movements at 26 weeks in HD1 pridopidine 45 mg bid treated patients. P means “placebo”, 45 means “45 mg bid”.

TABLE 16
Change from baseline in UHDRS TMS Involuntary
Movements Week 26 Stage 1 BL TFC 11-43

| | Placebo | 45 mg bid |
|---|---|---|
| N | 12 | 17 |
| Baseline | 11.5 | 12 |
| Δ to placebo | | −2.49 |
| p value | | 0.0469 |

FIG. 8O: Change from baseline in UHDRS TMS Involuntary Movements Week 52. Table 17 below and FIG. 8O show no improvement in UHDRS TMS Involuntary Movements in all pridopidine 45 mg bid patients at 52 weeks. P means “placebo”, 45 means “45 mg bid”.

TABLE 17
Change from baseline in UHDRS TMS
Involuntary Movements Week 52 all HD stages

| | Placebo | 45 mg bid |
|---|---|---|
| N | 81 | 75 |
| Baseline | 15.6 | 14.4 |
| Δ to placebo | | 0.02 |
| p value | | 0.9867 |

FIG. 8P: Change from baseline in UHDRS TMS Involuntary Movements Week 52 Stage 1 BL TFC 11-13. Table 18 below and FIG. 8P show a trend towards improvement in UHDRS TMS Involuntary Movements in HD1 pridopidine 45 mg bid treated patients at 52 weeks. P means “placebo”, 45 means “45 mg bid”.

TABLE 18
Change from baseline in UHDRS TMS Involuntary
Movements Week 52, Stage 1 BL TFC 11-43

| | Placebo | 45 mg bid |
|---|---|---|
| N | 12 | 17 |
| Baseline | 11.5 | 12 |
| Δ to placebo | | −2.73 |
| p value | | 0.1487 |

FIG. 8Q: Change from baseline in UHDRS TMS Excluding Chorea Week 52. Table 19 below and FIG. 8Q show no significant improvement in UHDRS TMS excluding chorea in all pridopidine 45 mg bid treated patients at 52 weeks. P means “placebo”, 45 means “45 mg bid”.

TABLE 19
Change from baseline in UHDRS TMS
Excluding Chorea Week 52, all HD stages

| | Placebo | 45 mg bid |
|---|---|---|
| N | 81 | 75 |
| Baseline | 35.5 | 33.6 |
| Δ to placebo | | 0.05 |
| p value | | 0.9693 |

FIG. 8R: Change from baseline in UHDRS TMS Excluding Chorea Week 52 Stage 1 BL TFC 11-13. Table 20 below and FIG. 8R show a trend towards improvement in UHDRS TMS excluding chorea in HD1 pridopidine 45 mg bid treated patients at 52 weeks. P means “placebo”, 45 means “45 mg bid”.

TABLE 20
Change from baseline in UHDRS TMS Excluding
Chorea Week 52, Stage 1 HD BL TFC 11-13

| | Placebo | 45 mg bid |
|---|---|---|
| N | 12 | 17 |
| Baseline | 28.6 | 25.5 |
| Δ to placebo | | −4.09 |
| p value | | 0.083 |

FIG. 8S: Change from baseline in UHDRS TMS Excluding Dystonia Week 26 ALL. Table 21 below and FIG. 8S show no significant improvement in UHDRS TMS excluding dystonia in pridopidine 45 mg bid treated patients at 26 weeks in all HD stages. P means “placebo”, 45 means “45 mg bid”.

TABLE 21
Change from baseline in UHDRS TMS
Excluding Dystonia Week 26 all HD stages.

| | Placebo | 45 mg bid |
|---|---|---|
| N | 81 | 75 |
| Baseline | 42.7 | 40.9 |
| Δ to placebo | | 1.39 |
| p value | | 0.2733 |

FIG. 8T: Change from baseline in UHDRS TMS Excluding Dystonia Week 26 Stage 1 BL TFC 11-13. Table 22 below and FIG. 8T show a trend towards improvement in UHDRS TMS excluding dystonia in HD1 pridopidine treated patients, at 26 weeks. P means “placebo”, 45 means “45 mg bid”.

TABLE 22
Change from baseline in UHDRS TMS Excluding
Dystonia Week 26 Stage 1 BL TFC 11-13

| | Placebo | 45 mg bid |
|---|---|---|
| N | 12 | 17 |
| Baseline | 34.6 | 33.4 |
| Δ to placebo | | −3.6 |
| p value | | 0.1594 |

FIG. 9A: Change from baseline in UHDRS Functional Assessment (FA) Week 26 ALL. Table 23 below and FIG. 9A show no significant improvement in UHDRS FA in pridopidine 45 mg bid treated patients at 26 weeks, all HD stages. Improvement is evidenced by a higher FA score. P means “placebo”, 45 means “45 mg bid”.

TABLE 23
Change from baseline in UHDRS Functional
Assessment Week 26, all HD stages

| | Placebo | 45 mg bid |
|---|---|---|
| N | 81 | 75 |
| Baseline | 18.6 | 19 |
| Δ to placebo | | 0.02 |
| p value | | 0.9511 |

FIG. 9B: Change from baseline in UHDRS Functional Assessment (FA) Week 26 Stage 1 BL TFC 11-13. Table 24 below and FIG. 9B show a trend towards improvement in UHDRS FA in HD1 pridopidine 45 mg bid treated patients, at 52 weeks.

TABLE 24
Change from baseline in UHDRS Functional
Assessment Week 52 Stage 1 BL TFC 11-13

| | Placebo | 45 mg bid |
|---|---|---|
| N | 12 | 17 |
| Baseline | 22.8 | 23.9 |
| Δ to placebo | | 1.23 |
| p value | | 0.0516 |

FIG. 9C: Change from baseline in UHDRS Independence Scale Week 26 ALL. Table 25 below and FIG. 9C show significant improvement in UHDRS IS in 45 mg bid pridopidine treated patients at 26 weeks.

TABLE 25
Change from baseline in UHDRS
Independence Scale Week 26 all HD stages.

| | Placebo | 45 mg bid |
|---|---|---|
| N | 81 | 75 |
| Baseline | 76.4 | 76.1 |
| Δ to placebo | | 1.79 |
| p value | | 0.0328 |

FIG. 9D: Change from baseline in UHDRS Independence Scale (IS) Week 26 Stage 1 BL TFC 11-13. Table 26 below and FIG. 9D show a stronger, more significant improvement in UHDRS IS in 45 mg bid treated HD1 patients compared to all HD stages, at 26 weeks.

TABLE 26
Change from baseline in UHDRS Independence
Scale Week 26 Stage 1 BL TFC 11-13

| | Placebo | 45 mg bid |
|---|---|---|
| N | 12 | 17 |
| Baseline | 83.8 | 84.1 |
| Δ to placebo | | 4.94 |
| p value | | 0.001 |

FIG. 9E: Change from baseline in UHDRS Independence Scale (IS) Week 52 ALL. Table 27 below and FIG. 9E show no significant improvement in UHDRS IS in all patients treated patients after 52 weeks.

TABLE 27
Change from baseline in UHDRS
Independence Scale Week 52 all RD stages

| | Placebo | 45 mg bid |
|---|---|---|
| N | 81 | 75 |
| Baseline | 76.4 | 76.1 |
| Δ to placebo | 0.86 | |
| p value | 0.5082 | |

FIG. 9F: Change from baseline in UHDRS Independence Scale (IS) Week 52 Stage 1 BL TFC 11-13. Table 28 below and FIG. 9F show a trend towards improvement in UHDRS IS in 45 mg bid treated HD1 patients, after 52 weeks.

TABLE 28
Change from baseline in UHDRS Independence
Scale Week 52 Stage 1 BL TFC 11-13

| | Placebo | 45 mg bid |
|---|---|---|
| N | 12 | 17 |
| Baseline | 83.8 | 84.1 |
| Δ to placebo | | 3.05 |
| p value | | 0.1289 |

FIG. 9G: Domestic Chores at 52 weeks, Early Stage HD (TFC≥7). Table 29 below provides data and the P-Values corresponding to FIG. 9G. Significant improvement in TFC domestic chores was observed in 45 mg bid pridopidine administered HD1 and HD2, TFC 7-13 patients, for 52 weeks.

TABLE 29
Change from baseline in TFC domestic chores
at week 52 in HD1 and HD2 (BL TFC ≥ 7)

| | Placebo | 45 mg bid |
|---|---|---|
| N | 62 | 59 |
| Baseline | 1.4 | 1.5 |
| Δ to placebo | | 0.24 |
| p value | | 0.0196 |

FIG. 9H: Care Level at 52 weeks, Early Stage HD (TFC≥7). Table 30 below provides data and the P-values corresponding to FIG. 9H. Significant improvement in TFC Care level was observed in 45 mg bid pridopidine administered HD1 and HD2, TFC 7-13 patients for 52 weeks.

TABLE 30
Change from baseline in TFC care level
at week 52 in HD1 and HD2 (BL TFC ≥ 7)

| | Placebo | 45 mg bid |
|---|---|---|
| N | 62 | 59 |
| Baseline | 2 | 1.9 |
| Δ to placebo | | 0.12 |
| p value | | 0.0044 |

FIG. 10A: Change from baseline in UHDRS Total Functional Capacity (TFC) Week 26 ALL. Table 31 below and FIG. 10A show a trend toward improvement in UHDRS TFC pridopidine 45 mg bid treated patients after 26 weeks.

TABLE 31
Change from baseline in TFC
at week 26 in all HD stages

| | Placebo | 45 mg bid |
|---|---|---|
| N | 81 | 75 |
| Baseline | 7.9 | 8.1 |
| Δ to placebo | | 0.34 |
| p value | | 0.1474 |

FIG. 10B: Change from baseline in UHDRS Total Functional Capacity (TFC) Week 26 Stage 1 BL TFC 11-13. Table 32 below and FIG. 10B show improvement in UHDRS TFC in 45 mg bid bid HD1 pridopidine treated patients, for 26 weeks.

TABLE 32
Change from baseline in UHDRS Total Functional
Capacity Week 26 Stage 1 BL TFC 11-13.

| | Placebo | 45 mg bid |
|---|---|---|
| N | 17 | 17 |
| Baseline | 11.8 | 11.5 |
| Δ to placebo | | 1.65 |
| p value | | 0.004 |

FIG. 10C: Change from baseline in UHDRS Total Functional Capacity (TFC) Week 52. Table 33 below and FIG. 10C show maintenance in functional capacity as measured by TFC score in patients receiving 45 mg bid pridopidine for 52 weeks, all HD stages.

TABLE 33
Change from baseline in UHDRS Total
Functional Capacity Week 52, all HD stages

| | Placebo | 45 mg bid |
|---|---|---|
| N | 81 | 75 |
| Baseline | 7.9 | 8.1 |
| Δ to placebo | | 0.87 |
| p value | | 0.0032 |

FIG. 10D: Change from baseline in UHDRS Total Functional Capacity (TFC) Week 52 Stage 1 BL TFC 11-13. Table 34 below and FIG. 10D show statistically significant maintenance of functional capacity as measured by TFC in HD1 patients receiving 45 mg bid pridopidine for 52 weeks.

TABLE 34
Change from baseline in UHDRS Total Functional
Capacity Week 52 Stage 1 BL TFC 11-13

| | Placebo | 45 mg bid |
|---|---|---|
| N | 12 | 17 |
| Baseline | 11.8 | 11.5 |
| Δ to placebo | | 1.89 |
| p value | | 0.0059 |

FIG. 10E: Change from baseline in UHDRS Total Functional Capacity (TFC) Week 52 Stage 2 BL TFC 7-10. Table 35 below and FIG. 10E show statistically significant maintenance of functional capacity as measured by TFC in HD2 patients receiving 45 mg bid pridopidine for 52 weeks.

TABLE 35
Change from baseline in UHDRS Total Functional
Capacity Week 52 Stage 2 BL TFC 7-10

| | Placebo | 45 mg bid |
|---|---|---|
| N | 50 | 42 |
| Baseline | 8.3 | 8.2 |
| Δ to placebo | | 0.94 |
| p value | | 0.009 |

FIG. 11A: Change from baseline in UHDRS TFC Finance ADL Week 26 ALL. Table 36a below and FIG. 11A show a trend towards improvement in finance ADL as measured as part of the UHDRS TFC score in all patients receiving pridopidine for 26 weeks.

TABLE 36A
Change from baseline in UHDRS TFC
Finance ADL Week 26, all HD stages

| | Placebo | 45 mg bid |
|---|---|---|
| N | 81 | 75 |
| Baseline | 4 | 4.1 |
| Δ to placebo | | 0.22 |
| p value | | 0.1782 |

FIG. 11B: Change from baseline in UHDRS TFC Finance ADL Week 26 Stage 1 BL TFC 11-13. Table 36B below and FIG. 11B show statistically significant improvement in finance ADL as measured as part of the TFC score in HD1 patients receiving pridopidine 45 mg bid for 26 weeks.

TABLE 36B
Change from baseline in UHDRS TFC
Finance ADL Week 26, Stage 1 BL TFC 11-13

| | Placebo | 45 mg bid |
|---|---|---|
| N | 12 | 17 |
| Baseline | 5.8 | 5.7 |
| Δ to placebo | | 0.92 |
| p value | | 0.0012 |

FIG. 11C: Change from baseline in UHDRS TFC Finance ADL Week 52 ALL. Table 37 below and FIG. 11C show a statistically significant improvement in finance ADL as measured as part of the UHDRS TFC score in all patients receiving 45 mg bid pridopidine for 52 weeks.

TABLE 37
Change from baseline in UHDRS TFC
Finance ADL Week 52, all HD stages

| | Placebo | 45 mg bid |
|---|---|---|
| N | 81 | 75 |
| Baseline | 4 | 4.1 |
| Δ to placebo | | 0.46 |
| p value | | 0.0164 |

FIG. 11D: Change from baseline in UHDRS TFC Finance ADL Week 52 Stage 1 BL TFC 11-13. Table 38 below and FIG. 11D show statistically significant improvement in finance ADL as measured as part of the TFC score in HD1 patients, receiving 45 mg bid pridopidine for 26 weeks.

TABLE 38
Change from baseline in UHDRS TFC
Finance ADL Week 52 Stage 1 BL TFC 11-13

| | Placebo | 45 mg bid |
|---|---|---|
| N | 12 | 17 |
| Baseline | 5.8 | 5.7 |
| Δ to placebo | | 0.77 |
| p value | | 0.0277 |

FIG. 11E: Change from baseline in UHDRS TFC Finance ADL Week 26 Stage 2 BL TFC 7-10. Table 39 below and FIG. 11E show statistically significant improvement in finance ADL as measured as part of the TFC score in HD2 patients receiving 45 mg bid pridopidine for 26 weeks.

TABLE 39
Change from baseline in UHDRS TFC
Finance ADL Week 26 Stage 2 BL TFC 7-10.

| | Placebo | 45 mg bid |
|---|---|---|
| N | 50 | 42 |
| Baseline | 4.4 | 4.3 |
| Δ to placebo | | 0.7 |
| p value | | 0.0045 |

FIG. 12A: Change from baseline in UHDRS TFC Finances Week 26 ALL. Table 40 below and FIG. 12A show trend for improvement in UHDRS TFC finances in pridopidine 45 mg bid treated patients at 26 weeks, all HD patients.

TABLE 40
Change from baseline in UHDRS
TFC Finances Week 26 ALL

| | Placebo | 45 mg bid |
|---|---|---|
| N | 81 | 75 |
| Baseline | 1.6 | 1.8 |
| Δ to placebo | | 0.1 |
| p value | | 0.3629 |

FIG. 12B: Change from baseline in UHDRS TFC Finances Week 26 Stage 1 BL TFC 11-13. Table 41 below and FIG. 12B show a trend for improvement in UHDRS TFC finances in HD1 patients receiving 45 mg bid pridopidine for 26 weeks.

TABLE 41
Change from baseline in UHDRS TFC
Finances Week 26 Stage 1 BL TFC 11-13

| | Placebo | 45 mg bid |
|---|---|---|
| N | 12 | 17 |
| Baseline | 2.8 | 2.9 |
| Δ to placebo | | 0.25 |
| p value | | 0.1183 |

FIG. 12C: Change from baseline in UHDRS TFC Finances Week 52. Table 42 below and FIG. 12C show statistically significant improvement in TFC finances in HD1 patients receiving 45 mg bid pridopidine for 52 weeks.

TABLE 42
Change from baseline in UHDRS
TFC Finances Week 52, all HD stages

| | Placebo | 45 mg bid |
|---|---|---|
| N | 81 | 75 |
| Baseline | 1.6 | 1.8 |
| Δ to placebo | | 0.31 |
| p value | | 0.0143 |

FIG. 12D: Change from baseline in UHDRS TFC Finances Week 52 Stage 2 BL TFC 7-10. Table 43 below and FIG. 12D show statistically significant improvement in UHDRS TFC finances in HD2 patients receiving 45 mg bid pridopidine for 26 weeks.

TABLE 43
Change from baseline in UHDRS TFC
Finances Week 52 Stage 2 BL TFC 7-10.

| | Placebo | 45 mg bid |
|---|---|---|
| N | 50 | 42 |
| Baseline | 1.8 | 1.9 |
| Δ to placebo | | 0.39 |
| p value | | 0.0336 |

FIG. 13A: Change from baseline in UHDRS TFC Domestic Chores Week 26 Stage 1 BL TFC 11-13. The table below and FIG. 13A show a trend towards improvement in TFC domestic chores in HD1 patients receiving pridopidine 45 mg bid for 26 weeks.

TABLE 44
Change from baseline in UHDRS TFC
Domestic Chores Week 26 Stage 1 BL TFC 11-13

| | Placebo | 45 mg bid |
|---|---|---|
| N | 12 | 17 |
| Baseline | 2 | 1.8 |
| Δ to placebo | | 0.34 |
| p value | | 0.0589 |

FIG. 13B: Change from baseline in UHDRS TFC Domestic Chores Week 52 ALL. Table 45 below and FIG. 13B show a trend for improvement in UHDRS TFC domestic chores in all pridopidine 45 mg bid treated patients at 52 weeks.

TABLE 45
Change from baseline in UHDRS
TFC Domestic Chores Week 52, all HD stages.

| | Placebo | 45 mg bid |
|---|---|---|
| N | 59 | 56 |
| Baseline | 1.3 | 1.3 |
| Δ to placebo | | 0.23 |
| p value | | 0.0647 |

FIG. 13C: Change from baseline in UHDRS TFC Domestic Chores Week 52 Stage 1 BL TFC 11-13. Table 45 below and FIG. 13C show statistically significant improvement in TFC domestic chores in HD1 patients receiving 45 mg bid pridopidine for 52 weeks.

TABLE 45
Change from baseline in UHDRS TFC
Domestic Chores Week 52 Stage 1 BL TFC 11-13.

| | Placebo | 45 mg bid |
|---|---|---|
| N | 12 | 17 |
| Baseline | 2 | 1.8 |
| Δ to placebo | | 0.49 |
| p value | | 0.0161 |

FIG. 14A: Change from baseline in UHDRS TFC ADL Week 26 ALL. Table 46 below and FIG. 14A show a trend for improvement in TFC ADL in all pridopidine treated patients at 26 weeks.

TABLE 46
Change from baseline in UHDRS
TFC ADL Week 26, all HD patients

| | Placebo | 45 mg bid |
|---|---|---|
| N | 81 | 75 |
| Baseline | 2.4 | 2.3 |
| Δ to placebo | | 0.12 |
| p value | | 0.205 |

FIG. 14B: Change from baseline in UHDRS TFC ADL Week 26 Stage 1 BL TFC 11-13. Table 47 below and FIG. 14B show statistically significant improvement in UHDRS TFC ADL in HD1 patients receiving 45 mg bid pridopidine for 26 weeks.

TABLE 47
Change from baseline in UHDRS TFC
ADL Week 26 Stage 1 BL TFC 11-13

| | Placebo | 45 mg bid |
|---|---|---|
| N | 12 | 17 |
| Baseline | 2.9 | 2.8 |
| Δ to placebo | | 0.65 |
| p value | | 0.0011 |

FIG. 14C: Change from baseline in UHDRS TFC ADL Week 52 ALL. Table 48 below and FIG. 14C show a trend for improvement in UHDRS TFC ADL in all pridopidine 45 mg bid treated patients at 52 weeks.

TABLE 48
Change from baseline in UHDRS
TFC ADL Week 52, all HD patients

| | Placebo | 45 mg bid |
|---|---|---|
| N | 81 | 75 |
| Baseline | 2.4 | 2.3 |
| Δ to placebo | | 0.14 |
| p value | | 0.2216 |

FIG. 14D: Change from baseline in UHDRS TFC ADL Week 52 Stage 1 BL TFC 11-13. Table 49 below and FIG. 14D show statistically significant improvement in UHDRS TFC ADL in HD1 patients receiving 45 mg bid pridopidine for 52 weeks.

TABLE 49
Change from baseline in UHDRS TFC
ADL Week 52 Stage 1 BL TFC 11-13

| | Placebo | 45 mg bid |
|---|---|---|
| N | 12 | 17 |
| Baseline | 2.9 | 2.8 |
| Δ to placebo | | 0.62 |
| p value | | 0.0044 |

FIG. 14E: Change from baseline in UHDRS TFC ADL Week 52 Stage 2 BL TFC 7-10. Table 50 below and FIG. 14E show statistically significant improvement in UHDRS TFC ADL in HD2 patients receiving 45 mg bid pridopidine for 52 weeks.

TABLE 50
Change from baseline in UHDRS TFC
ADL Week 52 Stage 2 BL TFC 7-10

| | Placebo | 45 mg bid |
|---|---|---|
| N | 50 | 42 |
| Baseline | 2.6 | 2.5 |
| Δ to placebo | | 0.27 |
| p value | | 0.0356 |

FIG. 15A: Change from baseline in UHDRS TFC Care Level Week 52 ALL. The table below and FIG. 15A show a trend for improvement in UHDRS TFC care level in all pridopidine 45 mg bid treated patients at 52 weeks.

TABLE 51
Change from baseline in UHDRS
TFC Care Level Week 52, all HD stages

| | Placebo | 45 mg bid |
|---|---|---|
| N | 59 | 56 |
| Baseline | 1.9 | 1.9 |
| Δ to placebo | | 0.09 |
| p value | | 0.1153 |

FIG. 15B: Change from baseline in UHDRS TFC Care Level Week 52 Stage 2 BL TFC 7-10. Table 52 below and FIG. 15B show statistically significant improvement in UHDRS TFC care level in HD2 patients receiving 45 mg bid pridopidine for 52 weeks.

TABLE 52
Change from baseline in UHDRS TFC
Care Level Week 52 Stage 2 BL TFC 7-10.

| | Placebo | 45 mg bid |
|---|---|---|
| N | 50 | 47 |
| Baseline | 1.9 | 1.9 |
| Δ to placebo | | 0.13 |
| p value | | 0.0156 |

FIG. 16A: Change from baseline in PBA Total Score Week 26 Stage 1 BL TFC 11-13. Table 53 below and FIG. 16A show a trend towards improvement in PBA total score in HD1 patients receiving pridopidine 45 mg bid for 26 weeks (negative change indicates an improvement).

TABLE 53
Change from baseline in PBA Total
Score Week 26 Stage 1 BL TFC 11-13

| | Placebo | 45 mg bid |
|---|---|---|
| N | 12 | 17 |
| Baseline | 8.8 | 8.1 |
| Δ to placebo | | −4.83 |
| p value | | 0.319 |

FIG. 16B: PBA Change from baseline in Total Score Week 52 Full analysis set. The table below and FIG. 16B show trend to improvement in PBA total score in all 45 mg bid pridopidine treated patients at 52 weeks.

TABLE 54
PBA Change from baseline in Total
Score Week 52, all HD stages

| | Placebo | 45 mg bid |
|---|---|---|
| N | 81 | 75 |
| Baseline | 12 | 10.9 |
| Δ to placebo | | −3.98 |
| p value | | 0.0603 |

FIG. 16C: Change from baseline in PBA Total Score Week 52 BL TFC≥7. The table below and FIG. 16C show a trend for improvement in PBA total score in 45 mg bid pridopidine treated HD1 and HD2, TFC 7-13 patients at 52 weeks.

TABLE 55
Change from baseline in PBA Total
Score Week 52 BL TFC ≥7

| | Placebo | 45 mg bid |
|---|---|---|
| N | 62 | 59 |
| Baseline | 11.4 | 10.1 |
| Δ to placebo | | −2.74 |
| p value | | 0.1911 |

FIG. 16D: Change from baseline PBA Irritability, Severity×Frequency Week 52 ALL. Table 56 below and FIG. 16d show significant improvement in PBA irritability in all pridopidine 45 mg bid treated patients at 52 weeks.

TABLE 56
Change from baseline PBA Irritability,
Severity × Frequency Week 52, all HD patients

| | Placebo | 45 mg bid |
|---|---|---|
| N | 81 | 75 |
| Baseline | 2 | 1.6 |
| Δ to placebo | | −1.03 |
| p value | | 0.0126 |

FIG. 16E: Change from baseline in PBA Lack of Initiative (Apathy), Severity×Frequency Week 26 ALL. The table below and FIG. 16E show a trend for improvement in PBA apathy in all pridopidine treated patients at 26 weeks.

TABLE 57
Change from baseline in PBA Lack
of Initiative (Apathy), Severity ×
Frequency Week 26, all HD stages

| | Placebo | 45 mg bid |
|---|---|---|
| N | 81 | 75 |
| Baseline | 2.6 | 2.5 |
| Δ to placebo | | −0.87 |
| p value | | 0.1235 |

FIG. 16F: Change from baseline in PBA Lack of Initiative (Apathy), Severity×Frequency Week 26 Stage 1 BL TFC 11-13. The table below and FIG. 16F show a trend for improvement in PBA apathy in HD1 patients receiving pridopidine 45 mg bid for 26 weeks.

TABLE 58
Change from baseline in PBA Lack
of Initiative (Apathy), Severity ×
Frequency Week 26 Stage 1 BL TFC 11-13

| | Placebo | 45 mg bid |
|---|---|---|
| N | 12 | 17 |
| Baseline | 1.2 | 1 |
| Δ to placebo | | −1.85 |
| p value | | 0.0703 |

FIG. 16G: Change from baseline in PBA Lack of Initiative (Apathy), Severity×Frequency Week 52 Full analysis set. The table below and FIG. 16G show a trend for improvement in PBA apathy in BL stage 1 patients receiving pridopidine for 52 weeks.

TABLE 59
Change from baseline in PBA Lack
of Initiative (Apathy), Severity ×
Frequency Week 52, all HD stages

| Placebo | 45 mg bid | 67.5 mg bid |
|---|---|---|
| N | 81 | 75 |
| Baseline | 2.6 | 2.5 |
| Δ to placebo | | −1.27 |
| p value | | 0.0704 |

FIG. 16H: PBA Change from baseline in PBA Lack of Initiative (Apathy), Severity×Frequency Week 52 1 BL TFC≥7. Table 60 below and FIG. 16H show a trend for improvement in PBA apathy in HD1 and HD2, TFC 7-13 pridopidine treated patients for 52 weeks.

TABLE 60
PBA Change from baseline in PBA
Lack of Initiative (Apathy), Severity ×
Frequency Week 52 1 BL TFC ≥7

| Placebo | 45 mg bid | 67.5 mg bid |
|---|---|---|
| N | 62 | 59 |
| Baseline | 2.5 | 2 |
| Δ to placebo | | −1.39 |
| p value | | 0.0608 |

FIG. 16I: Change from baseline in PBA Obsessive-Compulsive, Severity×Frequency Week 26 Stage 1 BL TFC 11-13. The table below and FIG. 16I show a statistically significant improvement in PBA O-C in HD1 patients receiving pridopidine 45 mg bid for 26 weeks.

TABLE 61
Change from baseline in PBA Obsessive-
Compulsive, Severity × Frequency
Week 26 Stage 1 BL TFC 11-13

| | Placebo | 45 mg bid |
|---|---|---|
| N | 12 | 17 |
| Baseline | 0 | 1 |
| Δ to placebo | | −2.11 |
| p value | | 0.0035 |

FIG. 16J: Change from baseline in PBA Obsessive-Compulsive, Severity×Frequency Week 52 Stage 1 BL TFC 11-13. The table below and FIG. 16J show statistically significant improvement in PBA O-C in HD1 patients receiving pridopidine 45 mg bid for 52 weeks.

TABLE 62
Change from baseline in PBA Obsessive-
Compulsive, Severity × Frequency
Week 52 Stage 1 BL TFC 11-13

| | Placebo | 45 mg bid |
|---|---|---|
| N | 12 | 17 |
| Baseline | 0 | 1 |
| Δ to placebo | | −2.73 |
| p value | | 0.007 |

FIG. 16K: Change from baseline in PBA Disoriented Behavior, Severity×Frequency Week 26 ALL. The table below and FIG. 16K show trend for improvement in PBA Disoriented Behavior in all pridopidine treated patients at 26 weeks.

TABLE 63
Change from baseline in PBA
Disoriented Behavior, Severity ×
Frequency Week 26, all HD patients

| | Placebo | 45 mg bid |
|---|---|---|
| N | 81 | 75 |
| Baseline | 0.6 | 0.4 |
| Δ to placebo | | −0.2 |
| p value | | 0.2864 |

FIG. 16L: Change from baseline in PBA Disoriented Behavior, Severity×Frequency Week 26 Stage 1 BL TFC 11-13. The table below and FIG. 16L show significant improvement in PBA Disoriented Behavior in HD1 patients receiving 45 mg bid pridopidine at 26 weeks.

TABLE 64
Change from baseline in PBA
Disoriented Behavior, Severity ×
Frequency Week 26 Stage 1 BL TFC 11-13

| | Placebo | 45 mg bid |
|---|---|---|
| N | 12 | 17 |
| Baseline | 0.1 | 0 |
| Δ to placebo | | −0.19 |
| p value | | 0.0381 |

FIG. 17A: Change from baseline in Timed Up and Go Test (sec) Week 26 ALL. The table below and FIG. 17A show trend for improvement in Timed up and go test in all pridopidine 45 mg bid treated patients at 26 weeks.

TABLE 65
Change from baseline in Timed Up and
Go Test (sec) Week 26, all HD patients

| | Placebo | 45 mg bid |
|---|---|---|
| N | 81 | 75 |
| Baseline | 12.1 | 12.1 |
| Δ to placebo | | −2.16 |
| p value | | 0.1765 |

FIG. 17B: Change from baseline in Timed Up and Go Test (sec) Week 26 Stage 1 BL TFC 11-13. The table below and FIG. 17B show a trend for improvement in the Timed up and go test in pridopidine treated HD1 patients at 26 weeks.

TABLE 66
Change from baseline in Timed Up and Go
Test (sec) Week 26 Stage 1 BL TFC 11-13

| | Placebo | 45 mg bid |
|---|---|---|
| N | 12 | 17 |
| Baseline | 9.7 | 8.6 |
| Δ to placebo | | −6.98 |
| p value | | 0.0612 |

FIG. 17C: Change from baseline in Timed Up and Go Test (sec) Week 52. The table below and FIG. 17C show a trend for improvement in the Timed up and go test in all pridopidine treated patients at 52 weeks.

TABLE 67
Change from baseline in Timed Up and Go
Test (sec) Week 52, all HD stages

| | Placebo | 45 mg bid |
|---|---|---|
| N | 81 | 75 |
| Baseline | 12.1 | 12.1 |
| Δ to placebo | | −1.49 |
| p value | | 0.0899 |

FIG. 17D: Change from baseline in Timed Up and Go Test (sec) Week 52 Stage 1 BL TFC 11-13. The table below and FIG. 17D show trend toward improvement in the Timed up and go test in pridopidine treated HD1 patients at 52 weeks.

TABLE 68
Change from baseline in Timed Up and Go
Test (sec) Week 52 Stage 1 BL TFC 11-13.

| | Placebo | 45 mg bid |
|---|---|---|
| N | 12 | 17 |
| Baseline | 9.7 | 8.6 |
| Δ to placebo | | −5.26 |
| p value | | 0.0627 |

FIG. 18A: Change from baseline in Walk-12 Total Score Week 26 ALL. The table below and FIG. 18A show a trend for improvement in the Walk-12 TS in all pridopidine treated patients at 26 weeks.

TABLE 69
Change from baseline in Walk-12
Total Score Week 26, all HD stages

| | Placebo | 45 mg bid |
|---|---|---|
| N | 81 | 75 |
| Baseline | 31.5 | 32.1 |
| Δ to placebo | | −2.45 |
| p value | | 0.3359 |

FIG. 18B: Change from baseline in Walk-12 Total Score Week 26 Stage 1 BL TFC 11-13. The table below and FIG. 18B show statistically significant improvement in the Walk-12 TS in pridopidine treated HD1 patients having at 26 weeks.

TABLE 70
Change from baseline in Walk-12 Total
Score Week 26 Stage 1 BL TFC 11-13

| | Placebo | 45 mg bid |
|---|---|---|
| N | 12 | 17 |
| Baseline | 21.2 | 6.3 |
| Δ to placebo | | −9.63 |
| p value | | 0.0241 |

FIG. 18C: Change from baseline in Walk-12 Total Score Week 26 Stage 3-5 BL TFC 0-6. The table below and FIG. 18C show no significant improvement in the Walk-12 TS in late stage pridopidine treated patients (BL TFC 0-6) at 26 weeks.

TABLE 71
Change from baseline in Walk-12 Total
Score Week 26 Stage 3-5 BL TFC 0-6

| | Placebo | 45 mg bid |
|---|---|---|
| N | 19 | 16 |
| Baseline | 56.6 | 55.4 |
| Δ to placebo | | −1.97 |
| p value | | 0.7524 |

FIG. 18D: Change from baseline in Walk-12 Total Score Week 52 Stage 1 BL TFC 11-13. The table below and FIG. 18D show a trend for improvement in the Walk-12 TS in 45 mg bid pridopidine treated HD1 patients at 52 weeks.

TABLE 72
Change from baseline in Walk-12 Total
Score Week 52 Stage 1 BL TFC 11-13

| | Placebo | 45 mg bid |
|---|---|---|
| N | 12 | 17 |
| Baseline | 21.2 | 6.3 |
| Δ to placebo | | −5.86 |
| p value | | 0.3018 |

FIG. 19A: Change from baseline in UHDRS Independence Scale Week 26 in late stage HD, BL TFC<7. The table below and FIG. 19A show no significant improvement in the UHDRS IS in pridopidine treated patients having BL TFC<7 at 26 weeks.

TABLE 73
Change from baseline in UHDRS Independence
Scale Week 26 in late stage HD BL TFC <7

| | Placebo | 45 mg bid |
|---|---|---|
| N | 19 | 16 |
| Baseline | 65.5 | 63.8 |
| Δ to placebo | | 0.3 |
| p value | | 0.8796 |

FIG. 19B: Change from baseline in UHDRS Independence Scale Week 26 BL TFC≥7. The table below and FIG. 19B show statistically significant improvement in the UHDRS IS in 45 mg bid pridopidine treated HD1 and HD2, TFC 7-13 patients at 26 weeks.

TABLE 74
Change from baseline in UHDRS Independence
Scale Week 26 in early stage HD, BL TFC ≥7

| | 45 mg bid |
|---|---|
| N | 59 |
| Wk26 Δ to placebo | 2.22 |
| p value | 0.0128 |

FIG. 19C: Change from baseline in UHDRS Independence Scale Week 52 BL TFC<7. The table below and FIG. 19C show no significant improvement in the UHDRS IS in pridopidine treated patients having baseline TFC<7 at 52 weeks.

TABLE 75
Change from baseline in UHDRS Independence
Scale Week 52, late HD, BL TFC <7.

| | Placebo | 45 mg bid |
|---|---|---|
| N | 19 | 16 |
| Baseline | 65.5 | 63.8 |
| Δ to placebo | | −1.85 |
| p value | | 0.5799 |

FIG. 19D: Change from baseline in UHDRS Independence Scale Week 52 BL TFC≥7. The table below and FIG. 19D show a trend for improvement in the UHDRS IS in 45 mg bid pridopidine treated HD1 and HD2, TFC 7-13 patients at 52 weeks.

TABLE 76
Change from baseline in UHDRS Independence
Scale Week 52, early HD, BL TFC ≥7

| | Placebo | 45 mg bid |
|---|---|---|
| N | 62 | 59 |
| Baseline | 79.8 | 79.4 |
| Δ to placebo | | 1.99 |
| p value | | 0.1047 |

FIGS. 20A-20P, 22, 23A-23B, 24A-24C are graphs comparing characteristics in early stage (TFC≥7, HD1 and HD2, TFC 7-13) or late stage (TFC<7) HD patients.

FIG. 20A: Change from baseline in UHDRS Total Functional Capacity Week 26 BL TFC<7. Table 77 below and FIG. 20A show no improvement in the UHDRS TFC in pridopidine treated late stage patients at 26 weeks.

TABLE 77
Change from baseline in
UHDRS Total Functional Capacity
Week 26, late HD BL TFC <7

| | Placebo | 45 mg bid |
|---|---|---|
| N | 19 | 16 |
| Baseline | 4.5 | 4.1 |
| Δ to placebo | | −0.22 |
| p value | | 0.6478 |

FIG. 20B: Change from baseline in UHDRS Total Functional Capacity Week 26 BL TFC≥7. Table 78 below and FIG. 20B show statistically significant improvement in the UHDRS TFC in 45 mg bid treated HD1 and HD2, TFC 7-13 patients, at 26 weeks.

TABLE 78
Change from baseline in
UHDRS Total Functional Capacity
Week 26, early HD, BL TFC ≥7

| | Placebo | 45 mg bid |
|---|---|---|
| N | 62 | 59 |
| Baseline | 8.9 | 9.2 |
| Wk26 Δ to placebo | | 0.56 |
| p value | | 0.0359 |

FIG. 20C: Change from baseline in UHDRS TFC Finance ADL Week 26 BL TFC<7. Table 79 below and FIG. 20C show no improvement in the UHDRS TFC Finance ADL in pridopidine treated late stage patients at 26 weeks.

TABLE 79
Change from baseline in
UHDRS TFC Finance ADL
Week 26, late HD. BL TFC <7.

| | Placebo | 45 mg bid |
|---|---|---|
| N | 19 | 16 |
| Baseline | 2 | 2 |
| Δ to placebo | | −0.34 |
| p value | | 0.3239 |

FIG. 20D: Change from baseline in UHDRS TFC Finance ADL Week 26 BL TFC≥7. Table 80 below and FIG. 20D show statistically significant improvement in the UHDRS Finance ADL in 45 mg bid pridopidine treated HD1 and HD2, TFC 7-13 patients at 26 weeks.

TABLE 80
Change from baseline in UHDRS TFC
Finance ADL Week 26 BL TFC ≥7.

| | Placebo | 45 mg bid |
|---|---|---|
| N | 62 | 59 |
| Baseline | 4.6 | 4.7 |
| Δ to placebo | | 0.46 |
| p value | | 0.0114 |

FIG. 20E: Change from baseline in UHDRS TFC Finances Week 26 BL TFC<7. Table 81 below and FIG. 20E show no improvement in the UHDRS TFC finances in pridopidine treated late stage patients at 26 weeks.

TABLE 81
Change from baseline in UHDRS
TFC Finances Week 26 BL TFC <7

| | Placebo | 45 mg bid |
|---|---|---|
| N | 19 | 16 |
| Baseline | 0.5 | 0.5 |
| Δ to placebo | | −0.19 |
| p value | | 0.3508 |

FIG. 20F: Change from baseline in UHDRS TFC Finances Week 26 BL TFC≥7. Table 82 below and FIG. 20F show a trend for improvement in the UHDRS TFC finances in 45 mg bid HD1 and HD2, TFC 7-13 pridopidine treated patients at 26 weeks.

TABLE 82
Change from baseline in UHDRS
TFC Finances Week 26 BL TFC ≥7.

| | Placebo | 45 mg bid |
|---|---|---|
| N | 62 | 59 |
| Baseline | 2 | 2.2 |
| Δ to placebo | | 0.2 |
| p value | | 0.0853 |

FIG. 20G: Change from baseline in UHDRS TFC ADL Week 26 BL TFC<7. Table 83 below and FIG. 20G show no improvement in the UHDRS TFC ADL in pridopidine treated late stage patients at 26 weeks.

TABLE 83
Change from baseline in UHDRS
TFC ADL Week 26 BL TFC <7.

| | Placebo | 45 mg bid |
|---|---|---|
| N | 19 | 16 |
| Baseline | 1.5 | 1.5 |
| Δ to placebo | | −0.19 |
| p value | | 0.3596 |

FIG. 20H: Change from baseline in UHDRS TFC ADL Week 26 BL TFC≥7. Table 84 below and FIG. 20H show statistically significant improvement in the UHDRS TFC ADL in 45 mg bid pridopidine treated HD1 and HD2, TFC 7-13 patients, at 26 weeks.

TABLE 84
Change from baseline in UHDRS
TFC ADL Week 26 BL TFC ≥7.

| | Placebo | 45 mg bid |
|---|---|---|
| N | 62 | 59 |
| Baseline | 2.6 | 2.6 |
| Δ to placebo | | 0.24 |
| p value | | 0.0176 |

FIG. 20L Change from baseline in UHDRS Total Functional Capacity Week 52 BL TFC<7. Table 85 below and FIG. 20I show no improvement in the UHDRS Total Functional Capacity in pridopidine treated late stage patients at 52 weeks.

TABLE 85
Change from baseline in UHDRS Total
Functional Capacity Week 52 BL TFC <7.

| | Placebo | 45 mg bid |
|---|---|---|
| N | 19 | 16 |
| Baseline | 4.5 | 4.1 |
| Δ to placebo | | 0.07 |
| p value | | 0.9108 |

FIG. 20J: Change from baseline in UHDRS Total Functional Capacity Week 52 BL TFC≥7. Table 86 below and FIG. 20J show significant maintenance of functional capacity as measured by UHDRS TFC in 45 mg bid pridopidine treated HD1 and HD2, TFC 7-13 patients at 52 weeks.

TABLE 86
Change from baseline in UHDRS Total
Functional Capacity Week 52 BL TFC ≥7

| | Placebo | 45 mg bid |
|---|---|---|
| N | 62 | 59 |
| Baseline | 8.9 | 9.2 |
| Wk52 Δ to placebo | | 1.16 |
| p value | | 0.0003 |

FIG. 20K: Change from baseline in UHDRS TFC Finance ADL Week 52 BL TFC<7. Table 87 below and FIG. 20K show no improvement in the UHDRS TFC finance ADL in late stage pridopidine treated patients at 52 weeks.

TABLE 87
Change from baseline in UHDRS TFC
Finance ADL Week 52 BL TFC <7

| | Placebo | 45 mg bid |
|---|---|---|
| N | 19 | 16 |
| Baseline | 2 | 2 |
| Δ to placebo | | 0.01 |
| p value | | 0.9863 |

FIG. 20L: Change from baseline in UHDRS TFC Finance ADL week 52 BL TFC≥7. Table 88 below and FIG. 20L show statistically significant improvement in the UHDRS TFC finance ADL in 45 mg bid pridopidine treated HD1 and HD2, TFC 7-13 patients at 52 weeks.

TABLE 88
Change from baseline in UHDRS TFC
Finance ADL Week 52 BL TFC ≥7

| | Placebo | 45 mg bid |
|---|---|---|
| N | 62 | 59 |
| Baseline | 4.6 | 4.7 |
| Δ to placebo | | 0.72 |
| p value | | 0.0004 |

FIG. 20M: Change from baseline in UHDRS TFC Finances Week 52 BL TFC<7. Table 89 below and FIG. 20M show no improvement in the UHDRS TFC finances in pridopidine treated late stage patients at 52 weeks.

TABLE 89
Change from baseline in UHDRS TFC
Finances Week 52 BL TFC <7

| | Placebo | 45 mg bid |
|---|---|---|
| N | 19 | 16 |
| Baseline | 0.5 | 0.5 |
| Δ to placebo | | 0.29 |
| p value | | 0.2468 |

FIG. 20N: Change from baseline in UHDRS TFC Finances Week 52 BL TFC≥7. Table 90 below and FIG. 20N show statistically significant improvement in the UHDRS IS in 45 mg bid pridopidine treated HD1 and HD2, TFC 7-13 patients at 52 weeks.

TABLE 90
Change from baseline in UHDRS TFC
Finances Week 52 BL TFC ≥7.

| | Placebo | 45 mg bid |
|---|---|---|
| N | 62 | 59 |
| Baseline | 2 | 2.2 |
| Δ to placebo | | 0.35 |
| p value | | 0.0171 |

FIG. 20O: Change from baseline in UHDRS TFC ADL Week 52 BL TFC<7. Table 91 below and FIG. 20O show no improvement in the UHDRS TFC ADL in pridopidine treated late stage patients at 52 weeks.

TABLE 91
Change from baseline in UHDRS
TFC ADL Week 52 BL TFC <7.

| | Placebo | 45 mg bid |
|---|---|---|
| N | 19 | 16 |
| Baseline | 1.5 | 1.5 |
| Δ to placebo | | −0.33 |
| p value | | 0.178 |

FIG. 20P: Change from baseline in UHDRS TFC ADL Week 52 BL TFC≥7. Table 92 below and FIG. 20P show statistically significant improvement in the UHDRS TFC ADL in 45 mg bid pridopidine treated HD1 and HD2, TFC 7-13 patients at 52 weeks.

TABLE 92
Change from baseline in UHDRS
TFC ADL Week 52 BL TFC ≥7.

| | Placebo | 45 mg bid |
|---|---|---|
| N | 62 | 59 |
| Baseline | 2.6 | 2.6 |
| Wk 52 Δ to placebo | | 0.35 |
| p value | | 0.0019 |

FIGS. 21A-21E show bar graphs of changes in UHDRS TMS Finger Tap scores in 26- and 52-week patient groups.

FIG. 21A: Change from Baseline in UHDRS TMS Finger Taps ALL Week 26. Table 93 below provides P-Values corresponding to FIG. 21A. Table 93 below and FIG. 21A show a trend for improvement in the UHDRS TMS finger taps in pridopidine 45 mg bid treated patients, at 26 weeks.

TABLE 93
Change from Baseline in UHDRS
TMS Finger Taps, all HD stages, Week 26

| | Placebo | 45 mg bid |
|---|---|---|
| N | 81 | 75 |
| Baseline | 3.8 | 3.5 |
| Δ to placebo | | −0.3 |
| p value | | 0.1466 |

FIG. 21B: Change from Baseline in UHDRS TMS Finger Taps: Week 26 patients with baseline total functional capacity (BL TFC)≥9 and CAG Repeats >44. Table 94 below provides the P-values corresponding to FIG. 21B. The table below and FIG. 21B show statistically significant improvement in the UHDRS TMS finger taps in 45 mg bid pridopidine treated patients having BL TFC≥9 and greater than 44 CAG repeats in their Htt gene at 26 weeks.

TABLE 94
Change from Baseline in UHDRS TMS Finger Taps:
Week 26 patients with baseline total functional
capacity (BL TFC) ≥9 and CAG Repeats >44

| | Placebo | 45 mg bid |
|---|---|---|
| N | 13 | 15 |
| Baseline | 2.6 | 2.7 |
| Δ to placebo | | −0.86 |
| p value | | 0.0499 |

FIG. 21C: Change from baseline in UHDRS TMS Finger Taps: Week 26 patients with BL TFC≥9, CAG Repeats <44 and patients who represent three least severe TMS quarters (BL TMS 1st 3 Qs). Table 95 below provides the P-values corresponding to FIG. 21C. The table below and FIG. 21C show statistically significant improvement in the UHDRS TMS finger taps in 45 mg bid pridopidine treated patients having BL TFC≥9 and less than 44 CAG repeats in their Htt gene at 26 weeks.

TABLE 95
Change from baseline in UHDRS TMS
Finger Taps: Week 26 patients with
BL TFC ≥9, CAG Repeats <44
and patients who represent three least
severe TMS quarters (BL TMS 1st 3 Qs)

| | Placebo | 45 mg bid |
|---|---|---|
| N | 13 | 15 |
| Baseline | 2.6 | 2.7 |
| Δ to placebo | | −0.87 |
| p value | | 0.05 |

FIG. 21D: Change from baseline in UHDRS TMS Finger Taps: Patients who have completed 52 weeks of treatment: UHDRS TMS Finger Tap score at week 26. Table 96 below provides the P-values corresponding to FIG. 21D. Table 96 below and FIG. 21D show statistically significant improvement in the UHDRS TMS finger taps in 45 mg bid pridopidine treated patients who completed 52 weeks, at 26 weeks.

TABLE 96
Change from baseline in UHDRS TMS Finger
Taps: Patients who have completed 52 weeks of
treatment: UHDRS TMS Finger Tap score at week 26.

| | Placebo | 45 mg bid |
|---|---|---|
| N | 52 | 43 |
| Baseline | 3.8 | 3.2 |
| Δ to placebo | | −0.59 |
| p value | | 0.0182 |

FIG. 21E: Change from baseline in UHDRS TMS Finger Taps: Patients who have completed 52 weeks of treatment: UHDRS TMS Finger Tap score at week 52. Table 97 below provides the P-values corresponding to FIG. 21E. Table 97 below and FIG. 21E show a trend for improvement in the UHDRS TMS finger taps in ALL pridopidine treated patients at 52 weeks.

TABLE 97
Change from baseline in UHDRS TMS Finger
Taps: Patients who have completed 52 weeks of
treatment: UHDRS TMS Finger Tap score at week 52

| | Placebo | 45 mg bid |
|---|---|---|
| N | 52 | 43 |
| Baseline | 3.8 | 3.2 |
| Δ to placebo | | −0.31 |
| p value | | 0.2091 |

FIG. 22: Change from baseline in UHDRS TMS Finger Tapping+Pronate-Supinate Hands: Patients who have completed 52 weeks of treatment—score at week 26. Table 98 below provides the P-values corresponding to FIG. 22. Table 98 below and FIG. 22 show statistically significant improvement in the UHDRS TMS finger taps and Pronate-Supinate Hands in 45 mg bid pridopidine treated patients who completed 52 weeks, at 26 weeks.

TABLE 98
Change from baseline in UHDRS TMS
Finger Tapping + Pronate-Supinate Hands:
Patients who have completed 52
weeks of treatment-score at week 26

| | Placebo | 45 mg bid |
|---|---|---|
| N | 52 | 43 |
| Baseline | 7.1 | 6.1 |
| Δ to placebo | | −0.79 |
| p value | | 0.0294 |

FIG. 23A: Change from baseline in UHDRS TMS Gait and Balance: Gait and balance scores at week 26 for patients with BL TFC≥7. Table 99 below provides the P-values corresponding to FIG. 23A. Table 99 below and FIG. 23A show a trend for improvement in the UHDRS TMS gait and balance in 45 mg bid pridopidine treated HD1 and HD2, TFC 7-13 patients at 26 weeks.

TABLE 99
Change from baseline in UHDRS TMS
Gait and Balance: Gait and balance scores
at week 26 for patients with BL TFC ≥7

| | Placebo | 45 mg bid |
|---|---|---|
| N | 62 | 59 |
| Baseline | 3.7 | 3.7 |
| Δ to placebo | | −0.48 |
| p value | | 0.0563 |

FIG. 23B: Change from baseline in UHDRS TMS Gait and Balance: Gait and balance scores at week 52 for patients with BL TFC≥7. Table 100 below provides the P-values corresponding to FIG. 23B. Table 100 below and FIG. 23B show a trend for improvement in the UHDRS TMS gait and balance in pridopidine treated HD1 and HD2, TFC 7-13 patients at 52 weeks.

TABLE 100
Change from baseline in UHDRS TMS
Gait and Balance: Gait and balance scores
at week 52 for patients with BL TFC ≥7.

| | Placebo | 45 mg bid |
|---|---|---|
| N | 62 | 59 |
| Baseline | 3.2 | 3.7 |
| Δ to placebo | | −0.41 |
| p value | | 0.1811 |

FIG. 24A: Change from baseline in UHDRS TMS Dystonia: UHDRS TMS Dystonia scores for patients with BL TFC≥9 AND CAG Repeats <44 at week 26. Table 101 below provides the P-values corresponding to FIG. 24A. Patients with baseline TFC≥9, show statistically significant improvement in the UHDRS TMS Dystonia score at 45 mg bid pridopidine for 26 weeks.

TABLE 101
Change from baseline in UHDRS
TMS Dystonia scores for patients with BL
TFC ≥9 AND CAG Repeats <44 at week 26

| | Placebo | 45 mg bid |
|---|---|---|
| N | 13 | 15 |
| Baseline | 3.8 | 1.7 |
| Δ to placebo | | −1.54 |
| p value | | 0.0313 |

FIG. 24B: Change from baseline in UHDRS TMS Dystonia: UHDRS TMS Dystonia scores for patients with CAG Repeats <44 AND BL TMS 1st 3 Qs at week 26. Table 102 below provides the P-values corresponding to FIG. 24B. Patients with baseline TMS who represent three least severe TMS quarters and less than 44 CAG repeats in their Htt gene, show statistically significant improvement in the UHDRS TMS Dystonia score at 45 mg bid pridopidine for 26 weeks.

TABLE 102
Change from baseline in UHDRS TMS Dystonia:
UHDRS TMS Dystonia scores for patients with
CAG Repeats <44 AND BL TMS 1st 3 Qs at week 26.

| | Placebo | 45 mg bid |
|---|---|---|
| N | 29 | 29 |
| Baseline | 3 | 2.6 |
| Δ to placebo | | −1.04 |
| p value | | 0.0437 |

FIG. 24C: Change from baseline in UHDRS TMS Dystonia: UHDRS TMS Dystonia scores for patients with BL TFC≥9 and CAG Repeats <44 and BL TMS 1st 3 Qs at week 26. Table 103 below provides the P-Values corresponding to FIG. 24C. Patients with baseline TFC≥9, baseline TMS representing three least severe TMS quarters and less than 44 CAG repeats in their Htt gene, show statistically significant improvement in the UHDRS TMS Dystonia score at 45 mg bid pridopidine for 26 weeks.

TABLE 103
Change from baseline in UHDRS
TMS Dystonia: UHDRS TMS Dystonia scores
for patients with BL TFC ≥9 and CAG
Repeats <44 and BL TMS 1st 3 Qs at week 26

| | Placebo | 45 mg bid |
|---|---|---|
| N | 13 | 15 |
| Baseline | 3.8 | 1.7 |
| Δ to placebo | | −1.53 |
| p value | | 0.0349 |

FIGS. 25A, 25B and 25C are bar graphs showing changes from baseline in Gait and Balance scores at week 12 (FIG. 25A); week 20 (FIG. 25B); and week 26 (FIG. 25C). Y-axes are changes in UHDRS Gait and Balance score.

FIGS. 26A-26F provide bar graphs or line graphs showing changes from baseline of UHDRS TFC scores in 26- and 52-week patient groups.

FIGS. 26A and 26B show change from baseline in UHDRS TFC score over time. Y axes represents change in TFC score, X axes represents pridopidine treatment time, in weeks. FIG. 26A shows the trend in full analysis set after 52 weeks. FIG. 26B shows trends in patients having BL TFC≥7 (n=54-62).

FIG. 26C: Change from baseline in UHDRS Total Functional Capacity for patients with BL CAG Repeats <44 at week 26. Table 104 below provides the P-values corresponding to FIG. 26C. There is a trend for improvement in the 45 mg bid treated group compared to placebo.

TABLE 104
Change from baseline in UHDRS Total
Functional Capacity for patients with BL
CAG Repeats <44 at week 26

| | Placebo | 45 mg bid |
|---|---|---|
| N | 37 | 37 |
| Baseline | 7.4 | 7.9 |
| Δ to placebo | | 0.6 |
| p value | | 0.056 |

FIG. 26D: Change from baseline in UHDRS Total Functional Capacity for patients with BL TFC≥9 or CAG Repeats <44 at week 26. Table 105 below provides the P-values corresponding to FIG. 26D. Significant improvement is observed in patients treated with 45 mg bid compared to placebo.

TABLE 105
Change from baseline in UHDRS Total
Functional Capacity for patients with BL
TFC ≥9 or CAG Repeats <44 at week 26

| | Placebo | 45 mg bid |
|---|---|---|
| N | 56 | 56 |
| Baseline | 8.5 | 8.8 |
| Δ to placebo | | 0.56 |
| p value | | 0.0321 |

FIG. 26E: Change from baseline in UHDRS Total Functional Capacity for patients with BL CAG Repeats <44 AND BL TMS 1st 3 Qs at week 26. Table 106 below provides the P-values corresponding to FIG. 26E. Significant improvement is observed in patients treated with 45 mg bid compared to placebo.

TABLE 106
Change from baseline in UHDRS Total
Functional Capacity for patients with BL CAG
Repeats <44 AND BL TMS 1st 3 Qs at week 26

| | Placebo | 45 mg bid |
|---|---|---|
| N | 29 | 29 |
| Baseline | 8 | 8.7 |
| Δ to placebo | | 0.73 |
| p value | | 0.0469 |

FIG. 26F: Change from baseline in UHDRS Total Functional Capacity for patients with BL TFC≥9 or BL TMS 1st 3 Qs at week 26. Table 107 below provides the P-values corresponding to FIG. 26F. A trend for improvement is observed in patients treated with 45 mg bid compared to placebo.

TABLE 107
Change from baseline in UHDRS Total
Functional Capacity for patients with BL
TFC ≥9 or BL TMS 1st 3 Qs at week 26.

| | Placebo | 45 mg bid |
|---|---|---|
| N | 30 | 32 |
| Baseline | 10.3 | 10.5 |
| Δ to placebo | | 0.69 |
| p value | | 0.0601 |

FIGS. 27A, 27B, and 27C: Change from baseline in TFC score in all pridopidine treated HD patients. Doses at week 12 (FIG. 27A), week 20 (FIG. 27B) and week 26 (FIG. 27C). Score is adjusted means+SE of change in TFC for full analysis set.

FIGS. 28A, 28B, and 28C: Change from baseline in TFC ADL & Finances score in all pridopidine treated HD patients. Doses at week 12 (FIG. 28A), week 20 (FIG. 28B) and week 26 (FIG. 28C). Score is adjusted means+SE of change in TFC Finance and ADL for full analysis set.

FIGS. 29A-29R are bar graphs showing changes from baseline of UHDRS TFC Finances and UHDRS TFC Finances and ADL scores in 26- and 52-week patient groups by quartiles.

FIG. 29A: Change from baseline in UHDRS TFC Finances score for patients with TMS 1st Q (first least severe TMS quarter) at week 26. Table 108 below provides the P-values corresponding to FIG. 29A. Significant improvement in TFC finances in 45 mg bid pridopidine administered first least severe TMS quarter patients for 26 weeks.

TABLE 108
Change from baseline in UHDRS TFC
Finances score for patients with TMS 1st Q
(first least severe TMS quarter) at week 26

| | Placebo | 45 mg bid |
|---|---|---|
| N | 21 | 24 |
| Baseline | 2.2 | 2.1 |
| Δ to placebo | | 0.38 |
| p value | | 0.0347 |

FIG. 29B: Change from baseline in UHDRS TFC Finances score for patients with TMS 1st Q at week 52. Table 109 below provides the P-values corresponding to FIG. 29B. A trend towards improvement in TFC finances was observed in 45 mg bid pridopidine administered first least severe TMS quarter patients for 52 weeks.

TABLE 109
Change from baseline in UHDRS
TFC Finances score for
patients with TMS 1st Q at week 52

| | Placebo | 45 mg bid |
|---|---|---|
| N | 21 | 24 |
| Baseline | 2.2 | 2.1 |
| Δ to placebo | | 0.43 |
| p value | | 0.0673 |

FIG. 29C: Change from baseline in UHDRS TFC Finances score for patients with TMS 1st 2Qs (first two least severe TMS quarters) at week 26. Table 110 below provides the P-values corresponding to FIG. 29C. A trend towards improvement in TFC finances is observed in 45 mg bid pridopidine administered first two least severe TMS quarter patients for 26 weeks.

TABLE 110
Change from baseline in UHDRS TFC
Finances score for patients with TMS 1st 2 Qs
(first two least severe TMS quarters) at week 26

| | Placebo | 45 mg bid |
|---|---|---|
| N | 42 | 44 |
| Baseline | 2 | 2.1 |
| Δ to placebo | | 0.33 |
| p value | | 0.0566 |

FIG. 29D: Change from baseline in UHDRS TFC Finances score for patients with TMS 1st 2Qs at week 52. Table 111 below provides the P-values corresponding to FIG. 29D. Significant improvement in TFC finances was observed in 45 mg bid pridopidine administered first two least severe TMS quarters patients for 52 weeks.

TABLE 111
Change from baseline in UHDRS
TFC Finances score for patients
with TMS 1st 2 Qs at week 52.

| | Placebo | 45 mg bid |
|---|---|---|
| N | 42 | 44 |
| Baseline | 2 | 2.1 |
| Δ to placebo | | 0.29 |
| p value | | 0.0299 |

FIG. 29E: Change from baseline in UHDRS TFC Finances score for patients with TMS 1st 3Qs at week 26. Table 112 below provides the P-values corresponding to FIG. 29E. A trend towards improvement is observed in TFC finances in 45 mg bid pridopidine administered first three least severe TMS quarter patients for 26 weeks.

TABLE 112
Change from baseline in UHDRS
TFC Finances score for patients
with TMS 1st 3 Qs at week 26.

| | Placebo | 45 mg bid |
|---|---|---|
| N | 58 | 59 |
| Baseline | 1.8 | 2 |
| Δ to placebo | | 0.12 |
| p value | | 0.315 |

FIG. 29F: Change from baseline in UHDRS TFC Finances score for patients with TMS 1st 3Qs at week 52. Table 113 below provides the P-values corresponding to FIG. 29F. Significant improvement in TFC finances was observed in 45 mg bid pridopidine administered first three least severe TMS quarter patients for 52 weeks.

TABLE 113
Change from baseline in UHDRS
TFC Finances score for patients
with TMS 1st 3 Qs at week 52.

| | Placebo | 45 mg bid |
|---|---|---|
| N | 58 | 59 |
| Baseline | 1.8 | 2 |
| Δ to placebo | | 0.39 |
| p value | | 0.0072 |

FIG. 29G: Change from baseline in UHDRS TFC Finance and ADL score for patients with BL TFC≥9 at week 26. Table 114 below provides the P-values corresponding to FIG. 29G. Significant improvement in TFC finance and ADL was observed in 45 mg bid pridopidine administered patients having with baseline TFC≥9 for 26 weeks.

TABLE 114
Change from baseline in UHDRS TFC
Finance and ADL score for patients
with BL TFC ≥9 at week 26

| | Placebo | 45 mg bid |
|---|---|---|
| N | 32 | 34 |
| Baseline | 5.2 | 5.1 |
| Δ to placebo | | 0.53 |
| p value | | 0.0143 |

FIG. 29H: Change from baseline in UHDRS TFC Finance and ADL score for patients with BL CAG Repeat >44 at week 26. Table 115 below provides the P-values corresponding to FIG. 29H. Significant improvement in TFC finance and ADL was observed in 45 mg bid pridopidine administered patients having more than 44 CAG repeats in their Htt gene for 26 weeks.

TABLE 115
Change from baseline in UHDRS TFC
Finance and ADL score for patients
with BL CAG Repeat >44 at week 26.

| | Placebo | 45 mg bid |
|---|---|---|
| N | 37 | 37 |
| Baseline | 3.7 | 4.1 |
| Δ to placebo | | 0.55 |
| p value | | 0.017 |

FIG. 29L Change from baseline in UHDRS TFC Finance and ADL score for patients with BL TFC≥9 and CAG Repeat >44 at week 26. Table 116 below provides the P-Values corresponding to FIG. 29I. Significant improvement in TFC finance and ADL was observed in 45 mg bid pridopidine administered patients having baseline TFC≥9 and more than 44 CAG repeats in their HYtt gene, for 26 weeks.

TABLE 116
Change from baseline in UHDRS TFC Finance
and ADL score for patients with BL
TFC ≥9 and CAG Repeat >44 at week 26.

| | Placebo | 45 mg bid |
|---|---|---|
| N | 13 | 15 |
| Baseline | 5.1 | 5.2 |
| Δ to placebo | | 0.74 |
| p value | | 0.0296 |

FIG. 29J: Change from baseline in UHDRS TFC Finance and ADL score for patients with BL TFC≥9 or CAG Repeat >44 at week 26. Table 117 below provides the P-values corresponding to FIG. 29J Significant improvement in TFC finance and ADL was observed in 45 mg bid pridopidine administered patients having baseline TFC≥9 or more than 44 CAG repeats in their Htt gene, for 26 weeks.

TABLE 117
Change from baseline in UHDRS TFC Finance
and ADL score for patients with BL
TFC ≥9 or CAG Repeat >44 at week 26.

| | Placebo | 45 mg bid |
|---|---|---|
| N | 56 | 56 |
| Baseline | 4.2 | 4.5 |
| Δ to placebo | | 0.5 |
| p value | | 0.0055 |

FIG. 29K: Change from baseline in UHDRS TFC Finance and ADL score for patients with CAG Repeats <44 and BL TMS 1st 3 Qs at week 26. Table 118 below provides the P-values corresponding to FIG. 29K. Significant improvement in TFC finance and ADL was observed in 45 mg bid pridopidine administered patients having baseline TMS first 3 quarters and less than 44 CAG repeats in their Htt gene, for 26 weeks.

TABLE 118
Change from baseline in UHDRS TFC Finance
and ADL score for patients with CAG
Repeats <44 and BL, TMS 1st 3 Qs at week 26.

| | Placebo | 45 mg bid |
|---|---|---|
| N | 29 | 29 |
| Baseline | 4.1 | 4.6 |
| Δ to placebo | | 0.59 |
| p value | | 0.0236 |

FIG. 29L: Change from baseline in UHDRS TFC Finance and ADL score for patients with BL TFC≥9 and CAG Repeats <44 and BL TMS 1st 3 Qs at week 26. Table 119 below provides the P-values corresponding to FIG. 29L. Significant improvement in TFC finance and ADL was observed in 45 mg bid pridopidine administered patients having baseline TFC≥9 and less than 44 CAG repeats in their Htt gene, for 26 weeks.

TABLE 119
Change from baseline in UHDRS
TFC Finance and ADL score for
patients with BL TFC ≥9 and CAG
Repeats <44 and BL TMS 1st 3 Qs at week 26.

| | Placebo | 45 mg bid |
|---|---|---|
| N | 13 | 15 |
| Baseline | 5.1 | 5.2 |
| Δ to placebo | | 0.74 |
| p value | | 0.0315 |

FIG. 29M: Change from baseline in UHDRS TFC Finance and ADL score for patients with BL TFC≥9 and BL TMS 1st 3 Qs at week 26. Table 120 below provides the P-values corresponding to FIG. 29M. Significant improvement in TFC finance and ADL was observed in 45 mg bid pridopidine administered patients having baseline TFC≥9 or less than 44 CAG repeats in their Htt gene or baseline TMS first three quarters, for 26 weeks.

TABLE 120
Change from baseline in UHDRS TFC Finance
and ADL score for patients with BL
TFC ≥9 and BL TMS 1st 3 Qs at week 26

| | Placebo | 45 mg bid |
|---|---|---|
| N | 30 | 32 |
| Baseline | 5.1 | 5.1 |
| Δ to placebo | | 0.53 |
| p value | | 0.018 |

FIG. 29N: Change from baseline in UHDRS TFC Finance and ADL score for patients with TMS 1st Q at week 26. Table 121 below provides the P-values corresponding to FIG. 29N. Significant improvement in TFC finance and ADL was observed in 45 mg bid pridopidine administered patients with TMS first three quarters, for 26 weeks.

TABLE 121
Change from baseline in UHDRS
TFC Finance and ADL score for
patients with TMS 1st Q at week 26.

| | Placebo | 45 mg bid |
|---|---|---|
| N | 21 | 24 |
| Baseline | 4.9 | 4.8 |
| Δ to placebo | | 0.63 |
| p value | | 0.038 |

FIG. 29O: Change from baseline in UHDRS TFC Finance and ADL score for patients with TMS 1st Q at week 52. Table 122 below provides the P-Values corresponding to FIG. 29O. Significant improvement in TFC finance and ADL was observed in 45 mg bid pridopidine administered patients with TMS first quarter, for 52 weeks.

TABLE 122
Change from baseline in UHDRS
TFC Finance and ADL score for
patients with TMS 1st Q at week 52

| | Placebo | 45 mg bid |
|---|---|---|
| N | 21 | 24 |
| Baseline | 4.9 | 4.8 |
| Δ to placebo | | 0.71 |
| p value | | 0.0319 |

FIG. 29P: Change from baseline in UHDRS TFC Finance and ADL score for patients with TMS 1st 2Qs at week 26. Table 123 below provides the P-values corresponding to FIG. 29P. Significant improvement in TFC finance and ADL was observed in 45 mg bid pridopidine administered patients with TMS first two quarters, for 26 weeks.

TABLE 123
Change from baseline in UHDRS
TFC Finance and ADL score for
patients with TMS 1st 2 Qs at week 26.

| | Placebo | 45 mg bid |
|---|---|---|
| N | 42 | 44 |
| Baseline | 4.5 | 4.7 |
| Δ to placebo | | 0.48 |
| p value | | 0.045 |

FIG. 29Q: Change from baseline in UHDRS TFC Finance and ADL score for patients with TMS 1st 2Qs at week 52. Table 124 below provides the P-values corresponding to FIG. 29Q. Significant improvement in TFC finance and ADL was observed in 45 mg bid pridopidine administered patients with TMS first two quarters, for 52 weeks.

TABLE 124
Change from baseline in UHDRS
TFC Finance and ADL score for
patients with TMS 1st 2 Qs at week 52.

| | Placebo | 45 mg bid |
|---|---|---|
| N | 42 | 44 |
| Baseline | 4.5 | 4.7 |
| Δ to placebo | | 0.47 |
| p value | | 0.0294 |

FIG. 29R: Change from baseline in UHDRS TFC Finance and ADL score for patients with TMS 1st 3Qs at week 52. Table 125 below provides the P-values corresponding to FIG. 29R. Significant improvement in TFC finance and ADL was observed in 45 mg bid pridopidine administered patients with TMS first three quarters, for 52 weeks.

TABLE 125
Change from baseline in UHDRS
TFC Finance and ADL score for
patients with TMS 1st 3 Qs at week 52

| | Placebo | 45 mg bid |
|---|---|---|
| N | 58 | 59 |
| Baseline | 4.3 | 4.5 |
| Δ to placebo | | 0.52 |
| p value | | 0.0122 |

FIGS. 30A and 30B: General information regarding Finger tapping (Q-Motor tap measurements). FIG. 30A shows a drawing of subject's arm with tapper. FIG. 30B shows normal and aberrant tapping measurements.

FIGS. 31A and 31B: Q-Motor tap measurements: A well-validated objective measure. (Bechtel 2010).

FIG. 32A: Improvement in objective pharmacodynamic measures of motor control: change from baseline in Q-Motor: Tap-Speed-Inter-Onset-interval-MN-Hand (sec), Week 52 FAS. Table 126 below provides data and the P-values corresponding to FIG. 32A. A trend towards improvement was noted in 45 mg bid treated patients.

TABLE 126
Change from baseline in
Tap-Speed-Inter-Onset-interval-MN-Hand
(sec), Week 52, all HD stages

| | Placebo | 45 mg bid |
|---|---|---|
| N | 81 | 75 |
| Baseline | 0.4065 | 0.4154 |
| Δ to placebo | | −0.0402 |
| p value | | 0.1956 |

FIG. 32B: Improvement in objective pharmacodynamic measures of motor control: change from baseline in Q-Motor: Tap-Speed-Inter-Onset-interval-MN-Hand (sec), Week 52 in pridopidine treated HD1 and HD2, TFC 7-13 patients. Table 127 below provides the data and P-values corresponding to FIG. 32B. A trend towards improvement was noted in the 45 mg bid treatment arm.

TABLE 127
Tap-Speed-Inter-Onset-interval-MN-Hand (sec),
Week 52 in pridopidine treated HD1
and HD2, TFC 7-13 patients

| | Placebo | 45 mg bid |
|---|---|---|
| N | 62 | 59 |
| Baseline | 0.3725 | 0.3605 |
| Δ to placebo | | −0.0351 |
| p value | | 0.1347 |

FIG. 32C: Improvement in objective pharmacodynamic measures of motor control, change from baseline in Q-Motor: Pro-Sup-Frequency-MN-Hand (Hz), Week 52 FAS. Table 128 below provides the data and P-values corresponding to FIG. 32C. A trend towards improvement was noted in 45 mg bid treated patients.

TABLE 128
Change from baseline in Pro-Sup-Frequency-MN-Hand (Hz),
Week 52, all HD stages

| | Placebo | 45 mg bid |
|---|---|---|
| N | 81 | 75 |
| Baseline | 1.6686 | 1.7789 |
| Wk 52 Δ to placebo | | 0.0599 |
| p value | | 0.3122 |

FIG. 32D: Improvement in objective pharmacodynamic measures of motor control, change from baseline in Q-Motor: Pro-Sup-Frequency-MN-Hand (Hz), Week 52 in pridopidine treated HD1 and HD2, TFC 7-13 patients. Table 129 below provides the data and P-values corresponding to FIG. 32D. A trend towards improvement was noted in 45 mg bid treated patients.

TABLE 129
Pro-Sup-Frequency-MN-Hand (Hz),
Week 52 in pridopidine treated HD1
and HD2, TFC 7-13 patients

| | Placebo | 45 mg bid |
|---|---|---|
| N | 62 | 59 |
| Baseline | 1.77 | 1.8513 |
| Wk 52 Δ to placebo | | 0.1195 |
| p value | | 0.0692 |

FIG. 33: Change from baseline in Cognitive Assessment Battery Hopkins Verbal Learning Test, revised (CAB HVLT-R) score for patients at week 52. Table 130 below provides the P-values corresponding to FIG. 33. A trend towards improvement in CAB HVLT-R score was observed in 45 mg bid pridopidine administered patients for 52 weeks.

TABLE 130
Change from baseline in
Cognitive Assessment Battery
Hopkins Verbal Learning
Test, revised (CAB HVLT-R)
score for all HD stages at week 52

| | Placebo | 45 mg bid |
|---|---|---|
| N | 81 | 75 |
| Baseline | 19.3 | 19.5 |
| Δ to placebo | | −2.21 |
| p value | | 0.0517 |

FIG. 34A: Change from baseline in Cognitive Assessment Battery CAB Trail Making Test score for patients at week 52. Table 131 below provides the P-values corresponding to FIG. 34A. A trend towards improvement in CAB Trail making test score was observed in pridopidine 45 mg bid administered patients, for 52 weeks.

TABLE 131
Change from baseline in Cognitive
Assessment Battery CAB Trail Making
Test score at week 52, all HD stages

| | Placebo | 45 mg bid |
|---|---|---|
| N | 81 | 75 |
| Baseline | −184.7 | −181.6 |
| Δ to placebo | | −13.56 |
| p value | | 0.0773 |

FIG. 34B: Change from baseline in Cognitive Assessment Battery CAB Paced Tapping at 3 Hz at 52 weeks. Table 132 below provides data and the P-values corresponding to FIG. 34B. A significant improvement vs placebo was observed for was in pridopidine 45 mg bid administered patients for 52 weeks.

TABLE 132
Change from baseline in Cognitive Assessment
Battery CAB Paced Tapping at 3 Hz at 52 weeks.

| | Placebo | 45 mg bid |
|---|---|---|
| N | 81 | 75 |
| Baseline | 5.935 | 6.035 |
| Δ to placebo | | 1.3234 |
| p value | | 0.0402 |

FIG. 35: Annual rates of decline (y axis) in TFC are higher in earlier stages of disease (Marder 2000).

FIG. 36A: Mean change in TFC from baseline in (1) Open-label Extension Study of Pridopidine (ACR16, 45 mg bid) in the Symptomatic Treatment of Huntington Disease (OPEN-HART) (n=50), (2) Co-Enzyme Q10 And Remacemide: Evaluation in HD (CARE-HD) (n=80) (Kieburtz 2001) and (3) Coenzyme Q10 in Huntington Disease (HD) (2CARE) (n=213): TFC Score Change From Baseline (non-matched cohorts). The circle over the 12 months points reflects ˜1-point difference showing less functional decline in Open-HART subjects treated with Pridopidine 45 mg bid compared to patients in the 2CARE and CARE-HD studies. Less functional decline (˜1-point difference) in OPEN-HART (pridopidine 45 mg bid) compared to 2CARE and CARE-HD was maintained up to 36 months

FIG. 36B: Change from baseline in TFC score plotted over time in Week 52 in pridopidine treated HD1 and HD2, TFC 7-13 treated subjects (n=59-62) in PRIDE-HD trial. The dark line with diamond represents placebo; line with open circle represents 45 mg bid, Y axis represents change from baseline in TFC score from baseline, x axis represents treatment time in weeks.

FIGS. 37A-37C are graphs which show multiple ambulation-related endpoints demonstrating trends favoring pridopidine in early HD (early HD stage 1-2 patients).

FIG. 37A: UHDRS TMS Gait: Early HD at 52 weeks. Table 133 below provides data and the P-values corresponding to FIG. 37A.

TABLE 133
Change in UHDRS TMS Gait: Early
HD at 52 weeks, early HD

| | Placebo | 45 mg bid |
|---|---|---|
| N | 62 | 59 |
| Baseline | 0.9 | 1.1 |
| Wk 52 Δ to placebo | | −0.21 |
| p value | | 0.0855 |

FIG. 37B: Timed Up and Go Test (sec): Pridopidine treated HD1 and HD2, TFC 7-13 patients at 52 weeks. Table 134 below provides data and the P-values corresponding to FIG. 37B. A trend for improvement is observed for patients treated with pridopidine 45 mg bid vs placebo.

TABLE 134
Change in Timed Up and Go Test
(sec) at 52 weeks in early HD

| | Placebo | 45 mg bid |
|---|---|---|
| N | 62 | 59 |
| Baseline | 10 | 11.7 |
| Wk 52 Δ to placebo | | −1.61 |
| p value | | 0.1348 |

FIG. 37C: Walk-12 improved in pridopidine treated HD1 patients at 52 weeks. Table 135 below provides data and the P-values corresponding to FIG. 37C.

TABLE 135
Change in Walk-12 at week 52
in HD1 (TFC 11-13) patients

| | Placebo | 45 mg bid |
|---|---|---|
| N | 12 | 17 |
| Baseline | 21.2 | 6.3 |
| Δ to placebo | | −5.86 |
| p value | | 0.3018 |

FIG. 37D Week 26 week 52, Pridopidine treated HD1 patients for Involuntary movements: Total Maximal Chorea (TMC). Table 136 below provides the data and P-values corresponding to FIG. 37D. A trend for improvement is observed for patients treated with pridopidine 45 mg bid vs placebo.

TABLE 136
Change in Involuntary movements:
Total Maximal Chorea (TMC).
At week 26 in HD1 (TFC 11-13) patients

| | Placebo | 45 mg bid |
|---|---|---|
| N | 12 | 17 |
| Baseline | 12 | 17 |
| Δ of placebo | | −1.4 |
| p value | | 0.1805 |

FIG. 38: Change from baseline in TMS plotted over time in HD1 patients. Line with open circle represents 45 mg bid. 45 mg bid shows improvement in TMS score after 52 weeks. Y axis represents change from baseline in TMS from baseline, x axis represents treatment time in weeks.

FIGS. 39A-39D. TFC change from baseline vs. placebo for pridopidine 45 mg bid at weeks 26 and 52 in all HD stages (FIG. 39A 26 weeks and 39B 52 weeks) and early HD (TFC>=7) participants (FIG. 39C 26 weeks and 39D 52 weeks). Full analysis set with MMRM analysis; Mean±SEM. Tables 137(A-D) below provide the data and P-values corresponding to FIGS. 39A-D.

TABLE 137A
TFC change at 26 weeks in all
HD stages, corresponds to FIG. 39A

| | Placebo | 45 mg bid |
|---|---|---|
| N | 81 | 75 |
| Wk 26 Δ to placebo | | 0.34 |
| P value | | 0.15 |

TABLE 137B
TFC change at 52 weeks, in all
HD stages, corresponds to FIG. 39B

| | Placebo | 45 mg bid |
|---|---|---|
| N | 81 | 75 |
| Wk 52 Δ to placebo | | 0.87 |
| P value | | 0.0032 |

TABLE 137C
TFC change at 26 weeks, in early
HD patients corresponds to FIG. 39C

| | Placebo | 45 mg bid |
|---|---|---|
| N | 62 | 59 |
| Wk 26 Δ to placebo | | 0.56 |
| P value | | 0.036 |

TABLE 137D
TFC change at 52 weeks, in early HD
patients corresponds to FIG. 39D

| | Placebo | 45 mg bid |
|---|---|---|
| N | 62 | 59 |
| Wk 52 Δ to placebo | | 1.16 |
| P value | | 0.0003 |

FIG. 40. Mean TFC change from baseline vs. placebo at Week 52 for participants at all HD stages and early HD patients (TFC 7-13): comparison of MMRM to MNAR. Table 138 shows the magnitude and p-value for change in TFC at 52 weeks, 45 mg bid vs placebo. Both MMRM and MNAR analyses show a statistically significant improvement in TFC in all HD stages and in early HD patients treated with pridopidine 45 mg bid vs placebo at week 52.

TABLE 138
TFC change from baseline vs. placebo at Week 52

| | Early HD | | All HD | |
|---|---|---|---|---|
| MMRM | 1.16 | (p = 0.0003) | 0.87 | (p = 0.0032) |
| MNAR | 0.79 | (p = 0.016) | 0.58 | (p = 0.057) |

FIG. 41 shows flow of the study.

FIG. 42 pridopidine slows progression in HD patients on UHDRS-TFC scale. The figure shows the change from baseline to Weeks 26, 39 and 52 in UHDRS-TFC score. mITT group excluding patients using neuroleptic medications anytime during the study.

FIG. 43A pridopidine slows progression in HD patients on UHDRS-TFC scale. The figure shows Change from baseline to Weeks 26, 39 and 52 in UHDRS-TFC score. excluding patients using neuroleptic and chorea medications anytime during the study.

FIG. 43B pridopidine slows progression in HD patients on UHDRS-TFC scale. The figure shows change from baseline to Weeks 26, 39, 52, 65 and 78 UHDRS-TFC score in Per protocol group excluding patients using neuroleptic and chorea medications anytime during the study.

FIG. 44 pridopidine slows progression in HD patients on cUHDRS scale. The figure shows change from baseline to Weeks 26 and 39 in cUHDRS score in mITT group.

FIG. 45 pridopidine slows progression in HD patients on cUHDRS scale. The figure shows change from baseline to Weeks 26, 39 52 and 65 in cUHDRS score mITT group excluding patients using neuroleptic medications anytime during the study.

FIG. 46A pridopidine shows improvement and slows progression in HD patients on cUHDRS scale. The figure shows change from baseline to Weeks 26, 39, 52, 65 and 78 in cUHDRS score mITT group in patients not using neuroleptics and chorea medications anytime during the study.

FIG. 46B pridopidine shows improvement and slows progression in HD patients on cUHDRS scale. The figure shows change from baseline to Weeks 26, 39, 52, 65 and 78 in cUHDRS score Per protocol group in patients not using neuroleptics and chorea medications anytime during the study.

FIG. 47 pridopidine shows no decline or slows progression in HD patients on cognition as measured by the SWR scale. The figure shows change from baseline to Weeks 26, 39, 52 and 65 in SWR score mITT group.

FIG. 48 pridopidine shows improvement and no decline in HD patients on cognition as measured by the SWR scale. The figure shows change from baseline to Weeks 26, 39, 52 and 65 in SWR score mITT group in participants not taking neuroleptic medications anytime during the study.

FIG. 49A pridopidine shows improvement in HD patients on cognition as measured by the SWR scale. The figure shows change from baseline to Weeks 26, 39, 52, 65 and 78 in SWR score mITT group in participants not taking neuroleptic and chorea medications anytime during the study.

FIG. 49B pridopidine shows improvement in HD patients on cognition as measured by the SWR scale. The figure shows change from baseline to Weeks 26, 39, 52, 65 and 78 in SWR score. Per protocol group in participants not taking neuroleptic and chorea medications anytime during the study.

FIG. 50 Effect vs placebo on SWR in all patient's mITT group, excluding neuroleptics and excluding neuroleptics and chorea medication.

FIG. 51 pridopidine slows progression in HD patients on TMS scale. The figure shows change from baseline to Weeks 26, 39, 52 in TMS score mITT group

FIG. 52 pridopidine slows progression in HD patients on TMS scale. The figure shows change from baseline to Weeks 26, 39, 52 and 65 in TMS score mITT group in participants not taking neuroleptic medications anytime during the study.

FIG. 53 pridopidine slows progression in HD patients on TMS scale. The figure shows change from baseline to Weeks 26, 39, 52 and 65 in TMS score mITT group in participants not taking neuroleptic and chorea medications anytime during the study.

FIG. 54 pridopidine slow progression in HD patients on Q-Motor Finger Tap Inter Onset Interval (IOI)-Mean score. The figure shows change from baseline to Weeks 26, 52 and 65 in Q-Motor Finger tap inter onset interval (IOI)-Mean mITT group.

FIG. 55 pridopidine shows improvement and slows progression in HD patients on Q-Motor Finger tap inter onset interval (IOI)-mean score. The figure shows change from baseline to Weeks 26, 52 and 65 in Q-Motor Finger tap inter onset interval (IOI)-mean score mITT group in participants not taking neuroleptic medications anytime during the study.

FIG. 56A pridopidine shows improvement and slows progression in HD patients on Q-Motor Finger tap inter onset interval (IOI)-mean score. The figure shows change from baseline to Weeks 26, 52 and 65 in Q-Motor Finger tap inter onset interval (IOI)-mean score mITT group in participants not taking neuroleptic and chorea medications anytime during the study.

FIG. 56B pridopidine shows improvement and slow progression in HD patients on Q-Motor Finger tap inter onset interval (IOI)-mean score. The figure shows change from baseline to Weeks 26, 52, 65 and 78 in Q-Motor Finger tap inter onset interval (IOI)-mean score Per protocol group in participants not taking neuroleptic and chorea medications anytime during the study.

FIG. 57 pridopidine slows progression in HD patients on Q-Motor Finger tap inter onset interval (IOI)-SD score. The figure shows change from baseline to Weeks 26, 52 and 65 in Q-Motor Finger tap inter onset interval (IOI)-SD score mITT group.

FIG. 58 pridopidine shows improvement and slows progression in HD patients on Q-Motor Finger tap inter onset interval (IOI)-SD score. The figure shows change from baseline to Weeks 26, 52 and 65 in Q-Motor Finger tap inter onset interval (IOI)-SD score mITT group in participants not taking neuroleptic medications anytime during the study.

FIG. 59A pridopidine shows improvement and slow progression in HD patients on Q-Motor Finger tap inter onset interval (IOI)-SD score. The figure shows change from baseline to Weeks 26, 52 and 65 in Q-Motor Finger tap inter onset interval (IOI)-SD score mITT group in participants not taking neuroleptic and chorea medications anytime during the study.

FIG. 59B pridopidine shows improvement and slow progression in HD patients on Q-Motor Finger tap inter onset interval (IOI)-SD score. The figure shows change from baseline to Weeks 26, 52, 65 and 78 in Q-Motor Finger tap inter onset interval (IOI)-SD score. Per protocol group in participants not taking neuroleptic and chorea medications anytime during the study.

FIG. 60 pridopidine slow progression in HD patients on Q-Motor Finger tap inter tap interval (ITI)-SD score. The figure shows change from baseline to Weeks 26, 52 and 65 in Q-Motor Finger tap inter tap interval (ITI)-SD score mITT group in participants not taking neuroleptic medications anytime during the study.

FIG. 61 pridopidine slow progression in HD patients on Q-Motor Pronation Supination inter onset interval (IOI)-Mean score. The figure shows change from baseline to Weeks 26 and 52 in Q-Motor Pronation Supination inter onset interval (IOI)-Mean mITT group in all patients.

FIG. 62 pridopidine slow progression in HD patients on Q-Motor Pronation Supination inter onset interval (IOI)-Mean score. The figure shows the change from baseline to Weeks 26, 52 and 65 in Q-Motor Pronation Supination inter onset interval (IOI)-Mean score mITT group in participants not taking neuroleptic medications anytime during the study.

FIG. 63A pridopidine slow progression in HD patients on-Motor Pronation Supination inter onset interval (IOI)-Mean score. The figure shows the change from baseline to Weeks 26, 52 and 65 in Q-Motor Pronation Supination inter onset interval (IOI)-Mean score mITT group in participants not taking neuroleptic and chorea medications anytime during the study.

FIG. 63B pridopidine shows improvement and slow progression in HD patients on Q-Motor Pronation Supination inter onset interval (IOI)-Mean score. The figure shows the change from baseline to Weeks 26, 52, 65 and 78 in Q-Motor Pronation Supination inter onset interval (IOI)-Mean score Per protocol group in participants not taking neuroleptic and chorea medications anytime during the study.

FIG. 64 pridopidine slow progression in HD patients on -Motor Pronation Supination inter onset interval (IOI)-SD score. The figure shows the change from baseline to Weeks 26, 52 and 65 in Q-Motor Pronation Supination inter onset interval (IOI)-SD-mITT group in all patients.

FIG. 65 pridopidine slow progression in HD patients on Q-Motor Pronation Supination inter onset interval (IOI)-SD-score. The figure shows the change from baseline to Weeks 26, 52 and 65 in Q-Motor Pronation Supination inter onset interval (IOI)-SD-score mITT group in participants not taking neuroleptic medications anytime during the study.

FIG. 66A pridopidine slow progression in HD patients on Pronation Supination inter onset interval (IOI)-SD-score. The figure shows the change from baseline to Weeks 26, 52 and 65 in Q-Motor Pronation Supination inter onset interval (IOI)-SD-score mITT group in participants not taking neuroleptic and chorea medications anytime during the study.

FIG. 66B pridopidine slow progression in HD patients on Q-Motor Pronation Supination inter onset interval (IOI)-SD-score. The figure shows the change from baseline to Weeks 26, 52, 65 and 78 in Q-Motor Pronation Supination inter onset interval (IOI)-SD-score Per protocol group in participants not taking neuroleptic and chorea medications anytime during the study.

FIG. 67 pridopidine shows improvement and slow progression in HD patients on Q-Motor Pronation Supination inter tap interval (ITI)-Mean score. The figure shows the change from baseline to Weeks 26, 52 and 65 in Q-Motor Pronation Supination inter tap interval (ITI)-Mean-mITT group in all patients.

FIG. 68 pridopidine shows improvement and slow progression in HD patients on Q-Motor Pronation Supination inter tap interval (ITI)-Mean-score. The figure shows the change from baseline to Weeks 26, 52 and 65 in Q-Motor Pronation Supination inter tap interval (ITI)-Mean-score mITT group in participants not taking neuroleptic medications anytime during the study.

FIG. 69 pridopidine shows improvement and slow progression in HD patients on Q-Motor Pronation Supination inter tap interval (ITI)-Mean-score. The figure shows the change from baseline to Weeks 26, 52 and 65 in Q-Motor Pronation Supination inter tap interval (ITI)-Mean-score mITT group in participants not taking neuroleptic and chorea medications anytime during the study.

FIG. 70 pridopidine slow progression in HD patients on Q-Motor Pronation Supination inter tap interval (ITI)-SD score. The figure shows the change from baseline to Weeks 26, 52 and 65 in Q-Motor Pronation Supination inter tap interval (ITI)-SD-mITT group in all patients.

FIG. 71 pridopidine slow progression in HD patients on Q-Motor Pronation Supination inter tap interval (ITI)-SD-score. The figure shows the change from baseline to Weeks 26, 52 and 65 in Q-Motor Pronation Supination inter tap interval (ITI)-SD-score mITT group in participants not taking neuroleptic medications anytime during the study.

FIG. 72A pridopidine slow progression in HD patients on Q-Motor Pronation Supination inter tap interval (ITI)-SD-score. The figure shows the change from baseline to Weeks 26, 52 and 65 in Q-Motor Pronation Supination inter tap interval (ITI)-SD-score mITT group in participants not taking neuroleptic and chorea medications anytime during the study.

FIG. 72B pridopidine shows improvement and slow progression in Q-Motor Pronation Supination inter tap interval (ITI)-SD-score. The figure shows the change from baseline to Weeks 26, 52, 65 and 78 in Q-Motor Pronation Supination inter tap interval (ITI)-SD-score Per protocol group in participants not taking neuroleptic and chorea medications anytime during the study.

FIG. 73 Strong association between changes of Q-Motor and cUHDRS from baseline

FIG. 74 Strong association between changes of Q-Motor and TFC from baseline

FIG. 75A pridopidine slow progression in HD patients on QoL score. The figure shows the change from baseline to Weeks 26, 52, 65 and 78 in QoL. mITT group in participants not taking neuroleptic and chorea medications anytime during the study.

FIG. 75B pridopidine slow progression in HD patients on QoL score. The figure shows the change from baseline to Weeks 26, 52, 65 and 78 in QoL. Per protocol group in participants not taking neuroleptic and chorea medications anytime during the study.

FIG. 76A pridopidine slow progression in HD patients on TMS Gait and balance subdomain. The figure shows the change from baseline to Weeks 26, 52 and 65 in gait and balance test. mITT group in participants not taking neuroleptic and chorea medications anytime during the study.

FIG. 76B pridopidine slow progression in HD patients on TMS Gait and balance subdomain. The figure shows the change from baseline to Weeks 26, 52, 65 and 78 in gait and balance test. Per protocol group in participants not taking neuroleptic and chorea medications anytime during the study.

### DETAILED DESCRIPTION OF THE INVENTION

Disclosed herein in some embodiments is a method of maintaining, improving, or lessening the decline of motor function and functional capacity in a human patient afflicted with early-stage Huntington disease (HD) [early HD, TFC is 7-13] (HD1 [TFC is 11-13] and HD2 [TFC is 7-10]). In certain embodiments, the method comprises orally administering to the patient with early HD, TFC 7-13, a pharmaceutical composition comprising pridopidine or a pharmaceutically acceptable salt thereof, wherein assessment of said maintaining, improving, or lessening the decline of motor and functional capacity comprises using a composite Unified Huntington Disease rating scale (cUHDRS), wherein said cUHDRS comprises measurement of the total functional capacity (TFC), total motor score (TMS), symbol digital modalities test (SDMT), and Stroop Word Reading Test (SWR) of the patient according to the following equation:

$$cUHDRS = [ ( \frac{T F C - 10.4}{1.9} ) - ( \frac{TMS - 29.7}{14.9} ) + ( \frac{S D M T - 28.4}{11.3} ) + ( \frac{SWR - 66.1}{20.1} ) ] + 10.$$

A skilled artisan would appreciate that in certain embodiments, the term early stage HD encompasses HD stage 1, wherein TFC is between 11-13, and HD stage 2, wherein TFC is between 7-10. Thus, in some embodiments, a measure of early stage HD in a patient afflicted with HD is when TFC is between 7-13.

In some embodiments, use of composite Unified Huntington Disease rating scale (cUHDRS) produces an improved measurement values compared with any one of the independent UHDRS clinical measures of Total Functional Capacity (TFC), Total Motor Score (TMS), Symbol Digit Modality Test (SDMT), or Stroop Word Reading (SWR). In some embodiments, use of composite Unified Huntington Disease rating scale (cUHDRS) produces an improved measurement values compared with any two of the independent UHDRS clinical measures of TFC, TMS, SDMT, or SWR. In some embodiments, use of composite Unified Huntington Disease rating scale (cUHDRS) produces improved measurement values compared with any three of the independent UHDRS clinical measures of TFC, TMS, SDMT, or SWR. In some embodiments, use of cUHDRS produces improved measurement values compared with the independent UHDRS clinical measure of TFC. In some embodiments, use of cUHDRS produces an improved measurement values compared with the independent UHDRS clinical measure of TMS. In some embodiments, use of cUHDRS produces an improved measurement values compared with the independent UHDRS clinical measure of SDMT. In some embodiments, use of cUHDRS produces an improved measurement values compared with the independent UHDRS clinical measure of SWR. In some embodiments, assessment using cUHDRS for clinical change in early symptomatic HD provides enhanced assessment.

Signal-to-noise ratio (S/R) may be used as an index of a measurement's reproducibility. A skilled artisan would appreciate that S/R encompasses the mean change from baseline to a given time divided by the corresponding standard deviate. Thus, the S/N ratio is a measure of the strength of a longitudinal change relative to the random variability of change for a given measure. A larger S/N ratio indicates greater reliable variance, which is a desirable characteristic for the general use of a clinical endpoint. In some embodiments, assessment using cUHDRS to clinical change in early symptomatic HD provides an improved measure of clinical progression. In certain embodiments, use of composite Unified Huntington Disease rating scale (cUHDRS) produces an improved longitudinal Signal to Noise (S/N) ratio compared with a longitudinal S/N ratio of at least one of the independent UHDRS clinical measures of TFC, TMS, SDMT, or SWR. In certain embodiments, use of composite Unified Huntington Disease rating scale (cUHDRS) produces an improved longitudinal Signal to Noise (S/N) ratio compared with a longitudinal S/N ratio of the independent UHDRS clinical measures of TFC. In certain embodiments, use of composite Unified Huntington Disease rating scale (cUHDRS) produces an improved longitudinal Signal to Noise (S/N) ratio compared with a longitudinal S/N ratio of the independent UHDRS clinical measures of TMS. In certain embodiments, use of composite Unified Huntington Disease rating scale (cUHDRS) produces an improved longitudinal Signal to Noise (S/N) ratio compared with a longitudinal S/N ratio of the independent UHDRS clinical measures of SDMT. In certain embodiments, use of composite Unified Huntington Disease rating scale (cUHDRS) produces an improved longitudinal Signal to Noise (S/N) ratio compared with a longitudinal S/N ratio of the independent UHDRS clinical measures of SWR.

In some embodiments, cUHDRS can assist in ensuring that trials targeting clinical progression are maximally sensitive to detect clinical change and maximally protected from failure due to measurement insensitivity. Moreover, trials can be conducted more efficiently and with smaller sample sizes when cUHDRS is utilized as a measure of clinical change. The benefits of using cUHDRS include a lessening of participant burden, using fewer resources to test hypotheses, and potentially enhancing the quality of results obtained through limiting the overall size of the trial.

In some embodiments, of a method of maintaining, improving, or lessening the decline of motor function and functional capacity in a human patient afflicted with early stage Huntington disease (HD).

This invention provides a method of maintaining functional capacity, improving functional capacity, or lessening the decline of functional capacity in a human patient in need thereof comprising periodically orally administering to the patient a pharmaceutical composition comprising pridopidine or a pharmaceutically acceptable salt thereof such that a dose of 90 mg of pridopidine or pharmaceutically acceptable salt thereof is administered to the patient per day (e.g. 45 mg bid), so as to thereby maintain functional capacity, improve functional capacity, or lessening the decline of functional capacity in the human patient. In an embodiment, the method comprises maintaining functional capacity, improving functional capacity, or lessening the decline of functional capacity.

This invention provides a method of maintaining functional capacity, improving functional capacity, reducing the rate of decline of functional capacity, or slowing the rate of functional decline in a human patient in need thereof comprising periodically orally administering to the patient a pharmaceutical composition comprising pridopidine or a pharmaceutically acceptable salt thereof such that a dose of 90 mg of pridopidine is administered to the patient per day (e.g. 45 mg bid), so as to thereby maintain functional capacity, improve functional capacity, reduce the rate of decline of functional capacity or slow the rate of functional decline in the human patient. In an embodiment, the method comprises maintaining functional capacity, improving functional capacity, or reducing the rate of decline of functional capacity.

In an embodiment, the method comprises maintaining function capacity. In another embodiment, the method comprises improving functional capacity. In another embodiment, the method comprises lessening the decline of functional capacity In some embodiments, the functional capacity is maintained or improved or show less decline, for at least 12 weeks, at least 20 weeks, at least 26 weeks, at least 52 weeks, at least 65 weeks, at least 78 weeks, at least 2 years, at least 3 years, at least 4 years or at least 5 years.

In certain embodiment, a method of maintaining, improving, or lessening the decline of motor function and functional capacity comprises maintaining, improving, or lessening the decline of motor function and functional capacity for at least 6 months, at least 12 months, at least 65 weeks, at least 78 weeks, at least 24 months, at least 36 months, at least 48 months, or 60 months. In another embodiment, maintaining, improving, or lessening the decline of motor function and functional capacity is for between about at least 6 months-60 months. In another embodiment, maintaining, improving, or lessening the decline of motor function and functional capacity is for between about at least 12 months-60 months. In another embodiment, maintaining, improving, or lessening the decline of motor function and functional capacity is for between about at least 24 months-60 months. In another embodiment, maintaining, improving, or lessening the decline of motor function and functional capacity is for between about at least 36 months-60 months. In another embodiment, maintaining, improving, or lessening the decline of motor function and functional capacity is for between about at least 48 months-60 months.

In certain embodiments, maintaining, improving, or lessening the decline of motor function and functional capacity is for at least 26 weeks, at least 52 weeks, at least 65 weeks, at least 78 weeks, at least 24 months, at least 36 months, at least 48 months, or at least 60 months. In one embodiment, maintaining, improving, or lessening the decline of motor function and functional capacity is for at least 26 weeks. In another embodiment, maintaining, improving, or lessening the decline of motor function and functional capacity is for at least 52 weeks. In one embodiment, maintaining, improving, or lessening the decline of motor function and functional capacity is for at least 65 weeks. In one embodiment, maintaining, improving, or lessening the decline of motor function and functional capacity is for at least 78 weeks. In one embodiment, maintaining, improving, or lessening the decline of motor function and functional capacity is for at least 24 months. In one embodiment, maintaining, improving, or lessening the decline of motor function and functional capacity is for at least 36 months In one embodiment, maintaining, improving, or lessening the decline of motor function and functional capacity is for at least 48 months. In one embodiment, maintaining, improving, or lessening the decline of motor function and functional capacity is for at least 60 months.

In another embodiment, the functional capacity is total functional capacity (TFC) measured by UHDRS-TFC and the human patient has an improvement of one (1) or more points in the UHDRS TFC. In some embodiments, the human patient has an improvement of one (1) or more points in the UHDRS TFC after 52 weeks or after 65 weeks of administration of pridopidine. In another embodiment, the rate of functional decline is less than one (1) point as measured by the UHDRS TFC after 52 weeks or after 65 weeks of administration of pridopidine.

In one embodiment, the method comprises lessening the decline of functional capacity. In another embodiment, the method comprises lessening the decline of functional capacity and (a) the pharmaceutical composition is administered for more than 26 weeks or (b) the human patient is afflicted with early stage HD. In one embodiment, the method comprises reducing the rate of decline of functional capacity. In another embodiment, the method comprises reducing the rate of decline of functional capacity and (a) the pharmaceutical composition is administered for more than 26 weeks or (b) the human patient is afflicted with early stage HD. In some embodiments, the method comprises lessening functional decline. In some embodiments, the decline in functional capacity is lessened by or the rate of functional decline is slowed for at least 5%, at least 10%, at least 20%, at least 30%, at least 40%, at least 50%, or at least 80%. In another embodiment, the rate of the decline in functional capacity is slowed for at least 12 weeks, at least 20 weeks, at least 26 weeks, at least 39 weeks, at least 52 weeks, at least 65 weeks, at least 78 weeks, at least 3 years, or at least 5 years. In another embodiment, rate of functional decline is slowed in functional capacity is reduced for at least 12 weeks, at least 20 weeks, at least 26 weeks, at least 52 weeks, 65 weeks, at least 78 weeks, at least 3 years, at least 4 years, or at least 5 years.

In another embodiment, the rate of functional decline is slower and functional capacity is reduced for at least 6 months, at least 12 months, at least 26 weeks, at least 39 weeks, at least 52 weeks, at least 65 weeks, at least 78 weeks, at least 24 months, at least 36 months, at least 48 months, or 60 months. In another embodiment, the rate of functional decline is slowed, and functional capacity is reduced for between about at least 6 months-60 months. In another embodiment, the rate of functional decline is slowed, and functional capacity is reduced for between about at least 12 months-60 months. In another embodiment, the rate of functional decline is slowed, and functional capacity is reduced for between about at least 24 months-60 months. In another embodiment, the rate of functional decline is slowed, and functional capacity is reduced for between about at least 36 months-60 months. In another embodiment, the rate of functional decline is slowed, and functional capacity is reduced for between about at least 48 months-60 months.

In certain embodiments, a method comprises maintaining, improving, or lessening the decline of total functional capacity (TFC) of an early HD patient. In one embodiment, a method comprises maintaining, improving, or lessening the decline of total functional capacity (TFC) of an early HD patient for at least 26 weeks, at least 39 weeks, at least 52 weeks, at least 65 weeks, at least 78 weeks, at least 24 months, at least 36 months, at least 48 months, or at least 60 months. In one embodiment, a method comprises maintaining, improving, or lessening the decline of total functional capacity (TFC) of an early HD patient for at least 26 weeks. In one embodiment, a method comprises maintaining, improving, or lessening the decline of total functional capacity (TFC) of an early HD patient for at least 52 weeks. In one embodiment, a method comprises maintaining, improving, or lessening the decline of total functional capacity (TFC) of an early HD patient for at least 65 weeks, at least 78 weeks. In one embodiment, a method comprises maintaining, improving, or lessening the decline of total functional capacity (TFC) of an early HD patient for at least 24 months. In one embodiment, a method comprises maintaining, improving, or lessening the decline of total functional capacity (TFC) of an early HD patient for at least 36 months. In one embodiment, a method comprises maintaining, improving, or lessening the decline of total functional capacity (TFC) of an early HD patient for at least 48 months. In one embodiment, a method comprises maintaining, improving, or lessening the decline of total functional capacity (TFC) of an early HD patient for at least 60 months.

In one embodiment, the functional capacity is total functional capacity (TFC). The total functional capacity may be measured by UHDRS-TFC. The total functional capacity may also be measured by the UHDRS Functional Assessment Scale (UHDRS-FAS). In an embodiment the functional capacity is maintained for at least 12 weeks, at least 20 weeks, at least 26 weeks, at least 39 weeks, at least 52 weeks, at least 65 weeks, or at least 78 weeks. In another embodiment, the functional capacity is maintained for at least 6 months, at least 12 months, at least 65 weeks, at least 78 weeks, at least 24 months, at least 36 months, at least 48 months, or 60 months. In another embodiment, the functional capacity is maintained for between about at least 6 months-60 months. In another embodiment, the functional capacity is maintained for between about at least 12 months-60 months. In another embodiment, the functional capacity is maintained for between about at least 24 months-60 months. In another embodiment, the functional capacity is maintained for between about at least 36 months-60 months. In another embodiment, the functional capacity is maintained for between about at least 48 months-60 months.

In an embodiment, the human patient has no deterioration of functional capacity. In other embodiments, the human patient has no deterioration of functional capacity for at least 26 weeks. In other embodiments, the human patient has no deterioration of functional capacity for at least 39 weeks. In other embodiments, the human patient has no deterioration of functional capacity for at least 52 weeks. In other embodiments, the human patient has no deterioration of functional capacity for at least 65 weeks. In other embodiments, the human patient has no deterioration of functional capacity for at least 78 weeks.

In another embodiment, the human patient has no deterioration of functional capacity for at least 6 months, at least 12 months, at least 65 weeks, at least 78 weeks, at least 24 months, at least 36 months, at least 48 months, or 60 months. In another embodiment, the human patient has no deterioration of functional capacity for between about at least 6 months-60 months. In another embodiment, the human patient has no deterioration of functional capacity for between about at least 12 months-60 months. In another embodiment, the human patient has no deterioration of functional capacity for between about at least 24 months-60 months. In another embodiment, the human patient has no deterioration of functional capacity for between about at least 36 months-60 months. In another embodiment, the human patient has no deterioration of functional capacity for between about at least 48 months-60 months.

The invention additionally provides a method of slowing the clinical progression of HD in a human patient comprising periodically orally administering to the patient afflicted with HD a pharmaceutical composition comprising pridopidine or a pharmaceutically acceptable salt thereof such that a dose of 90-mg of pridopidine is administered to the patient per day, so as to thereby slow the clinical progression of HD in the patient (e.g. 45 mg bid).

In an embodiment, the clinical progression of HD is measured by total functional capacity. In one embodiment, the clinical progression of HD is slowed by at least 5%, at least 10%, at least 20%, at least 30%, at least 50%, at least 80%, or between 20% and 90%. In another embodiment, the clinical progression of HD is slowed for at least 12 weeks, at least 20 weeks, at least 26 weeks, at least 39 weeks, at least 52 weeks, at least 65 weeks, at least 78 weeks, at least 3 years, at least 4 years, or at least 5 years. In another embodiment, the clinical progression of HD is slowed at least 26 weeks. In another embodiment, the clinical progression of HD is slowed for at least 52 weeks. In another embodiment, the clinical progression of HD is slowed for at least 65 weeks. In another embodiment, the clinical progression of HD is slowed for at least 78 weeks. In another embodiment, the clinical progression of HD is slowed for at least 3 years. In another embodiment, the clinical progression of HD is slowed for at least 4 years. In another embodiment, the clinical progression of HD is slowed for or at least 5 years.

In another embodiment, the clinical progression of HD is slowed for at least 6 months, at least 12 months, at least 65 weeks, at least 78 weeks, at least 24 months, at least 36 months, at least 48 months, or 60 months. In another embodiment, the clinical progression of HD is slowed for between about at least 6 months-60 months. In another embodiment, the clinical progression of HD is slowed for between about at least 12 months-60 months. In another embodiment, the clinical progression of HD is slowed for between about at least 24 months-60 months. In another embodiment, the clinical progression of HD is slowed for between about at least 36 months-60 months. In another embodiment, the clinical progression of HD is slowed for between about at least 48 months-60 months.

In a further embodiment, the total functional capacity is measured by the UHDRS-TFC. In certain embodiments, the total functional capacity is measured as part of an assessment of maintaining, improving, or lessening the decline of motor and functional capacity in a subject afflicted with HD, wherein said measurement comprises use of cUHDRS. In certain embodiments, the total functional capacity is measured as part of an assessment of maintaining, improving, or lessening the decline of motor and functional capacity in a subject afflicted with early stage HD, wherein said measurement comprises use of cUHDRS.

This invention also provides a method of reducing functional decline as measured by UHDRS Total Functional Capacity, in a human patient in need thereof comprising periodically orally administering to the human patient a pharmaceutical composition comprising pridopidine or a pharmaceutically acceptable salt thereof such that a dose of 90 mg of pridopidine is administered to the patient per day (e.g. 45 mg bid), so as to thereby reduce functional decline in the human patient, wherein the human patient is afflicted with HD and has a baseline TFC score of 11-13.

In an embodiment, functional decline is measured by UHDRS-TFC. In certain embodiments, functional decline is measured by UHDRS-TFC as part of an assessment of maintaining, improving, or lessening the decline of motor and functional capacity in a subject afflicted with HD, wherein said measurement comprises use of cUHDRS. In certain embodiments, functional decline is measured by UHDRS-TFC as part of an assessment of maintaining, improving, or lessening the decline of motor and functional capacity in a subject afflicted with early stage HD, wherein said measurement comprises use of cUHDRS. In another embodiment, the method comprises reducing functional decline for at least 12 weeks, at least 20 weeks, at least 26 weeks, at least 39 weeks, at least 52 weeks, at least 65 weeks, at least 78 weeks, at least 3 years, at least 4 years, or at least 5 years.

In an embodiment, lessening the decline of functional capacity is measured by UHDRS-TFC. In certain embodiments, lessening the decline of functional capacity is measured by UHDRS-TFC as part of an assessment of maintaining, improving, or lessening the decline of motor and functional capacity in a subject afflicted with HD, wherein said measurement comprises use of cUHDRS. In certain embodiments, lessening the decline of functional capacity is measured by UHDRS-TFC as part of an assessment of maintaining, improving, or lessening the decline of motor and functional capacity in a subject afflicted with early stage HD, wherein said measurement comprises use of cUHDRS. In another embodiment, the method comprises lessening the decline of functional capacity for at least 12 weeks, at least 20 weeks, at least 26 weeks, at least 39 weeks, at least 52 weeks, at least 65 weeks, at least 78 weeks, at least 3 years, at least 4 years, or at least 5 years. In another embodiment, the method comprises lessening the decline of motor function and functional capacity in a subject suffering from early stage HD for at least 12 weeks, at least 20 weeks, at least 26 weeks, at least 52 weeks, at least 65 weeks, at least 78 weeks, at least 3 years, at least 4 years, or at least 5 years.

In another embodiment, the method comprises lessening the decline of motor function and functional capacity for at least 6 months, at least 12 months, at least 65 weeks, at least 78 weeks, at least 24 months, at least 36 months, at least 48 months, or 60 months. In another embodiment, the method comprises lessening the decline of motor function and functional capacity for between about at least 6 months-60 months. In another embodiment, the method comprises lessening the decline of motor function and functional capacity for between about at least 12 months-60 months. In another embodiment, the method comprises lessening the decline of motor function and functional capacity for between about at least 24 months-60 months. In another embodiment, the method comprises lessening the decline of motor function and functional capacity for between about at least 36 months-60 months. In another embodiment, the method comprises lessening the decline off motor function and functional capacity for between about at least 48 months-60 months.

This invention also provides a method of maintaining, improving, or lessening the decline of, a human patient's ability to perform activities of daily living, comprising periodically orally administering to the human patient in need thereof a pharmaceutical composition comprising pridopidine or a pharmaceutically acceptable salt thereof such that a dose of 90 mg of pridopidine is administered to the patient per day (e.g. 45 mg bid), so as to thereby maintain, improve, or lessen the decline of the human patient's ability to perform activities of daily living.

This invention also provides a method of maintaining, improving, or reducing the rate of decline of, a human patient's ability to perform activities of daily living, comprising periodically orally administering to the human patient in need thereof a pharmaceutical composition comprising pridopidine or a pharmaceutically acceptable salt thereof such that a dose of 90 mg of pridopidine is administered to the patient per day (e.g. 45 mg bid), so as to thereby maintain, improve, or reduce the rate of decline of the human patient's ability to perform activities of daily living.

In one embodiment, the human patient's ability to perform activities of daily living is maintained, improved, or the decline is lessened over a period of at least 12 weeks, at least 20 weeks, at least 26 weeks, at least 39 weeks, at least 52 weeks, or at least 65 weeks, or at least 78 weeks. In one embodiment, the human patient's ability to perform activities of daily living is maintained, improved, or the rate of decline is reduced for at least 12 weeks, at least 20 weeks, at least 26 weeks, at least 52 weeks, or at least 65 weeks, or at least 78 weeks. In another embodiment, the method comprises maintaining the human patient's ability to perform activities of daily living. In an embodiment, the ability to perform activities of daily living is measured by the Activities of Daily Living (ADL) domain of the TFC.

The invention also provides a method of maintaining, improving, or lessening the decline of, a human patient's ability to manage finances, comprising periodically orally administering to the human patient in need thereof a pharmaceutical composition comprising pridopidine or a pharmaceutically acceptable salt thereof such that a dose of 90 mg of pridopidine is administered to the patient per day (e.g. 45 mg bid), so as to thereby maintain, improve, or lessen the rate of decline of the human patient's ability to manage finances.

The invention also provides a method of maintaining, improving, or reducing the rate of decline of, a human patient's ability to manage finances, comprising periodically orally administering to the human patient in need thereof a pharmaceutical composition comprising pridopidine or a pharmaceutically acceptable salt thereof such that a dose of 90 mg of pridopidine is administered to the patient per day (e.g. 45 mg bid), so as to thereby maintain, improve, or reduce the rate of decline of the human patient's ability to manage finances.

In another embodiment, administering further maintains, improves, or lessens the decline of the human patient's ability to manage finances. In an embodiment, the human patient's ability to manage finances is maintained, improved, or the decline of is lessened for at least 12 weeks, at least 20 weeks, at least 26 weeks, at least 39 weeks, at least 52 weeks, at least 65 weeks, or at least 78 weeks. In another embodiment, administering further maintains, improves, or reduces the rate of decline of the human patient's ability to manage finances. In an embodiment, the human patient's ability to manage finances is maintained, improved, or the rate of decline is reduced for at least 12 weeks, at least 20 weeks, at least 26 weeks, at least 52 weeks, at least 65 weeks, or at least 78 weeks. In one embodiment, the method comprises maintaining the human patient's ability to manage finances. In another embodiment, the method comprises improving the human patient's ability to manage finances. In some embodiments, the ability to manage finances is measured by the Managing Finances domain of the TFC.

In one embodiment, administering further maintains, improves, or reduces the rate of decline of the human patient's ability to perform domestic chores. In another embodiment, administering further maintains, improves, or lessens the decline of the human patient's ability to perform domestic chores.

The invention also provides a method of maintaining, improving, or lessening the decline of, a human patient's ability to perform domestic chores, comprising periodically orally administering to the human patient in need therefore a pharmaceutical composition comprising pridopidine or a pharmaceutically acceptable salt thereof such that a dose of 90-225 mg of pridopidine is administered to the patient per day, so as to thereby maintain, improve, or lessen the decline of the human patient's ability to perform domestic chores.

The invention also provides a method of maintaining, improving, or reducing the rate of decline of, a human patient's ability to perform domestic chores, comprising periodically orally administering to the human patient in need therefore a pharmaceutical composition comprising pridopidine or a pharmaceutically acceptable salt thereof such that a dose of 90 mg of pridopidine is administered to the patient per day (e.g. 45 mg bid), so as to thereby maintain, improve, or reduce the rate of decline of the human patient's ability to perform domestic chores.

In an embodiment, the ability to perform domestic chores is measured by the Domestic Chores domain of the UHDRS TFC. In another embodiment, the human patient's ability to perform domestic chores is maintained or improved for at least 12 weeks, at least 20 weeks, at least 26 weeks, at least 39 weeks, at least 52 weeks, at least 65 weeks, or at least 78 weeks. In another embodiment, the method comprises maintaining the human patient's ability to perform domestic chores. In one embodiment, the method comprises improving the human patient's ability to perform domestic chores. In another embodiment, the human patient's ability to perform domestic chores is maintained or improved, or the rate of decline is reduced for at least 12 weeks, at least 20 weeks, at least 26 weeks, at least 52 weeks, at least 65 weeks, or at least 78 weeks. In a further embodiment, the human patient's ability to perform domestic chores is maintained or improved, or the decline is lessened for at least 12 weeks, at least 20 weeks, at least 26 weeks, at least 52 weeks, at least 65 weeks, or at least 78 weeks.

In one embodiment, administering further maintains, improves, or reduces the rate of decline of, the care level of the human patient. In another embodiment, administering further maintains, improves, or lessens the decline of, the care level of the human patient.

The invention also provides, a method of maintaining, improving, or lessening the decline of, a human patient's care level, comprising periodically orally administering to the human patient in need thereof a pharmaceutical composition comprising pridopidine or pharmaceutically acceptable salt thereof such that a dose of 90 mg of pridopidine is administered to the patient per day (e.g. 45 mg bid), so as to thereby maintain, improve, or lessen the decline of the human patient's care level.

The invention also provides, a method of maintaining, improving, or reducing the rate of decline of, a human patient's care level, comprising periodically orally administering to the human patient in need thereof a pharmaceutical composition comprising pridopidine such that a dose of 90-mg of pridopidine is administered to the patient per day, so as to thereby maintain, improve, or reduce the rate of decline of the human patient's care level.

In an embodiment, the care level is measured by the Care level domain of the TFC. In another embodiment, the human patient's care level is maintained, improved, or the rate of decline is reduced for at least 12 weeks, at least 20 weeks, at least 26 weeks, at least 39 weeks, at least 52 weeks, at least 65 weeks, or at least 78 weeks. In another embodiment, the human patient's care level is maintained, improved, or the decline of is lessened for at least 12 weeks, at least 20 weeks, at least 26 weeks, at least 52 weeks, at least 65 weeks, or at least 78 weeks. In another embodiment, the method comprises maintaining the care level of the human patient.

In one embodiment, a dose of 90 mg of pridopidine is administered to the patient per day. In another embodiment, a dose of 90 mg of pridopidine is administered to the patient per day in unit doses of 45 mg twice per day (bid).

In some embodiments, a method of maintaining, improving, or lessening the decline of motor and functional capacity in a human patient afflicted with early-stage Huntington disease (HD1 and HD2, TFC 7-13) comprises orally administering to the patient a pharmaceutical composition comprising pridopidine or a pharmaceutically acceptable salt thereof. In some embodiments, pridopidine or pharmaceutically acceptable salt thereof is administered at a dose of 90 mg per day (90 mg/day). In some embodiments, a composition is administered twice per day, wherein pridopidine or pharmaceutically acceptable salt thereof is administered at a dose of 45 mg bid.

In some embodiments, administration of pridopidine or a pharmaceutically acceptable salt thereof is for at least 26 weeks, at least 39 weeks, at least 52 weeks, at least 65 weeks, at least 78 weeks, at least 24 months, at least 36 months, at least 48 months, or at least 60 months. In some embodiments, administration of pridopidine or a pharmaceutically acceptable salt thereof is for at least 26 weeks. In some embodiments, administration of pridopidine or a pharmaceutically acceptable salt thereof is for at least 52 weeks. In some embodiments, administration of pridopidine or a pharmaceutically acceptable salt thereof is for at least 65 weeks. In some embodiments, administration of pridopidine or a pharmaceutically acceptable salt thereof is for at least 78 weeks. In some embodiments, administration of pridopidine or a pharmaceutically acceptable salt thereof is for at least 24 months. In some embodiments, administration of pridopidine or a pharmaceutically acceptable salt thereof is for at least 36 months. In some embodiments, administration of pridopidine or a pharmaceutically acceptable salt thereof is for at least 48 months. In some embodiments, administration of pridopidine or a pharmaceutically acceptable salt thereof is for at least 60 months.

In some embodiments, pridopidine or a pharmaceutically acceptable salt thereof is administered at a dose of 90 mg/day for a period of at least 26 weeks, at least 39 weeks, at least 52 weeks, at least 65 weeks, at least 78 weeks, at least 24 months, at least 36 months, at least 48 months, or at least 60 months. In some embodiments, pridopidine or a pharmaceutically acceptable salt thereof is administered at a dose of 90 mg/day for a period of at least 26 weeks. In some embodiments, pridopidine or a pharmaceutically acceptable salt thereof is administered at a dose of 90 mg/day for a period of at least 39 weeks. In some embodiments, pridopidine or a pharmaceutically acceptable salt thereof is administered at a dose of 90 mg/day for a period of at least 52 weeks. In some embodiments, pridopidine or a pharmaceutically acceptable salt thereof is administered at a dose of 90 mg/day for a period of at least 65 weeks. In some embodiments, pridopidine or a pharmaceutically acceptable salt thereof is administered at a dose of 90 mg/day for a period of at least 78 weeks. In some embodiments, pridopidine or a pharmaceutically acceptable salt ther embodiments, pri eof is administered at a dose of 90 mg/day for a period of at least 24 months. In some embodiments pridopidine or a pharmaceutically acceptable salt thereof is administered at a dose of 90 mg/day for a period of at least 36 months. In some embodiments, pridopidine or a pharmaceutically acceptable salt thereof is administered at a dose of 90 mg/day for a period of at least 48 months. In some embodiments, pridopidine or a pharmaceutically acceptable salt thereof is administered at a dose of 90 mg/for a period of at least 60 months.

In some embodiments, pridopidine or a pharmaceutically acceptable salt thereof is administered at a dose of 45 mg bid for a period of at least 26 weeks, at least 39 weeks at least 52 weeks, at least 65 weeks, at least 78 weeks, at least 24 months, at least 36 months, at least 48 months, or at least 60 months. In some embodiments, pridopidine or a pharmaceutically acceptable salt thereof is administered at a dose of 45 mg bid for a period of at least 26 weeks. In some embodiments, pridopidine or a pharmaceutically acceptable salt thereof is administered at a dose of 45 mg bid for a period of at least 39 weeks. In some embodiments, pridopidine or a pharmaceutically acceptable salt thereof is administered at a dose of 45 mg bid for a period of at least 52 weeks. In some embodiments, pridopidine or a pharmaceutically acceptable salt thereof is administered at a dose of 45 mg bid for a period of at least 65 weeks. In some embodiments, pridopidine or a pharmaceutically acceptable salt thereof is administered at a dose of 45 mg bid for a period of at least 78 weeks. In some embodiments, pridopidine or a pharmaceutically acceptable salt thereof is administered at a dose of 45 mg bid for a period of at least 24 months. In some embodiments, pridopidine or a pharmaceutically acceptable salt thereof is administered at a dose of 45 mg bid for a period of at least 36 months. In some embodiments, pridopidine or a pharmaceutically acceptable salt thereof is administered at a dose of 45 mg bid for a period of at least 48 months. In some embodiments, pridopidine or a pharmaceutically acceptable salt thereof is administered at a dose of 45 mg bid for a period of at least 60 months.

The invention further provides a method of reducing dystonia or maintaining a level of dystonia in a human patient in need thereof comprising periodically orally administering to the patient a pharmaceutical composition comprising pridopidine such that a dose of 90 mg of pridopidine is administered to the patient per day (e.g. 45 mg bid), so as to thereby reduce dystonia or maintain a level of dystonia in the human patient.

In one embodiment, dystonia is measured by the UHDRS TMS Dystonia score. In another embodiment, the level of dystonia in the human patient is reduced or maintained for at least 12 weeks, at least 20 weeks, at least 26 weeks, at least 39 weeks, at least 52 weeks, at least 65 weeks, or at least 78 weeks.

In some embodiment the dystonia is limb dystonia.

The invention also provides a method of treating limb dystonia in a human patient in need thereof comprising periodically orally administering to the patient a pharmaceutical composition comprising pridopidine such that a dose of 90 mg of pridopidine is administered to the patient per day, so as to thereby treat the limb dystonia in the human patient. In many embodiments, (a) the pharmaceutical composition is administered for more than 26 weeks or (b) a titration dose of an amount different from the intended dose is administered for a period of time at the start of the periodic administration or (c) the human patient is afflicted with early-stage HD.

In another embodiment, a dose of 45 mg bid of pridopidine is administered to the patient per day.

In another embodiment, the pharmaceutical composition is administered for at least 12 weeks, at least 20 weeks, at least 26 weeks, more than 26 weeks, at least 39 weeks, at least 52 weeks, at least 54 weeks, at least 65 weeks, at least 78 weeks, at least 104 weeks or more. In another embodiment, the treating limb dystonia comprises preventing the slowing, the reduction in amplitude, or the impairment of the human patient's finger tapping ability and/or preventing the slowing or the irregular performance of the Pronate-Supinate Hands test in the human patient.

This invention also provides a method of preventing the slowing, the reduction in amplitude, or the impairment of the human patient's finger tapping ability and/or preventing the slowing or the irregular performance of the Pronate-Supinate Hands test in a human HD patient comprising periodically orally administering to the patient a pharmaceutical composition comprising pridopidine such that a dose of 90 mg of pridopidine is administered to the patient per so as to thereby prevent the slowing, the reduction in amplitude, or the impairment of the human patient's finger tapping ability and/or prevent the slowing or the irregular performance of the Pronate-Supinate Hands test in the human patient.

In another embodiment, the treating limb dystonia comprises preventing the impairment of the human patient's finger tapping ability and/or preventing the slowing or the irregular performance of the Q-Motor: Pro-Sup-Frequency-MN-Hand (Hz) test. In another embodiment, the treatment comprises improving the human patient's Q-Motor tap speed frequency. In another embodiment, the treatment comprises improving the human patient's Q-Motor tap speed inter onset interval (IOI).

The invention further provides a method of improving or maintaining, a human patient's gait and balance comprising periodically orally administering to the human patient in need thereof a pharmaceutical composition comprising pridopidine such that a dose of 90 mg of pridopidine is administered to the patient per day (e.g. 45 mg bid), so as to thereby improve or maintain, a human patient's gait and balance.

In one embodiment, a dose of 90 mg, pridopidine or pharmaceutically acceptable salt thereof is administered to the patient per day. In one embodiment, a dose of 45 mg bid of pridopidine is administered to the patient.

Additionally provided is a method of improving, maintaining, or lessening the decline of, a human patient's gait and balance comprising periodically orally administering to the human patient in need thereof a pharmaceutical composition comprising pridopidine or pharmaceutically acceptable salt thereof such that a dose of 90 mg of pridopidine is administered to the patient per day (e.g. 45 mg bid), so as to thereby improve, maintain, or lessen the decline of, a human patient's gait and balance.

Also provided is a method of improving, maintaining, or slowing the decline of, a human patient's gait and balance comprising periodically orally administering to the human patient in need thereof a pharmaceutical composition comprising pridopidine or pharmaceutically acceptable salt thereof, such that a dose of 90 mg of pridopidine is administered to the patient per day (e.g. 45 mg bid), so as to thereby improve, maintain, or slow the decline of, a human patient's gait and balance.

In an embodiment, the human patient's gait and balance is measured by the UHDRS gait and balance score. In some embodiments, the human patient's gait and balance is improved or maintained or the decline is lessened for at least 12 weeks, at least 20 weeks, at least 26 weeks, at least 39 weeks, at least 52 weeks, at least 65 weeks, or at least 78 weeks.

In an embodiment, the human patient's gait and balance is measured by the UHDRS gait and balance score. In some embodiments, the human patient's gait and balance is improved or maintained or the decline is slowed for at least 12 weeks, at least 20 weeks, at least 26 weeks, at least 39 weeks, at least 52 weeks, at least 65 weeks, or at least 78 weeks.

The invention also provides a method of improving or maintaining, a human patient's independence comprising periodically orally administering to the human patient in need thereof a pharmaceutical composition comprising pridopidine such that a dose of 90-mg of pridopidine is administered to the patient per day, so as to thereby improve or maintain a human patient's independence.

In one embodiment, a dose of 90 mg, of pridopidine is administered to the patient per day. In another embodiment, a dose of 45 mg bid of pridopidine is administered to the patient per day.

The invention also provides a method of improving, maintaining, or lessening the decline of, a human patient's independence comprising periodically orally administering to the human patient in need thereof a pharmaceutical composition comprising pridopidine such that a dose of 90 mg of pridopidine is administered to the patient per day (e.g. 45 mg bid), so as to thereby improve, maintain, or lessen the decline of, a human patient's independence.

The invention also provides a method of improving, maintaining, or slowing the decline of, a human patient's independence comprising periodically orally administering to the human patient in need thereof a pharmaceutical composition comprising pridopidine such that a dose of 90 mg of pridopidine is administered to the patient per day (e.g. 45 mg bid), so as to thereby improve, maintain, or slow the decline of, a human patient's independence.

In an embodiment, the human patient's independence is measured by the UHDRS Independence score. In some embodiments, the human patient's independence is improved or maintained, or the decline is slowed for at least 12 weeks, at least 20 weeks, at least 26 weeks, at least 39 weeks, at least 52 weeks, at least 65 weeks, or at least 78 weeks. In one embodiment, the human patient's independence is improved or maintained, or the decline is lessened for at least 12 weeks, at least 20 weeks, at least 26 weeks, at least 39 weeks, at least 52 weeks, at least 65 weeks, or at least 78 weeks.

The invention also provides a method of improving or maintaining a human patient's cognitive domains comprising periodically orally administering to the human patient in need thereof a pharmaceutical composition comprising pridopidine such that a dose of 90 mg of pridopidine is administered to the patient per day (e.g. 45 mg bid), so as to thereby improve or maintain the human patient's cognitive domains. A patient's cognitive domains may also be the patient's cognitive performance across a variety of domains.

In one embodiment, a dose of 90 mg of pridopidine is administered to the patient per day. In another embodiment, a dose of 45 mg bid, of pridopidine is administered to the patient per day.

Further provided is a method of improving, maintaining, or lessening the decline of, a human patient's cognitive domains comprising periodically orally administering to the human patient in need thereof a pharmaceutical composition comprising pridopidine such that a dose of 90 mg of pridopidine is administered to the patient per day (e.g. 45 mg bid), so as to thereby improve, maintain, or lessen the decline of, a human patient's cognitive domains. Cognitive domains may be understood as cognitive performance across a variety of domains.

Further provided is a method of improving, maintaining, or slowing the decline of, a human patient's cognitive domains comprising periodically orally administering to the human patient in need thereof a pharmaceutical composition comprising pridopidine such that a dose of 90 mg of pridopidine is administered to the patient per day (e.g. 45 mg bid), so as to thereby improve, maintain, or slow the decline of, a human patient's cognitive domains. Cognitive domains may be understood as cognitive performance across a variety of domains.

The human patient's cognitive domains may be measured, for example, by the cognitive assessment battery (CAB). The human patient's cognitive domains may also be measured by the Hopkins Verbal Learning Test—Revised (HVLT-R). The human patient's cognitive domains may additionally be measured by the Paced Tapping test, the Montreal Cognitive Assessment (MoCA) scale or the Symbol Digit Modalities Test (SDMT). The human patient's cognitive domains may additionally be measured by trail making test B (TMT-B). In one embodiment, the human patient's cognitive domains are maintained or improved, or the decline is slowed for at least 12 weeks, at least 20 weeks, at least 26 weeks, at least 39 weeks, at least 52 weeks, at least 65 weeks, or at least 78 weeks. In some embodiments, slowing the decline of a human patient's cognitive domains comprises slowing the rate of cognitive decline. In an embodiment, the human patient's cognitive domains are maintained or improved, or the decline is lessened for at least 12 weeks, at least 20 weeks, at least 26 weeks, at least 39 weeks, at least 52 weeks, at least 65 weeks, or at least 78 weeks. In one embodiment, the human patient's cognitive domains are maintained or improved, or the decline is slowed for at least 6 months, at least 39 weeks, at least 52 weeks at least 65 weeks, at least 78 weeks, at least 24 months, at least 36 months, at least 48 months or at least 60 months. In an embodiment, the human patient's cognitive domains are maintained or improved, or the decline is lessened for at least 6 months, at least 39 weeks, at least 52 weeks at least 65 weeks, at least 78 weeks, at least 12 months, at least 24 months, at least 36 months, at least 48 months or at least 60 months.

The invention also provides a method of reducing the severity of the sustained or intermittent muscle contractions associated with dystonia in a human patient in need thereof comprising periodically orally administering to the patient a pharmaceutical composition comprising pridopidine such that a dose of 90 mg of pridopidine is administered to the patient per (e.g. 45 mg bid), so as to thereby reduce the severity of the sustained or intermittent muscle contractions associated with dystonia in the human patient. In many embodiments, (a) the pharmaceutical composition is administered for more than 26 weeks or (b) a titration dose of an amount different from the intended dose is administered for a period of time at the start of the periodic administration and/or (c) the human patient is afflicted with early stage HD.

The severity of the sustained or intermittent muscle contractions associated with dystonia in a human patient may be measured by, for example, the UHDRS TMS Dystonia score.

Further provided is a method of improving or maintaining motor ability in a human patient in need thereof comprising periodically orally administering to the patient a pharmaceutical composition comprising pridopidine such that a dose of 90-mg of pridopidine is administered to the patient per day (e.g. 45 mg bid), so as to thereby maintain or improve motor ability in the human patient. In certain embodiments, a method of maintaining, improving, or lessening the decline of motor function and functional capacity in a human patient afflicted with HD comprises maintaining, improving, or lessening the decline of motor function in said patient. In certain embodiments, a method of maintaining, improving, or lessening the decline of motor function and functional capacity in a human patient afflicted with early stage HD (HD1 and HD2, TFC 7-13) comprises maintaining, improving, or lessening the decline of motor function in said patient.

The motor ability may be measured, for example, by the UHDRS TMS score, the UHDRS TMS score excluding chorea or UHDRS TMS score excluding dystonia. In some embodiments, a UHDRS TMS score is measured as part of an assessment using the cUHDRS.

In an embodiment, a dose of 90 mg, g of pridopidine is administered to the patient per day. In another embodiment, a dose of 45 mg of pridopidine is administered to the patient twice per day. In another embodiment, a dose of 180 mg of pridopidine is administered to the patient per day. In another embodiment, the motor ability is maintained or improved for at least 12 weeks, at least 20 weeks, at least 26 weeks, at least 39 weeks, at least 52 weeks, at least 65 weeks, at least 78 weeks. In another embodiment, the motor function is maintained, improved, or the decline is lessened for at least 12 weeks, at least 20 weeks, at least 26 weeks, at least 52 weeks, at least 65 weeks, at least 78 weeks, at least 24 months, at least 36 months, at least 48 months, or at least 60 months. In another embodiment, the motor function is maintained, improved, or the decline is lessened for at least 12 weeks. In another embodiment, the motor function is maintained, improved, or the decline is lessened for at least 20 weeks. In another embodiment, the motor function is maintained, improved, or the decline is lessened for at least 26 weeks. In another embodiment, the motor function is maintained, improved, or the decline is lessened for at least 39 weeks. In another embodiment, the motor function is maintained, improved, or the decline is lessened for at least 52 weeks. In another embodiment, the motor function is maintained, improved, or the decline is lessened for at least 65 weeks. In another embodiment, the motor function is maintained, improved, or the decline is lessened for at least 78 weeks. In another embodiment, the motor function is maintained, improved, or the decline is lessened for at least 24 months. In another embodiment, the motor function is maintained, improved, or the decline is lessened for at least 36 months, In another embodiment, the motor function is maintained, improved, or the decline is lessened for at least 48 months. In another embodiment, the motor function is maintained, improved, or the decline is lessened for at least 60 months. The invention also provides a method of reducing or maintaining the level of chorea in a human patient in need thereof comprising periodically orally administering to the patient a pharmaceutical composition comprising pridopidine such that a dose of 90 mg of pridopidine is administered to the patient per day (e.g. 45 mg bid), so as to thereby reduce or maintain the level of chorea in a human patient.

In one embodiment, a dose of 90 mg, of pridopidine is administered to the patient per day. In another embodiment, a dose of 45 mg of pridopidine is administered to the patient twice per day. The level of chorea may also be reduced.

The invention also provides a method of reducing, maintaining, or lessening the increase of, chorea in a human patient in need thereof comprising periodically orally administering to the patient a pharmaceutical composition comprising pridopidine such that a dose of 90 mg of pridopidine is administered to the patient per day (e.g. 45 mg bid), so as to thereby reduce, maintain, or lessen the increase of, chorea in a human patient.

The invention also provides a method of reducing, maintaining, or slowing the increase of, chorea in a human patient in need thereof comprising periodically orally administering to the patient a pharmaceutical composition comprising pridopidine such that a dose of 90 mg of pridopidine is administered to the patient per day (e.g. 45 mg bid), so as to thereby reduce, maintain, or slow the increase of, chorea in a human patient.

In one embodiment, the chorea in the human patient is improved, or maintained, or the increase is slowed for at least 12 weeks, at least 20 weeks, at least 26 weeks, at least 39 weeks, at least 52 weeks, at least 65 weeks, or at least 78 weeks. In an embodiment, the chorea in the human patient is improved, or maintained, or the increase is lessened for at least 12 weeks, at least 20 weeks, at least 26 weeks, at least 52 weeks, at least 65 weeks, or at least 78 weeks. The human patient's chorea may be measured by the UHDRS TMS chorea score.

The invention further provides a method of improving, maintaining, reducing or lessening the decline of a human patient's behavior and/or psychiatric state comprising periodically orally administering to the human patient in need thereof a pharmaceutical composition comprising pridopidine such that a dose of 90-mg of pridopidine is administered to the patient per day, so as to thereby improve, maintain, reduce, or lessen the decline of the human patient's behavior and/or psychiatric state.

In one embodiment, the method comprises maintaining a human patient's behavior and/or psychiatric state. In another embodiment, the method comprises improving the human patient's behavior and/or psychiatric state. In another embodiment, the human patient's behavior and/or psychiatric state is improved, maintained or the decline is reduced or lessened for at least 12 weeks, at least 20 weeks, at least 26 weeks, at least 39 weeks, at least 52 weeks, at least 65 weeks, or at least 78 weeks. In another embodiment, the human patient's behavior and/or psychiatric state is improved, maintained or the decline is reduced or lessened for at least 6 months, at least 12 months, at least 65 weeks, at least 78 weeks, at least 24 months, at least 36 months, at least 48 months or at least 60 months.

The human patient's behavior and/or psychiatric state may be measured by the Problem Behaviors Assessment (PBA) total score. The human patient's behavior and/or psychiatric state may also be measured by the Problem Behaviors Assessment-short form (PBA-s). The human patient's behavior and/or psychiatric state may also be measured by the Problem Behaviors Assessment for depressed mood. The human patient's behavior and/or psychiatric state may also be measured by the Problem Behaviors Assessment for irritability. The human patient's behavior and/or psychiatric state may also be measured by the Problem Behaviors Assessment for lack of initiative or apathy. The human patient's behavior and/or psychiatric state may also be measured by the Problem Behaviors Assessment short form apathy sub-item. The human patient's behavior and/or psychiatric state may also be measured by the Apathy Evaluation Scale (AES). The human patient's behavior and/or psychiatric state may be measured by the Problem Behaviors Assessment for obsessive-compulsiveness. The human patient's behavior and/or psychiatric state may also be measured by the Problem Behaviors Assessment for disoriented behavior. In some embodiments, the human patient's behavior and/or psychiatric state is measured by the Problem Behaviors Assessment short form apathy sub-item or the Problem Behaviors Assessment-short form (PBA-s).

The invention also provides a method of reducing or maintaining a human patient's involuntary movements comprising periodically orally administering to the human patient in need thereof a pharmaceutical composition comprising pridopidine such that a dose of 90 mg of pridopidine is administered to the patient per day (e.g. 45 mg bid), so as to thereby reduce or maintain a human patient's involuntary movements.

In one embodiment, the human patient's involuntary movements are reduced or maintained for at least 12 weeks, at least 20 weeks, at least 26 weeks, at least 39 weeks, at least 52 weeks, at least 65 weeks, or at least 78 weeks. In one embodiment, the human patient's involuntary movements are reduced or maintained for at least 6 months, at least 12 months, at least 65 weeks, at least 78 weeks, at least 24 months, at least 36 months, at least 48 months or at least 60 months. The patient's involuntary movements may be measured by UHDRS TMS Involuntary Movements score.

The invention further provides method of improving or maintaining a human patient's mobility comprising periodically orally administering to the human patient in need thereof a pharmaceutical composition comprising pridopidine such that a dose of 90-mg of pridopidine is administered to the patient per day, so as to thereby improve or maintain the human patient's mobility.

In one embodiment, the human patient's mobility is improved, or maintained for at least 12 weeks, at least 20 weeks, at least 26 weeks, at least 39 weeks, at least 52 weeks, at least 65 weeks, or at least 78 weeks. In one embodiment, the human patient's mobility is improved, or maintained for at least 6 months, at least 12 months, at least 65 weeks, at least 78 weeks, at least 24 months, at least 36 months, at least 48 months or at least 60 months. The human patient's mobility may be measured by the Timed Up and Go Test. The human patient's mobility may also be measured by the Walk-12 Total Score. The human patient's mobility may further be measured by the patient's walking ability.

This invention also provides a method of improving or maintaining a human patient's ability to perform physical tasks comprising periodically orally administering to the human patient in need thereof a pharmaceutical composition comprising pridopidine such that a dose of 90 mg of pridopidine is administered to the patient per day, so as to thereby improve or maintain the human patient's ability to perform physical tasks.

The invention also provides a method of improving or maintaining a human patient's quality of life comprising periodically orally administering to the patient a pharmaceutical composition comprising pridopidine such that a dose of 90 mg of pridopidine is administered to the patient per day, so as to thereby improve or maintain the human patient's quality of life.

In one embodiment, the human patient's quality of life is improved, or maintained for at least 12 weeks, at least 20 weeks, at least 26 weeks, at least 39 weeks, at least 52 weeks, at least 65 weeks, or at least 78 weeks. In one embodiment, the human patient's quality of life is improved, or maintained for at least 6 months, at least 12 months, at least 65 weeks, at least 78 weeks, at least 24 months, at least 36 months, at least 48 months or at least 60 months. In another embodiment, the human patient's quality of life is maintained. In another embodiment, the human patient's quality of life is measured by the Huntington's Disease Quality of Life (HD-QoL) score.

The invention further provides a method of reducing the natural decline in the total functional capacity of a HD patient, comprising periodically orally administering to the patient a pharmaceutical composition comprising pridopidine such that a dose of 90 mg of pridopidine is administered to the patient per day, so as to thereby reduce the natural decline in the total functional capacity in the human patient. In another embodiment, periodically orally administering to the patient a pharmaceutical composition comprising pridopidine such that a dose of 45 mg bid of pridopidine is administered to the patient per day. In one embodiment, the natural decline is reduced by 20-70%, 30%-60%, or 35%-45%. In another embodiment, the natural decline is reduced by 20%, 30%, 40%, 50%, 60%, 70, 71%, 80%, 90%, 95%, 96%, 100%. In another embodiment, the natural decline versos the placebo is reduced by 20%, 30%, 40%, 50%, 60%, 70, 71%, 80%, 90%, 95%, 96%, 100%. In an embodiment, the natural decline is lessened by 0.16-0.56, 0.24-0.48, 0.28-0.36, 0.14-0.27, 0.23-0.45, 0.07-0.23, 0.23-0.53 points per year as measured by the UHDRS-TFC. In another embodiment, the natural decline is lessened by 0.14, 0.16, 0.18, 0.24, 0.27, 0.32, 0.4, 0.45, 0.48 or 0.56 points per year as measured by UHDRS-TFC. The invention further provides a method of improvement in cUHDRS compared to participants in the placebo group by at least 45%, at least 68%, at least 100%, or by at least by 200%, comprising periodically orally administering to the patient a pharmaceutical composition comprising pridopidine such that a dose of 45 mg bid of pridopidine is administered to the patient per day, for at least 26 weeks, or at least 39 weeks, or at least 52 weeks, or at least 65 weeks, or at least 78 weeks, wherein the patients not administering neuroleptics or Chorea Medications.

Neurofilament light protein (NfL) levels may be used as a biomarker of neurodegeneration in HD patients (Byrne 2017). NfL concentrations in plasma were found to increase with advancing HD disease. Thus, NfL concentrations in plasma of HD patients may provide a means for assessing and predicting neural damage in patients with HD (Byrne 2017). Additionally, results suggest that NfL in the blood or plasma could provide a reliable estimate of the concentration of NfL in the CSF (Byrne 2017).

The invention further provides a method of maintaining, reducing, or lessening the increase of, the concentration of neurofilament light protein in a HD patient, comprising periodically orally administering to the patient a pharmaceutical composition comprising pridopidine such that a dose of 90 mg of pridopidine is administered to the patient per day, so as to thereby maintain, decrease, or lessen the increase of, the concentration of neurofilament light protein in the human patient. In one embodiment, the increase of the concentration of neurofilament light protein is lessened in the human patient. In another embodiment, the concentration of neurofilament light protein is maintained or decreased in the human patient.

The invention further provides a method of predicting clinical responsiveness to pridopidine therapy in a subject afflicted with HD, the method comprising administering an amount of pridopidine and evaluating the amount of a neurofilament light protein in the subject, so as to thereby predict clinical responsiveness to pridopidine. In another embodiment, the neurofilament light protein amount is measured in plasma or in cerebrospinal fluid (CSF).

In one embodiment, the method further comprising predicting positive clinical responsiveness to pridopidine if the amount of the neurofilament light protein is decreased in the subject after administration of pridopidine compared to baseline. In one embodiment, the method further comprising predicting positive clinical responsiveness to pridopidine if the amount of the neurofilament light protein is maintained in the subject after administration of pridopidine relative to baseline. In another embodiment, the method further comprising predicting positive clinical responsiveness to pridopidine if the amount of the neurofilament light protein shows less increase in the subject after administration of pridopidine compared to baseline. Baseline, in this paragraph, is the amount of the neurofilament light protein prior to administration of pridopidine.

In one embodiment, the subject is identified as a pridopidine responder if the amount of the biomarker is higher than a reference value. In another embodiment, the subject is identified as a pridopidine responder if amount of the biomarker is lower than a reference value.

In another embodiment, if the subject is identified as a pridopidine responder, the subject is thereafter administered a pharmaceutical composition comprising pridopidine.

In another embodiment, a dose of 90 mg, of pridopidine or pharmaceutically acceptable salt thereof is administered to the patient per day. In another embodiment, a dose of 45 mg bid of pridopidine or pharmaceutically acceptable salt thereof is administered to the patient per day.

In an embodiment, the human patient is afflicted with HD. In some embodiments, the human patient is afflicted with early stage HD (HD1 and HD2, TFC 7-13).

In some embodiments, a unit dose of the pharmaceutical composition contains 45 mg, of pridopidine.

In an embodiment, the pharmaceutical composition is administered twice per day (bid). In another embodiment, an equal amount of the pharmaceutical composition is administered at each administration. In an embodiment, the two doses are administered at least 6 hours apart, at least 7 hours, at least 8 hours, at least 9 hours, at least 10 hours, at least 11 hours apart. In some embodiments, the pharmaceutical composition is administered for at least 12 weeks, at least 20 weeks, at least 26 weeks, more than 26 weeks, at least 39 weeks, at least 52 weeks, at least 65 weeks, or at least 78 weeks. In some embodiments, the pharmaceutical composition is administered for at least 39 weeks. In some embodiments, the pharmaceutical composition is administered for at least 65 weeks. In some embodiments, the pharmaceutical composition is administered for at least 78 weeks. In some embodiments, the pharmaceutical composition is administered for at least 24 months. In some embodiments, the pharmaceutical composition is administered for at least 36 months. In some embodiments, the pharmaceutical composition is administered for at least 48 months. In some embodiments, the pharmaceutical composition is administered for at least 60 months. In another embodiment, the composition is administered for at least 6 months, at least 12 months, at least 65 weeks, at least 78 weeks, at least 24 months, at least 36 months, at least 48 months, or 60 months. In another embodiment, the composition is administered for between about at least 6 months-60 months. In another embodiment, the composition is administered for between about at least 12 months-60 months. In another embodiment, the composition is administered for between about at least 24 months-60 months. In another embodiment, the composition is administered for between about at least 36 months-60 months. In another embodiment, the composition is administered for between about at least 48 months-60 months.

In one embodiment, the patient has a UHDRS-TMS score ≥20 before beginning treatment. In another embodiment, the patient has a UHDRS-IS (UHDRS-Independence Scale) score below or equal to 90% before beginning treatment. In another embodiment, the patient has greater than or equal to 36 CAG repeats in the huntingtin gene. In another embodiment, the human patient has greater than 44 CAG repeats in the huntingtin gene. In another embodiment, the human patient has less than 44 CAG repeats in the huntingtin gene. In another embodiment, the human patient is afflicted with early stage HD. In another embodiment, the human patient has a baseline TFC score which is greater than or equal to 9. In another embodiment, the human patient has a baseline TFC score which is greater than or equal to 7. In another embodiment, the human patient has a baseline TFC score of 11-13. In another embodiment, the human patient has a baseline TFC score of 7-10. In another embodiment, the human patient is afflicted with HD and has a baseline TMS score which is in the least severe quarter of the overall population of patients afflicted with HD. In another embodiment, the human patient is afflicted with HD and has a baseline TMS score which is in the two least severe quarters of the overall population of patients afflicted with HD. In another embodiment, the human patient is afflicted with HD and has a baseline TMS score which is in the three least severe quarters of the overall population of patients afflicted with HD. In another embodiment, the human patient is afflicted with HD and has a baseline TMS score which is in the three least severe quarters of the overall population of patients afflicted with HD or a baseline TFC score which is greater than or equal to 9. In another embodiment, the human patient is afflicted with HD and has a baseline TMS score which is in the three least severe quarters of the overall population of patients afflicted with HD or a baseline TFC score which is greater than or equal to 9 or less than 44 CAG repeats in the huntingtin gene. In another embodiment, the human patient is afflicted with HD and has a baseline TMS score which is in the two least severe quarters of the overall population of patients afflicted with Huntington's disease. In another embodiment, the human patient is afflicted with HD and has a baseline TFC score which is greater than or equal to 9 or greater than 44 CAG repeats in the huntingtin gene. In another embodiment, the human patient is afflicted with HD and has a baseline TMS score which is in the three least severe quarters of the overall population of patients afflicted with HD or less than 44 CAG repeats in the huntingtin gene. In another embodiment, the human patient is afflicted with HD and has a baseline TFC score which is greater than or equal to 9 or a baseline TMS score which is in the three least severe quarters of the overall population of patients afflicted with HD.

In some embodiments, of a method of maintaining, improving, or lessening the decline of motor function and functional capacity in a human patient afflicted with HD, and assessed using cUHDRS, the patient has greater than or equal to 36 CAG repeats in the Huntingtin gene. In another embodiment, the human patient has greater than 44 CAG repeats in the Huntingtin gene. In another embodiment, the human patient has less than 44 CAG repeats in the Huntingtin gene. In another embodiment, the human patient is afflicted with early stage HD. In another embodiment, the human patient is afflicted with early stage HD, wherein early stage HD comprises HD1 or HD2. In another embodiment, the human patient is afflicted with early stage HD, wherein early stage HD comprises HD1 or HD2 or/and TFC 7-13. In another embodiment, the human patient has a baseline TFC score which is greater than or equal to 7. In another embodiment, the human patient has a baseline TFC score which is greater than or equal to 8. In another embodiment, the human patient has a baseline TFC score of 11-13. In another embodiment, the human patient has a baseline TFC score of 7-10. In another embodiment, the human patient has a baseline TFC score of 0-6. In another embodiment, the human patient has a baseline TFC score of 7-13. In one embodiment, the pridopidine is pridopidine hydrochloride.

In an embodiment, a titration dose of an amount different from the intended dose is administered for a period of time at the start of the periodic administration. In some embodiments, the titration dose is half the amount of the intended dose. In another embodiment, the titration dose is administered in one administration per day and the intended dose is administered in two administrations per day. In one embodiment, the titration dose is administered for 7-21 or 7-14 days prior to the administration of the intended dose. In another embodiment, the titration dose is administered for 7 days, 14 days, or 21 days prior to the administration of the intended dose. The titration dose is preferably administered for fourteen days prior to the administration of the intended dose.

In an embodiment, the method further comprises no worsening of the human patient's other HD symptoms compared to baseline. In an embodiment, the method further comprises no worsening of another symptom of HD in comparison to a human patient not administered pridopidine. In another embodiment, the symptoms are not worsened for at least 12 weeks, at least 20 weeks, at least 26 weeks, at least 39 weeks, at least 52 weeks, at least 65 weeks, or at least 78 weeks. In another embodiment, the symptoms are not worsened for at least 6 months, at least 12 months, at least 65 weeks, at least 78 weeks, at least 24 months, at least 36 months, at least 48 months, or 60 months. In another embodiment, the symptoms are not worsened for between about at least 6 months-60 months.

In another embodiment, the symptoms are not worsened for between about at least 12 months-60 months. In another embodiment, the symptoms are not worsened for between about at least 24 months-60 months. In another embodiment, the symptoms are not worsened for between about at least 36 months-60 months. In another embodiment, the symptoms are not worsened for between about at least 48 months-60 months.

In some embodiments, provided herein a method maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof. In some embodiments, the administration is for at least 26 weeks, at least 39 weeks, or at least 52 weeks. In some embodiments, the pridopidine or a pharmaceutically acceptable salt thereof is administered at a dose 45 mg bid for a period of at least 26 weeks, at least 39 weeks, or at least 52 weeks. In another embodiment, the motor function is assessed by Q-motor. In another embodiment, the maintaining, improving, or lessening the decline of motor function and functional capacity does not include administering neuroleptics medications including olanzapine, risperidone, tiapride, aripiprazole, quetiapine, fluphenazine, haloperidol, lithium, ziprasidone, amisulpride, asenapine, cariprazine, chlorpromazine, clotiapine, cyamemazine, flupentixol, prochlorperazine, promazine, and prothipendyl.

In another embodiment, the maintaining, improving, or lessening the decline of motor function and functional capacity does not include administering chorea medications.

In some embodiments the SWR (Stroop Word Reading) was improved after at least 26 weeks of treatment, at least 39 weeks of treatment, 52 weeks of treatment or 65 weeks of treatment.

In another related aspect, the method comprises maintaining, improving, or lessening the decline of Stroop Word Reading (SWR) test of said patient.

In some embodiment, provided herein a method of improvement in a validated measure of cognitive function, the Stroop word reading (SWR) test by administering 90 mg/day (e.g. 45 mg bid) for up to 78 weeks, or at least 26 weeks, or at least 39 weeks, or at least 53 weeks, in HD patients, wherein the improvement in the SWR is at least by 100%-450%, 130%-450%, or 300%-450%. In some embodiments, provided herein a method of improvement in a validated measure of cognitive function, the Stroop word reading (SWR) test by administering 90 mg/day (e.g. 45 mg bid) for up to 78 weeks, or at least 26 weeks, or at least 39 weeks, or at least 53 weeks in HD patients without antidopaminergic drugs, wherein the improvement showed less decline in TMS.

In some embodiments provided herein a method of maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof wherein assessment of said maintaining, improving, or lessening the decline of motor function and functional capacity comprises using a composite Unified Huntington Disease rating scale (cUHDRS), wherein said cUHDRS comprises measurement of the total functional capacity (TFC), total motor score (TMS), symbol digital modalities test (SDMT), and Stroop Word Reading Test (SWR) of the patient according to the following equation:

$$cUHDRS = [ ( \frac{T F C - 10.4}{1.9} ) - ( \frac{TMS - 29.7}{14.9} ) + ( \frac{S D M T - 28.4}{11.3} ) + ( \frac{SWR - 66.1}{20.1} ) ] + 10.$$

In some embodiments, the administration is for at least 26 weeks or at least 52 weeks. In some embodiments, the pridopidine or a pharmaceutically acceptable salt thereof is administered at a dose 45 mg bid for a period of at least 26 weeks, at least 39 weeks, or at least 52 weeks. In another embodiment, the motor function is assessed by Q-motor.

In another embodiment, the maintaining, improving, or lessening the decline of motor function and functional capacity does not include administering neuroleptics medications including olanzapine, risperidone, tiapride, aripiprazole, quetiapine, fluphenazine, haloperidol, lithium, ziprasidone, amisulpride, asenapine, cariprazine, chlorpromazine, clotiapine, cyamemazine, flupentixol, prochlorperazine, promazine, and prothipendyl.

Chorea medications include VMAT inhibitors such as Tetrabenazine and deutetrabenazine. In another embodiment, the maintaining, improving, or lessening the decline of motor function and functional capacity does not include administering chorea medications.

In some embodiments the SWR (Stroop Word Reading) was improved after at least 26 weeks of treatment, at least 39 weeks of treatment, 52 weeks of treatment or 65 weeks of treatment. In another related aspect, the method comprises maintaining, improving, or lessening the decline of Stroop Word Reading (SWR) test of said patient.

Provided herein is a pharmaceutical composition comprising pridopidine for use in maintaining functional capacity in a human patient wherein the pharmaceutical composition is to be periodically orally administered to the patient such that a dose of 90 mg of pridopidine is to be administered to the patient per day (e.g. 45 mg bid). In some embodiments functional capacity includes ADL.

Provided herein is use of an amount of pridopidine in the manufacture of a medicament maintaining functional capacity in a human patient wherein the medicament is formulated for periodic oral administration to the patient such that a dose of 90 mg of pridopidine is to be administered to the patient per day (e.g. 45 mg bid). In some embodiments functional capacity includes ADL.

Provided herein is a pharmaceutical composition comprising pridopidine for use in slowing the clinical progression of HD as measured by total functional capacity in a human patient wherein the pharmaceutical composition is to be periodically orally administered to the patient such that a dose of 90 mg of pridopidine is to be administered to the patient per day (e.g. 45 mg bid).

Provided herein is a use of an amount of pridopidine in the manufacture of a medicament for slowing the clinical progression of HD as measured by total functional capacity in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in maintaining a human patient's ability to perform activities of daily living in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in maintaining a human patient's ability to perform activities of daily living in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in reducing dystonia or maintaining a level of dystonia in a human patient wherein the pharmaceutical composition is to be periodically orally administered to the patient such that a dose of 90 mg of pridopidine is to be administered to the patient per day (e.g. 45 mg bid). In some embodiments, dystonia includes limb dystonia.

Provided herein is use of an amount of pridopidine in the manufacture of a medicament for use in reducing dystonia or maintaining a level of dystonia in a human patient wherein the medicament is formulated for periodic oral administration to the patient such that a dose of 90 mg of pridopidine is to be administered to the patient per day (e.g. 45 mg bid). In some embodiments, dystonia includes limb dystonia.

Provided herein is a pharmaceutical composition comprising pridopidine for use in treating limb dystonia in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in treating limb dystonia in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining gait and balance in a human patient wherein the pharmaceutical composition is to be periodically orally administered to the patient such that a dose of 90 mg of pridopidine is to be administered to the patient per day (e.g. 45 mg bid). In some embodiments the administration slows the decline of a patient's gait and balance.

Provided herein is use of an amount of pridopidine in the manufacture of a medicament for use in improving or maintaining, a human patient's gait and balance in a human patient wherein the medicament is formulated for periodic oral administration to the patient such that a dose of 90 mg of pridopidine is to be administered to the patient per day. In some embodiments the administration slows the decline of a patient's gait and balance.

Provided herein is a pharmaceutical composition comprising pridopidine for use in improving, maintaining, or slowing the decline of gait and balance in a human patient wherein the pharmaceutical composition is to be periodically orally administered to the patient such that a dose of 90 mg of pridopidine is to be administered to the patient per day (e.g. 45 mg bid). In some embodiments the administration slows the decline of a patient's gait and balance.

Provided herein is use of an amount of pridopidine in the manufacture of a medicament for use in improving, maintaining, or slowing the decline of, a human patient's gait and balance in a human patient wherein the medicament is formulated for periodic oral administration to the patient such that a dose of 90 mg of pridopidine is to be administered to the patient per day (e.g. 45 mg bid). In some embodiments the administration slows the decline of a patient's gait and balance.

Provided herein is a pharmaceutical composition comprising pridopidine for use in improving or maintaining independence in a human patient wherein the pharmaceutical composition is to be periodically orally administered to the patient such that a dose of 90-mg of pridopidine is to be administered to the patient per day (e.g. 45 mg bid).

Provided herein is use of an amount of pridopidine in the manufacture of a medicament for use in improving or maintaining, a human patient's independence wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining or slowing the decline of a human patient's independence wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining, or slowing the decline of a human patient's independence wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining a human patient's cognitive domains wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining a human patient's cognitive domains wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining or slowing the decline of a human patient's cognitive domains wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in improving or maintaining or slowing the decline of a human patient's cognitive domains wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in reducing the severity of the sustained or intermittent muscle contractions associated with dystonia in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in reducing the severity of the sustained or intermittent muscle contractions associated with dystonia in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining motor ability in a human patient wherein the pharmaceutical composition is to be periodically orally administered to the patient such that a dose of 90-225 mg of pridopidine is to be administered to the patient per day. In another embodiment, the dose is 45 bid of pridopidine per day. Provided herein is use of an amount of pridopidine in the manufacture of a medicament for use in improving or maintaining motor ability in a human patient wherein the medicament is formulated for periodic oral administration to the patient such that a dose of 90-225 mg of pridopidine is to be administered to the patient per day. In another embodiment, the dose is 45 bid of pridopidine per day. Provided herein is a pharmaceutical composition comprising pridopidine for use in reducing or maintaining the level of chorea in a human patient wherein the pharmaceutical composition is to be periodically orally administered to the patient such that a dose of 90 mg of pridopidine is to be administered to the patient per day. In another embodiment, the dose is 45 bid of pridopidine per day (e.g. 45 mg bid).

Provided herein is use of an amount of pridopidine in the manufacture of a medicament for use in reducing or maintaining the level of chorea in a human patient wherein the medicament is formulated for periodic oral administration to the patient such that a dose of 90 mg of pridopidine is to be administered to the patient per day. In another embodiment, the dose is 45 bid of pridopidine per day (e.g. 45 mg bid).

Provided herein is a pharmaceutical composition comprising pridopidine for use in reducing or maintaining or slowing the increase of chorea in a human patient wherein the pharmaceutical composition is to be periodically orally administered to the patient such that a dose of 90 mg of pridopidine is to be administered to the patient per day (e.g. 45 mg bid).

Provided herein is use of an amount of pridopidine in the manufacture of a medicament for use in reducing or maintaining or slowing the increase of chorea in a human patient wherein the medicament is formulated for periodic oral administration to the patient such that a dose of 90 mg of pridopidine is to be administered to the patient per day (e.g. 45 mg bid).

Provided herein is a pharmaceutical composition comprising pridopidine for use in improving or maintaining a human patient's behavior and/or psychiatric state wherein the pharmaceutical composition is to be periodically orally administered to the patient such that a dose of 90 mg of pridopidine is to be administered to the patient per day.

Provided herein is use of an amount of pridopidine in the manufacture of a medicament for use in improving or maintaining a human patient's behavior and/or psychiatric state wherein the medicament is formulated for periodic oral administration to the patient such that a dose of 90-mg of pridopidine is to be administered to the patient per day. In another embodiment, the dose is 45 bid of pridopidine per day. Provided herein is a pharmaceutical composition comprising pridopidine for use in reducing or maintaining a human patient's involuntary movements wherein the pharmaceutical composition is to be periodically orally administered to the patient such that a dose of 90 mg of pridopidine is to be administered to the patient per day. In another embodiment, the dose is 45 bid of pridopidine per day.

Provided herein is use of an amount of pridopidine in the manufacture of a medicament for use in reducing or maintaining a human patient's involuntary movements wherein the medicament is formulated for periodic oral administration to the patient such that a dose of 90 mg of pridopidine is to be administered to the patient per day. In another embodiment, the dose is 45 bid of pridopidine per day.

Provided herein is a pharmaceutical composition comprising pridopidine for use in improving or maintaining a human patient's mobility wherein the pharmaceutical composition is to be periodically orally administered to the patient such that a dose of 90-225 mg of pridopidine is to be administered to the patient per day. In another embodiment, the dose is 45 bid of pridopidine per day.

Provided herein is use of an amount of pridopidine in the manufacture of a medicament for use in improving or maintaining a human patient's mobility wherein the medicament is formulated for periodic oral administration to the patient such that a dose of 90 mg of pridopidine is to be administered to the patient per day. In another embodiment, the dose is 45 bid of pridopidine per day.

The subject invention also provides a package comprising:
a) a pharmaceutical composition comprising pridopidine; and
b) instructions for use of the pharmaceutical composition according to the methods of the present invention.

Provided herein is a pharmaceutical composition comprising pridopidine for use in improving or maintaining a human patient's ability to perform physical tasks wherein the pharmaceutical composition is to be periodically orally administered to the patient such that a dose of 90 mg of pridopidine is to be administered to the patient per day. In another embodiment, the dose is 45 bid of pridopidine per day.

Provided herein is a use of an amount of pridopidine in the manufacture of a medicament for use in improving or maintaining a human patient's ability to perform physical tasks wherein the medicament is formulated for periodic oral administration to the patient such that a dose of 90-mg of pridopidine is to be administered to the patient per day. In another embodiment, the dose is 45 bid of pridopidine per day.

The invention also provides, a method of maintaining or improving total functional capacity, in a human patient afflicted with HD comprising periodically orally administering to the patient a pharmaceutical composition comprising pridopidine such that a dose of 45 mg bid of pridopidine is administered to the patient per day, so as to thereby maintain functional capacity, or improve total functional capacity, in the human patient as measured by the UHDRS-TFC for at least 26, at least 39 weeks, or 52 weeks.

In one embodiment, a dose of 90 mg of pridopidine is administered to the patient per day (e.g. 45 mg bid). In another embodiment, the human patient has a baseline TFC score of 11-13. In another embodiment, the human patient has a baseline TFC score of 7-10. In another embodiment, the human patient has a baseline TMS score which is in the two least severe quarters of the overall population of patients afflicted with HD. In another embodiment, the human patient has a baseline TMS score which is in the three least severe quarters of the overall population of patients afflicted with HD. In another embodiment, the human patient has less than 44 CAG repeats in the Huntingtin gene.

The invention also provides, a method of maintaining, or improving a human patient's ability to perform activities of daily living, comprising periodically orally administering to the patient a pharmaceutical composition comprising pridopidine such that a dose of 90 mg of pridopidine is administered to the patient per day (e.g. 45 mg bid), so as to thereby maintain, or improve the human patient's ability to perform activities of daily living as measured by Activities of Daily Living domain of the UHDRS-TFC for at least 26 or 52 weeks, wherein the human patient is afflicted with HD.

In one embodiment, administering further maintains or improves the human patient's ability to manage finances as measured by measured by the Managing Finances domain of the UHDRS-TFC for at least 26, at least 39 weeks, or 52 weeks.

The invention also provides, a method of maintaining, or improving a human patient's ability to manage finances, comprising periodically orally administering to the patient a pharmaceutical composition comprising pridopidine such that a dose of 90 or 180 mg of pridopidine is administered to the patient per day, so as to thereby maintain, or improve the human patient's ability to manage finances as measured by Managing Finances domain of the UHDRS-TFC for at least 26, at least 39 weeks, or 52 weeks, wherein the human patient is afflicted with HD.

In one embodiment, a dose of 90 mg of pridopidine is administered to the patient per day (e.g. 45 mg bid). In another embodiment, the human patient has a baseline UHDRS-TFC score of 11-13. In another embodiment, the human patient has a baseline UHDRS-TFC score of 7-10. In another embodiment, the human patient has a baseline TMS score which is in the two least severe quarters of the overall population of patients afflicted with HD. In another embodiment, the human patient has a baseline TMS score which is in the three least severe quarters of the overall population of patients afflicted with HD.

The invention also provides, a method of maintaining, improving, or the rate of decline of, a human patient's ability to perform domestic chores as measured by the Domestic Chores domain of the UHDRS-TFC, comprising periodically orally administering to the patient a pharmaceutical composition comprising pridopidine such that a dose of 90 mg of pridopidine is administered to the patient per day (e.g. 45 mg bid), so as to thereby maintain, improve, or reduce the rate of decline of the human patient's ability to perform domestic chores, wherein the human patient is afflicted with HD and has a baseline TFC score of 11-13.

The invention also provides, a method of maintaining, improving, or lessening the decline of, a human patient's ability to perform domestic chores as measured by the Domestic Chores domain of the UHDRS TFC, comprising periodically orally administering to the patient a pharmaceutical composition comprising pridopidine such that a dose of 90 mg of pridopidine is administered to the patient per day (e.g. 45 mg bid), so as to thereby maintain, improve, or lessen the decline of the human patient's ability to perform domestic chores, wherein the human patient is afflicted with HD and has a baseline TFC score of 11-13.

The invention also provides, a method of maintaining, improving, or reducing the rate of decline of, a human patient's care level as measured by the Care Level of the UHDRS TFC, comprising periodically orally administering to the patient a pharmaceutical composition comprising pridopidine such that a dose of 90 mg of pridopidine is administered to the patient per day (e.g. 45 mg bid), so as to thereby maintain, improve, or reduce the rate of decline of the human patient's care level, wherein the human patient is afflicted with HD and has a baseline TFC score of 11-13.

The invention also provides, a method of maintaining, improving, or lessening the decline of, a human patient's care level as measured by the Care Level of the UHDRS TFC, comprising periodically orally administering to the patient a pharmaceutical composition comprising pridopidine such that a dose of 90 mg of pridopidine is administered to the patient per day (e.g. 45 mg bid), so as to thereby maintain, improve, or lessen the decline of, the human patient's care level, wherein the human patient is afflicted with HD and has a baseline TFC score of 11-13.

The invention also provides, a method of improving or maintaining, a human patient's gait and balance comprising periodically orally administering to the patient a pharmaceutical composition comprising pridopidine such that a dose of 90 mg of pridopidine is administered to the patient per day (e.g. 45 mg bid), so as to thereby improve, or maintain, a human patient's gait and balance as measured by the UHDRS gait and balance score for at least 52 weeks, wherein the human patient is afflicted with HD and has a baseline TFC score of 11-13.

The invention also provides, a method of improving or maintaining, a human patient's gait and balance comprising periodically orally administering to the patient a pharmaceutical composition comprising pridopidine such that a dose of 90 mg of pridopidine is administered to the patient per day (e.g. 45 mg bid), so as to thereby improve, or maintain, a human patient's gait and balance as measured by the UHDRS gait and balance score for at least 39 weeks, wherein the human patient is afflicted with HD and has a baseline TFC score of 11-13. The invention also provides, a method of reducing dystonia or maintaining a level of dystonia in a human patient afflicted with HD comprising periodically orally administering to the patient a pharmaceutical composition comprising pridopidine such that a dose of 90 or 180 mg of pridopidine is administered to the patient per day, so as to thereby reduce or maintain a level of dystonia as measured by the UHDRS TMS Dystonia score and the human patient has a baseline TFC score of 11-13. The invention also provides, a method of reducing dystonia or maintaining a level of dystonia in a human patient afflicted with HD comprising periodically orally administering to the patient a pharmaceutical composition comprising pridopidine such that a dose of 90 mg of pridopidine is administered to the patient per day (e.g. 45 mg bid), so as to thereby reduce or maintain a level of dystonia as measured by the UHDRS TMS Dystonia score and the human patient has a baseline TFC score of 11-13.

The invention also provides, a method of improving, maintaining, or slowing the decline of, a human patient's independence comprising periodically orally administering to the patient a pharmaceutical composition comprising pridopidine such that a dose of 90 mg of pridopidine is administered to the patient per day (e.g. 45 mg bid), so as to thereby improve, maintain, or slow the decline of, a human patient's independence as measured by the UHDRS Independence Score for at least 26 weeks, or at least 39 weeks wherein the human patient is afflicted with HD.

The invention also provides, a method of improving, maintaining, or lessening the decline of, a human patient's independence comprising periodically orally administering to the patient a pharmaceutical composition comprising pridopidine such that a dose of 90 mg of pridopidine is administered to the patient per day (e.g. 45 mg bid), so as to thereby improve, maintain, or lessen the decline of, a human patient's independence as measured by the UHDRS Independence Score for at least 26 weeks, at least 39 weeks, or at least 65 weeks, wherein the human patient is afflicted with early HD.

In one embodiment, the human patient has a baseline TFC score of 11-13. In another embodiment, the human patient has a baseline TFC score of greater than or equal to 7.

The invention also provides, a method of preventing the slowing, the reduction in amplitude, or the impairment of the human patient's finger tapping ability in a human patient afflicted with HD comprising periodically orally administering to the patient a pharmaceutical composition comprising pridopidine such that a dose of 90 mg of pridopidine is administered to the patient per day (e.g. 45 mg bid), so as to thereby prevent the slowing, the reduction in amplitude, or the impairment of the human patient's finger tapping ability.

In one embodiment, the method further comprises preventing the slowing or the irregular performance of the Pronate-Supinate Hands test in the human patient.

The invention also provides a method of improving or maintaining a human patient's behavior and/or psychiatric state comprising periodically orally administering to the human patient afflicted with HD a pharmaceutical composition comprising pridopidine such that a dose of 90 mg of pridopidine is administered to the patient per day (e.g. 45 mg bid), so as to thereby improve or maintain the human patient's behavior and/or psychiatric state for at least 26 weeks, or at least 39 weeks, or at least 52 weeks or at least 65 weeks, as measured by the Problem Behaviors Assessment for irritability or for disoriented behavior.

In one embodiment, the human patient has a baseline TFC score of 0-6, the human patient's behavior and/or psychiatric state is measured by the Problem Behaviors Assessment for irritability and the human patient's behavior and/or psychiatric state is improved or maintained for at least 39 weeks, at least 52 weeks, at least 65 weeks, or at least 78 weeks. In another embodiment, the human patient has a baseline TFC score of 11-13, the human patient's behavior and/or psychiatric state is measured by the Problem Behaviors Assessment for disoriented behavior and the human patient's behavior and/or psychiatric state is improved or maintained for at least 26 weeks, at least 39 weeks, at least 52 weeks, or at least 65 weeks, or at least 78 weeks.

The invention also provides, a method of maintaining:
a) functional capacity in a human patient;
b) a human patient's ability to perform activities of daily living;
c) a human patient's ability to manage finances;
d) a human patient's ability to perform domestic chores;
e) the human patient's care level;
f) Dystonia in a human patient;
g) a human patient's Gait and balance;
h) a human patient's independence;
i) a human patient's cognitive domains;
j) chorea in a human patient;
k) a human patient's behavior and/or psychiatric state;
l) motor ability in a human patient;
m) a human patient's mobility;
n) a human patient's ability to perform physical tasks; or
o) a human patient's Quality of Life;
   comprising periodically orally administering to the patient a pharmaceutical composition comprising pridopidine such that a dose of 90 mg of pridopidine or 45 mg bid of pridopidine is administered to the patient per day.

The invention also provides, a method of improving:
a) functional capacity;
b) a human patient's ability to perform activities of daily living;
c) a human patient's ability to manage finances;
d) a human patient's ability to perform domestic chores;
e) a human patient's care level;
f) a human patient's gait and balance;
g) a human patient's independence;
h) a human patient's cognitive domains;
i) motor ability in a human patient;
j) chorea in a human patient;
k) a human patient's behavior and/or psychiatric state;
l) a human patient's mobility; or
m) a human patient's ability to perform physical tasks;
   comprising periodically orally administering to the patient a pharmaceutical composition comprising pridopidine such that a dose of 90 mg of pridopidine is administered to the patient per day (e.g. 45 mg bid).

The invention also provides, a method of reducing:
a) dystonia in a human patient;
b) a human patient's involuntary movements; or
c) the severity of the sustained or intermittent muscle contractions associated with dystonia in a human patient,
   comprising periodically orally administering to the patient a pharmaceutical composition comprising pridopidine such that a dose of 90 mg of pridopidine or 45 mg bid of pridopidine is administered to the patient per day.

The invention also provides a method of reducing:
a) the decline of functional capacity in a human patient;
b) the rate of decline of a human patient's ability to perform activities of daily living;
c) the rate of decline of a human patient's ability to manage finances;
d) the rate of decline of a human patient's ability to perform domestic chores;
e) the rate of decline of a human patient's care level;
f) the decline of a human patient's behavior and/or psychiatric state;
g) the rate of decline of a human patient's Quality of Life;
h) the rate of decline of a human patient's Cognitive domains; or
i) the rate of decline of a human patient's Motor domains;
   comprising periodically orally administering to the patient a pharmaceutical composition comprising pridopidine such that a dose of 90 of pridopidine is administered to the patient per day (e.g. 45 mg bid).

The invention also provides a method of lessening:
a) the decline of functional capacity in a human patient;
b) the decline of a human patient's ability to perform activities of daily living;
c) the decline of a human patient's ability to manage finances;
d) the decline of a human patient's ability to perform domestic chores;
e) the decline of a human patient's care level;
f) the decline of a human patient's behavior and/or psychiatric state; or
g) the decline of a human patient's Quality of Life;
h) the decline of a human patient's Cognitive domains; or
i) the decline of a human patient's Motor domains;
   comprising periodically orally administering to the patient a pharmaceutical composition comprising pridopidine such that a dose of 90 mg of pridopidine is administered to the patient per day (e.g. 45 mg bid).

The invention also provides a method of:
a) slowing the decline of a human patient's gait and balance;
b) slowing the decline of a human patient's independence;
c) slowing the decline of a human patient's cognitive domains;
i) slowing the decline of a human patient's Motor domains;
g) slowing the decline of a human patient's Quality of Life;
   comprising periodically orally administering to the patient a pharmaceutical composition comprising pridopidine such that a dose of 90 mg of pridopidine is administered to the patient per day (e.g. 45 mg bid).

The invention also provides a method of:
a) lessening the decline of a human patient's gait and balance;
b) lessening the decline of a human patient's independence; or
c) lessening the decline of a human patient's cognitive domains;
   comprising periodically orally administering to the patient a pharmaceutical composition comprising pridopidine such that a dose of 90 mg of pridopidine is administered to the patient per day (e.g. 45 mg bid).

The invention also provides a method of improving:
a) a human patient's cognitive domains;
b) a human patient's Motor domains; or
c) a human patient's Quality of Life;
   comprising periodically orally administering to the patient a pharmaceutical composition comprising pridopidine such that a dose of 90 mg of pridopidine is administered to the patient per day (e.g. 45 mg bid).

In some embodiments, provided herein, a method of improving or maintaining or slowing the decline of a human patient's Motor domains comprising periodically orally administering to the patient a pharmaceutical composition comprising pridopidine such that a dose of 90 mg of pridopidine is administered to the patient per day (e.g. 45 mg bid), so as to thereby improve or maintain or slow the decline of the human patient's Motor domains.

In some embodiments, provided herein, a method of improving, maintaining, or slowing the decline of, a human patient's Cognitive domains comprising periodically orally administering to the patient a pharmaceutical composition comprising pridopidine such that a dose of 90 mg of pridopidine is administered to the patient per day (e.g. 45 mg bid), so as to thereby improve, maintain, or slow the decline of, a human patient's cognitive function.

In some embodiments, provided herein, a method of improving or maintaining or slow the decline of a human patient's Quality of Life comprising periodically orally administering to the patient a pharmaceutical composition comprising pridopidine such that a dose of 90 mg of pridopidine is administered to the patient per day (e.g. 45 mg bid), so as to thereby improve or maintain the human patient's Quality of Life.

In some embodiments, provided herein, method of improving, maintaining, or slowing the decline of, a human patient's Quality of Life comprising periodically orally administering to the patient a pharmaceutical composition comprising pridopidine such that a dose of 90 mg of pridopidine is administered to the patient per day (e.g. 45 mg bid), so as to thereby improve, maintain, or slow the decline of, a human patient's Quality of Life.

In some embodiments, provided herein, a method of improving or maintaining or slow the decline of a human patient's cognitive domains comprising periodically orally administering to the patient a pharmaceutical composition comprising pridopidine such that a dose of 90 mg of pridopidine is administered to the patient per day (e.g. 45 mg bid), so as to thereby improve or maintain the human patient's cognitive domains.

The invention also provides a method of:
a) slowing the clinical progression of HD as measured by total functional capacity in a human patient; or
b) treating limb dystonia, preferably, wherein treating comprises
i. preventing the slowing, the reduction in amplitude, or the impairment of the human patient's finger tapping ability and preventing the slowing or irregular performance of the Pronate-Supinate Hands test in the human patient;
ii. preventing the slowing or the irregular performance of the Pronate-Supinate Hands test in the human patient;
iii. improving the human patient's Q-motor tap speed frequency; or
iv. improving the human patient's Q-motor tap speed inter onset interval (IOI);
   comprising periodically orally administering to the patient a pharmaceutical composition comprising pridopidine such that a dose of 90 mg of pridopidine is administered to the patient per day (e.g. 45 mg bid).

The invention further provides a pharmaceutical composition comprising pridopidine for use in (1) (a) maintaining functional capacity, improving functional capacity, or lessening functional decline in a human patient in need thereof, (b) slowing the clinical progression of HD, (c) reducing dystonia or maintaining a level of dystonia in a human patient in need thereof, (d) treating limb dystonia in a human patient in need thereof, (e) preventing the slowing, the reduction in amplitude, or the impairment of the human patient's finger tapping ability and/or preventing the slowing or the irregular performance of the Pronate-Supinate Hands test, (f) improving or maintaining, a human patient's gait and balance in a human patient in need thereof, (g) improving or maintaining, a human patient's independence in a human patient in need thereof, (h) improving or maintaining a human patient's cognitive performance across a variety of domains in a human patient in need thereof, (i) lessening the severity of the sustained or intermittent muscle contractions associated with dystonia in a human patient in need thereof, (j) improving or maintaining motor ability in a human patient in need thereof, (k) reducing or maintaining the level of chorea in a human patient in need thereof, (1) improving, maintaining, or lessening the decline of a human patient's behavior and/or psychiatric state in a human patient in need thereof, (m) reducing or maintaining a human patient's involuntary movements in a human patient in need thereof, (n) improving or maintaining a human patient's mobility in a human patient in need thereof, (o) improving or maintaining a human patient's ability to perform physical tasks, (p) improving or maintaining a human patient's quality of life wherein the pharmaceutical composition is to be periodically orally administered to the patient such that a dose of 90-225 mg of pridopidine or 45 mg bid of pridopidine is to be administered to the patient per day, or (2) (a) improving, maintaining, or lessening the decline of gait and balance in a human patient in need thereof (b) improving, maintaining, or lessening the decline of, a human patient's independence in a human patient in need thereof, (c) improving, maintaining, or lessening the decline of, a human patient's cognitive performance across a variety of domains in a human patient in need thereof, (d) reducing, maintaining, or lessening the increase of, chorea, in a human patient in need thereof, wherein the pharmaceutical composition is to be periodically orally administered to the patient such that a dose of 90 mg of pridopidine (e.g. 45 mg bid) is to be administered to the patient per day.

The invention also provides the use of an amount of pridopidine in the manufacture of a medicament for (1) (a) maintaining functional capacity, improving functional capacity, or lessening functional decline in a human patient in need thereof, (b) slowing the clinical progression of HD, (c) reducing dystonia or maintaining a level of dystonia in a human patient in need thereof, (d) treating limb dystonia in a human patient in need thereof, (e) preventing the slowing, the reduction in amplitude, or the impairment of the human patient's finger tapping ability and/or preventing the slowing or the irregular performance of the Pronate-Supinate Hands test, (f) improving or maintaining, a human patient's gait and balance in a human patient in need thereof, (g) improving or maintaining, a human patient's independence in a human patient in need thereof, (h) improving, or maintaining, or slowing the decline a human patient's cognitive performance across a variety of domains in a human patient in need thereof, (i) lessening the severity of the sustained or intermittent muscle contractions associated with dystonia in a human patient in need thereof, (j) improving or maintaining motor ability in a human patient in need thereof, (k) reducing or maintaining the level of chorea in a human patient in need thereof, (1) improving, maintaining, or lessening the decline of a human patient's behavior and/or psychiatric state in a human patient in need thereof, (m) reducing or maintaining a human patient's involuntary movements in a human patient in need thereof, (n) improving or maintaining a human patient's mobility in a human patient in need thereof, (o) improving or maintaining a human patient's ability to perform physical tasks, (p) improving or maintaining, or slowing the decline a human patient's quality of life, (q) improving or maintaining or slowing the decline of a human patient's Motor domains wherein the pharmaceutical composition is to be periodically orally administered to the patient such that a dose of 90-225 mg of pridopidine or 45 mg bid of pridopidine is to be administered to the patient per day, or (2) (a) improving, maintaining, or lessening the decline of gait and balance in a human patient in need thereof (b) improving, maintaining, or lessening the decline of, a human patient's independence in a human patient in need thereof, (c) improving, maintaining, or lessening the decline of, a human patient's cognitive performance across a variety of domains in a human patient in need thereof, (d) reducing, maintaining, or lessening the increase of, chorea, in a human patient in need thereof wherein the medicament is formulated for periodic oral administration to the patient such that a dose of 90 mg of pridopidine is to be administered to the patient per day (e.g. 45 mg bid).

The invention additionally provides the use of an amount of pridopidine for (1) (a) maintaining functional capacity, improving functional capacity, or lessening functional decline in a human patient in need thereof, (b) slowing the clinical progression of HD, (c) reducing dystonia or maintaining a level of dystonia in a human patient in need thereof, (d) treating limb dystonia in a human patient in need thereof, (e) preventing the slowing, the reduction in amplitude, or the impairment of the human patient's finger tapping ability and/or preventing the slowing or the irregular performance of the Pronate-Supinate Hands test, (f) improving or maintaining, a human patient's gait and balance in a human patient in need thereof, (g) improving or maintaining, a human patient's independence in a human patient in need thereof, (h) improving or maintaining a human patient's cognitive performance across a variety of domains in a human patient in need thereof, (i) lessening the severity of the sustained or intermittent muscle contractions associated with dystonia in a human patient in need thereof, (j) improving or maintaining motor ability in a human patient in need thereof, (k) reducing or maintaining the level of chorea in a human patient in need thereof, (1) improving, maintaining, or lessening the decline of a human patient's behavior and/or psychiatric state in a human patient in need thereof, (m) reducing or maintaining a human patient's involuntary movements in a human patient in need thereof, (n) improving or maintaining a human patient's mobility in a human patient in need thereof, (o) improving or maintaining a human patient's ability to perform physical tasks, (p) improving or maintaining a human patient's quality of life wherein the pharmaceutical composition is to be periodically orally administered to the patient such that a dose of 90-225 mg of pridopidine is to be administered to the patient per day, or (2) (a) improving, maintaining, or lessening the decline of gait and balance in a human patient in need thereof (b) improving, maintaining, or lessening the decline of, a human patient's independence in a human patient in need thereof, (c) improving, maintaining, or lessening the decline of, a human patient's cognitive performance across a variety of domains in a human patient in need thereof, (d) reducing, maintaining, or lessening the increase of, chorea, in a human patient in need thereof wherein the medicament is formulated for periodic oral administration to the patient such that a dose of 90 mg of pridopidine is to be administered to the patient per day.

Each embodiment disclosed herein is contemplated as being applicable to each of the other disclosed embodiments. For instance, all combinations of the various elements described herein are within the scope of the invention. Additionally, the elements recited in the packaging and pharmaceutical composition embodiments can be used in the method and use embodiments described herein.

### Terms

As used herein, and unless stated otherwise, each of the following terms shall have the definition set forth below.

The articles “a”, “an” and “the” are non-limiting. For example, “the method” includes the broadest definition of the meaning of the phrase, which can be more than one method.

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to maintain functional capacity or lessen decline in functional capacity. The specific effective amount varies with such factors as the particular condition being treated, the physical condition of the patient, the type of mammal being treated, the duration of the treatment, the nature of concurrent therapy (if any), and the specific formulations employed and the structure of the compounds or its derivatives.

As used herein, to “treat” or “treating” encompasses, e.g., reducing a symptom, inducing inhibition, regression, or stasis of the disorder and/or disease. As used herein, “inhibition” of disease progression or disease complication in a subject means preventing or reducing the disease progression and/or disease complication in the subject.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration.

As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times a week and so on, etc.

As used herein, “maintaining functional capacity in a human patient” means that the functional capacity score after a period of administration of pridopidine (“the after administration score”) is unchanged compared to the human patient's functional capacity score immediately prior to the period of administration (“the baseline score”). The after administration score is considered to be unchanged if the difference between the baseline score and the after administration score is not statistically significant. The functional capacity score can be measured as described herein and includes subsets of the functional capacity score.

As used herein, “improving functional capacity in a human patient” means that the functional capacity score after a period of administration of pridopidine (“the after administration score”) is improved compared to the human patient's functional capacity score immediately prior to the period of administration (“the baseline score”).

The functional capacity score of a human patient afflicted with HD can decrease over time. The rate of such decrease can be referred to as a rate of decline of the functional capacity score or a rate of decline of functional capacity or a rate of functional decline. For example, on average the rate of functional decline or the reduction in TFC score is faster for early stage HD patients (TFC score 7-13) than for advanced stage patients (TFC score of <7). On average the decline is about 0.8-1.2 points per year in early stage HD patients, less than ½ (about 0.2-0.3) point per year for patients with TFC 3-6; and less than 0.1 for patients with TFC 0-2 (Marder 2000). Therefore, TFC is most sensitive to change in the earlier stages of disease. The total functional capacity score can be measured as described herein and includes subsets of the functional capacity score. This decline may also be referred to as the natural decline or the untreated decline in functional capacity.

Accordingly, as used herein, “reducing the rate of decline of functional capacity”, “slowing the rate of functional decline”, “reducing the rate of functional decline”, “decreased functional decline”, or “slowing functional decline” means that the rate of decline of the functional capacity score after a period of administration of pridopidine (“the after administration score”) is slowed, reduced or decreased compared to the functional capacity score of a patient who has not received the same treatment with pridopidine.

As used herein, “lessening the decline of functional capacity” or “reducing the decline of functional capacity” means that the decrease in a functional capacity score in a patient after a period of administration of pridopidine is less than the decrease in the functional capacity score of a patient who has not received the same treatment with pridopidine over the same period.

As used herein, “lessening the decline of HD” encompasses assessing the progression of HD using any of the scales and scores disclosed herein, including the cUHDRS measurements or the individual measurements thereof, e.g., TFC, TMS, SDMT, SWR or any combination thereof, wherein after a period of administration of pridopidine, the rate of progression of HD as assessed by a method described herein, is decreased in a patient after a period of administration of pridopidine compared with a patient who has not received the same treatment with pridopidine over the same period. cUHDRS, TFC, TMS, SDMT, and SWR may be measured as described herein and includes subsets of the score or scores being measured. In some embodiments, the patient is suffering from early stage HD (HD1 or HD2). In some embodiments, the patient is suffering from early stage HD (HD1 or HD2 or/and TFC 7-13).

In some embodiments, lessening the decline of the progression of HD comprises slowing the motor deterioration in said patient compared with a patient who has not received the same treatment with pridopidine over the same period. In some embodiments, lessening the decline of the progression of HD comprises improving the functional capacity (TFC) in said patient compared with a patient who has not received the same treatment with pridopidine over the same period. In some embodiments, lessening the decline of the progression of HD comprises slowing the motor deterioration and improving the functional capacity (TFC) in said patient compared with a patient who has not received the same treatment with pridopidine over the same period. In some embodiments, the period of time is between 6-60 months. In some embodiments, the period of time is between 12-60 months. In some embodiments, the period of time is between 24-60 months. In some embodiments, the period of time is between 36-60 months. In some embodiments, the period of time is between 48-60 months. In some embodiments, the period of time is between 24-48 months. In some embodiments, the period of time is between 36-48 months. In some embodiments, the period of time is between 36-60 months. In some embodiments, the period of time is between 48-60 months. In some embodiments, the period of time is 6 months. In some embodiments, the period of time is 12 months. In some embodiments, the period of time is 24 months. In some embodiments, the period of time is 36 months. In some embodiments, the period of time is 48 months. In some embodiments, the period of time is 60 months. In some embodiments, the period of time is at least 6 months. In some embodiments, the period of time is at least 12 months. In some embodiments, the period of time is at least 24 months. In some embodiments, the period of time is at least 36 months. In some embodiments, the period of time is at least 48 months. In some embodiments, the period of time is at least 60 months.

In some embodiments, a method of assessing HD progression comprises using cUHDRS.

In some embodiments, a method of assessing HD progression comprises using TFC or TMS or SDMT or SWR, or any combination thereof. In some embodiments, when assessing HD progression comprises use of cUHDRS, an improved longitudinal S/N ratio is observed. In some embodiments, when assessing HD progression comprises use of cUHDRS, an improved longitudinal S/N ratio is observed, wherein the cUHDRS measurement shows improved measurement values compared with the independent UHDRS clinical measures of TFC, TMS, SDMT, or SWR, or any combination thereof. In some embodiments, when assessing HD progression comprises use of cUHDRS, an improved longitudinal S/N ratio is observed, wherein the cUHDRS measurement shows improved measurement values compared with the independent UHDRS clinical measures of TFC, TMS, SDMT, and SWR. In some embodiments, when assessing HD progression comprises use of cUHDRS, the use provides improved statistical significant values compared to significance values of an independent TFC or TMS or SDMT or SWR analysis. In some embodiments, when assessing HD progression comprises use of cUHDRS, the use provides improved statistical significant values compared to significance values of an independent TFC analysis. In some embodiments, when assessing HD progression comprises use of cUHDRS, the use provides improved statistical significant values compared to significance values of an independent TMS analysis. In some embodiments, when assessing HD progression comprises use of cUHDRS, the use provides improved statistical significant values compared to significance values of an independent SDMT analysis. In some embodiments, when assessing HD progression comprises use of cUHDRS, the use provides improved statistical significant values compared to significance values of an independent SWR analysis.

As used herein, “maintaining a human patient's ability to perform activities of daily living” means that the activities of daily living (ADL) score after a period of administration of pridopidine (“the after administration score”) is unchanged compared to the human patient's activities of daily living score immediately prior to the period of administration (“the baseline score”). The after administration score is considered to be unchanged if the difference between the baseline score and the after administration score is not statistically significant. The activities of daily living score is a subset of the total functional capacity score and can be measured as described herein.

There are six basic ADLs: eating, bathing, dressing, toileting, transferring (functional mobility) and continence. ADL is scored as follows: a patient requiring total care=0, a patient able to carry out gross tasks only=1, a patient having minimal impairment=2, a patient with no impairment (normal)=3.

As used herein, “maintaining a human patient's ability to manage finances” means that the finances score after a period of administration of pridopidine (“the after administration score”) is unchanged compared to the human patient's finances score immediately prior to the period of administration (“the baseline score”). The after administration score is considered to be unchanged if the difference between the baseline score and the after administration score is not statistically significant. The finances score is a subset of the total functional capacity score and can be measured as described herein.

Finance is scored as follows: a patient unable to manage finances=0, a patient requiring major assistance=1, a patient requiring minor assistance=2, a patient a patient requiring no assistance (normal)=3.

As used herein, “maintaining functional capacity, motor function and cognitive function” encompasses assessing the progression of HD using any of the scales and scores disclosed herein, including the cUHDRS measurements or the individual measurements thereof, e.g., TFC, TMS, SDMT, SWR or any combination thereof, wherein after a period of administration of pridopidine, the “after administration score” is unchanged compared to the human patient's scale or score immediately prior to the period of administration (“the baseline score”). In some embodiments, “maintaining” comprises halting the progression of HD. The “after administration score” is considered to be unchanged if the difference between the baseline score and the “after administration score” is not statistically significant. The cUHDRS, TFC, TMS, SDMT, and SWR may be measured as described herein and includes subsets of the score or scores being measured. In some embodiments, the patient is suffering from early stage HD (HD1 or HD2 or/and TFC 7-13).

In some embodiments, maintaining motor function comprises maintaining and stabilizing the motor deterioration in said patient compared with a patient who has not received the same treatment with pridopidine over the same period. In some embodiments, maintaining functional capacity comprises maintaining and stabilizing the functional capacity (TFC) in said patient compared with a patient who has not received the same treatment with pridopidine over the same period. In some embodiments, the period of time is between 6-60 months. In some embodiments, the period of time is between 12-60 months. In some embodiments, the period of time is between 24-60 months. In some embodiments, the period of time is between 36-60 months. In some embodiments, the period of time is between 48-60 months. In some embodiments, the period of time is between 24-48 months. In some embodiments, the period of time is between 36-48 months. In some embodiments, the period of time is between 36-60 months. In some embodiments, the period of time is between 48-60 months. In some embodiments, the period of time is 6 months. In some embodiments, the period of time is 12 months. In some embodiments, the period of time is 24 months. In some embodiments, the period of time is 36 months. In some embodiments, the period of time is 48 months. In some embodiments, the period of time is 60 months. In some embodiments, the period of time is at least 6 months. In some embodiments, the period of time is at least 12 months. In some embodiments, the period of time is at least 24 months. In some embodiments, the period of time is at least 36 months. In some embodiments, the period of time is at least 48 months. In some embodiments, the period of time is at least 60 months.

As used herein, “no worsening of other HD symptoms compared to baseline” means that the severity of each of the human patient's HD symptoms after a period of administration of pridopidine is equal to or less than the severity of the symptom immediately prior to the start of the period of administration (baseline).

For each baseline score discussed above, in one embodiment, there is no administration of pridopidine to the patient prior to attainment of the baseline score. In another embodiment, an amount of pridopidine is administered to the patient prior to attainment of the baseline score. In a further embodiment, the amount of pridopidine administered to the patient prior to attainment of the baseline score is less than or more than the amount of pridopidine administered to the patient after the attainment of the baseline score.

As used herein, “no worsening of other HD symptoms compared to a human patient not treated with pridopidine” means that the severity of each of the human patient's HD symptoms after a period of administration of pridopidine is equal to or less than the severity of the symptom compared with a human HD patient not being administered pridopidine. In some embodiments, the stage of HD of both HD patients is comparable, for example but not limited to both patients are at an early stage of HD (HD1 or HD2). In some embodiments, the stage of HD of both HD patients is comparable, for example but not limited to both patients are at an early stage of HD (HD1 or HD2 and/or TFC 7-13). In some embodiments, the stage of HD of both HD patients is comparable, for example but not limited to both patients are at a late stage of HD (HD3 or HD4). In some embodiments, the stage of HD of both HD patients is comparable, for example but not limited to both patients are at a stage of HD being HD1, HD2, HD3, or HD4, or a combination thereof.

As used herein, an “amount” or “dose” of pridopidine as measured in milligrams refers to the milligrams of pridopidine present in a preparation, regardless of the form of the preparation. A “dose of 90 mg pridopidine” means the amount of pridopidine acid in a preparation is 90 mg, regardless of the form of the preparation. Thus, when in the form of a salt, e.g. a pridopidine hydrochloride, the weight of the salt form necessary to provide a dose of 90 mg pridopidine would be greater than 90 mg due to the presence of the additional salt ion.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1; 0.2 . . . 0.9; and 1, 2 . . . 49 mg unit amounts are included as embodiments of this invention.

As used herein, “pridopidine” means pridopidine base or a pharmaceutically acceptable salt thereof, as well as derivatives, for example deuterium-enriched version of pridopidine and salts. Examples of deuterium-enriched pridopidine and salts and their methods of preparation may be found in U.S. Application Publication Nos. 2013-0197031, 2016-0166559 and 2016-0095847, the entire content of each of which is hereby incorporated by reference. In certain embodiments, pridopidine is a pharmaceutically acceptable salt, such as the HCl salt or tartrate salt. Preferably, in any embodiments of the invention as described herein, the pridopidine is in the form of its hydrochloride salt.

“Deuterium-enriched” means that the abundance of deuterium at any relevant site of the compound is more than the abundance of deuterium naturally occurring at that site in an amount of the compound. The naturally occurring distribution of deuterium is about 0.0156%. Thus, in a “deuterium-enriched” compound, the abundance of deuterium at any of its relevant sites is more than 0.0156% and can range from more than 0.0156% to 100%. Deuterium-enriched compounds may be obtained by exchanging hydrogen with deuterium or synthesizing the compound with deuterium-enriched starting materials.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the L-tartrate, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

### Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

In an embodiment, the invention provides pharmaceutical compositions comprising the active compounds or pharmaceutically acceptable salts or derivatives thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. In some embodiments, in a method of maintaining, improving, or lessening the decline of motor function and functional capacity in a human patient afflicted with early stage Huntington disease (HD1 and HD2, TFC 7-13), comprising orally administering to the patient a pharmaceutical composition comprising pridopidine or a pharmaceutically acceptable salt thereof, oral administration comprises administration by a capsule. The pharmaceutical composition of the invention can be manufactured by the skilled person by use of standard methods and conventional techniques appropriate to the desired formulation. When desired, compositions adapted to give sustained release of the active ingredient may be employed.

In some embodiments the methods of this invention make use of a pharmaceutical composition comprising pridopidine or pharmaceutically acceptable salt thereof and at least one analog compound 1-7 or pharmaceutically acceptable salt thereof; wherein analog compounds 1-7 are represented by the following structures:

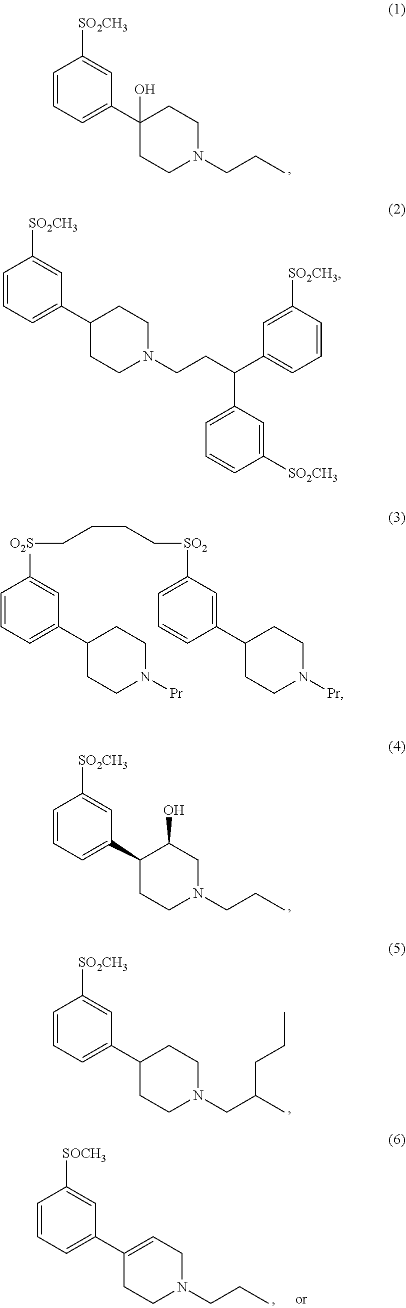

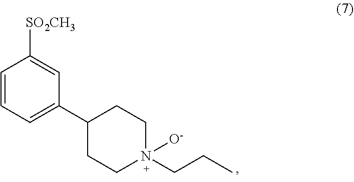

In other embodiments this invention provides a pharmaceutical composition comprising pridopidine or pharmaceutically acceptable salt thereof and an analog compound 1 or pharmaceutically acceptable salt thereof. In other embodiments this invention provides a pharmaceutical composition comprising pridopidine or pharmaceutically acceptable salt thereof and an analog compound 2 or pharmaceutically acceptable salt thereof. In other embodiments this invention provides a pharmaceutical composition comprising pridopidine or pharmaceutically acceptable salt thereof and an analog compound 3 or pharmaceutically acceptable salt thereof. In other embodiments this invention provides a pharmaceutical composition comprising pridopidine or pharmaceutically acceptable salt thereof and an analog compound 4 or pharmaceutically acceptable salt thereof. In other embodiments this invention provides a pharmaceutical composition comprising pridopidine or pharmaceutically acceptable salt thereof and an analog compound 5 or pharmaceutically acceptable salt thereof. In other embodiments this invention provides a pharmaceutical composition comprising pridopidine or pharmaceutically acceptable salt thereof and an analog compound 6 or pharmaceutically acceptable salt thereof. In other embodiments this invention provides a pharmaceutical composition comprising pridopidine or pharmaceutically acceptable salt thereof and an analog compound 7 or pharmaceutically acceptable salt thereof. In other embodiments this invention provides a pharmaceutical composition comprising pridopidine or pharmaceutically acceptable salt thereof and an analog compound 1 and an analog compound 4 or pharmaceutically acceptable salt thereof.

In other embodiments, the analog compounds 1-7 of pridopidine and their methods of preparation may be found in U.S. Pat. Nos. 10,130,621 and 10,406,145 the entire content of each of which is hereby incorporated by reference.

In another embodiment the pharmaceutical composition for use in the methods of this invention comprises pridopidine or pharmaceutically acceptable salt thereof with at least one of compounds 1-7, wherein the at least one of compounds 1-7 are in a weight percentage of between 0.01% w/w to 5% w/w of pridopidine. In another embodiment the composition for use in the methods of this invention comprises pridopidine or pharmaceutically acceptable salt thereof with at least one of compounds 1-7, wherein the at least one of compounds 1-7 are in a weight percentage of between 0.01% to 1% w/w, 0.05 to 0.5% w/w or 0.05% w/w to 1% w/w of pridopidine. In another embodiment the composition for use in the methods of this invention comprises pridopidine or pharmaceutically acceptable salt thereof with compound 1 or pharmaceutically acceptable salt thereof, wherein compound 1 is in a weight percentage of between 0.01% w/w to 5% w/w, 0.01% w/w to 1% w/w, 0.05% w/w to 0.1% w/w, 0.05% w/w to 0.5% w/w or 0.05% w/w to 1% w/w of the composition. In another embodiment the composition for use in the methods of this invention comprises pridopidine or pharmaceutically acceptable salt thereof with compound 4 or pharmaceutically acceptable salt thereof, wherein compound 4 is in a weight percentage of between 0.01% w/w to 5% w/w, 0.01% w/w to 1% w/w, 0.05% w/w to 0.5% w/w, 0.1 w/w to 0.2 w/w, or 0.05% w/w to 1 w/w of pridopidine.

In some embodiments provided herein a method of maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof and Compound 1, Compound 4, combination thereof or pharmaceutically acceptable salt thereof, wherein Compound 1 and Compound 4 are represented by the following structures:

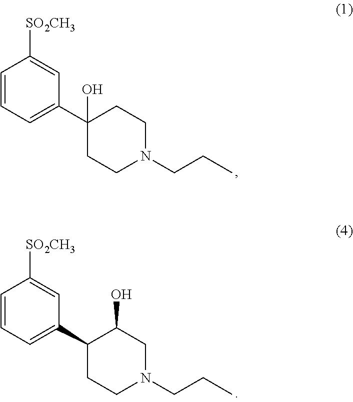 [SMILES: CCCN1CCC(O)(c2cccc(S(C)(=O)=O)c2)CC1 ; CCCN1CC[C@@H](c2cccc(S(C)(=O)=O)c2)[C@@H](O)C1]

In some embodiments provided herein a method of maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof and Compound 1, Compound 4, combination thereof or pharmaceutically acceptable salt thereof, wherein Compound 1 and Compound 4 are represented by the following structures:

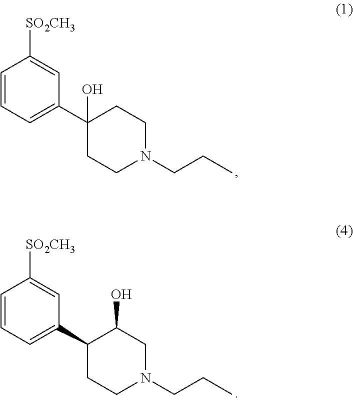 [SMILES: CCCN1CCC(O)(c2cccc(S(C)(=O)=O)c2)CC1 ; CCCN1CC[C@@H](c2cccc(S(C)(=O)=O)c2)[C@@H](O)C1]

wherein assessment of said maintaining, improving, or lessening the decline of motor function and functional capacity comprises using a composite Unified Huntington Disease rating scale (cUHDRS), wherein said cUHDRS comprises measurement of the total functional capacity (TFC), total motor score (TMS), symbol digital modalities test (SDMT), and Stroop Word Reading Test (SWR) of the patient according to the following equation:

$$cUHDRS = [ ( \frac{T F C - 10.4}{1.9} ) - ( \frac{TMS - 29.7}{14.9} ) + ( \frac{S D M T - 28.4}{11.3} ) + ( \frac{SWR - 66.1}{20.1} ) ] + 10.$$

In some embodiments, the administration is for at least 26 weeks or at least 52 weeks. In some embodiments, the pridopidine or a pharmaceutically acceptable salt thereof is administered at a dose 45 mg bid for a period of at least 26 weeks or at least 52 weeks. In another embodiment, the motor function is assessed by Q-motor. In another embodiment, the maintaining, improving, or lessening the decline of motor function and functional capacity does not include administering neuroleptics medications including olanzapine, risperidone, tiapride, aripiprazole, quetiapine, fluphenazine, haloperidol, lithium, ziprasidone, amisulpride, asenapine, cariprazine, chlorpromazine, clotiapine, cyamemazine, flupentixol, prochlorperazine, promazine, and prothipendyl.

Chorea medications include VMAT inhibitors such as Tetrabenazine and deutetrabenazine. In another embodiment, the maintaining, improving, or lessening the decline of motor function and functional capacity does not include administering chorea medications.

In some embodiments the SWR (Stroop Word Reading) was improved after at least 26 weeks of treatment, at least 39 weeks of treatment, 52 weeks of treatment or 65 weeks of treatment. In another related aspect, the method comprises maintaining, improving, or lessening the decline of Stroop Word Reading (SWR) test of said patient.

In some embodiments, provided herein a method of maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof and Compound 1 or pharmaceutically acceptable salt thereof. In some embodiments, the administration is for at least 26 weeks or at least 52 weeks. In some embodiments, the pridopidine or a pharmaceutically acceptable salt thereof is administered at a dose 45 mg bid for a period of at least 26 weeks or at least 52 weeks. In another embodiment, the motor function is assessed by Q-motor. In another embodiment, the maintaining, improving, or lessening the decline of motor function and functional capacity does not include administering neuroleptics medications. In another embodiment, the maintaining, improving, or lessening the decline of motor function and functional capacity does not include administering chorea medications such as VMAT inhibitors such as Tetrabenazine and deutetrabenazine.

In some embodiments, provided herein a method of maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof and Compound 4 or pharmaceutically acceptable salt thereof. In some embodiments, the administration is for at least 26 weeks, at least 39 weeks, or at least 52 weeks. In some embodiments, the pridopidine or a pharmaceutically acceptable salt thereof is administered at a dose 45 mg bid for a period of at least 26 weeks, at least 39 weeks, or at least 52 weeks. In another embodiment, the motor function is assessed by Q-motor. In another embodiment, the maintaining, improving, or lessening the decline of motor function and functional capacity does not include administering neuroleptics medications. In another embodiment, the maintaining, improving, or lessening the decline of motor function and functional capacity does not include administering chorea medications such as VMAT inhibitors such as Tetrabenazine and deutetrabenazine.

In some embodiments, provided herein a method of maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof, Compound 1 or pharmaceutically acceptable salt thereof and Compound 4 or pharmaceutically acceptable salt thereof. In some embodiments, the administration is for at least 26 weeks, at least 39 weeks, or at least 52 weeks. In some embodiments, the pridopidine or a pharmaceutically acceptable salt thereof is administered at a dose 45 mg bid for a period of at least 26 weeks, at least 39 weeks, or at least 52 weeks. In another embodiment, the motor function is assessed by Q-motor. In another embodiment, the maintaining, improving, or lessening the decline of motor function and functional capacity does not include administering neuroleptics medications. In another embodiment, the maintaining, improving, or lessening the decline of motor function and functional capacity does not include administering chorea medications such as VMAT inhibitors such as Tetrabenazine and deutetrabenazine.

In another some embodiments, the pharmaceutically acceptable salt of Compound 1, Compound 4 or pridopidine comprises a hydrochloride, hydrobromide, hydroiodide, nitrate, perchlorate, phosphate, acid-phosphate, sulphate, bisulfate, formate, gluconate, glucaronate, saccharate, isonicotinate, acetate, aconate, ascorbate, benzenesulphonate, benzoate, cinnamate, citrate, embonate, enantate, fumarate, glutamate, glycolate, lactate, maleate, gentisinate, malonate, mandelate, methanesulfonate, ethanesulfonate, naphthalene-2-sulphonate, phthalate, salicylate, sorbate, stearate, succinate, tartrate, pantothenate, bitartrate, and toluene-p-sulfonate or pamoate (i.e., 1,1′-methylene-bis-(2-hydroxy-3-naphthoate)) salt. In another embodiment, the pharmaceutically acceptable salt is HCl salt.

Further details on techniques for formulation and administration may be found in the latest edition of Remington's Pharmaceutical Sciences (Mack Publishing Co., Easton, PA).

### Listing of Abbreviations

The following abbreviations are used throughout this application:

ALT: alanine aminotransferase; ADL: Activities of Daily Living; AR: Autoregressive; AUC: area under the concentration-time curve; bid: twice daily; BL=Baseline; CAB: cognitive assessment battery; CAB HVLT-R: Cognitive Assessment Battery Hopkins Verbal Learning Test, revised; CGI-C: Clinical Global Impression of Change; CGI-S: Clinical Global Impression of Severity; CI: confidence interval; CIBIC-Plus: Clinician's Interview-based Impression of Change plus Caregiver Input; CIBIS: Clinician's Interview-based Impression of Severity; CIOMS: Council for International Organizations of Medical Sciences; Cmax: maximum observed plasma drug concentration; CNS: central nervous system; CRF: case report form; CRO: contract research organization; CS: Compound Symmetry; C-SSRS: Columbia-Suicide Severity Rating Scale; CYP: cytochrome P450; DSM-IV TR: Diagnostic and Statistical Manual—Fourth Edition Text Revision; EM: extensive metabolizers; EU: European Union; FA: Functional Assessment; FAS: full analysis set; Freq: tapping frequency; GCP: Good Clinical Practice; GFV-C: grip force variability in the static phase; GGT: gamma-glutamyl transpeptidase; HART: Huntington's disease ACR16 Randomized Trial; HCG: human chorionic gonadotropin; HD: Huntington's disease; HD-QoL=Huntington's disease Quality of Life; HVLT-R: HAD-CAB Hopkins Verbal Learning Test-Revised; ICH: International Conference on Harmonisation; IEC: Independent Ethics Committee; IOI: inter onset interval; IPI: inter peak interval; IRB: Institutional Review Board; IRT: interactive response technology; IS: Independence Score; ITI: inter tap interval; ITT: intent-to-treat; LSO: local safety officer; MAD: multiple ascending dose; MedDRA: Medical Dictionary for Regulatory Activities; MermaiHD: Multinational European Multicentre ACR16 study in HD; ML: Maximum-Likelihood; mMS: Modified Motor Score; MoCA: Montreal cognitive assessment; MS: Multiple sclerosis; MTD: maximum tolerated dose; NMDA: N-methyl-D-aspartate; NOAEL: no observed adverse effect level; PBA-s: Problem Behaviors Assessment-Short form; PD: pharmacodynamic(s); PDS: Physical disability scale; PK: pharmacokinetic(s); PM: poor metabolizer; PPT: physical performance test; qd: once daily; Q-Motor: Quantitative motor; QoL: Quality of life; QTcF: Fridericia-corrected QT interval; RBC: red blood cell; REML: Restricted Maximum-Likelihood; SAE: serious adverse event; SD: standard deviation; SDMT: symbol digit modalities test; SOC: system organ class; SOP: standard operating procedure; SUSAR: suspected unexpected serious adverse reaction; t½: half life; TC=telephone call; TD: tap duration; TF: tapping force; TFC: Total Functional Capacity; TMS: Total Motor Score; TMS Involuntary Movements=TMS for performance of Domestic Chores and Dystonia scores combined. TUG: timed up and go; UHDRS: Unified Huntington's Disease Rating Scale; cUHDRS: composite Unified Huntington's Disease Rating Scale; ULN: upper limit of the normal range; US: United States; WBC: white blood cell; WHO: World Health Organization; WHO: Drug World Health Organization (WHO) drug dictionary; AHR: change from baseline in heart rate; AQTcF: change from baseline in QTcF; AAHR: placebo-corrected change from baseline in heart rate; Placebo-Controlled Study—Huntington's Disease; AAQTcF: placebo-corrected change from baseline in QTcF, wk: week; EQ5D-5L European Quality of Life-5 Dimensions (5 levels).

### Clinical Studies

To date, 22 clinical studies with pridopidine have been completed. A total of 8 studies have been conducted in healthy subjects, 1 study in healthy subjects and patients with HD, 1 study in healthy subjects and patients with schizophrenia, 1 study in patients with schizophrenia, 1 study in patients with Parkinson's disease (PD) and 10 studies in patients with HD (of which 3 were open-label extension studies and 1 was an extended/early access program [EAS]). Three phase 2/3 studies in patients with HD were randomized, double-blind, parallel group, placebo-controlled studies: Study ACR16C009, the Huntington Disease (HD) ACR16 Randomized Trial (HART); Study ACR16C008, the Multinational European Multicenter ACR16 study in Huntington Disease (MermaiHD); and Study TV7820-CNS-2002, the Pridopidine Dose Evaluation in Huntington Disease study (PRIDE-HD) with corresponding open-label extensions, which have completed.

This invention will be better understood by reference to the Experimental Details which follow, but those skilled in the art will readily appreciate that the specific experiments detailed are only illustrative of the invention as described more fully in the claims which follow thereafter.

### EXAMPLES

### Example 1: A Phase II, Dose-finding, Randomized, Parallel-Group, Double-Blind, Placebo-Controlled Study, Evaluating the Safety and Efficacy of Pridopidine 45 mg, 67.5 mg, 90 mg, and 112.5 mg Twice-Daily Versus Placebo for Symptomatic Treatment in Patients with HD (“PRIDE-HD”)

The presented study (PRIDE-HD) assessed the efficacy of pridopidine 45 mg to 112.5 mg twice daily (bid) on motor impairment in patients with HD over at least 52 weeks of treatment using the UHDRS TMS. The study also assessed the effect of at least 52 weeks of treatment with pridopidine on UHDRS measures for total function capacity (TFC) and cognitive assessment battery (CAB). The study also compared data from all patients to those obtained in HD subpopulations. The study also (i) evaluated the safety and tolerability of a range of pridopidine doses in patients with HD during at least 52 weeks of treatment, (ii) explored the pharmacokinetics (PK) of pridopidine in the study population and (iii) investigated the relationship between exposure to pridopidine and outcome measures (e.g., clinical efficacy and toxicity parameters).

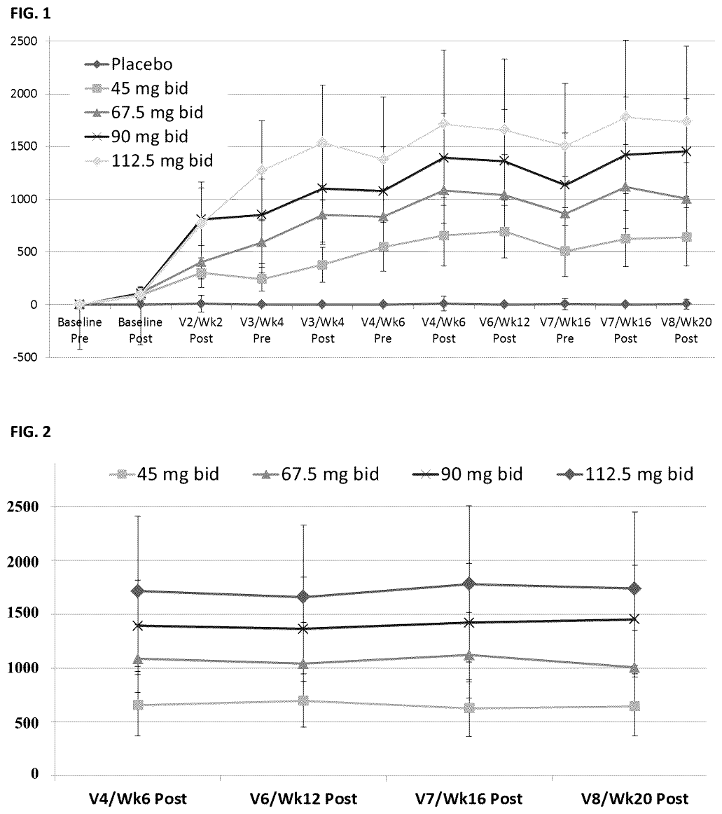

The results of this example are shown in FIGS. 1-40 and described below.

### Description of Clinical Endpoints:

### UHDRS Total Functional Capacity (TFC)

The TFC scale of the UHDRS is a standardized scale used to assess 5 functional domains associated with disability shown below (occupation, finances, domestic chores (e.g. laundry, washing dishes), activities of daily living, and care level). The TFC score has a range of 0-13 and is a well-established endpoint for trials assessing disease functional progression. The TFC score has been developed and deployed by the Huntington Study Group (HSG, 1996) in multiple trials over 2 decades. The TFC score is accepted by regulators and often considered the most widely accepted tool for disease procession in HD patients. Additionally, TFC is considered the gold standard for measuring the rate of functional decline in HD. Currently, no drug has been shown to slow the decline of TFC despite numerous attempts. The floor and ceiling effects make TFC Scores more sensitive to change in early stage HD than in late stage HD.

### Functional Capacity:

Occupation: 0=unable, 1=marginal work only, 2=reduced capacity for usual job, 3=normal.

Finances: 0=unable, 1=major assistance, 2=slight assistance, 3=normal.

Domestic Chores: 0=unable, 1=impaired, 2=normal.

Activities of Daily Living (ADL): 0=total care, 1=gross tasks only, 2=minimal impairment, 3=normal.

Care level: 0=fill time skill nursing, 1=home or chronic care, 2=home.

### UHDRS Total Motor Score (TMS)

The TMS component of UHDRS comprises 31 assessments from the 15 items of the UHDRS from 0 (normal) to 4 (maximal abnormal)

### Total Motor Score Subscores

### UHDRS Hand Movement Score or UHDRS TMS Hand Movement Score

The hand movement score is defined as the sum of UHDRS domains finger taps, pronate-supinate hands and luria (fist-hand-palm test).

### UHDRS Gait and Balance Score or UHDRS TMS Gait and Balance Score

The gait and balance score is defined as the sum of UHDRS domains gait, tandem walking and retropulsion pull test.

### UHDRS Chorea or UHDRS TMS Chorea

In the UHDRS, maximal chorea was scored from 0 (absent) to 4 (marked/prolonged) on each of the following items: face, mouth, trunk, right upper extremity, left upper extremity, right lower extremity, and left lower extremity. Maximal chorea is the sum of all scores.

### UHDRS Dystonia or UHDRS TMS Dystonia

In the UHDRS, maximal dystonia was scored from 0 (absent) to 4 (marked/prolonged) on each of the following items: trunk, right upper extremity, left upper extremity, right lower extremity, and left lower extremity. Maximal dystonia is the sum of all scores.

### Clinician Interview Based Impression of Change Plus Caregiver Input

The CIBIC-Plus (version ADCS-CGIC) was developed, validated, and is commonly used in studies of anti-dementia drugs in Alzheimer's disease (Joffres 2000). An independent rater evaluated the patient's overall disease severity prior to the initiation of pridopidine or placebo. This assessment, known as the CIBIS, rates the patient on a 7-point Likert scale from extremely severe HD to no symptoms of HD. At each subsequent visit in which the evaluation is performed, the CIBIC-Plus was administered by the same independent rater, but without knowledge of other endpoint assessments or the AEs experienced by the patient during the study (so as not to confound the rating of CIBIC as an efficacy measure or to unblind the study). The independent rater exclusively considers observations of the patient's cognitive, functional, and behavioral performance obtained through interviewing the patient and the caregiver.

cUHDRS: Composite Unified Huntington Disease Rating Scale

The cUHDRS scoring system combines existing measurement scales to holistically measure patient experience: TMS, TFC, SDMT (Symbol Digit Modality Test, Schobet et al, 2017) and SWR (Stroop Word Reading Test).

The enhanced sensitivity of the cUHDRS to clinical change in early symptomatic HD and its strong relationship to underlying brain changes relative to TFC and the TMS suggests that the cUHDRS is an improved measure of clinical progression.

### Global/Functional Scales

### Huntington Disease Quality of Life (QoL)

The HD-QoL is a standardized instrument for measuring health-related quality of life. (Hocaoglu 2012). It is a validated disease-specific measure designed for HD and can provide a summary score of overall health-related quality of life, as well as scores on several discrete scales. HD-QoL is for people who are living with HD. This includes people who are at risk for HD, people who have tested positive for the huntingtin gene but do not have symptoms, and also for people at early through to late stages of disease. HD-QoL can be used across the full spectrum of HD.

### Other Motor Assessments

### Multiple Sclerosis Walking Scale

The Multiple Sclerosis Walking Scale (MSWS-12) was adapted to become a generic measure of walking and mobility and renamed the Walk-12.

### Quantitative Motor (Q-Motor) Assessments

Motor deficits can be objectively assessed using different Q-Motor assessments. All Q-Motor assessments are based on the application of precalibrated and temperature-controlled force transducers and 3-dimensional position sensors with very high sensitivity and test-retest reliability across sessions and sites in a multicenter clinical study. Q-Motor measures thus aim to reduce the limited sensitivity of categorical clinical rating scales, the intra- and inter-rater variability, and placebo effects observed in scales such as UHDRS-TMS. In addition, Q-Motor assessments allow for the objective monitoring of unintended motor side-effects in clinical studies. Thus, Q-Motor is an objective, reliable, and sensitive measure of motor function that is free of rater bias and limits placebo effect influence. FIG. 31 shows the Q-motor tap measurements for a normal patient, a patient with mild defects and a patient with severe defects. In Track-HD, the largest natural history study of pre-manifest and early stage HD, Q-motor tapping deficits correlated with clinical scores as well as with regional brain atrophy (FIGS. 31A and 31B and Bechtel 2010).

### Digitomotography (Speeded Index Finger Tapping)

The patient places their hand on a hand rest with their index finger positioned above a force-transducer. Recordings start after practice runs. The patient is instructed to finger tap as fast as possible between 2 auditory cues. The beginning of a tap is defined as a rise of the force by 0.05 N above maximal baseline level. The tap ends when it drops to 0.05 N before the maximal baseline level is reached again. The duration and variability of tap durations (TD), inter onset intervals (101), inter peak intervals (IPI), and inter tap intervals (ITT) are the exploratory outcome measures for speeded tapping. In addition, variability of peak tapping forces (TF) is calculated as coefficient of variation, and the tapping frequency (Freq), i.e., the number of taps between the onsets of the first and the last tap divided by the time in between, is determined. Five trials of 10 second duration are performed with each hand.

### Dysdiadochomotography (Pronation/Supination Hand Tapping)

This task assesses the regularity of hand taps performed when alternating between the palm and dorsal surface of the hand performing a repetitive pronation/supination movement. The force and duration of the hand taps are recorded similarly to the speeded tapping task. A tone cues the start and end of an assessment. Five trials of 10 second duration are performed with each hand.

### UHDRS Pronation/Supination Assessment

An assessment of the ability to rotate the forearm and hand such that the palm is down (pronation) and to rotate the forearm and hand such that the palm is up (supination) on both sides of the body.

### Manumotography and Choreomotography (Grip Force and Chorea Analysis)

This task assessed the coordination of isometric grip forces in the precision grip between the thumb and index finger. Grip forces are assessed during grip initiation, object transport, and in a static holding phase. Patients are instructed to grasp and lift a device equipped with a force transducer and 3-dimensional position sensor in the precision grip between thumb and index finger and hold it stable adjacent to a marker 10-cm high. Grip forces and 3-dimensional position and orientation of the object are recorded. Mean isometric grip forces and grip force variability in the static phase (expressed as coefficient of variation=standard deviation [SD]/mean×100) (GFV-C) are calculated during a 15-second period starting 8 seconds after the first cueing tone. Five trials of 20 seconds duration are performed with each hand. Chorea is assessed calculating a “position-index” and “orientation-index”. Start and end of assessment are signaled by a cueing tone.

### Timed Up and Go Test

The TUG is a simple test used to assess a person's mobility and requires both static and dynamic balance. It measures the time that a person takes to rise from a chair, walk 3 meters, turn around, walk back to the chair, and sit down. During the test, the person is expected to wear their regular footwear and use any mobility aids that they would normally require. The TUG is used frequently in the elderly population, as it is easy to administer and can generally be completed by the majority of older adults. The test is quick, requires no special equipment or training, and is easily included as part of the routine medical examination (Podsiadlo 1991). The use of the TUG test in conjunction with UHDRS has been recommended for clinical studies of HD (Rao 1991).

### HD-Cognitive Assessment Battery (CAB)

The CAB may be used to detect symptomatic, “pro-cognitive” effects (6 months-1 year) and slowing rate of cognitive decline (>1 year). In the 6-12 months after commencing treatment, the CAB is especially useful to measure “pro-cognitive” effects. Over 1 year from commencing treatment, the CAB is especially useful to detect the slowing rate of cognitive decline. The CAB covers domains most impacted in HD, using tests with good psychometric properties. The following six sections describe the tests that are part of the CAB brief.

### 1. Symbol Digit Modalities Test (SDMT)

The SDMT is a paper-and-pencil test of psychomotor speed and working memory. Participants view a ‘key’ at the top of the page containing symbols paired with numbers. The remainder of the page displays rows of symbols, and the participant has 90 seconds to write the corresponding number that matches each symbol.

### 2. Emotion Recognition

Recognition of facial expressions of emotions is examined using computerized presentations of photographs depicting 6 basic emotions or a neutral expression. Participants are asked to indicate the emotion expressed in each photograph by selecting from the words fear, disgust, happy, sad, surprise, angry, and neutral (10 stimuli per emotion).

### 3. Trail Making Tests A and B

Visual attention and task switching are assessed using the Trail Making test, which consists of 25 circles on a standard sheet of paper. For Trail A, participants are required to connect, as quickly as possible, circles containing numbers in ascending numerical order. For Trail B, participants are to connect, as quickly as possible, circles containing numbers and letters, alternating between numbers and letters in ascending order (e.g., 1, A, 2, B, 3, C, etc.). Trail A is used only as part of the training (Bowie 2006).

### 4. Hopkins Verbal Learning Test, Revised

The HVLT-R offers a brief assessment of verbal learning and memory (recognition and recall). It is easy to administer and score and is well tolerated even by significantly impaired individuals. Its use has been validated in brain-disordered populations (e.g., Alzheimer's disease, HD, amnestic disorders) as a measure of verbal learning and memory. Each form consists of a list of 12 nouns (targets) with 4 words drawn from each of 3 semantic categories. The semantic categories differ across the 6 forms, but the forms are very similar in their psychometric properties. Raw scores are derived for Total Recall, Delayed Recall, Retention (% retained), and a Recognition Discrimination Index. The HVLT-R has high test-retest reliability, and its construct, concurrent, and discriminant validity have been well established. Raw scores are derived for Learning Trials 1-3 (i.e., Total Recall) and Trial 4 (e.g., Delayed Recall Trial).

### 5. Paced Tapping Test

Psychomotor function is assessed in a Paced Tapping test (also known as PTAP). Participants tap on left and right mouse buttons, alternating between thumbs, at 3.0 Hz. They first listen to a tone presented at the desired tapping rate, and then begin tapping to the tone. After 11 taps with the tone, the repetition of the tone is discontinued, and participants attempt to continue tapping at the same rate until the end of the trial (31 taps later).

### 6. One Touch Stockings of Cambridge (OTS)

OTS is a spatial planning task which gives a measure of frontal lobe function. OTS is a variant of the Stockings of Cambridge task, and places greater demands on working memory as the participant has to visualize the solution. As with Stockings of Cambridge, the participant was shown 2 displays containing 3 colored balls. The displays are presented in such a way that they can easily be perceived as stacks of colored balls held in stockings or socks suspended from a beam. This arrangement makes the 3-dimensional concepts involved apparent to the participant, and fits with the verbal instructions. There is a row of numbered boxes along the bottom of the screen. The test administrator first demonstrates to the participant how to use the balls in the lower display to copy the pattern in the upper display, and completes 1 demonstration problem, where the solution requires 1 move. The participant must then complete 3 further problems, 1 each of 2 moves, 3 moves, and 4 moves. Next, the participant is shown further problems, and must work out in their head how many moves the solutions to these problems require, then touch the appropriate box at the bottom of the screen to indicate their response.

### Problem Behaviors Assessment-Short Form (PBA-s)

Because of the prominence of psychiatric symptoms in HD, it was recommended that the PBA-s form be used in all HD studies with any need for behavioral assessment as a comprehensive screen for the most common psychiatric symptoms in HD (Craufurd 2001, Kingma 2008). The PBA-s also includes questions concerning suicidal behavior, a particular concern in HD. The PBA-s is based on the same set of core behavioral symptoms as the UHDRS Behavioral questions, which were used previously as the global psychiatric measure in most HD studies. The PBA-s has more detailed questions and more specific guidance on administration and scoring

The PBA-s is a brief semi-structured interview covering the most common behavioral and psychiatric manifestations of HD. The interview is not restricted to a single construct, but rather covers several broad symptom domains relevant to HD, comprising 11 items: low mood, suicidal ideation, anxiety, irritability, anger/aggressive behavior, loss of motivation, perseverative thinking or behavior, obsessive-compulsive behaviors, paranoid thinking, hallucinations, behavior suggestive of disorientation. Each symptom is rated for severity on a 5-point scale according to detailed scoring criteria which roughly correspond to the following: 0=“not at all”; 1=trivial; 2=mild; 3=moderate (disrupting everyday activities) and 4=severe or intolerable. Each symptom is also scored for frequency on a 5-point scale as follows: 0=symptom absent; 1=less than once weekly; 2=at least once a week; 3=most days (up to and including some part of everyday); and 4=all day, every day. Severity and frequency scores are multiplied to produce an overall ‘PBA score’ for each symptom.

### Stroop Word Reading Test—SWR

SWR is used to assess the ability to inhibit cognitive interference, which occurs when the processing of a stimulus feature affects the simultaneous processing of another attribute of the same stimulus.

In the most common version, which was originally proposed by Stroop in 1935, subjects are required to read three different tables as fast as possible. Two of them represent the “congruous condition” in which participants are required to read names of colors (henceforth referred to as color-words) printed in black ink (W) and name different color patches (C). Conversely, in the third table, named color-word (CW) condition, color-words are printed in an inconsistent color ink (for instance the word “red” is printed in green ink). Thus, in this incongruent condition, participants are required to name the color of the ink instead of reading the word. In other words, the participants are required to perform a less automated task (i.e., naming ink color) while inhibiting the interference arising from a more automated task (i.e., reading the word). This difficulty in inhibiting the more automated process is called the Stroop effect.

### Stages of Huntington's Disease

Many clinicians and diagnosticians adopt the Shoulson and Fahn rating scale, based on TFC scores, to follow progression of HD. This rating scale groups total TFC scores into five stages of disease, with lower stages indicating more intact functioning. Table 139 below provides the TFC scores, average years from diagnosis and broad guidelines for typical care level for each stage of disease. (Johnson 2014).

TABLE 139
HD Disease stages

| | | Years | |
|---|---|---|---|
| | | since | |
| | TFC | motor | |
| Stage | score | diagnosis | Typical abilities and care level |
| 1 | 11-13 | 0-8 | Able to work at least part time, may require |
| | | | slight assistance in one of finances, |
| | | | domestic chores or ADL basic functions |
| 2 | 7-10 | 3-13 | Unable to work, requires some assistance in |
| | | | some basic functions |
| 3 | 3-6 | 5-16 | Unable to work, requires major assistance in |
| | | | most basic functions |
| 4 | 1-2 | 9-21 | Requires major assistance in all basic |
| | | | functions and although comprehension may |
| | | | be intact, requires assistance to act. |
| 5 | 0 | 11-26 | Requires major assistance in all basic |
| | | | functions and full-time nursing care |

Patients with stage 1 or 2 have the steepest rate of natural decline and are the most sensitive to the clinical measure described in this application. TFC assessments are designed specifically for patients with stage 1 or 2 and earlier. Patients with stage 3, 4 or 5 often have difficulty completing assessments, the floor and ceiling limit the ability to track change and have very significant brain tissue loss.

Early stage HD, as used herein, means stage 1 or stage 2 HD (BL TFC≥7) as defined by Table 139 above.

Analysis of Pridopidine on Functional Capacity in Early-Stage Participants from the PRIDE-HD Study

### Methods

A mixed model of repeated measures (MMRM) was used to evaluate data from the full analysis set at 26 and 52 weeks for all HD and for the early HD groups. A sensitivity analysis was performed using the multiple imputation method assuming Missing Not At Random (MNAR) and the ‘worst-case scenario’ where placebo values were imputed for missing data from all patients who discontinued therapy. p-values are presented for descriptive purposes only.

### Results

Total Functional Capacity scores for all recipients of the 45 mg bid dosage (N=75) at 26 and 52 weeks compared to placebo (N=81) are displayed in FIGS. 39A-39B. The results show that, in comparison to placebo, the 45 mg bid dose demonstrates a beneficial effect on TFC for the entire population at weeks 26 and 52. At 26 weeks, a trend towards improvement in change from baseline vs. placebo is seen in the pridopidine group (placebo: −0.49, SE 0.16 vs. treatment: −0.15, SE 0.17; difference between groups 0.34, nominal p=0.15, FIG. 39A). At 52 weeks the difference between groups was nominally significant; placebo declined by −0.83 points (SE 0.20), while the treatment group was essentially unchanged from baseline (+0.04, SE 0.22; difference between groups of 0.87, nominal p=0.0032, FIG. 39B). The effect was most pronounced for early HD patients (HD1/HD2, TFC=7-13). At 26 weeks, the difference between groups was 0.56 with a significant nominal p=0.036 (FIG. 39C). At 52 weeks, the effect on TFC was stronger with treatment difference of 1.16 and nominal p=0.0003 (FIG. 39D). Baseline demographic characteristics were similar between early HD participants (HD1+HD2, TFC 7-13) who completed 26 weeks of treatment and those who completed 52 weeks of treatment (Table 140).

TABLE 140
Demographic Characteristics of Early HD participants (baseline TFC 7-13)
completing 26 and 52 weeks of treatment and those who did not complete 52 weeks.

| | Early HD 26-weeks | | Early HD 52-weeks | | Early HD 52-weeks | |
|---|---|---|---|---|---|---|
| | Completers | | Completers | | Non-completers | |
| | Placebo | 45 mg bid | Placebo, | 45 mg BID, | Placebo, | 45 mg BID, |
| Parameter | n = 55 | n = 48 | n = 41 | n = 37 | n = 21 | n = 22 |
| Baseline TFC mean | 9.0 (1.8) | 9.3 (1.8) | 8.9 (1.7) | 9.2 (1.9) | 8.9 (1.9) | 9.0 (1.7) |
| (SD) | | | | | | |
| CAG mean (SD) | 44.7 (3.4) | 44.2 (4.7) | 45.0 (3.8) | 43.7 (4.4) | 43.8 (1.8) | 44.5 (4.2) |
| Age (Y) mean (SD) | 49.2 (11.8) | 50.2 (12.6) | 48.3 (12.7) | 51.4 (12.5) | 50.8 (8.0) | 50.6 (12.6) |
| Gender | M, 26 (47.3%) | M, 22 (45.8%) | F, 23 (56%) | F, 19 (51.4%) | F, 8 (38%) | F, 11 (50%) |
| N (%) | F, 29 (52.7%) | F, 26 (54.2%) | M, 18 (44%) | M, 18 (48.6%) | M, 13 (62%) | M, 11 (50%) |
| Height (cm) | 170.4 (9.6) | 170.5 (10.2) | 169.5 (9.5) | 170.3 (8.0) | 174.5 (10.1) | 170.6 (13.8) |
| mean (SD) | | | | | | |
| Weight (kg) | 73.0 (12.7) | 70.8 (15.0) | 73.2 (13.1) | 71.2 (15.2) | 72.8 (9.7) | 69.3 (13.4) |
| mean (SD) | | | | | | |
| BMI mean (SD) | 25.2 (4.3) | 24.2 (4.0) | 25.5 (4.3) | 24.5 (4.4) | 24.1 (4.0) | 23.7 (2.0) |
| Neuroleptics | Yes, 21 | Yes, 15 | Yes, 17 | Yes, 11 | Yes, 6 | Yes, 8 |
| N (%) | (38.2%) | (31.3%) | (41.5)% | (29.7%) | (28.6%) | (36.4%) |
| | No, 34 | No, 33 | No, 24 | No. 26 | No, 15 | No, 14 |
| | (62%) | (68.7%) | (58.5%) | (70.3%) | (71.4%) | (63.6%) |

Source: PRIDE-HD data

There were no notable demographic differences between early HD participants who completed the full 52 weeks of treatment and those who dropped out (non-completers) (Table 140).

The dropout rates between the early HD (TFC 7-13) placebo and 45 mg bid groups were comparable. 89% (55/62) of the placebo early HD participants completed the first 26 weeks of the study, vs. 81% (48/59) patients in the 45 mg bid group. 76% (42/55) of early HD placebo participants and 77% (37/48) of 45 mg bid participants who completed 26 weeks started the second treatment period, while 97% (41/42) of the early HD placebo group and 100% (37/37) of the 45 mg BD group who initiated the second study period completed 52 weeks (Table 141).

TABLE 141
Early HD patients disposition

| | Placebo | | Pridopidine 45 mg bid | |
|---|---|---|---|---|
| | N = 62 | | N = 59 | |
| Completed 26 weeks | 55/62 | (89%) | 48/59 | (81%) |
| Started 2^{nd} study period | 42/55 | (76%) | 37/48 | (77%) |
| of 52 weeks | | | | |
| Completed 52 weeks | 41/42 | (97%) | 37/37 | (100%) |

Source: PRIDE-HD data

The observed beneficial effect in the combined HD1 and HD2, TFC 7-13 group is not driven by a subset of patients, as both HD1 and HD2, TFC 7-13 contributed to the overall effect on TFC in the early HD population (HD1 with ΔTFC 1.89, nominal P=0.0059 and HD2 with ΔTFC 0.94, nominal P=0.009). Early HD patients (TFC 7-13) decline at a mean rate of 0.97 points/year, while late stage patients with TFC 3-6 (HD3) and 0-3 (HD4) decline at 0.38 and 0.06 points/year, respectively, likely reflecting a floor effect in more advanced disease (Marder et al, 2020).

The effect of pridopidine on TFC in late stage HD patients (HD3 and HD4) could not be accurately assessed in this study. Due to the slow annual decline in TFC in late stage HD patients, to properly assess the effect of a drug on TFC in this population, a large number of subjects with a long follow-up duration is required. Therefore, no conclusions can be drawn at this time about the effect of pridopidine on TFC decline in subjects with late stage HD.

To further assess the effect of pridopidine 45 mg bid on functional decline in TFC in all HD and early HD patients at Week 52, a multiple imputation analysis assuming Missing Not At Random (MNAR) and using the “worst case scenario” was performed (FIG. 40). This method assumes that all missing data in the active treatment group follow the trajectory of the placebo group. Using MNAR for the entire population, the 45 mg bid pridopidine dose was superior to placebo at Week 52 (difference=0.58; nominal p=0.057). When this analysis was restricted to patients with early HD (HD1 and HD2, TFC 7-13), the MNAR analysis shows an effect size of 0.79 (nominal p=0.016) (FIG. 40).

A post-hoc analysis was performed to assess the effect of 45 mg bid on each of the five TFC sub-items in the early HD group (baseline TFC 7-13) (Table 142). Most TFC subscales contribute to the effect on total TFC score in early disease, with domestic chores, activity of daily living, care level, and finances each reaching nominal statistical significance.

TABLE 142
Change from baseline to Week 52 in TFC domains
for early HD subgroups (baseline TFC 7-13)

| Week 52 | |
|---|---|
| Placebo | 45 mg BID |

Activity of Daily Living

| | | |
|---|---|---|
| LS Mean change from baseline | −0.32 (0.08) | 0.03 (0.08) |
| (SE) | | |
| LS Mean difference | | 0.35 |
| p value | | 0.002 |

Domestic Chores

| | | |
|---|---|---|
| LS Mean change from baseline | −0.23 (0.07) | 0.01 (0.07) |
| (SE) | | |
| LS Mean difference | | 0.24 |
| p value | | 0.02 |

Finance

| | | |
|---|---|---|
| LS Mean change from baseline | −0.37 (0.10) | −0.02 (0.11) |
| (SE) | | |
| LS Mean difference | | 0.35 |
| p value | | 0.017 |

Care level

| | | |
|---|---|---|
| LS Mean change from baseline | −0.09 (0.03) | 0.03 (0.03) |
| (SE) | | |
| LS Mean difference | | 0.12 |
| p value | | 0.004 |

Occupation

| | | |
|---|---|---|
| LS Mean change from baseline | −0.20 (0.09) | −0.07 (0.09) |
| (SE) | | |
| LS Mean difference | | 0.13 |
| p value | | 0.279 |

LS Mean = least square mean
Included all randomized patients with baseline TFC >=7, who received at least one dose of study drug and had at least one post-baseline efficacy assesment; p-values are nominal and presented for descriptive purposes only

In an additional exploratory analysis, “responders” were defined as participants with a change from baseline in TFC≥0 at week 52 (i.e. no worsening), and “non-responders” as those with TFC decline of <0 points at Week 52 (i.e. worsening of any magnitude in TFC score) (Table 143A). For the entire cohort, 47.3% of patients in the placebo group had worsening in TFC compared to 23.4% patients in the pridopidine group, with a nominally significant odds ratio (95% CI) of 0.32 (0.13-0.79, p=0.01). In the early HD sub-group, 51.2% of patients in the placebo group showed worsening of TFC, compared to 18.9% of patients in the pridopidine group, with an odds ratio (95% CI) of 0.20 (0.07-0.56, nominal p=0.002) (Table 143A).

To determine the relationship between the beneficial effect on TFC and improvements in other clinical measures, we conducted an additional responder analysis. For that purpose, we compared the improvement on other measures between responders (ΔTFC≥0) and non-responders (ΔTFC<0) at week 52 (early HD from all arms combined). We evaluated measures representing different aspects of the disease. These include motor (UHDRS-TMS), severity assessment of HD using the Clinician Global Impression of Change (CGI-C), and the Clinician's Interview-Based Impression of Change Plus (CIBIC+), as well as an additional functional measure, the UHDRS Functional Assessment (UHDRS-FA). Among responders, there was a nominally significant improvement compared to non-responders in the UHDRS Total Motor Score (UHDRS-TMS), UHDRS Functional Assessment (UHDRS-FA), Clinician Global Impression of Change (CGI-C), and the Clinician's Interview-Based Impression of Change Plus (CIBIC+), demonstrating concordance between preservation of TFC and improvement in other clinical outcomes (Table 143B).

Tables 143A and 143B: Responder analyses of (A) N (%) of participants in placebo and 45 mg bid groups showing worsening of TFC and (B) changes from Baseline to Week 52 in motor and global assessments for responders and non-responders in early HD (TFC 7-13).

TABLE 143A
N (%) of participants with ATFC <0
(worsening/non-responders) at 52 weeks

| | | | Odds Ratio | |
|---|---|---|---|---|
| | | | (95% Cl) | |
| | | | (GLIMMIX | |
| | Placebo | 45 mg bid | model) | P-value* |
| ALL HD | | | | |
| Placebo, | 26 (47.3%) | 11 (23.4%) | 0.32 (0.13-0.79) | 0.01 |
| n = 55 | | | | |
| 45 mg bid, | | | | |
| n = 47 | | | | |
| Early HD | | | | |
| (TFC >7) | | | | |
| Placebo, | 21 (51.2%) | 7 (18.9%) | 0.20 (0.07-0.56) | 0.002 |
| n = 41 | | | | |
| 45 mg bid, | | | | |
| n = 37 | | | | |

*p-values are nominal and presented for descriptive purposes only.

TABLE 143B
Change in motor and global Functional assessments in responders
(ATFC >0) vs non-responders (ATFC <0) at 52 weeks

| | | |
|---|---|---|
| Assessment, | Responders ® | Non-Responders |
| Statistic | (N = 99) | (N = 77) |

UHDRS-TMS^{b}

| n | 99 | | 77 | |
|---|---|---|---|---|
| Mean (SD) | −4.6 | (8.04) | 2.5 | (10.54) |

| | |
|---|---|
| p-value | <0.001 |

UHDRS-FA^{b}

| n | 98 | | 77 | |
|---|---|---|---|---|
| Mean (SD) | −0.0 | (1.78) | −1.9 | (3.03) |

| | |
|---|---|
| p-value | <0.001 |

CGI-C Ratings⁰, n (%)

| | | | | |
|---|---|---|---|---|
| n | 94 | (100) | 75 | (100) |
| No change or | 68 | (72) | 34 | (45) |

| | |
|---|---|
| p-value | <0.001 |

CIBIC Ratings n (%)

| | | | | |
|---|---|---|---|---|
| n | 92 | (100) | 77 | (100) |
| No change or | 60 | (65) | 35 | (45) |

| | |
|---|---|
| p-value | 0.008 |

Source: PRIDE-HD CSR Post Hoc Summaries
^{a}A responder is defined as a patient with a change in TFC from baseline >=0.
^{b}The statistical test is an analysis of variance (ANOVA) with treatment group as a fixed effect.
^{c}The statistical test is a Cochran-Mantel-Haenszel (CMH) test.
^{d}This includes the following ratings: Very much improved; much improved; minimally improved, and no change.
Abbreviations: TFC = Total Functional Capacity; UHDRS = Unified Huntington’s Disease Rating Scale; TMS = Total Motor Score; FA = Functional Assessment; CGI-C = Clinical Global Impression of Change; CIBIC = Clinician’s Interview-based Impression of Change. P-values are nominal and presented for descriptive purposes only.

TABLE 144
Responder analysis in early HD patients

| | Observed Data Analysis | |
|---|---|---|
| | 45 mg bid | Placebo |
| Responder Analysis Questions | N = 37 | N = 41 |
| What proportion of early stage subjects | 30 (81%) | 20 (49%) |
| had no deterioration on TFC (score ≥0) | | |
| at 52 weeks? | | |
| p value (Chi-Square) | 0.003 | |
| What proportion of early stage subjects | 10 (27%) | 5 (12%) |
| had an improvement of ≥1 points on TFC | | |
| at 52 weeks? | | |

| | |
|---|---|
| p value (Chi-Square) | 0.099 |

### Summary

The follow-up analysis supports the earlier findings that pridopidine 45 mg bid provides a robust, nominally significant reduction in total functional capacity (TFC) at 52 weeks compared to placebo. This effect is evident particularly in patients with early stage HD (HD1 and HD2, TFC 7-13, baseline TFC 7-13).

HD patients treated with 45 mg pridopidine twice daily showed maintenance, improvement and less TFC decline than placebo at week 52, demonstrating an almost 1-point difference (0.87, nominal p=0.0032). A trend towards improvement was also noted at 26 weeks (difference of 0.37; nominal p=0.015). Beneficial effects at 26 and 52 weeks were more pronounced in early-stage participants, with differences from baseline between active and placebo groups of 0.56 (nominal p=0.036) and 1.16 points (nominal p=0.0003), respectively. These observations suggest that pridopidine at a dose of 45 mg bid is associated with maintenance of functional capacity in HD. Participants receiving pridopidine 45 mg bid displayed virtually no decline in mean TFC over the course of 1 year. This effect was particularly visible in patients with milder disease (TFC 7-13). This is very different than observations from placebo groups in previous clinical trials or in natural history studies. (FIG. 48).

Further, participants receiving 45 mg bid pridopidine showed less decline in TFC, as can be observed in the responder analysis (Table 143A). Once again, this effect is most noticeable in the early HD sub-group.

Demographic characteristics of participants who completed either the 26-week or 52-week study, or that dropped out over the course of the study are similar (Table 140). Dropout rates between the placebo and 45 mg bid groups are also similar (Table 141). Together, these suggest that the data is well-matched and valid for TFC comparison between the groups.

A positive responder analysis was also observed for participants in the 45 mg bid dosage group compared to placebo who did not deteriorate from baseline (change in UHDRS-TFC from baseline ≥0). Responders in the 45 mg bid group also show nominally significant improvement vs placebo in the UHDRS-TMS, UHDRS-FA, CGI-C and CIBIC+. This points to pridopidine having additional beneficial clinical effects beyond TFC.

Improvement in functional capacity—a measure which synthesizes motor, cognitive, and behavioral ability into relevant daily activities—is perhaps the most pressing unmet therapeutic need in HD. The UHDRS-TFC captures this therapeutic need, as it reflects elements of function with meaningful impact on patients' lives. Thus, a therapy with the ability to beneficially modify TFC decline would be of significant therapeutic value.

### Discussion

### Pridopidine Efficacy

PRIDE-HD was a phase 2b, double-blind, placebo-controlled study initially designed to assess the safety of pridopidine doses ranging from 45 mg to 112.5 mg bid, and its efficacy on TMS at 26 weeks. Due to the understanding that the primary target of pridopidine is the S1R, suggesting a therapeutic potential beyond motor function, the ongoing PRIDE-HD study was extended from 26 weeks to 52 weeks in order allow the assessment of pridopidine on total functional capacity (UHDRS-TFC). A minimum of 52 weeks is needed for the placebo group to decline in UHDRS-TFC and to allow a window for detection of the therapeutic effect of a drug. TFC change from baseline to week 52 was a pre-specified exploratory endpoint in the PRIDE-HD study.

The UHDRS-TFC scale is a validated and accepted tool used by clinicians to assess HD disease stage and the level of patient's functionality. A decline in UHDRS-TFC is associated with other aspects of disease progression including brain atrophy, motor, cognitive and behavioral functions.

In PRIDE-HD, pridopidine 45 mg bid demonstrated a statistically significant effect maintaining functional capacity compared to placebo at week 52 in all HD patients (p=0.0032) irrespective of stage of disease. The effect of pridopidine 45 mg bid on UHDRS-TFC was most evident in the early stage HD subpopulation (post hoc analysis, p=0.0003, HD1 and HD2, TFC 7-13, baseline UHDRS-TFC 7-13). Additional support for the observed effect of pridopidine on TFC is demonstrated by a post hoc responder analysis in early stage HD (HD1+HD2), showing a significant decrease in the proportion of patients with worsening of UHDRS-TFC (decline ≥1) at 52 weeks in the pridopidine treated group compared to placebo 18.9% vs 51.2% (pridopidine 45 mg bid vs placebo, respectively, p=0.002).

Historical data from the 2CARE study where early HD patients were followed for 60 months (baseline UHDRS-TFC 9-13) were compared to Open-HART in an exploratory analysis. Early HD patients (baseline TFC 9-13) treated with pridopidine 45 mg bid in the Open-HART trial were compared to this matched cohort of early HD patients receiving placebo in the 2CARE study, over 5 years. After 5 years, Open-HART patients treated with 45 mg bid pridopidine showed a UHDRS-TFC decline of −1.8 points compared to −5.0 points decline in patients receiving placebo from the 2CARE study. Thus, pridopidine's effect on UHDRS-TFC was maintained across 60 months. Limitations to these findings include comparison of data across studies, open label status in Open-HART and a small sample size.

To date, long-term (>12 months) observational and randomized clinical studies in HD patients did not show any effect on rate of decline in UHDRS-TFC (Waters et al 2018). PRIDE HD is the only study to date that shows an effect on UHDRS-TFC decline in HD patients. Treatment effect was seen following treatment with pridopidine 45 mg bid at Week 52 (0.04 points).

### Pridopidine Motor Effects

The UHDRS-TMS is the standard and most-accepted clinical tool for tracking the progression of motor symptoms in patients with HD (Huntington Study Group 1996). The motor section of the UHDRS assesses motor features of HD including oculomotor function, dystonia, gait, and postural stability. All of these are prominent features of HD, and are positively modulated by pridopidine. The UHDRS-TMS is the sum of 15 individual motor ratings, with each assessment rated on a 5-point scale from 0 (normal) to 4 (maximally abnormal). Higher scores indicate more severe motor impairment than lower scores.

The UHDRS-TMS is a highly relevant endpoint for the assessment of the effect of pridopidine in HD. Pridopidine previously demonstrated motor function benefit on the UHDRS-TMS compared to placebo, in 2 large, double-blind, placebo-controlled studies in patients with HD (TMS was a secondary endpoint measure in HART and MermaiHD (de Yebenes et al 2011; Huntington Study Group HART Investigators 2013). In the HART study, pridopidine 45 mg bid showed a statistically significant improvement in UHDRS-TMS of −2.78 points (p=0.039). In MermaiHD, pridopidine 45 mg bid showed a statistically significant improvement in TMS of −2.96 (p=0.004).

Furthermore, a pooled analysis from HART and MermaiHD demonstrates statistically significant differences between pridopidine treatment and placebo for changes in UHDRS-TMS from baseline to Week 12 (−2.10; p=0.0078) and Week 26 (−3.35; p=0.0006). These changes were seen across all motor features except for chorea (Landwehrmeyer et al 2011). The pooled analysis results were in line with those of the individual studies and indicate that pridopidine improves overall motor function in patients with HD, as measured by TMS.

Subsequent to these observations, another large, double-blind, placebo-controlled study was performed (PRIDE-HD) using change from baseline in UHDRS-TMS to week 26 as the primary endpoint to further evaluate the effects of pridopidine. Due to new insights regarding pridopidine mechanism of action (MOA) and therapeutic potential on total functional capacity, the PRIDE-HD protocol was amended, prior to the primary endpoint readout at week 26, to extend the double-blind placebo controlled study to 52 weeks. As previously discussed, a minimum of 52 weeks is necessary for the placebo group to deteriorate sufficiently to allow a window for assessment of an effect of drugs on UHDRS-TFC.

In the PRIDE-HD study, the 45 mg bid dose group showed an improvement in UHDRS-TMS at Week 26 and Week 52 compared to baseline. This improvement is comparable to the observed improvement in MermaiHD (A UHDRS-TMS from baseline in the 45 mg bid group was −3.39 at Week 26 in PRIDE-HD vs A UHDRS-TMS from baseline of −1.3 at Week 26 seen in MermaiHD). However, the PRIDE-HD result did not reach statistical significance over placebo due to the high and sustained placebo effect. Placebo effects in UHDRS-TMS have been noted previously in HD clinical studies, but these generally regressed prior to 6 months (Papapetropoulos et al 2014). The placebo effect observed in PRIDE-HD unexpectedly lasted for the full 52 weeks of the study, limiting the ability to draw meaningful conclusions from this endpoint. The placebo effect in PRIDE-HD was maintained to Week 52, in contrast with MermaiHD in which there was no placebo effect by Week 26. Contributing factors to the placebo effect in PRIDE-HD include the many active treatment arms (4:1 randomization), expectation bias based on previous results with pridopidine, limited site training and frequent changes in raters were allowed. Central quality monitoring was implemented only in the second half extension of the study.

### Total Functional Capacity

The UHDRS-TFC annual decline in the PRIDE-HD placebo arm (Reilmann et al, 2018) was comparable to that reported in the literature (Dorsey et al 2013) and observed in historical placebo arms (McGarry et al, 2020). Observational and randomized studies in HD show that that annual rate of UHDRS-TFC decline is related to the baseline UHDRS-TFC (disease stage), with faster rates observed in early stages of HD, corresponding to UHDRS-TFC scores of 7-13, or HD1 and HD2, TFC 7-13 (Marder et al 2000). Natural history studies in HD and clinical study placebo data show that the annual decline in early HD (UHDRS-TFC 7-13) patients of UHDRS-TFC is between 0.7 to 1.17 points (Hersch et al 2017; Reilmann et al 2018).

Further, natural history studies demonstrate that the decline in UHDRS-TFC parallels the decline in other disease measures, including motor, cognitive and neuropsychiatric endpoints (Tabrizi et al 2013; Dorsey et al 2013). The annual decline of UHDRS-TFC in placebo-treated and untreated (observational) patients is consistent across the totality of study data in HD patients (Marder et al 2000; Waters et al 2018; McGarry et al 2017; Reilmann et al 2019).

Huntington Disease Natural History: UHDRS-TFC
Annual Decline in Early HD Patients

| | CREST-E | | | PRIDE-HD | |
|---|---|---|---|---|---|
| | (placebo data) | | | (placebo data) | |
| | Hersch et al | Marder et al 2000 | | Reilmann et al | |
| | 2017 | (observational) | | 2019 | |
| UHDRS-TFC | 7-13 (N = 278) | 11-13 (N = 214) | 7-10 (N = 358) | 11-13 (N = 12) | 7-13 (N = 63) |
| at baseline | | | | | |
| Change in | −0.7 | −1.15 | −0.84 | −1.63 | −1.17 |
| UHDRS-TFC | | | | | |
| from baseline | | | | | |

Source: Hersch et al 2017; Marder et al 2000; Reilmann et al 2018

This was further corroborated by the PRIDE-HD study. The early HD placebo group showed the expected annual deterioration in UHDRS-TFC of −1.17 points decline from baseline at week 52 (Reilmann et al 2018).

In the PRIDE-HD study, the change from baseline in UHDRS-TFC to week 52 in all HD patients (irrespective of disease stage) and the early stage HD (HD1+HD2, baseline UHDRS-TFC 7-13)

Pridopidine 45 mg bid shows a statistically significant maintenance of UHDRS-TFC at week 52 compared to placebo in all HD patients irrespective of stage (N=75, UHDRS-TFC change from baseline vs placebo 0.87 [95% confidence interval: 0.29-1.45]; p=0.0032) at 52 weeks.

FIGS. 1 and 2 are graphs showing pridopidine concentration (ng/ml) measures in patients' blood through week 20 of treatment.

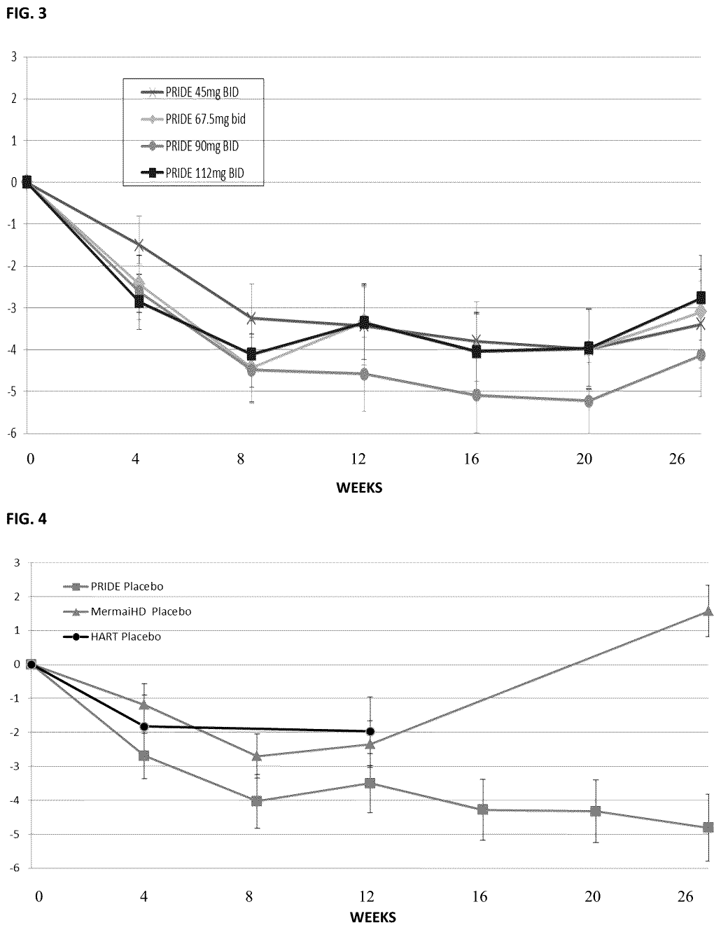

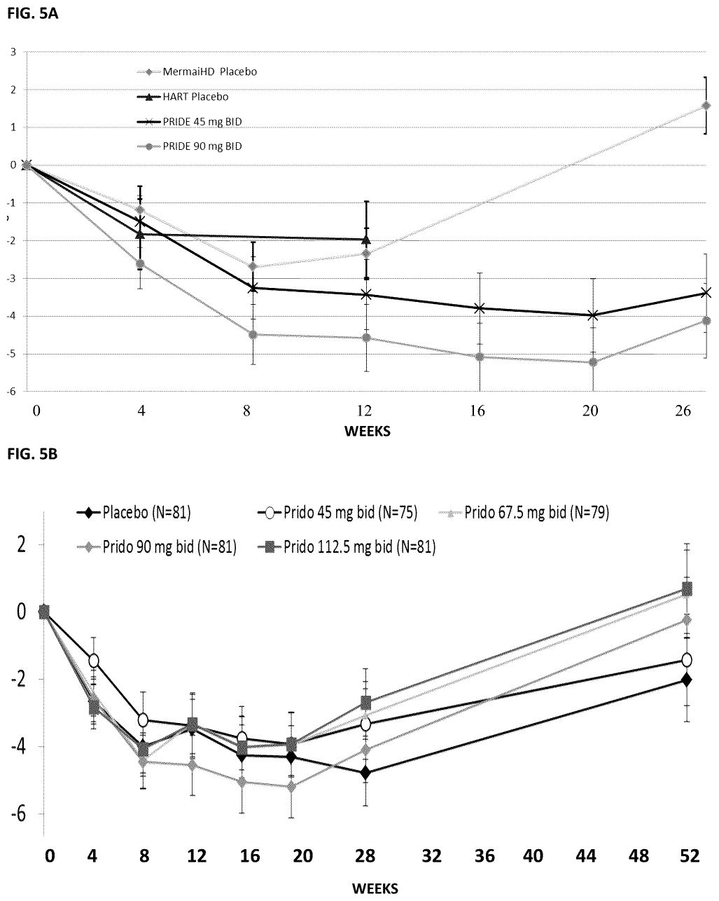

FIGS. 3-5B are graphs showing change in UHDRS TMS over time. A lower number represents improvement. FIG. 3 shows a comparison between doses in the PRIDE-HD study. FIG. 4 shows the placebo effect in the UHDRS TMS, which was greater in the PRIDE-HD study than in the MermaiHD or HART studies. FIG. 5A shows an improvement in UHDRS TMS for both 45 mg pridopidine bid and 90 mg pridopidine bid in the PRIDE-HD study compared to the placebo in MermaiHD and HART studies. FIG. 5B shows an overall improvement in UHDRS-TMS for pridopidine bid over 52 weeks.

### UHDRS Total Functional Capacity (UHDRS TFC)

The data in this application demonstrates that pridopidine shows an effect on progression of HD as measured by total functional capacity (TFC). This effect on TFC was statistically significant in the full analysis set and even more pronounced in early stage HD patients. Early stage HD patients are defined as those with a baseline (BL) TFC score of greater than or equal to 7 (Stage 1 and Stage 2).

There was significant maintenance in UHDRS TFC between patients administered pridopidine 45 mg bid compared to patients administered the placebo at 52 weeks in all HD stages and in the early stage HD sub-population. The effect of pridopidine on TFC was driven by patients with early stage HD (baseline 7-13) and not seen in patients with late stage HD (baseline 0-6) receiving pridopidine. Due to the floor effect of TFC in later stages of HD, in order to detect an effect on TFC in late stage HD patients longer studies with larger sample sizes are needed. The effect on TFC observed at 26 weeks reached significance in the early stage subpopulation (FIG. 39C). The effect on TFC observed at 52 weeks reached significance in all and early stage HD (FIGS. 39B, 39D).

The TFC annual decline of the placebo group shown in, for example, FIGS. 39A-39D, was comparable to the TFC annual decline reported in the literature and observed in historical placebo arms. These data show a slowing of clinical progression in HD as measured by TFC, and is the first clinical trial to do so among eleven (11) other clinical trials. Significance was observed in the UHDRS TFC at week 26 (FIG. 39B), TFC finance at week 26, TFC finance and ADL at week 26, TFC ADL at week 26, UHDRS TFC at week 52, TFC finance at week 52, TFC finance and ADL at week 52 (TFC ADL at week 52).

### UHDRS Independence Scale (UHDRS IS)

The UHDRS-IS comprises part of the UHDRS functional assessments (Huntington Study Group 1996). It is a rating scale where the patient's degree of independence is given in percentage, from 10% (tube fed, total bed care) to 100% (no special care needed). Scores must end in 0 or 5 (eg, 10%, 15%, 20% etc).

The change from baseline in the UHDRS-IS assessed at week 52 decreased across treatment groups, but was not statistically significant in any treatment group. For the placebo group, there was a decrease (indicating a trend towards decline) in IS at Week 52. Positive trends in the desired direction were observed in early-stage HD patients (baseline TFC score ≥7) at week 52. No clinically meaningful changes were noted for patients with a baseline TFC<7. The Independence scale supports the TFC effect, which provides a convergence of endpoints.

### UHDRS TMS and Motor Endpoints

Motor effects were statistically significant in stage 1 subpopulations. For example, statistically significant changes are seen in the HD Stage 1 patient subgroups for Total TMS, Involuntary movements (Dystonia, Chorea), Ambulation (TMS Gait and Balance, Timed Up and Go, Walk 12).

A large placebo response masked motor effects in the full analysis set. However, in early HD there was a statistically significant effect on TMS at weeks 26 and 52 driven by a lower placebo effect. Involuntary Movements (chorea and dystonia) as measured by TMS improved in HD stage 1 patients at 26 weeks. This effect persisted at 52 weeks as well.

In addition, positive effects on ambulation (such as gait, timed up and go, and stair climbing) were observed in early stage patients administered 45 mg pridopidine bid.

### PBA-s

The PBA-s is a brief semi-structured interview covering the most common behavioral and psychiatric manifestations of HD. The interview is not restricted to a single construct, but rather covers several broad symptom domains relevant to HD, comprising 11 items: low mood (depression), suicidal ideation, anxiety, irritability, anger/aggressive behavior, loss of motivation (apathy), perseverative thinking or behavior, obsessive-compulsive behaviors, paranoid thinking, hallucinations, and behavior suggestive of disorientation. Each symptom is rated for severity on a 5-point scale according to detailed scoring criteria, which roughly correspond to the following: 0=“not at all”; 1=trivial; 2=mild; 3=moderate (disrupting everyday activities) and 4=severe or intolerable. Each symptom is also scored for frequency on a 5-point scale as follows: 0=symptom absent; 1=less than once weekly; 2=at least once a week; 3=most days (up to and including some part of every day); and 4=all day, every day.

Severity and frequency scores are multiplied (after setting all values outside the range of 0-4 to missing) to produce an overall “PBA-s score” for each symptom. The total PBA score is calculated by the sum of all PBA-s scores across symptoms/domains.

The change from baseline to week 26 in the PBA-s domains and total scores did not show meaningful results. However, the change from baseline to week 52 in the PBA-s total score as well as several of the PBA-s domains showed a trend to improvement or significant improvement. In the full analysis set, the pridopidine 45 mg bid group showed a trend towards improvement in the PBA-s total score at 52 weeks compared with the placebo group. Pridopidine shows a trend to improvement in PBA apathy in early stage patients at 26 weeks and 52 weeks, respectively.

### HD-Cognitive Assessment Battery (HD-CAB)

The PRIDE-HD study was the first large study to include the HD-Cognitive Assessment Battery (HD-CAB) assessments (Stout et al 2014). The HD-CAB was designed to detect symptomatic, “pro-cognitive” effects (6 months-1 year) and slowing rate of cognitive decline (>1 year) in late pre-manifest, HD1 and HD2, TFC 7-13 patients. It covers cognitive domains most impacted in HD, using tests with good psychometric properties. The battery includes the following tests: Symbol Digit Modalities Test, Emotion Recognition, Trail Making Test B, Hopkins Verbal Learning Test (revised), Paced Tapping at 3 Hz, and One Touch Stockings of Cambridge.

Positive findings indicating potential improvement from baseline in the Paced Tapping at 3 Hz assessment (a measure of psychomotor function) were observed in the full analysis set at week 52 for the 45 mg bid treatment group

### Example 2: Long Term Effect of Pridopidine on Functional Capacity in Patients with Huntington Disease

### Objective

To explore functional decline measured by the Total Functional Capacity (TFC) scale in patients treated with open-label pridopidine 90 mg/day (45 bid; per day) for 36 months (OPEN-HART) and compare results to historical cohorts of placebo patients enrolled in HSG-sponsored trials (CARE-HD and 2CARE).

Follow-up objectives included to report additional safety and exploratory efficacy data for continued open-label use of 45 mg bid (90 mg/day) pridopidine at 48 and 60 months (4- and 5-year time points).

### Background

Patients with HD experience motor, cognitive and behavioral symptoms that lead to serious, long-term disability. TFC (range 0-13, high scores indicate greater capacity) evaluates patients' capacity to work, handle finances and domestic chores, perform activities of daily living and live independently, and is most sensitive to early changes in disability. TFC was utilized in OPEN-HART and the Coenzyme Q10 studies, CARE-HD and 2CARE.

### Methods

This analysis compared the OPEN-HART cohort (n=50) that received pridopidine 90 mg/day (45 mg bid) and the placebo arms of CARE-HD (n=80) and 2CARE (n=213) without matching on baseline characteristics. For this analysis, TFC scores at baseline, 12, 24, and 36 months from OPEN-HART and 2CARE, and TFC scores at baseline, 12, 25, and 30 months from CARE were utilized.

The methods for the open-label extension of the HART study are as follows.

### Study Population and Design

The open-label extension analysis included HD participants who successfully completed the HART study and continued into Open-HART described herein.

Study structure remained similar from 36 months to 60 months. Participant contact occurred every three months, alternating between the following types of visits: Safety Visits were held in-person or by phone, consisting of adverse effects (AE)/concomitant medication review, vital signs, blood draws for electrolytes/creatinine clearance, ECG, and PBA-s. Some data could be collected locally by primary care physicians if optimal for the participant, while Clinic Visits comprised compliance assessment, review of concomitant medication and AEs, physical examination, ECG, vital signs and weight, blood draw for electrolytes and creatinine clearance, pregnancy test yearly when applicable, PBA-s, C-SSRS, and UHDRS.

### Efficacy Assessments

The UHDRS-TFC was evaluated as an exploratory endpoint. This scale relies on clinician assessment of the patient's ability to perform across five categories (capacity to work, finances, domestic chores, activities of daily living, level of care). Total scores range from 0 to 13, with higher scores indicating a greater capacity for independent function. TFC change was analyzed for the entire cohort, as well as separately in participants able to complete 60 months. TFC changes over 48 and 60 months were also compared to historical placebo participants from the Huntington Study Group-sponsored clinical trial 2CARE (McGarry A, McDermott M, Kieburtz K, et al. A randomized, double-blind, placebo-controlled trial of coenzyme Q10 in Huntington disease. Neurology. 2017; 88(2):152-159.). An early HD group (baseline scores 9-13) was used for this comparison, as this was the cohort in 2CARE. Additional exploratory analyses were conducted comparing “early initiators” (patients randomized to any of the pridopidine arms in the original HART study who continued in Open-HART) to “late initiators” (patients randomized to the placebo arm in HART who received pridopidine only upon starting Open-HART).

The UHDRS-TMS was also used to evaluate efficacy in an exploratory manner. Each domain within the TMS was rated on a five-point scale from 0 (normal) to 4 (maximally abnormal) to generate a total score (Huntington Study Group (Kieburtz K, primary author). The Unified Huntington's Disease Rating Scale: Reliability and Consistency. Mov Dis 1996; 11:136-142.). Similar analyses evaluating TMS change in 60-month completers and comparing TMS performance in Open HART to an early TFC cohort from 2CARE were also done.

### Statistical Analysis

Efficacy analyses for TMS, TFC, and the five individual functional capacity categories (capacity to work, finances, domestic chores, activities of daily living, and level of care) included mean and standard error (SE) scores as well as mean (SE) change from baseline at each yearly time interval for the entire cohort and the subset who completed the 60-months study.

Mean change from baseline for the TFC score, the five individual domains comprising the TFC, and TMS in the early HD cohort were compared to those randomized to placebo from the large, long-term HD study, 2CARE. Repeated-measures analysis of covariance models were used to compare estimates of treatment effect (pridopidine versus historical placebo) for each visit. Accounting for multiple comparisons, p-values <0.01 were considered statistically significant.

In order to account for missing data after participant discontinued in both Open HART and 2CARE, a mixed model repeated measures (MMRM) with multiple imputations (100 datasets) approach was used to model missing data for subjects in Open HART and 2CARE using data available from other subjects within the study.

Exploratory analyses comparing “early initiator” and “late initiator” patients from the HART and Open-HART studies were performed as described above. These included summaries of the baseline characteristics mean (SE) TMS and TFC scores and mean change (SE) from baseline at each yearly timepoint for the entire cohort and the subset who completed the 60-months study period.

For this comparison, analysis was done on the entire Open HART cohort (all patients), patients who completed 60 months of treatment (completers), and for an early HD subgroup (baseline TFC 9-13). Exploratory analyses on early initiation of pridopidine (completion of HART and Open-HART) and later initiation of pridopidine (Open-HART only) were also conducted. To account for missing data, sensitivity analyses using a mixed model repeated measures (MMRM) with multiple imputation was performed on early HD (TFC 9-13) sub-groups from Open-HART and 2CARE.

### Results

Results from 36-Month OPEN-HART Time Point.

At baseline, the OPEN-HART cohort had the lowest absolute mean (SD) TFC score compared with the CARE-HD and 2CARE cohorts [9.14(2.78), 10.3(1.7) and 11.05(1.47), respectively].

The mean change from baseline in TFC at 12 months was OPEN-HART: −0.49 (1.60), CARE: −1.00 (1.48) and 2CARE: −1.11 (1.62); at 24 months (OPEN-HART and 2CARE) and 25 months (CARE) was: −1.00 (1.92), −1.80 (2.06) and −2.24 (1.91), respectively; at 36 months (OPEN-HART and 2CARE) was: −1.68 (2.22) and −2.54 (2.53), respectively; and at 30 months (CARE) was: −2.80(2.27).

The results show that the TFC decline over time was slower in patients who received pridopidine in OPEN-HART compared to those who received placebo in CARE-HD and 2CARE. A slowdown in TFC decline was observed, which suggests that pridopidine has neuroprotective and/or disease-modifying properties.

Results from 48- and 60-Month OPEN-HART Time Points.

118 participants who originally enrolled and successfully completed the HART study were re-enrolled into Open HART. FIG. 41 indicates participant disposition over the 60-month analysis period. Forty participants completed 48 months (33.9%) and 33 completed 60 months (30.0%).

Table 145 depicts baseline demographics and concomitant psychoactive medications taken during the study. The majority of psychoactive medications were antidepressants (notably sertraline and paroxetine, 26.3% and 16.1%), with anxiolytics also common (clonazepam and lorazepam, 22% and 12.7%, respectively). Sleep aids were frequently used (trazodone 10.2%; zolpidem 9.3%; mirtazapine 7.6%), as was the mood-stabilizing agent valproate (9.3%).

TABLE 145
Baseline characteristics and concomitant medications
Characteristic

| Age, years (SD) | | |
|---|---|---|
| All patients (n = 118) | 52.3 | (9.6) |
| 60 Month completers (n = 33) | 54.1 | (8.9) |
| Early HD (TFC 9-13) (n = 55) | 52.6 | (8.9) |
| Sex, n (%) | | |
| All patients (n = 118) | Male: 56 (47.5), Female: 62 (52.5) | |
| 60 Month completers (n = 33) | Male: 18 (54.5), Female: 15 (45.5) | |
| Early HD (TFC 9-13) (n = 55) | Male: 28 (50.9), Female: 27 (49.1) | |
| CAG Repeat Length (SD) | | |
| All patients (n = 47) | 43.7 | (2.9) |
| 60 Month completers (n = 8) | 42.5 | (1.6) |
| Early HD (TFC 9-13) (n = 28) | 43.1 | (2.2) |
| Weight, kilograns (SD) | | |
| All patients (n = 118) | 74.2 | (18.7) |
| 60 Month completers (n = 33) | 77.9 | (16.3) |
| Early HD (TFC 9-13) (n = 55) | 75.3 | (20.1) |
| Total Motor Score (SD) | | |
| All patients (n = 118) | 38.7 | (15.7) |
| 60 Month completers (n = 33) | 32.4 | (13.6) |
| Early HD (TFC 9-13) (n = 55) | 32.9 | (12.5) |
| Total Functional Capacity (SD) | | |
| All patients (n = 118) | 8.4 | (2.6) |
| 60 Month completers (n = 33) | 8.8 | (2.9) |
| Early HD (TFC 9-13) (n = 55) | 10.6 | (1.3) |
| Psychoactive concomitant | | |
| medications taken by ≥5% | | |
| Subjects, n (%) | | |
| All patients (n = 118) | | |
| Sertraline | 31 | (26.3) |
| Clonazepam | 26 | (22.0) |
| Paroxetine | 19 | (16.1) |
| Lorazepam | 15 | (12.7) |
| Escitalopram | 12 | (10.2) |
| Trazodone | 12 | (10.2) |
| Citalopram | 12 | (10.2) |
| Valproate | 11 | (9.3) |
| Zolpidem | 11 | (9.3) |
| Mirtrazapine | 9 | (7.6) |
| Memantine | 8 | (6.8) |
| Fluoxetine | 8 | (6.8) |

Table 145 includes results for All patients (participants who were originally enrolled and successfully completed the double-blind HART Study and reenrolled into the Open Hart Study), 60 Months completers and early HD subgroup with baseline TFC 9-13; CAG not available for all participants. SD, standard deviation; TFC, total functional capacity.

### Exploratory Efficacy

Exploratory efficacy measures for Total Functional Capacity (TFC) and Total Motor Scale (TMS) at baseline, 24 months, 36 months, 48 months and 60 months data are indicated in Tables 146A and 146B.

TABLE 146A
Efficacy Analyses for UHDRS TFC

| | | | | |
|---|---|---|---|---|
| Baseline | Month 24 | Month 36 | Month 48 | Month 60 |

Total functional capacity (TFC)

| | | | | | |
|---|---|---|---|---|---|
| Open-HART | | | | | |
| Entire cohort | | | | | |
| Mean (SE) | 8.4 (0.2), n = 118 | 7.6 (0.4), n = 58 | 7.5 (0.4), n = 50 | 6.8 (0.6), n = 40 | 6.8 (0.6), n = 32 |
| Mean change (SE) from baseline | | −1.1 (0.3) | −1.7 (0.3) | −2.0 (0.4) | −1.9 (0.4) |
| Open-HART | | | | | |
| Cohort who completed 60 months | | | | | |
| Mean (SE) | 8.8 (0.5), n = 33 | 7.8 (0.5), n = 33 | 7.4 (0.6), n = 33 | 7.1 (0.6), n = 33 | 6.8 (0.6), n = 32 |
| Mean change (SE) from baseline | | −1.0 (0.4) | −1.4 (0.4) | −1.7 (0.4) | −1.9 (0.4) |
| Open-HART | | | | | |
| Early HD cohort (baseline TFC 9-13) | | | | | |
| Mean (SE) | 10.6 (0.2), n = 55 | 9.7 (0.4), n = 32 | 8.8 (2.3), n = 31 | 8.9 (0.5), n = 23 | 8.9 (0.5), n = 19 |
| Mean change (SE) from baseline | | −1.2 (0.4) | −2.1 (0.4) | −2.0 (0.6) | −1.8 (0.5) |
| 2CARE | | | | | |
| Early HD cohort (baseline TFC 9-13) | | | | | |
| Mean (SE) | 11.1 (0.1), n = 303 | 9.1 (0.2), n = 262 | 8.3 (0.2), n = 215 | 7.2 (0.3), n = 166 | 5.9 (0.4), n = 123 |
| Mean change (SE) from baseline | | −2.0 (0.1) | −2.7 (0.2) | −3.8 (0.2) | −5.0 (0.3) |
| | | p = 0.08 | p = 0.21 | p = 0.01 | p = 0.001 |

TABLE 146B
Efficacy Analyses for UHDRS TMS

| | Month | Month | Month | Month |
|---|---|---|---|---|
| Baseline | 24 | 36 | 48 | 60 |

Total motor score (TMS)
Total motor score (TMS)

| | | | | | |
|---|---|---|---|---|---|
| Open-HART | | | | | |
| Entire cohort | | | | | |
| Mean (SE) | 38.7 (1.5), | 42.5 (2.7), | 41.5 (2.6), | 45.0 (3.3), | 44.9 (3.3), |
| Mean change (SE) from | n = 118 | n = 58 | n = 50 | n = 40 | n = 32 |
| baseline | | 5.8 (1.5) | 7.8 (1.6) | 12.2 (2.2) | 12.2 (2.3) |
| Open-HART | | | | | |
| Cohort who completed | | | | | |
| 60 months | | | | | |
| Mean (SE) | 32.4 (2.4), | 37.6 (3.4), | 39.7 (3.6), | 43.4 (3.5), | 44.9 (3.7), |
| Mean change (SE) from | n = 33 | n = 33 | n = 33 | n = 33 | n = 32 |
| baseline | | 5.2 (2.1) | 7.3 (2.2) | 11.0 (2.1) | 12.2 (2.3) |
| Open-HART | | | | | |
| Early HD cohort (baseline | | | | | |
| TFC 9-13) | | | | | |
| Mean (SE) | 32.9 (1.7), | 32.6 (2.4), | 36.5 (2.6), | 35.9 (3.3), | 36.9 (3.7), |
| Mean change (SE) from | n = 55 | n = 32 | n = 31 | n = 23 | n = 19 |
| baseline | | 2.9 (1.7) | 7.0 (2.0) | 9.1 (2.5) | 10.5 (2.6) |
| 2CARE | | | | | |
| Early HD cohort (baseline | | | | | |
| TFC 9-13) | | | | | |
| Mean (SE) | 27.4 (0.8), | 34.4 (1.1), | 36.9 (1.3), | 41.5 (1.5), | 46.0 (2.0), |
| Mean change (SE) from | n = 300 | n = 252 | n = 215 | n = 166 | n = 113 |
| baseline | | 7.4 (0.7) | 9.8 (0.8) | 14.3 (1.1) | 18.5 (1.5) |
| | | p = 0.05 | p = 0.23 | p = 0.05 | p = 0.06 |

Tables 146 A-B include results for all patients (participants who were originally enrolled and successfully completed the double-blind HART Study and reenrolled into the Open-HART Study), 60 Months completers and early HD subgroup with baseline TFC 9-13. p-values shown compare the mean change from baseline between the Open-HART and 2CARE studies at each time-point. n value represents the number of patients who completed the indicated year of the study. SE, standard error; UHDRS, Unified Huntington's Disease Rating Scale.

Tables 146A and 146B include results for all patients (participants who were originally enrolled and successfully completed the double-blind HART Study and reenrolled into the Open Hart Study), 60 Months completers and early HD subgroup with baseline TFC 9-13. p-values shown compare the mean change from baseline between the Open-HART and 2CARE studies at each time-point. n value represents the number of patients who completed the indicated year of the study. SE, standard error; UHDRS, Unified Huntington's Disease Rating Scale.

The rates of decline in TFC (lower scores indicate worsening) and TMS (higher scores indicate worsening) were evaluated for the entire cohort (N=118), for the 60 months completers (N=33), and for the early HD subgroup (N=55, baseline TFC 9-13) in Open-HART. For comparison, an early HD cohort (TFC 9-13) from 2CARE participants receiving placebo was also analyzed for TFC change over 60 months (Table 146A and 146B).

The baseline mean (SE) TFC was 8.4 (0.2) for the entire cohort, 8.8 (0.5) for the 60 months completers, and 10.6 (0.2) for the early HD subgroup (TFC 9-13). Mean baseline TFC (SE) was 11.1 (0.1) for the 2CARE placebo group (n=303, early HD TFC 9-13). Comparing the early HD subgroups (baseline TFC 9-13) between Open-HART patients receiving pridopidine 45 mg bid and patients from 2CARE receiving placebo, mean change from baseline in TFC at 24, 36, 48 and 60 months was −1.2 (0.4), −2.1 (0.4), −2.0 (0.6) and −1.8 (0.5) for Open HART and −2.0 (0.1), −2.7 (0.2), −3.8 (0.2) and −5.0 (0.3) for 2CARE.

Open-HART had nominally significantly slower TFC decline than 2CARE at 48 months (−2.0 (0.6) vs. −3.8 (0.2), p=0.01) and 60 months (−1.8 (0.5) vs. −5.0 (0.3), p=0.001) (Table 10A). In Open-HART, TFC remained stable (no worsening) between 48 and 60 months, with a mean (SE) of 6.8 (0.6) for the entire cohort and 8.9 (0.5) for the early HD subgroup. Over this same interval, the 2CARE early cohort was not stable, deteriorating by −1.2 points.

To address the influence of dropouts on TFC over the course of the study, a sensitivity analysis was conducted using MMRM and multiple imputation for earlier-stage participants (TFC 9-13) from both Open-HART and 2CARE (Table 147).

TABLE 147
Exploratory Efficacy Analyses for UHDRS TFC and TMS with Multiply Imputed Data

| | | | | |
|---|---|---|---|---|
| Baseline | Month 24 | Month 36 | Month 48 | Month 60 |

Total functional capacity (TFC)

| | | | | | |
|---|---|---|---|---|---|
| Open-HART | | | | | |
| Early HD cohort (baseline TFC 9-13) | | | | | |
| Mean (SE) | 10.6 (0.2), n = 55 | 9.4 (0.4), n = 55 | 8.5 (0.4), n = 55 | 8.4 (0.5), n = 55 | 8.2 (0.5), n = 55 |
| Mean change (SE) from baseline | | −1.4 (0.4) | −2.3 (0.4) | −2.3 (0.5) | −2.4 (0.5) |
| 2CARE | | | | | |
| Early HD cohort (baseline TFC 9-13) | | | | | |
| Mean (SE) | 11.1 (0.1), n = 303 | 9.1 (0.2), n = 303 | 8.4 (0.2), n = 303 | 7.4 (0.2), n = 303 | 6.5 (0.2), n = 303 |
| Mean change (SE) from baseline | | −2.0 (0.1) | −2.8 (0.2) | −3.7 (0.2) | −4.6 (0.2) |
| | | p* = 0.08 | p = 0.30 | p = 0.01 | p < 0.001 |

Total motor score (TMS)

| | | | | | |
|---|---|---|---|---|---|
| Open-HART | | | | | |
| Early HD cohort (baseline TFC 9-13) | | | | | |
| Mean (SE) | 32.9 (1.7), n = 55 | 35.4 (2.5), n = 55 | 39.2 (2.6), n = 55 | 42.4 (2.9), n = 55 | 46.9 (3.2), n = 55 |
| Mean change (SE) from baseline | | 3.8 (1.7) | 8.3 (1.8) | 10.6 (2.2) | 14.2 (2.5) |
| 2CARE | | | | | |
| Early HD cohort (baseline TFC 9-13) | | | | | |
| Mean (SE) | 27.4 (0.8), n = 300 | 34.6 (1.1), n = 300 | 37.4 (1.2), n = 300 | 42.3 (1.3), n = 300 | 46.3 (1.4), n = 300 |
| Mean change (SE) from baseline | | 7.3 (0.7) | 10.1 (0.7) | 15.0 (0.9) | 19.0 (3.0) |
| | | p = 0.05 | p = 0.36 | p = 0.07 | p = 0.08 |

p-values shown compare the mean change from baseline between the Open-HART and 2CARE studies at each time-point. SE, standard error; UHDRS, Unified Huntington’s Disease Rating Scale.

With this approach, observations for the baseline TFC 9-13 group achieving nominal significance in the non-imputed analysis remained nominally significant, again showing less TFC decline for Open HART participants at 48 and 60 months compared to 2CARE (p=0.01, 0.001, respectively). MMRM analysis indicated minimal deterioration in TFC between 36 and 60 months, with mean TFC (SE) of 8.5 (0.4), 8.4 (0.5) and 8.2 (0.5) at 36, 48 and 60 months respectively.

Less decline was seen in Open HART participants at 60 months in each of the TFC domains; when corrected for multiple comparisons, ADLs, finances, care level, and domestic chores were significantly different than 2CARE early TFC cohorts (−0.3 vs. −1.0; −0.4 vs −1.3; −0.1 vs. −0.5; and −0.4 vs −1.0, respectively, all p<0.01) (Table 148).

TABLE 148
Mean Change in Total Functional Capacity Domains for Early HD Cohorts (TFC 9-13)
Completing 60 Months From Open-HART and 2CARE

| | | | | |
|---|---|---|---|---|
| Baseline | Month 24 | Months 36 | Months 48 | Month 60 |

Total Functional Capacity-Occupation

| | | | | | | |
|---|---|---|---|---|---|---|
| Open-HART | Mean (SE) | 1.3 (0.1), | 0.9 (0.2) n = 32 | 0.8 (0.2) n = 31 | 0.6 (0.2) n = 23 | 0.5 (0.2), |
| | Mean Change | n = 55 | −0.5 (0.2) | −0.6 (0.2) | −0.7 (0.2) | n = 19 |
| | from baseline | | | | | −0.7 (0.3) |
| 2CARE | Mean (SE) | 1.7 (0.1), | 1.0 (0.1), n = 262 | 0.9 (0.1), n = 215 | 0.7 (0.1), n = 166 | 0.5 (0.1), n = 123 |
| | Mean Change | n = 303 | −0.7 (0.1) | −0.8 (0.1) | −1.0 (0.1) | −1.1 (0.1) |
| | from baseline | | p = 0.51 | p = 0.48 | p = 0.58 | p = 0.60 |

Total Functional Capacity-Activity of Daily living

| | | | | | | |
|---|---|---|---|---|---|---|
| Open-HART | Mean (SE) | 2.9 (0.05), | 2.8 (0.1), n = 32 | 2.7 (0.1), n = 31 | 2.7 (0.2), n = 23 | 2.7 (0.1), |
| | Mean Change | n = 55 | −0.1 (0.1) | −0.2 (0.1) | −0.3 (0.2) | n = 19 |
| | from baseline | | | | | −0.3 (0.1) |
| 2CARE | Mean (SE) | 2.9 (0.02), | 2.6 (0.04), n = 262 | 2.5 (0.1), n = 215 | 2.2 (0.1), n = 166 | 1.9 (0.1), n = 123 |
| | Mean Change | n = 303 | −0.3 (0.04) | −0.4 (0.1) | −0.7 (0.1) | −1.0 (0.1) |
| | from baseline | | p = 0.20 | p = 0.13 | p = 0.03 | p = 0.002 |

Total Functional Capacity-Finances

| | | | | | | |
|---|---|---|---|---|---|---|
| Open-HART | Mean (SE) | 2.6 (0.1), | 2.3 (0.2), n = 32 | 1.8 (0.2), n = 31 | 2.1 (0.2), n = 23 | 2.2 (0.2), |
| | Mean Change | n = 55 | −0.3 (0.1) | −0.8 (0.2) | −0.6 (0.2) | n = 19 |
| | from baseline | | | | | −0.4 (0.3) |
| 2CARE | Mean (SE) | 2.6 (0.03), | 2.0 (0.1), n = 262 | 1.8 (0.1), n = 215 | 1.4 (0.1), n = 166 | 1.2 (0.1), n = 123 |
| | Mean Change | n = 303 | −0.6 (0.1) | −0.8 (0.1) | −1.1 (0.1) | −1.3 (0.1) |
| | from baseline | | p = 0.19 | p = 0.73 | p = 0.03 | p = 0.01 |

Total Functional Capacity-Care level

| | | | | | | |
|---|---|---|---|---|---|---|
| Open-HART | Mean (SE) | 2.0 (0.02), | 20 (0.0), n = 32 | 2.0 (0.03), | 2.0 (0.0), n = 23 | 1.9 (0.1), |
| | Mean Change | n = 55 | 0.0 (0.0) | n = 31 | 0.0 (0.0) | n = 19 |
| | from baseline | | | −0.03 (0.03) | | −0.1 (0.1) |
| 2CARE | Mean (SE) | 2.0 (0.0), | 1.9 (0.02), | 1.8 (0.03), | 1.7 (0.1), | 1.5 (0.1), |
| | | n = 303 | n = 262 | n = 215 | n = 166 | n = 123 |
| | Mean Change | | −0.1 (0.02) | −0.2 (0.03) | −0.3 (0.1) | −0.5 (0.1) |
| | from baseline | | p = 0.19 | p = 0.15 | p = 0.03 | p = 0.01 |

Total Functional Capacity-Domestic Chores

| | | | | | | |
|---|---|---|---|---|---|---|
| Open-HART | Mean (SE) | 1.9 (0.5) | 1.7 (0.1), n = 32 | 1.5 (0.1), n = 31 | 1.5 (0.1), n = 23 | 1.5 (0.1), |
| | Mean Change | n = 55 | −0.2 (0.1) | −0.4 (0.1) | −0.5 (0.1) | n = 19 |
| | from baseline | | | | | −0.4 (0.1) |
| 2CARE | Mean (SE) | 1.9 (0.02), | 1.6 (0.04), n = 262 | 1.4 (0.1), n = 215 | 1.2 (0.1), n = 166 | 0.9 (0.1), n = 123 |
| | Mean Change | n = 303 | −0.3 (0.03) | −0.5 (0.04) | −0.7 (0.1) | −1.0 (0.1) |
| | from baseline | | p = 0.67 | p = 0.49 | p = 0.15 | p = 0.003 |

Table includes results for early HD participants from the Open-HART study and 2CARE trial with baseline TFC 9-13.
p-values shown compare the change from baseline between the Open-HART and 2CARE studies at each time point.

Sensitivity analyses for baseline TFC 9-13 participants from both Open HART and 2CARE resulted in preservation of these same nominally significant findings (Table 149).

TABLE 149
Exploratory Efficacy Analyses for UHDRS TFC Domains with Multiply Imputed Data

| | | | | |
|---|---|---|---|---|
| Baseline | Month 24 | Months 36 | Months 48 | Month 60 |

Total Functional Capacity-Occupation

| | | | | | | |
|---|---|---|---|---|---|---|
| Open- | Mean (SE) | 1.3 (0.1), n = 55 | 0.9 (0.1) n = 55 | 0.7 (0.1) n = 55 | 0.6 (0.2) n = 55 | 0.5 (0.1), n = 55 |
| HART | Mean Change from baseline | | −0.6 (0.1) | −0.7 (0.1) | −0.9 (0.1) | −0.9 (0.1) |
| 2CARE | Mean (SE) | 1.7 (0.1), | 1.0 (0.1), | 0.9 (0.1), | 0.7 (0.1), | 0.6 (0.1), |
| | Mean Change from baseline | n = 303 | n = 303 | n = 303 | n = 303 | n = 303 |
| | | | −0.7 (0.1) | −0.8 (0.1) | −1.0 (0.1) | −1.1 (0.1) |
| | | | p = 0.46 | p = 0.49 | p = 0.44 | p = 0.43 |

Total Functional Capacity-Activity of Daily living

| | | | | | | |
|---|---|---|---|---|---|---|
| Open- | Mean (SE) | 2.9 (0.05), n = 55 | 2.7 (0.1), n = 55 | 2.7 (0.1), n = 55 | 2.6 (0.2), n = 55 | 2.6 (0.2), n = 55 |
| HART | Mean Change from baseline | | −0.2 (0.1) | −0.2 (0.1) | −0.2 (0.2) | −0.2 (0.1) |
| 2CARE | Mean (SE) | 2.9 (0.02), | 2.6 (0.04), | 2.5 (0.05), | 2.2 (0.1), | 1.9 (0.1), |
| | Mean Change from baseline | n = 303 | n = 303 | n = 303 | n = 303 | n = 303 |
| | | | −0.3 (0.04) | −0.4 (0.05) | −0.7 (0.1) | −1.0 (0.1) |
| | | | p = 0.17 | p = 0.09 | p = 0.04 | p < 0.001 |

Total Functional Capacity-Finances

| | | | | | | |
|---|---|---|---|---|---|---|
| Open- | Mean (SE) | 2.6 (0.1). n = 55 | 2.2 (0.1), n = 55 | 1.7 (0.2), n = 55 | 2.0 (0.2), n = 55 | 2.0 (0.2), n = 55 |
| HART | Mean Change from baseline | | −0.4 (0.1) | −0.9 (0.2) | −0.6 (0.2) | −0.6 (0.2) |
| 2CARE | Mean (SE) | 2.6 (0.03), | 2.0 (0.1), | 1.8 (0.1), | 1.5 (0.1), | 1.3 (0.1), |
| | Mean Change from baseline | n = 303 | n = 303 | n = 303 | n = 303 | n = 303 |
| | | | −0.6 (0.1) | −0.8 (0.1) | −1.1 (0.1) | −1.3 (0.1) |
| | | | p = 0.23 | p = 0.47 | p = 0.01 | p = 0.01 |

Total Functional Capacity-Care level

| | | | | | | |
|---|---|---|---|---|---|---|
| Open- | Mean (SE) | 2.0 (0.02), n = 55 | 20 (0.4), n = 55 | 1.9 (0.1), n = 55 | 2.0 (0.0), n = 55 | 1.9 (0.1), n = 55 |
| HART | Mean Change from baseline | | −0.02 (0.1) | −0.05 (0.1) | −0.005 (0.1) | −0.1 (0.1) |
| 2CARE | Mean (SE) | 2.0 (0.0), | 1.9 (0.02), | 1.8 (0.03), | 1.7 (0.04), | 1.6 (0.05), |
| | Mean Change from baseline | n = 303 | n = 303 | n = 303 | n = 303 | n = 303 |
| | | | −0.1 (0.02) | −0.2 (0.03) | −0.3 (0.4) | −0.4 (0.5) |
| | | | p = 0.15 | p = 0.12 | p = 0.01 | p = 0.002 |

Total Functional Capacity-Domestic Chores

| | | | | | | |
|---|---|---|---|---|---|---|
| Open- | Mean (SE) | 1.9 (0.5) n = 55 | 1.6 (0.1), n = 55 | 1.4 (0.1), n = 55 | 1.3 (0.1), n = 55 | 1.4 (0.1), n = 55 |
| HART | Mean Change from baseline | | −0.3 (0.1) | −0.4 (0.1) | −0.5 (0.1) | −0.4 (0.1) |
| 2CARE | Mean (SE) | 1.9 (0.02), | 1.6 (0.04), | 1.4 (0.04), | 1.2 (0.05), | 1.0 (0.05), |
| | Mean Change from baseline | n = 303 | n = 303 | n = 303 | n = 303 | n = 303 |
| | | | −0.3 (0.03) | −0.5 (0.04) | −0.7 (0.05) | −0.9 (0.05) |
| | | | p = 0.69 | p = 0.58 | p = 0.13 | p < 0.001 |

Table includes results for early HD participants from the Open-HART study and 2CARE trial with baseline TFC 9-13.
* Indicates the p-value comparing the change from baseline between the Open-HART and 2CARE studies at each time-point.

In Open-HART, mean (SE) baseline TMS was 38.7 (1.5) for entire cohort, 32.4 (2.4) for the 60 months completers, and 32.9 (1.7) for the early HD cohort. The 60-month completers and early HD cohorts had lower mean baseline TMS scores compared to the entire cohort. The early-stage HD cohort in Open-HART had a higher (more impaired) mean (SE) TMS score at baseline compared to the early stage HD 2CARE cohort (32.9 (1.7) Open-HART vs 27.4 (0.8) 2CARE). In both the Open-HART and the 2CARE trials, TMS increased over 5 years, indicating motor deterioration. The mean TMS change for the early HD Open-HART cohort was consistently smaller compared to early HD in the 2CARE placebo arm. Early HD in Open-HART showed less TMS deterioration than 2CARE at 24 months (2.9 (1.7) vs 7.4 (0.7) p=0.05), 48 months (9.1(2.5) vs 14.3(1.1), p=0.05) and 60 months (10.5(2.6) vs 18.5(1.5), p=0.06). TMS scores remained stable in the early HD Open-HART cohort at months 36, 48 and 60 (mean TMS 36.5(2.6), 35.9(3.3) and 36.9(3.7) respectively), though the available sample was decreasing progressively with time. Over this same interval the 2CARE early cohort appeared to worsen, with TMS mean (SE) of 36.9 (1.3), 41.5 (1.5) and 46.0 (2.0) at 36, 48 and 60 months. To analyze longitudinal TMS change in the baseline TFC 9-13 group while accounting for dropouts, a MMRM sensitivity analysis with multiple imputation was again conducted. The mean TMS decline from baseline was again consistently smaller in Open-HART compared to 2CARE early HD participants using the MMRM analysis. Early HD in Open-HART showed less TMS decline than 2CARE at 24 months (3.8 (1.7) vs 7.3 (0.7)), 36 months (8.3(1.8) vs 10.1(0.7)), 48 months (10.6 (2.2) vs 15.0 (0.9)) and 60 months (14.2(2.5) vs 19.0 (1.0)). Sensitivity analyses for differences from baseline between Open-HART and 2CARE for the early cohort at 48 and 60 months again trended towards nominally significant values (p=0.07 at 48 months and p=0.08 at 60 months).

Additional exploratory analyses were conducted on “early initiators” (patients previously exposed to pridopidine in HART) and “late initiators” (patients starting pridopidine for the first time in Open-HART). The early cohort was substantially larger than the late cohort (n=87, vs. n=31), while the late cohort had relatively more female participants (64.5%, vs 48.3%). Psychoactive medications, adverse events and the proportion of subjects reporting serious adverse events were generally similar between groups (Data not shown). Early initiators showed less decline at Months 48 and 60 in TFC compared to the late initiators, though TMS scores were variable; small numbers in the late initiator group over time prevent meaningful comparisons (Table 150).

TABLE 150
Exploratory efficacy analyses for UHDRS TMS and TFC for early/late initiators
and 60-month completers*

| Baseline | Month 24 | Month 36 | Month 48 | Month 60 |
|---|---|---|---|---|
| Entire cohort N | | | | |
| 118 | 58 | 50 | 40 | 32 |
| Early initiators N | | | | |
| 87 | 46 | 41 | 31 | 26 |
| Late initiators N | | | | |
| 31 | 12 | 9 | 9 | 6 |

Total Motor Score (TMS)

| Entire cohort (n = 118) | | | | | | | | | | |
|---|---|---|---|---|---|---|---|---|---|---|
| Mean (SE) | 38.7 | (1.5) | 42.5 | (2.7) | 41.5 | (2.6) | 45.0 | (3.3) | 44.9 | (3.3) |
| Mean change (SE) from baseline | | | 5.8 | (1.5) | 7.3 | (1.6) | 12.2 | (2.2) | 12.2 | (2.3) |
| Early initiators cohort (n = 87) | | | | | | | | | | |
| Mean (SE) | 37.3 | (1.6) | 41.9 | (3.0) | 40.7 | (2.7) | 43.1 | (3.6) | 45.2 | (4.2) |
| Mean change (SE) from baseline | | | 4.6 | (1.6) | 6.1 | (1.4) | 9.4 | (2.2) | 11.0 | (2.7) |
| Late initiators cohort (n = 31) | | | | | | | | | | |
| Mean (SE) | 42.8 | (3.2) | 44.7 | (6.1) | 45.1 | (8.0) | 51.3 | (7.5) | 43.7 | (8.1) |
| Mean change (SE) from baseline | | | 10.7 | (3.4) | 15.4 | (5.9) | 21.7 | (5.2) | 17.3 | (4.6) |

Total Functional Capacity (TFC)

| | | | | | | | | | | |
|---|---|---|---|---|---|---|---|---|---|---|
| Entire cohort (n = 118) | | | | | | | | | | |
| Mean (SE) | 8.4 | (0.2) | 7.6 | (0.4) | 7.5 | (0.4) | 6.8 | (0.6) | 6.8 | (0.6) |
| Mean change (SE) from baseline | | | −1.1 | (0.3) | −1.7 | (0.3) | −2.0 | (0.4) | −1.9 | (0.4) |
| Early initiators cohort (n = 87) | | | | | | | | | | |
| Mean (SE) | 8.4 | (0.3) | 7.8 | (0.5) | 7.6 | (0.5) | 7.0 | (0.6) | 6.7 | (0.7) |
| Mean change (SE) from baseline | | | −1.1 | (0.3) | −1.6 | (0.3) | −1.7 | (0.4) | −1.9 | (0.4) |
| Late initiators cohort (n = 31) | | | | | | | | | | |
| Mean (SE) | 8.1 | (0.4) | 7.2 | (0.9) | 7.0 | (1.1) | 6.1 | (1.3) | 7.7 | (1.2) |
| Mean change (SE) from baseline | | | −1.4 | (0.6) | −1.9 | (1.0) | −2.8 | (0.9) | −2.0 | (1.0) |

Table 150 includes results for participants who were originally enrolled and successfully completed the double-blind HART Study and reenrolled into the Open Hart Study. SE, standard error; UHDRS, Unified Huntingdon’s Disease Rating Scale.

By 48 months, mean (SE) TFC change from baseline was −1.7 (0.4) for the early initiators (n=31) compared to −2.8 (0.9) in the late initiators group (n=9). Between Month 48 and 60, there was an apparent improvement in TFC in the late initiators group [−2.8 (0.9) decline at 48 months vs −2.0 (1.0) decline at 60 months], most likely due to the discontinuation of 3 out of 9 patients (33%). As a result, at 60 months, the change from baseline in TFC was comparable between early initiators [n=26, −1.9 (0.4)] and late initiators [n=6, −2.0 (1.0)].

Demographic characteristics (age, sex, weight) for early HD participants in Open-HART and 2CARE at baseline, 48 and 60 months were similar Table 151).

TABLE 151
Similar demographic characteristics for early HD participants
in Open-HART and 2CARE at baseline
Age, years

| | |
|---|---|
| Open-HART | |
| Early HD cohort (baseline TFC 9-13), | 52.6 (8.9, 1.2), n = 55 |
| Mean (SD, SE) | |
| 2CARE | |
| Early HD cohort (baseline TFC 9-13), | 50.7 (11.6, 0.7), n = 303 |
| Mean (SD, SE) | |

Gender

| | |
|---|---|
| Open-HART | |
| Early HD cohort (baseline TFC 9-13), | 50.9, n = 55 |
| % Male | |
| 2CARE | |
| Early HD cohort (baseline TFC 9-13), | 45.9, n = 303 |
| % Male | |

Weight kilograms

| | |
|---|---|
| Open-HART | |
| Early HD cohort (baseline TFC 9-13), | 75.3 (20.1, 2.7), n = 55 |
| Mean (SD, SE) | |
| 2CARE | |
| Early HD cohort (baseline TFC 9-13), | 76.1 (15.9, 0.9), n = 303 |
| Mean (SD, SE) | |

CAG repeat

| | |
|---|---|
| Open-HART | |
| Early HD cohort (baseline TFC 9-13), | 43.1 (2.2) n = 55 |
| Mean (SD) | |
| 2CARE | |
| Early HD cohort (baseline TFC 9-13), | 43.9 (3.8), n = 303 |
| Mean (SD) | |

Source: McGarry et al, JHD, 2020

### Summary

In this open label pridopidine extension study, representing the longest treatment duration to date, pridopidine remained safe and tolerable over 60 months. No unexpected safety or tolerability issues emerged in the period after the previous 36-month report. No apparent cumulative toxicity appeared to emerge over time, and laboratory, ECG, and vital signs remained stable over the study period. QTcF did not appear to worsen over time with concomitant use of QT-prolonging medications (antidepressants). The majority of adverse events noted by more than 10% of subjects were related to HD phenotype or likely secondary to trauma, an expected feature of HD.

Overall, the 5-year Open-HART data suggests that 45 mg bid (90 mg/daily) pridopidine has a benign safety and tolerability profile in HD patients, and is comparable with the safety profile reported in prior HD trials with pridopidine (HART, MermaiHD and PRIDE-HD).

Importantly, administration of 45 mg bid (90 mg/daily) pridopidine to HD patients showed maintenance or slowed the decline in TFC and motor deterioration, compared to HD patients receiving placebo (2CARE trial). The effect of pridopidine was most pronounced in early HD (TFC 9-13) subjects.

Mean annual TFC decline over 60 months was lower in pridopidine-treated participants (0.4 points/year) compared to the placebo group from 2CARE, where an expected average decline of 1 point per year was observed. TFC scores in Open-HART appeared to remain stable for all participants between 48 and 60 months at 6.8 (0.6) points, while also remaining stable for early HD participants (TFC 9-13) between 36, 48, and 60 months [mean (SE) of 8.8 (2.3), 8.9 (0.5), 8.9 (0.5), respectively]. This observation of stability in early HD between 36, 48 and 60 months remained after sensitivity analysis to account for dropouts [8.5 (0.4), 8.5 (0.5), and 8.2 (0.5), respectively] and was nominally significantly different from 2CARE at 48 months [(8.4 (0.5) Open-HART vs. 7.4 (0.2) 2CARE] and 60 months [(8.2 (0.5) Open-HART vs. 6.5 (0.2) 2CARE]. TFC stability in Open-HART compared to 2CARE at 48 and 60 months for the baseline TFC 9-13 group in both non-imputed and imputed analyses suggest this finding may be robust despite the progressive dropout of participants over the study period. TMS at 48 and 60 months also appeared to remain stable, averaging approximately 45 points. For early HD patients (TFC 9-13) from Open-HART, TMS appeared stable at 36, 48 and 60 months (mean (SE) 36.5(2.6), 35.9(3.3) and 36.9 (3.7), respectively). In 2CARE, TMS in the TFC 9-13 group continued to deteriorate, with respective mean (SE) of 36.9 (1.3), 41.5 (1.5) and 46.0 (2.0) at 36, 48 and 60 months. With sensitivity analysis to account for missing data, the trend toward nominally significant difference in TMS from baseline at 48 and 60 months remained similar to non-imputed data, although comparative sample sizes across the studies were still substantially different (N=303 2CARE, N=55 Open-HART).

The period from 48 to 60 months appears to demonstrate stable TFC and TMS scores. The relatively static TFC performance at Months 48 and 60 may be consistent with a stabilizing or protective effect of pridopidine, which is intriguing to consider given emerging preclinical evidence of neuroprotection via the S1R. Overall, 45 mg bid pridopidine continued to be safe and tolerable during long-term use in the HD population. These Open-HART data suggesting less TFC decline over time in early-stage participants compared to matched 2CARE placebo historical controls are consistent with observations from the recently completed randomized, double-blind, placebo-controlled PRIDE-HD trial, where pridopidine 45 mg bid was associated with less TFC decline at 52 weeks compared to placebo (Reilmann R, McGarry A, Grachev I D et al. Safety and efficacy of pridopidine in patients with Huntington's disease (PRIDE HD): a phase 2, randomized, placebo-controlled, multicenter, dose-ranging study. Lancet Neurol 2019 February; 18(2):165-176.).

Emerging data regarding potentially neuroprotective effects of pridopidine mediated by the S1R raise the possibility that some component of long-term TFC performance in Open-HART is driven by an underlying biological effect. This hypothesis, with converging trial data and a plausible mechanism substantiated by preclinical findings, warrants further testing of the 45 mg bid dosage in early-disease cohorts (i.e. TFC 9-13) in adequately powered, randomized, double-blind studies.

### Example 3: Comparison Measurements Between cUHDRS: Composite Unified Huntington Disease Rating Scale and Individual Component Scale Measurements

### Objective

To compare the holistic patient experience measured using the cUHDRS scoring system with individual clinical component measurements.

### Methods

cUHDRS combines the following clinical measurements: TMS, TFC, SDMT (Symbol Digit Modality Test) and SWR (Stroop Word Reading Test). Analysis was performed using data measurements from clinical trials either singly or combined in the cUHDRS.

### Results

S/N ratio is defined as the mean change from baseline to a given time divided by the corresponding SD. Therefore, the S/N ratio is a measure of the strength of the longitudinal change relative to the random variability of change for a given measure. A larger S/N ratio indicates greater reliable variance, which is a desirable characteristic for the general use of a clinical endpoint. The observed difference in the S/N ratio between the cUHDRS and each individual component in an HD clinical trial was shown to be numerically superior in 123 cases tested from the TRACK-HD study, and in 10 of 12 comparisons, the S/N ratio difference between the cUHDRS and its individual components was statistically significant (See, Schobel et al (2017) FIG. 2 therein).

Using a similar approach, the data presented in Table 152 comparing TFC measurements and cUHDRS measurements demonstrated that cUHDRS measurements indicated a strengthened pridopidine effect in early HD compared with the TFC measurement alone.

TABLE 152
HART: 45 mg bid TFC vs. cUHDRS at week 12. cUHDRS strengthens pridopidine
effect on TFC in early HD (HD1 + HD2)

| | | Placebo | Placebo | Pridopidine | Pridopidine | | |
|---|---|---|---|---|---|---|---|
| | Cohort | (n) | (mean) | (n) | (mean) | Δ | P-value |
| TFC | Early HD | 47 | −0.16 | 46 | 0.16 | 0.31 | 0.17 |
| cUHDRS | Early HD | 44 | 0.16 | 41 | 0.73 | 0.58 | 0.04 |

*Note also the improvement in statistical significance.

Pridopidine improves cUHDRS at week 52 as shown in Table 154.

Using cUHDRS as an analysis tool demonstrated the significance of pridopidine in early HD patients (TFC 7-13) as shown in Table 153.

TABLE 153
Pride-HD: 45 mg bid significantly improves
cUHDRS vs placebo at week 52 in early HD patients.

| | | | 95% CI | | |
|---|---|---|---|---|---|
| Comparison | Δ | SEM | Lower | Upper | p-value |
| 45 vs placebo | 0.6 | 0.29 | 0.03 | 1.17 | 0.04 |

### Summary

The cUHDRS score uses logical weighting for functional (TFC), motor (TMS) and cognitive scales (SWR and SD™) and produces a score that associates strongly with functional ability. Post hoc analysis demonstrates pridopidine significantly improves cUHDRS vs placebo at 52 weeks.

### Example 4: Results PROOF-HD Study

### Objective:

PROOF-HD was a Phase 3 study, 65 to 78-week, multicenter, randomized, double-blind, placebo controlled, parallel group study to evaluate the efficacy and safety of pridopidine HCl administered at a dose of 45 mg bid (the 45 mg refers to pridopidine base) in adult HD patients (TFC 7-13). The study evaluated the effect of pridopidine on HD diseases progression, functionality, motor function, behavioral and cognitive function.

The composition used in the study included pridopidine HCl and Compound 1 (in the range of 0.07% w/w-0.09% w/w from pridopidine) and Compound 4 (in the range of 0.10-0.12% w/w from pridopidine).

In the following Methods Section and Results 45 mg bid pridopidine refers to a composition comprising pridopidine HCl with Compound 1 (in the range of 0.07% w/w-0.09% w/w from pridopidine) and Compound 4 (in the range of 0.10-0.12% w/w from pridopidine). The 45 mg refers to the base form of pridopidine)

### Methods:

The study consisted of a screening period; a 2-week titration period; a 63-week, double-blind, full-dose treatment period; and a variable double-blind, full-dose treatment period up to 78 weeks, with a 2-week follow-up period. [001] Participants were those with stage 1-2 HD, which was defined as a UHDRS-TFC score of ≥7, at screening. Further, participants had to have an UHDRS-Independence scale (IS) score of ≤90% at screening and a UHDRS-TMS≥20. [002] During the screening period, patients provided informed consent and subsequently underwent assessments to determine eligibility for participation in the study. The stage of HD was established by the UHDRS TFC scale. The TMS and UHDRS-IS were assessed. [003] Eligible patients were invited to return for a baseline visit and baseline assessments. Those patients who remained eligible for study participation were randomly assigned (1:1 ratio) to 1 of the 2 treatment groups: 45 mg bid pridopidine or placebo bid. For patients assigned to receive pridopidine, the dose was titrated during the first 2 weeks from 45 mg qd to the final dose of 45 mg bid pridopidine. Overall Design of the Study: [004] The screening period was followed by a 65 to 78 weeks double-blind treatment period, composed of a 2-week titration period, a 63-week double-blind full-dose maintenance treatment period followed by a variable double-blind treatment period of up to 13 weeks (total of up to 78 weeks; Main study).

On Day 1 (Baseline visit), eligible participants were randomized in a 1:1 ratio to active (pridopidine 45 mg bid) or control (placebo) arm.

Starting on Day 1, during the titration period, all participants self-administered 1 capsule of study drug per os (PO-taken orally), once daily (qd), in the morning for 2 weeks. Thereafter, study drug was taken PO bid in the morning and in the afternoon for 63 weeks (full-dose maintenance double-blind treatment period). Participants who completed the maintenance period (63 weeks) continued into a variable double-blind period of up to 13 weeks or until the last participant randomized completed 65 weeks of treatment (2 weeks titration+63 weeks full dose), whichever came first.

The Open Label Extension (OLE) which is still ongoing consists of a 2-week up titration period and a maintenance period. During the up-titration period, participants self-administered 1 capsule of pridopidine 45 mg PO, qd, in the morning, for 2 weeks. Thereafter, pridopidine was taken PO, bid in the morning and in the afternoon.

Table 154 below presents the participants and study groups.

TABLE 154
Treatment Groups-Main Study

| | Dose and dose regimen | | |
|---|---|---|---|
| | Titration | Maintenance | Number |
| | Period | Period (65 to | of |
| Treatmant | (2 weeks) | 78 weeks) | participants |
| Active-pridopidine | 45 mg capsule | 45 mg capsule | 250 |
| | PO, qd | PO, bid (total | |
| | | daily dose of | |
| | | 90 mg) | |
| Control-matching | Capsule, PO, qd | Capsule, PO, qd | 249 |
| placebo | | | |

Pridopidine Dose Formulation, Route of Administration, Strength, and Levels: 45 mg Pridopidine was provided in the form of a hard gelatin capsule for oral administration. The titration period included administration of a 45 mg capsule qd (on-prescription; morning dose) for 2 weeks, followed by the main full-dose treatment period wherein participants took a 45 mg capsule bid (1 capsule in the morning and 1 capsule in the afternoon, 7 to 10 hours after morning dose) for a total daily dose of 90 mg.

The primary endpoint was the change from baseline in UHDRS-TFC to week 65 in patients treated with pridopidine 45 mg bid compared to patients receiving placebo.

TABLE 155
Primary and Secondary endpoints:

| | |
|---|---|
| Objectives | Endpoints |

Primary

| | |
|---|---|
| To assess the effect of pridopidine | Change from Baseline to Week |
| on functional capacity in | 65 in the UHDRS-TFC score |
| participants with Stage 1-2 HD | |

Multiplicity Adjusted Secondary Endpoint
Key Secondary (secondary endpoints are listed by order of hierarchy)

| | |
|---|---|
| To assess the effect of pridopidine | 1. Change from Baseline to Week |
| on a composite measure of | 65 in composite UHDRS |
| disease progression in | (cUHDRS) total score |
| participants with HD | |

Secondary (secondary endpoints are listed by order of hierarchy)

| | |
|---|---|
| To evaluate the effect of | 2. Proportion of participants with |
| pridopidine on functional | improvement or no worsening |
| capacity, motor function, | (change from Baseline ≥ 0 point) at |
| cognition, and other measures | Week 65 in UHDRS-TFC |
| of efficacy over time in | 3. Change from Baseline to Week 52 |
| participants with HD | in UHDRS-TFC score |
| | 4. Change from Baseline to Week 78 |
| | in UHDRS-TFC score |
| | 5. Change from Baseline to week 65 |
| | in Quantitative motor (Q-motor) |
| | finger tapping inter-onset interval |
| | (IOI) mean (Digitmotography) |
| | 6. Change from Baseline to Week 65 |
| | in UHDRS Total Motor Score (TMS) |
| | 7. Change from Baseline to Week 65 |
| | in Symbol Digit Modalities Test |
| | (SDMT) |
| | 8. Change from Baseline to Week 52 |
| | in UHDRS-TMS score |
| | 9. Proportion of participants with |
| | improvement or no worsening in |
| | Objectives and Endpoints (Primary, |
| | Secondary and Safety) Main Study |

TABLE 156
Study populations

| Population | Description |
|---|---|
| Intent-to-Treat | The ITT population includes all randomized |
| (ITT) | participants. The participants will be grouped as |
| population | randomized. |
| Modified | The mITT set is a subset of the ITT population and |
| Intent-to-Treat | includes all randomized participants who receive a |
| (mITT) | dose of study drug and have valid TFC efficacy |
| population | assessments both at the Baseline visit and at least one |
| | post-baseline in-clinic timepoint. The participants will |
| | be grouped as randomized. |
| Per Protocol | The per protocol Week 65 set is a subset of the mITT set |
| Week 65 | and includes all participants who have a valid in-clinic |
| (W65PP) | UHDRS-TFC at Week 65, were on study drug with |
| population | compliance >80% during the study and did not have |
| | any important protocol deviations impacting efficacy |
| | assessment. Per protocol variations will be defined prior |
| | to unblinding the data and participants subsequently |
| | grouped as treated. The per protocol set will be used for |
| | supplementary analysis of the primary and secondary |
| | endpoints. |
| Per Protocol | The per protocol Week 78 set is a subset of the mITT set |
| Week 78 | and includes all participants who have a valid in-clinic |
| (W78PP) | UHDRS-TFC at Week 78, were on study drug with |
| population | compliance >80% during the study and did not have any |
| | important protocol deviations impacting efficacy |
| | assessment. Per protocol variations will be defined prior |
| | to unblinding the data and participants subsequently |
| | grouped as treated. The per protocol set will be used for |
| | supplementary analysis of the primary and select |
| | secondary endpoints. |
| Safety | The safety set will include all participants receiving at |
| population | least one dose of study drug. The participants will be |
| | grouped according to the treatment regimen actually |
| | received. The safety set will be used for safety analyses. |
| PK | The PK population will include all randomized |
| population | participants who received at least 1 dose of pridopidine. |
| | and have at least 1 valid PK assessment. The participants |
| | will be grouped according to the treatment regimen |
| | actually received. |

The analysis of the results is presented in three main groups:
1. mITT—all patients group,
2. A group which excluded patients taking neuroleptics medication anytime during the trial.
3. The third group of analysis was done excluding patients taking neuroleptics and/or chorea medications.
4. A protocol group, participants who followed the specified treatment regimen, completed all required assessments. Analysis was done excluding patients taking neuroleptics and/or chorea medications.

Neuroleptics medications refers to: olanzapine, risperidone, tiapride, aripiprazole, quetiapine, fluphenazine, haloperidol, lithium, ziprasidone, amisulpride, asenapine, cariprazine, chlorpromazine, clotiapine, cyamemazine, flupentixol, prochlorperazine, promazine, and prothipendyl.

TABLE 157
Number of patients analyzed: mITT, mITT excluding neuroleptics,
mITT excluding neuroleptics and/or chorea medications.

| | Placebo | Pridopidine 45 mg bid |
|---|---|---|
| mITT | 247 | 243 |
| mITT excluding neuroleptics | 141 | 136 |
| mITT excluding neuroleptics | 112 | 97 |
| and/or chorea medications | | |

### Results:

There were 499 HD patients enrolled into PROOF-HD which made up the ITT population.

TABLE 158
Study performance, 458/499 participants completed 65 weeks.

| Study performance status | Placebo | | Pridopidine | | Total | |
|---|---|---|---|---|---|---|
| (Recruitment & Completion) | n (%) | | n (%) | | n (%) | |
| Patients Randomized | 249 | (100%) | 250 | (100%) | 499 | (100%) |
| (ITT/Safety population) | | | | | | |
| Modified ITT (mITT) | 247 | (99%) | 243 | (97%) | 490 | (98%) |
| Week 65 Completers | 233 | (94%) | 225 | (90%) | 458 | (92%) |
| Completers on study drug | 224 | (90%) | 213 | (85%) | 437 | (88%) |
| Completers off study drug | 9 | (3.6%) | 12 | (4.8%) | 21 | (4.2%) |
| Dropouts | 16 | (6.4%) | 25 | (10%) | 41 | (8%) |
| Uptake into OLE | | | | | 423* | (97%) |

*Out of eligible for OLE

TABLE 159
Baseline demographics mITT population well-balanced across groups

| | | Placebo | | Pridopidine | |
|---|---|---|---|---|---|
| Parameter | | n = 247 | | n = 243 | |
| Age (years), Mean (SD) | | 52.6 | (11.4) | 52.4 | (11.9) |
| Age Group, n (%) | <65 years | 212 | (86%) | 202 | (83%) |
| | ≥65 years | 35 | (14%) | 41 | (17%) |
| BMI (kg/m²), Mean (SD) | | 25.2 | (4.8) | 25.0 | (5.0) |
| Sex, n (%) | Female | 126 | (51%) | 130 | (53%) |
| | Male | 121 | (49%) | 113 | (47%) |
| Duration since onset (years) | Mean, (SD) | 4.6 | (4.6) | 4.3 | (3.2) |
| HD Stage (n, %) | HD1 (11-13) | 102 | (41%) | 100 | (41%) |
| | HD2 (7-10) | 145 | (59%) | 143 | (59%) |
| CAG Repeats, Mean (SD) | | 43.6 | (3.3) | 44.1 | (3.8) |
| CAP, Mean (SD) | | 494.4 | (92.8) | 513.2 | (83.3) |
| Neuroleptics during the study | Yes | 106 | (43%) | 107 | (44%) |
| (n, %) | No | 141 | (57%) | 136 | (56%) |
| Chorea meds during the | Yes | 47 | (19%) | 68 | (28%) |
| study (n, %) | No | 200 | (81%) | 175 | (72%) |
| Neuroleptics and/or Chorea | Yes | 135 | (55%) | 146 | (60%) |
| meds during the study (n, %) | No | 112 | (45%) | 97 | (40%) |
| UHDRS-TFC, Mean (SD) | | 9.9 | (1.7) | 9.8 | (1.7) |
| CUHDRS, Mean (SD) | | 8.9 | (2.7) | 8.7 | (2.6) |
| UHDRS-TMS, Mean (SD) | | 32.9 | (10.9) | 33.8 | (11.2) |
| SDMT, Mean (SD) | | 23.3 | (9.4) | 22.9 | (9.7) |
| SWR, Mean (SD) | | 61.9 | (18.2) | 60.6 | (18.0) |

TABLE 160
Safety data: Pridopidine 45 mg bid safety and
tolerability profile similar to placebo

| | Placebo, | | Pridopidine, | |
|---|---|---|---|---|
| | n = 249 | | n = 250 | |
| | n (%) | | n (%) | |
| Treatment Emergent AEs (TEAEs) | 214 | (86%) | 206 | (82%) |
| Related-TEAEs | 58 | (23%) | 60 | (24%) |
| Serious TEAEs (SAEs) (related/unrelated) | 21 | (8.4%) | 34 | (13.3%) |

| | | | |
|---|---|---|---|
| Related SAEs | 1 | (0.4%)* | 0 |

| | | | |
|---|---|---|---|
| Deaths until week 65 | 0 | 2 | (0.8%)** |

| | | |
|---|---|---|
| Adverse Event of Special Interest (AESI)- | 0 | 0 |
| ECG (QTcF >480 ms or change >60 ms) | | |

*Deemed related by the Investigator and not related by the sponsor
**One death is considered non-TEAE (Cardiac arrest, 1 year after stopping study drug)

UHDRS-TFC Score:

The UHDRS-TFC score is a clinical scale used for assessing the functional capacity of subjects with HD and monitoring the disease's progression and severity over time. This score ranges from 0 to 13 and higher scores indicate better functional capacity. A score of 13 means that the individual is entirely functional with no indications of impairment, while a score of 0 indicates complete dependence on others for daily activities.

UHDRS-TFC results from the PROOF-HD trial are shown in tables 161-162.

FIG. 42-43. In the group of HD participants not on neuroleptics, pridopidine treated patients show less decline in TFC compared to HD participants in the placebo group, up to 65 weeks (FIG. 42 and table 161). Moreover, a stronger effect of pridopidine showing less decline in TFC was measured in the group of HD participants not on neuroleptics and/or chorea medications (FIG. 43 and table 162).

In the group of patients not taking neuroleptics, pridopidine showed a positive trend for improvement in TFC at week 26 (change vs placebo 0.16, positive change indicates improvement), week 39 (change vs placebo 0.14) and week 52 (change vs placebo 0.18). The beneficial effect of pridopidine was stronger in patients not taking neuroleptics and/or chorea medications at 26 weeks (change vs placebo 0.14), 39 weeks (change vs placebo 0.29) and 52 weeks (change vs placebo 0.21).

TABLE 161
Change from baseline to Weeks 26, 39 and 52 in UHDRS-TFC score excluding
patients on neuroleptics medications anytime during the study.

| Week 26 | | | Week 39 | | | Week 52 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 141 (0) | 136 (0) | n | 141 (0) | 136 (0) | n | 141 (0) | 136 (0) |
| Baseline | 10.1 | 10.0 | Baseline | 10.1 | 10.0 | Baseline | 10.1 | 10.0 |
| mean (SD) | (1.69) | (1.63) | mean (SD) | (1.69) | (1.63) | mean (SD) | (1.69) | (1.63) |
| n | 141 (0) | 135 (1) | n | 136 (5) | 128 (8) | n | 134 (7) | 128 (8) |
| (missing) | | | (missing) | | | (missing) | | |
| Mean | −0.4 | −0.2 | Mean | −0.4 | −0.3 | Mean | −0.6 | −0.3 |
| (SD) | (1.09) | (1.50) | (SD) | (1.39) | (1.43) | (SD) | (1.51) | (1.60) |
| Median | 0 | 0 | Median | 0 | 0 | Median | 0 | 0 |
| Q1, Q3 | −1.0, 0.0 | −1.0, 0.0 | Q1, Q3 | −1.0, 0.0 | −1.0, 0.0 | Q1, Q3 | −1.0, 0.0 | −1.0, 0.0 |
| Min, | −6, 2 | −4, 5 | Min, | −5, 3 | −6, 5 | Min, | −6, 3 | −6, 5 |
| Max | | | Max | | | Max | | |
| LS Mean | −0.23 | 0.07 | LS Mean | −0.31 | −0.18 | LS Mean | −0.45 | −0.27 |
| (SE) | (0.290) | (0.301) | (SE) | (0.294) | (0.307) | (SE) | (0.300) | (0.312) |
| 95% CI | −0.80, 0.34 | 0.67, 0.52 | 95% CI | −0.89, 0.27 | −0.78, 0.43 | 95% CI | −1.04, 0.14 | −0.88, 0.35LS |
| LS Mean | | 0.16 | LS Mean | | 0.14 | LS Mean | | 0.18 |
| Difference | | (0.158) | Difference | | (0.174) | Difference | | (0.193) |
| (SE) vs | | | (SE) vs | | | (SE) vs | | |
| Placebo | | | Placebo | | | Placebo | | |
| 95% CI vs | | 0.15, 0.47 | 95% CI vs | | −0.21, 0.48 | 95% CI vs | | −0.20, 0.56 |
| Placebo | | | Placebo | | | Placebo | | |
| p-value vs | | 0.3192 | p-value vs | | 0.4328 | p-value vs | | 0.3456 |
| Placebo | | | Placebo | | | Placebo | | |

TABLE 162A
Change from baseline to Weeks 26, 39 and 52 in UHDRS-TFC score excluding
patients on neuroleptics and/or chorea medications anytime during the study.

| Week 26 | | | Week 39 | | | Week 52 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 112 (0) | 97 (0) | n | 112 (0) | 97 (0) | n | 112 (0) | 97 (0) |
| Baseline | 10.3 (1.60) | 10.2 (1.65) | Baseline | 10.3 (1.60) | 10.2 (1.65) | Baseline | 10.3 (1.60) | 10.2 (1.65) |
| mean (SD) | | | mean (SD) | | | mean (SD) | | |
| n | 112 (0) | 96 (1) | n | 107 (5) | 92 (5) | n | 106 (6) | 92 (5) |
| (missing) | | | (missing) | | | (missing) | | |
| Mean (SD) | −0.3 (1.08) | −0.1 (1.36) | Mean (SD) | −0.4 (1.31) | 0.0 (1.27) | Mean (SD) | −0.4 (1.38) | −0.1 (1.42) |
| Median | 0 | 0 | Median | 0 | 0 | Median | 0 | 0 |
| Q1, Q3 | −1.0, 0.0 | −1.0, 0.0 | Q1, Q3 | −1.0, 0.0 | −1.0, 1.0 | Q1, Q3 | −1.0, 0.0 | −1.0, 0.0 |
| Min, Max | −6, 2 | −4, 3 | Min, Max | −5, 3 | −5, 5 | Min, Max | −5, 3 | −5, 5 |
| LS Mean | −0.25 | −0.11 | LS Mean | −0.34 | −0.04 | LS Mean | −0.35 | −0.14 |
| (SE) | (0.115) | (0.123) | (SE) | (0.123) | (0.131) | (SE) | (0.134) | (0.143) |
| 95% CI | −0.48, −0.03 | −0.35, 0.13 | 95% CI | −0.58, −0.09 | −0.30, 0.21 | 95% CI | −0.61, −0.08 | −0.42, 0.14 |
| LS Mean | | 0.14 | LS Mean | | 0.29 | LS Mean | | 0.21 |
| Difference | | (0.168) | Difference | | (0.180) | Difference | | (0.196) |
| (SE) vs | | | (SE) vs | | | (SE) vs | | |
| Placebo | | | Placebo | | | Placebo | | |
| 95% CI vs | | −0.19, 0.47 | 95% CI vs | | −0.06, 0.65 | 95% CI vs | | −0.18, 0.59 |
| Placebo | | | Placebo | | | Placebo | | |
| p-value vs | | 0.4058 | p-value vs | | 0.1061 | p-value vs | | 0.2878 |
| Placebo | | | Placebo | | | Placebo | | |

TABLE 162B
Change from baseline to Weeks 26, 39, 52, 65 and 78 in UHDRS-TFC score Per
protocol group excluding patients on neuroleptics and/or chorea medications anytime during the study.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | −0.23 | 0.116 | 81 | −0.01 | 0.128 | 0.22 | 0.173 | 0.2128 | 95.6% |
| 39 | 102 | −0.29 | 0.126 | 80 | 0 | 0.139 | 0.3 | 0.187 | 0.1158 | 100 |
| 52 | 102 | −0.28 | 0.131 | 81 | −0.08 | 0.145 | 0.21 | 0.195 | 0.2875 | 71% |
| 65 | 102 | −0.5 | 0.139 | 82 | −0.43 | 0.153 | 0.07 | 0.207 | 0.7236 | 12.9% |
| 78 | 70 | −0.53 | 0.158 | 65 | −0.29 | 0.172 | 0.23 | 0.234 | 0.3172 | 44.5% |

Pridopidine improves functional capacity (TFC) in HD patients. At 26 weeks progression slowed by 95.6% vs placebo (change vs placebo 0.22), in 39 weeks progression slowed by 100% vs placebo (change vs placebo 0.3) in 52 weeks progression slowed by 71% vs placebo (change vs placebo 0.21) in 65 weeks progression slowed by 12.9% vs placebo (change vs placebo 0.07) and in 78 weeks progression slowed by 44.5% vs placebo (change vs placebo 0.23 (Positive change indicates improvement).

cUHDRS:

The composite Unified Huntington's Disease Rating Scale (cUHDRS) is a clinical scale used for evaluating the severity and progression of Huntington's disease (HD).

The cUHDRS is a composite scale measure comprising four subscales: Total Functional Capacity (TFC), Total Motor Score (TMS), and 2 cognitive measures: the Symbol-Digit Modality Test (SDMT), and Stroop Word Reading (SWR) Test. The TFC, TMS, SDMT, and SWR tests assess overall functional ability, motor symptoms, and cognitive function, respectively.

The cUHDRS score is validated scale used for tracking disease progression and evaluating treatment effectiveness in subjects with HD. Higher scores on TFC, SDMT and SWR, indicate better performance while lower scores on TMS indicate better motor function. By measuring multiple domains the cUHDRS provides a more global and comprehensive assessment of the disease stage.

In the mITT group, participants in the pridopidine group show less decline in cUHDRS compared to participants in the placebo group at 26 weeks (change vs placebo, 0.09, p=0.446) and 39 weeks (change vs placebo 0.11, p=0.4). In the group of participants not using neuroleptics, pridopidine shows maintenance or less decline in cUHDRS compared to participants in the placebo group (FIG. 45 and table 163) which was statistically significant at week 26 (change vs placebo 0.34, p=0.0172, positive change indicates improvement). Pridopidine shows less decline in cUHRDRS vs placebo was at 39 weeks (change vs placebo 0.28, p=0.0994) and 52 weeks (change vs placebo 0.26, p=0.1556). Notably, the positive effects of pridopidine treatment were even more pronounced in HD patients who were not on neuroleptics and/or chorea medications, as illustrated in FIG. 46 and table 165. participants in the pridopidine group show improvement cUHDRS compared to participants in the placebo group at 26 weeks (change vs placebo, 0.38, p=0.0147) and 39 weeks (change vs placebo 0.48, p=0.181).

TABLE 163
Change from baseline to Weeks 26 and 39 in cUHDRS score
in all patients (mITT group)

| Week 26 | | | | | Week 39 | | | | |
|---|---|---|---|---|---|---|---|---|---|
| | | | Pridopidine 45 | | | | | Pridopidine 45 | |
| | Placebo | | mg bid | | | Placebo | | mg bid | |
| n | 247 | (0) | 242 | (1) | n | 247 | (0) | 242 | (1) |
| Baseline mean (SD) | 8.87 | (2.666) | 8.68 | (2.566) | Baseline mean (SD) | 8.87 | (2.666) | 8.68 | (2.566) |
| n (missing) | 241 | (6) | 236 | (7) | n (missing) | 236 | (11) | 226 | (17) |
| Mean (SD) | 0.4 | (1.26) | 0.3 | (1.39) | Mean (SD) | −0.5 | (1.50) | −0.4 | (1.47) |
| Median | 0 | | 0 | | Median | 0 | | 0 | |
| Q1, Q3 | 1.0, 0.0 | | 1.0, 1.0 | | Q1, Q3 | −1.0, 00 | | −1.0, 1.0 | |
| Min, Max | −5, 3 | | 6, 3 | | Min, Max | −5, 3 | | −6, 4 | |
| LS Mean (SE) | −0.51 | (0.091) | 0.40 | (0.091) | LS Mean (SE) | −0.62 | (0.100) | −0.49 | (0.101) |

| 95% CI | −0.69, −0.33 | −0.57, −0.22 | | 95% CI | −0.82, −0.42 | −0.69, −0.29 | |
|---|---|---|---|---|---|---|---|
| LS Mean Difference | | 0.11 | (0.122) | LS Mean Difference | | 0.13 | (0.136) |
| (SE) vs Placebo | | | | (SE) vs Placebo | | | |

| | | | |
|---|---|---|---|
| 95% CI vs Placebo | −0.13, 0.35 | 95% CI vs Placebo | −0.14, 0.40 |
| p-value vs Placebo | 0.3559 | p-value vs Placebo | 0.3419 |

TABLE 164
Change from baseline to Weeks 26, 39 52 and 65 in cUHDRS score excluding patients
using neuroleptic medications anytime during the study.

| Week 26 | | | Week 39 | | | Week 52 | | | Week 65 | | |
|---|---|---|---|---|---|---|---|---|---|---|---|
| | | Pri- | | | Pri- | | | Pri- | | | Pri- |
| | | dopidine | | | dopidine | | | dopidine | | | dopidine |
| | Placebo | 45 mg bid | | Placebo | 45 mg bid | | Placebo | 45 mg bid | | Placebo | 45 mg bid |
| n | 141 (0) | 135 (1) | n | 141 (0) | 135 (1) | n | 141 (0) | 135 (1) | n | 141 (0) | 135 (1) |
| Baseline | 9.51 | 9.09 | Baseline | 9.51 | 9.09 | Baseline | 9.51 | 9.09 | Baseline | 9.51 | 9.09 |
| mean | (2.649) | (2.498) | mean | (2.649) | (2.498) | mean | (2.649) | (2.498) | mean | (2.649) | (2.498) |
| (SD) | | | (SD) | | | (SD) | | | (SD) | | |
| n | 139 (2) | 132 (4) | n | 135 (6) | 124 (12) | n | 130 | 124 (12) | n | 131 (10) | 124 (12) |
| (missing) | | | (missing) | | | (missing) | (11) | | (missing) | | |
| Mean | −0.36 | −0.04 | Mean | −0.35 | −0.06 | Mean | −0.54 | −0.28 | Mean | −0.58 | −0.42 |
| (SD) | (1.111) | (1.192) | (SD) | (1.340) | (1.380) | (SD) | (1.517) | (1.435) | (SD) | (1.486) | (1.536) |
| Median | −0.3 | 0 | Median | −0.3 | 0 | Median | −0.3 | −0.2 | Median | −0.3 | −0.35 |
| Q1, Q3 | −1.00, | −0.80, | Q1, Q3 | −1.00, | −0.85, | Q1, Q3 | −1.50, | −1.10, | Q1, Q3 | −1.50, | −1.50, |
| | 0.30 | 0.80 | | 0.60 | 0.80 | | 0.40 | 0.70 | | 0.30 | 0.30 |
| Min, Max | −3.6, | −3.5, | Min, Max | −4.5, | −5.5, | Min, Max | −4.9, | −4.1, | Min, Max | −4.5, | −4.1, |
| | 3.1 | 2.3 | | 3.5 | 3.7 | | 4.1 | 3.1 | | 4.8 | 3.7 |
| LS Mean | −0.37 | −0.03 | LS Mean | −0.37 | −0.09 | LS Mean | −0.55 | −0.29 | LS Mean | −0.60 | −0.46 |
| (SE) | (0.098) | (0.100) | (SE) | (0.117) | (0.121) | (SE) | (0.128) | (0.131) | (SE) | (0.129) | (0.132) |
| 95% CI | −0.56, | −0.23, | 95% CI | −0.60, | −0.33, | 95% CI | −0.81, | −0.55, | 95% CI | −0.85, | −0.72, |
| | −0.17 | 0.17 | | −0.14 | 0.14 | | −0.30 | −0.03 | | −0.34 | −0.20 |
| LS Mean | | 0.34 | LS Mean | | 0.28 | LS Mean | | 0.26 | LS Mean | | 0.13 |
| Difference | | (0.140) | Difference | | (0.168) | Difference | | (0.184) | Difference | | (0.185) |
| (SE) vs | | | (SE) vs | | | (SE) vs | | | (SE) vs | | |
| Placebo | | | Placebo | | | Placebo | | | Placebo | | |
| 95% CI vs | | 0.06, | 95% CI vs | | −0.05, | 95% CI vs | | −0.10, | 95% CI vs | | −0.23, |
| Placebo | | 0.61 | Placebo | | 0.61 | Placebo | | 0.63 | Placebo | | 0.50 |
| p-value vs | | 0.0172 | p-value vs | | 0.0994 | p-value vs | | 0.1556 | p-value vs | | 0.4688 |
| Placebo | | | Placebo | | | Placebo | | | Placebo | | |

TABLE 165A
Change from baseline to Weeks 26, 39, 52, 65 and 78 in cUHDRS score in
patients not using neuroleptics and/or chorea medications anytime during the study. mITT group.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 112 | −0.26 | 0.106 | 95 | 0.13 | 0.114 | 0.38 | 0.155 | 0.0147 | 152.5% |
| 39 | 107 | −0.28 | 0.123 | 90 | 0.2 | 0.132 | 0.48 | 0.181 | 0.0085 | 175.1% |
| 52 | 104 | −0.39 | 0.132 | 91 | −0.02 | 0.141 | 0.37 | 0.193 | 0.0604 | 94.6% |
| 65 | 106 | −0.45 | 0.143 | 91 | −0.28 | 0.153 | 0.17 | 0.21 | 0.4082 | 34.5% |
| 78 | 71 | −0.56 | 0.166 | 70 | −0.28 | 0.173 | 0.28 | 0.24 | 0.2451 | 47.4% |

TABLE 165B
Change from baseline to Weeks 26, 39, 52, 65 and 78 in cUHDRS score in
patients not using neuroleptics and/or chorea medications anytime during the study. Per protocol group.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | −0.21 | 0.111 | 80 | 0.23 | 0.123 | 0.44 | 0.166 | 0.0089 | 214% |
| 39 | 102 | −0.27 | 0.128 | 78 | 0.27 | 0.143 | 0.54 | 0.193 | 0.0056 | 204% |
| 52 | 100 | −0.34 | 0.138 | 80 | 0.04 | 0.153 | 0.39 | 0.206 | 0.063 | 112.2% |
| 65 | 102 | −0.4 | 0.15 | 81 | −0.21 | 0.167 | 0.19 | 0.225 | 0.3979 | 45.3% |
| 78 | 69 | −0.56 | 0.171 | 64 | −0.17 | 0.185 | 0.39 | 0.252 | 0.1231 | 68.2% |

Participants in the pridopidine group (Per protocol group) showed improvement in cUHDRS compared to participants in the placebo group at 26 weeks with 214% improvement compared to placebo (change vs placebo, 0.44, p=0.0089) and 39 weeks, with 204% improvement compared to placebo (change vs placebo 0.54, p=0.0056) in 52 weeks with 112.2% improvement compared to placebo (change vs placebo 0.39, p=0.063), in 65 weeks progression slowed by 45.3% compared to placebo (change vs placebo 0.19, p=0.3979) and in 78 weeks progression slowed by 68.2% compared to placebo (change vs placebo 0.39, p=0.1231). Significant up to 52 weeks. From the results presented Pridopidine Demonstrates Disease-Progression mitigation in patients not administering neuroleptics or Chorea Medications Throughout the study, as evidenced by cUHDRS measurements.

### SWR Score:

The Stroop Word Reading (SWR) test is a validated and widely used clinical measure of cognitive function. The Stroop Word Reading Test assesses the ability to inhibit cognitive interference and is commonly used to evaluate executive function, including cognitive flexibility and attentional control.

During the SWR test, individuals are asked to read a list of color names that are printed in colored ink, where the ink color does not match the word. This creates a cognitive conflict that requires the inhibition of a prepotent response to read the word, making the test challenging.

The SWR score is determined by assessing the time it takes to read the list of words and the number of errors made. Higher scores on the SWR test indicate better cognitive performance.

Pridopidine showed beneficial effects on SWR compared to placebo in all patients (mITT population, positive change indicates improvement) at week 26 (change vs placebo 1.54), week 39 (change vs placebo 1.62), week 52 (change vs placebo 0.66) and week 65 (change vs placebo 0.18) (table 166). In the overall study population, as demonstrated in FIG. 47, pridopidine treatment was associated with no decline in SWR scores up until week 39.

In the group of participants not taking neuroleptics, pridopidine treatment demonstrated stronger improvement vs placebo (FIG. 48 and table 166) at week 26, (change vs placebo 1.99), week 39 (change vs placebo 2.36), at week 52 (change vs placebo 1.95) and week 65 (change vs placebo 1.55).

Furthermore, the most pronounced and statistically significant improvement of pridopidine in SWR scores was observed in HD participants not taking neuroleptics and/or chorea medications up to 65 weeks (FIG. 49 and table 166). Pridopidine showed improvement in SWR at week 26 (change vs placebo 3.2, p=0.03), week 39 (change vs placebo 3.87, p=0.03), week 52 (change vs placebo 3.45, p=0.047) and week 65 (change vs placebo 1.53, p=0.44).

TABLE 166
Change from baseline to Weeks 26, 39, 52 and 65 in SWR score in all participants (mITT)

| Week 26 | | | Week 39 | | | Week 52 | | | Week 65 | | |
|---|---|---|---|---|---|---|---|---|---|---|---|
| | | Pri- | | | Pri- | | | Pri- | | | Pri- |
| | | dopidine | | | dopidine | | | dopidine | | | dopidine |
| | | 45 mg | | | 45 mg | | | 45 mg | | | 45 mg |
| | Placebo | bid | | Placebo | bid | | Placebo | bid | | Placebo | bid |
| n | 247 (0) | 242 (1) | n | 247 (0) | 242 (1) | n | 247 (0) | 242 (1) | n | 247 (0) | 242 (1) |
| Baseline | 61.9 | 60.6 | Baseline | 61.9 | 60.6 | Baseline | 61.9 | 60.6 | Baseline | 61.9 | 60.6 |
| mean | (18.23) | (18.03) | mean | (18.23) | (18.03) | mean | (18.23) | (18.03) | mean | (18.23) | (18.03) |
| (SD) | | | (SD) | | | (SD) | | | (SD) | | |
| n | 243 | 239 (4) | n | 236 | 228 (15) | n | 228 | 219 (24) | n | 230 | 222 (21) |
| (missing) | (4) | | (missing) | (11) | | (missing) | (19) | | (missing) | (17) | |
| Mean | −1.4 | 0.4 | Mean | −1.4 | 0.6 | Mean | −1.4 | −0.2 | Mean | −2.2 | −1.6 |
| (SD) | (10.68) | (10.86) | (SD) | (12.02) | (12.06) | (SD) | (11.24) | (12.70) | (SD | (12.69) | (13.38) |
| Median | −1 | 0 | Median | −1 | 0 | Median | −2 | 0 | Median | −2 | −1 |
| Q1, Q3 | −7.0, | −6.0, 6.0 | Q1, Q3 | −8.0, | −5.0, 6.0 | Q1, Q3 | −8.0, | −7.0, 6.0 | Q1, Q3 | −9.0, | −9.0, 6.0 |
| | 4.0 | | | 5.0 | | | 5.0 | | | 5.0 | |
| Min, Max | −48, 32 | −46, 57 | Min, Max | −59, 36 | −49, 54 | Min, Max | −44, 33 | −56, 46 | Min, Max | −50, 38 | −59, 52 |
| LS Mean | −1.50 | 0.04 | LS Mean | −1.64 | −0.02 | LS Mean | −1.54 | −0.88 | LS Mean | −2.35 | −2.17 |
| (SE) | (0.707) | (0.708) | (SE) | (0.793) | (0.798) | (SE) | (0.789) | (0.796) | (SE) | (0.858) | (0.865) |
| 95% CI | −2.89, | −1.35, | 95% CI | −3.20, | −1.59, | 95% CI | −3.09, | −2.45, | 95% CI | | −3.87, |
| | −0.11 | 1.43 | | −0.09 | 1.55 | | 0.01 | 0.68 | | | −0.47 |
| LS Mean | | 1.54 | LS Mean | | 1.62 | LS Mean | | 0.66 | LS Mean | | 0.18 |
| Difference | | (0.952) | Difference | | (1.082) | Difference | | (1.077) | Difference | | (1.178) |
| (SE) vs | | | (SE) vs | | | (SE) vs | | | (SE) vs | | |
| Placebo | | | Placebo | | | Placebo | | | Placebo | | |
| 95% CI vs | | −0.33, | 95% CI vs | | −0.50 | 95% CI vs | | −1.46, | 95% CI vs | | −2.14, |
| Placebo | | 3.41 | Placebo | | 3.75 | Placebo | | 2.78 | Placebo | | 2.50 |
| p-value vs | | 0.1066 | p-value vs | | 0.134 | p-value vs | | 0.5408 | p-value vs | | 0.8782 |
| Placebo | | | Placebo | | | Placebo | | | Placebo | | |

TABLE 166
Change from baseline to Weeks 26, 39, 52 and 65 in SWR score in participants not
taking neuroleptic medications anytime during the study.

| Week 26 | | | Week 39 | | | Week 52 | | | Week 65 | | |
|---|---|---|---|---|---|---|---|---|---|---|---|
| | | Pridopidine | | | Pridopidine | | | Pridopidine | | | Pridopidine |
| | Placebo | 45 mg bid | | Placebo | 45 mg bid | | Placebo | 45 mg bid | | Placebo | 45 mg bid |
| n | 141 (0) | 135 (1) | n | 141 (0) | 135 (1) | n | 141 (0) | 135 (1) | n | 141 (0) | 135 (1) |
| Baseline | 66.1 | 61.5 | Baseline | 66.1 | 61.5 | Baseline | 66.1 | 61.5 | Baseline | 66.1 | 61.5 |
| mean (SD) | (17.75) | (17.79) | mean (SD) | (17.75) | (17.79) | mean (SD) | (17.75) | (17.79) | mean (SD) | (17.75) | (17.79) |
| n | 139 | 133 (3) | n | 135 | 126 (10) | n | 131 | 125 (11) | n | 132 | 124 (12) |
| (missing) | (2) | | (missing) | (6) | | (missing) | (10) | | (missing) | (9) | |
| Mean (SD) | −1.8 | 1.1 | Mean (SD) | −1.0 | 2.2 | Mean (SD) | −1.1 | 1.8 | Mean (SD) | −1.8 | 1.0 |
| | (10.08) | (11.46) | | (12.76) | (11.74) | | (11.90) | (12.17) | | (13.41) | (13.92) |
| Median | −1 | 1 | Median | − 1 | 0 | Median | −2 | 1 | Median | −2 | 0 |
| Q1, Q3 | −7.0, | −6.0, 7.0 | Q1, Q3 | −7.0, | −4.0, 8.0 | Q1, Q3 | 8.0, | −5.0, 8.0 | Q1, Q3 | −8.0, | −7.0, 9.0 |
| | 4.0 | | | 5.0 | | | 5.0 | | | 4.0 | |
| Min, Max | −48, 32 | −46, 45 | Min, Max | −59, 36 | −21, 50 | Min, Max | −44, 33 | −35, 46 | Min, Max | −50, 38 | −59, 52 |
| LS Mean | −1.29 | 0.71 | LS Mean | −0.78 | 1.58 | LS Mean | −0.81 | 1.15 | LS Mean | −1.41 | 0.14 |
| (SE) | (0.899) | (0.919) | (SE) | (1.028) | (1.058) | (SE) | (1.007) | (1.029) | (SE) | (1.159) | (1.191) |
| 95% CI | 3.06, | −1.10, | 95% CI | 2.81, | 0.51, | 95% CI | 2.79, | −0.88, | 95% CI | −3.69, | 2.20, |
| | 0.48 | 2.52 | | 1.24 | 3.66 | | 1.18 | 3.18 | | 0.88 | 2.49 |
| LS Mean | | 1.99 | LS Mean | | 2.36 | LS Mean | | 1.95 | LS Mean | | 1.55 |
| Difference | | (1.291) | Difference | | (1.479) | Difference | | (1.444) | Difference | | (1.666) |
| (SE) vs | | | (SE) vs | | | (SE) vs | | | (SE) vs | | |
| Placebo | | | Placebo | | | Placebo | | | Placebo | | |
| 95% CI vs | | −0.55, | 95% CI vs | | −0.55, | 95% CI vs | | −0.89, | 95% CI vs | | −1.73, |
| Placebo | | 4.54 | Placebo | | 5.27 | Placebo | | 4.80 | Placebo | | 4.83 |
| p-value vs | | 0.1234 | p-value vs | | 0.1114 | p-value vs | | 0.1774 | p-value vs | | 0.3535 |
| Placebo | | | Placebo | | | Placebo | | | Placebo | | |

TABLE 167A
Change from baseline to Weeks 26, 39, 52 and 65 in SWR score in participants
not taking neuroleptic and chorea medications anytime during the study.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 112 | −0.86 | 1 | 95 | 2.34 | 1.076 | 3.2 | 1.474 | 0.0312 | 392.4% |
| 39 | 107 | −1.11 | 1.201 | 91 | 2.76 | 1.293 | 3.87 | 1.769 | 0.03 | 367.2% |
| 52 | 104 | −1.27 | 1.182 | 92 | 2.19 | 1.256 | 3.45 | 1.728 | 0.047 | 285.3% |
| 65 | 106 | −0.87 | 1.34 | 91 | 0.66 | 1.44 | 1.53 | 1.971 | 0.4396 | 181.5% |
| 78 | 72 | −2.06 | 1.316 | 70 | 0.92 | 1.374 | 2.98 | 1.906 | 0.1197 | 148% |

TABLE 167B
Change from baseline to Weeks 26, 39, 52, 65 and 78 in SWR score in participants not
taking neuroleptic and chorea medications anytime during the study, per protocol group.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | −0.84 | 1.067 | 80 | 2.77 | 1.183 | 3.62 | 1.6 | 0.0249 | 453.8% |
| 39 | 102 | −0.99 | 1.244 | 79 | 3.2 | 1.39 | 4.2 | 1.871 | 0.0261 | 446.8% |
| 52 | 100 | −1.36 | 1.246 | 81 | 2.67 | 1.374 | 4.03 | 1.861 | 0.0315 | 310.6% |
| 65 | 102 | −0.83 | 1.4 | 81 | 0.49 | 1.557 | 1.32 | 2.099 | 0.53 | 163.3% |
| 78 | 70 | −2.25 | 1.344 | 64 | 0.77 | 1.452 | 3.02 | 1.983 | 0.1301 | 136.4% |

The study showed that pridopidine 45 mg bid provided improvement in a validated measure of cognitive function, the Stroop word reading (SWR) test up to 78 weeks, in HD patients (per protocol group). In patients not taking neuroleptics and chorea the effect was most pronounced and reached statistical significance at weeks 26, 39 and 53. Pridopidine shows beneficial pronounced and statistically significant improvement on SWR compared to placebo in HD participants not taking neuroleptics and/or chorea meds up to 78 weeks. Pridopidine showed improvement in SWR at week 26 with 453.8% improvement (change vs placebo 3.62, p=0.0249), week 39 with 446.8% improvement (change vs placebo 4.2, p=0.0261), week 52 with 310.6% improvement (change vs placebo 4.03, p=0.0315) week 65 with 163% improvement (change vs placebo 1.32, p=0.53) and week 78 with 136.4% improvement (change vs placebo 3.02, p=0.1301). From the results presented Pridopidine shows beneficial pronounced and statistically significant Relative % improvement on SWR in patients not using neuroleptics and/or chorea medications anytime during the study.

### TMS Score:

The Total Motor Score (TMS) is a widely used clinical measure of motor function in HD patients. A higher score in the TMS indicates worsening. As shown in tables 168-170, In the overall study population (mITT), pridopidine showed less worsening in TMS compared to the placebo up to 65 weeks (FIG. 51 and table 168). Pridopidine shows less decline in TMS at week 26 (negative change indicates improvement) (change vs placebo −0.16), week 39 (change vs placebo −0.48), week 52 (change vs placebo −0.78) and week 65 (change vs placebo −0.12).

Furthermore, the beneficial effect of pridopidine on TMS was stronger in HD subjects who were not taking neuroleptics at any time during the study. Pridopidine shows less decline in TMS at week 26 (negative change indicates improvement) (change vs placebo −0.54), week 39 (change vs placebo −0.74), week 52 (change vs placebo −1.12) and week 65 (change vs placebo −0.57).

A strong beneficial effect of pridopidine on TMS was also observed in patients not taking neuroleptics and/or chorea medications (FIG. 53 and table 168). Pridopidine shows less decline in TMS at week 26 (negative change indicates improvement) (change vs placebo −0.2), week 39 (change vs placebo −0.93), week 52 (change vs placebo −0.34) and week 65 (change vs placebo −0.38).

TABLE 168
Change from baseline to Week 65 in TMS score Full analysis set (mITT)

| Week 26 | | | Week 39 | | | Week 52 | | | Week 65 | | |
|---|---|---|---|---|---|---|---|---|---|---|---|
| | | Pri- | | | Pri- | | | Pri- | | | Pri- |
| | | dopidine | | | dopidine | | | dopidine | | | dopidine |
| | | 45 mg | | | 45 mg | | | 45 mg | | | 45 mg |
| | Placebo | bid | | Placebo | bid | | Placebo | bid | | Placebo | bid |
| n | 247 (0) | 243 (0) | n | 247 (0) | 243 (0) | n | 247 (0) | 243 (0) | n | 247 (0) | 243 (0) |
| Baseline | 32.9 | 33.8 | Baseline | 32.9 | 33.8 | Baseline | 32.9 | 33.8 | Baseline | 32.9 | 33.8 |
| mean | (10.95) | (11.20) | mean | (10.95) | (11.20) | mean | (10.95) | (11.20) | mean | (10.95) | (11.20) |
| (SD) | | | (SD) | | | (SD) | | | (SD) | | |
| n | 246 (1) | 242 (1) | n | 236 (11) | 230 (13) | n | 231 (16) | 223 (20) | n | 232 (15) | 224 (19) |
| (missing) | | | (missing) | | | (missing) | | | (missing) | | |
| Mean | 0.6 | 0.4 | Mean | 1.9 | 1.2 | Mean | 3.0 | 2.0 | Mean | 2.7 | 2.3 |
| (SD) | (6.53) | (7.20) | (SD) | (7.52) | (8.39) | (SD) | (8.30) | (8.48) | (SD) | (9.37) | (9.09) |
| Median | 0 | 0 | Median | 2 | 0.5 | Median | 3 | 1 | Median | 3 | 1 |
| Q1, Q3 | −3.0, | −3.0, | Q1, Q3 | −3.0, | −4.0, | Q1, Q3 | −2.0, | −4.0, | Q1, Q3 | 3.0, | −3.0, |
| | 5.0 | 4.0 | | 6.0 | 7.0 | | 8.0 | 7.0 | | 8.0 | 7.5 |
| Min, Max | −20, 26 | −20, 24 | Min, Max | −18, 28 | −40, 29 | Min, Max | −22, 29 | −25, 25 | Min, Max | −44, 34 | −23, 35 |
| LS Mean | 0.61 | 0.45 | LS Mean | 1.87 | 1.39 | LS Mean | 2.93 | 2.15 | LS Mean | 2.63 | 2.51 |
| (SE) | (0.454) | (0.454) | (SE) | (0.538) | (0.540) | (SE) | (0.561) | (0.565) | (SE) | (0.613) | (0.619) |
| 95% CI | −0.28, | −0.44, | 95% CI | 0.81, | 0.33, | 95% CI | 1.83 | 1.04, | 95% CI | 1.43, | 1.29, |
| | 1.50 | 1.34 | | 2.93 | 2.45 | | 4.03 | 3.26 | | 3.84 | 3.72 |
| LS Mean | | −0.16 | LS Mean | | −0.48 | LS Mean | | −0.78 | LS Mean | | −0.12 |
| Difference | | (0.607) | Difference | | (0.734) | Difference | | (0.768) | Difference | | (0.846) |
| (SE) vs | | | (SE) vs | | | (SE) vs | | | (SE) vs | | |
| Placebo | | | Placebo | | | Placebo | | | Placebo | | |
| 95% CI vs | | −1.35, | 95% CI vs | | −1.92, | 95% CI vs | | −2.29, | 95% CI vs | | −1.79, |
| Placebo | | 1.03 | Placebo | | 0.96 | Placebo | | 0.73 | Placebo | | 1.54 |
| p-value vs | | 0.7923 | p-value vs | | 0.5154 | p-value vs | | 0.3124 | p-value vs | | 0.8839 |
| Placebo | | | Placebo | | | Placebo | | | Placebo | | |

TABLE 169
Change from baseline to Weeks 26, 39, 52 and 65 in TMS score in participants not
taking neuroleptic medications anytime during the study.

| Week 26 | | | Week 39 | | | Week 52 | | | Week 65 | | |
|---|---|---|---|---|---|---|---|---|---|---|---|
| | | Pri- | | | Pri- | | | Pri- | | | Pri- |
| | | dopidine | | | dopidine | | | dopidine | | | dopidine |
| | | 45 mg | | | 45 mg | | | 45 mg | | | 45 mg |
| | Placebo | bid | | Placebo | bid | | Placebo | bid | | Placebo | bid |
| n | 141 (0) | 137 (0) | n | 141 (0) | 137 (0) | n | 141 (0) | 137 (0) | n | 141 (0) | 137 (0) |
| Baseline | 31.0 | 32.2 | Baseline | 31.0 | 32.2 | Baseline | 31.0 | 32.2 | Baseline | 31.0 | 32.2 |
| mean | (10.25) | (10.40) | mean | (10.25) | (10.40) | mean | (10.25) | (10.40) | mean | (10.25) | (10.40) |
| (SD) | | | (SD) | | | (SD) | | | (SD) | | |
| n | 141 (0) | 136 (1) | n | 135 (6) | 129 (8) | n | 132 (9) | 127 (10) | n | 133 (8) | 126 (11) |
| (missing) | | | (missing) | | | (missing) | | | (missing) | | |
| Mean | 0.4 | −0.3 | Mean | 1.4 | 0.5 | Mean | 1.8 | 0.5 | Mean | 1.7 | 0.9 |
| (SD) | (5.71) | (6.61) | (SD) | (7.55) | (8.66) | (SD) | (7.96) | (7.81) | (SD) | (8.55) | (8.43) |
| Median | 0 | 0 | Median | 1 | 0 | Median | 1 | 0 | Median | 2 | 0 |
| Q1, Q3 | −3.0, | −3.5, | Q1, Q3 | −4.0, | −5.0, | Q1, Q3 | −2.0, | −5.0, 6.0 | Q1, Q3 | −4.0, 7.0 | −4.0, 6.0 |
| | 4.0 | 4.0 | | 5.0 | 6.0 | | 6.0 | | | | |
| Min, Max | −20, 16 | −20, 18 | Min, Max | −16, 28 | −40, 25 | Min, Max | −22, 29 | −25, 22 | Min, Max | −18 26 | −23, 24 |
| LS Mean | 0.50 | −0.04 | LS Mean | 1.50 | 0.76 | LS Mean | 1.86 | 0.74 | LS Mean | 1.75 | 1.18 |
| (SE) | (1.336) | (1.387) | (SE) | (1.417) | (1.474) | (SE) | (1.404) | (1.463) | (SE) | (1.428) | (1.485) |
| 95% CI | −2.14, | −2.77, | 95% CI | −1.29, | −2.14, | 95% CI | −0.90, | −2.13, | 95% CI | −1.06, | −1.74, |
| | 3.13 | 2.69 | | 4.29 | 3.66 | | 4.62 | 3.62 | | 4.56 | 4.10 |
| LS Mean | | −0.54 | LS Mean | | −0.74 | LS Mean | | −1.12 | LS Mean | | −0.57 |
| Difference | | (0.734) | Difference | | (0.999) | Difference | | (0.962) | Difference | | (1.031) |
| (SE) vs | | | (SE) vs | | | (SE) vs | | | (SE) vs | | |
| Placebo | | | Placebo | | | Placebo | | | Placebo | | |
| 95% CI vs | | −1.98, | 95% CI vs | | −2.71, | 95% CI vs | | −3.01, | 95% CI vs | | −2.60, |
| Placebo | | 0.91 | Placebo | | 1.22 | Placebo | | 0.78 | Placebo | | 1.46 |
| p-value vs | | 0.4651 | p-value vs | | 0.4577 | p-value vs | | 0.2461 | p-value vs | | 0.5831 |
| Placebo | | | Placebo | | | Placebo | | | Placebo | | |

TABLE 170
Change from baseline to Weeks 26, 39, 52 and 65 in TMS score in participants not
taking neuroleptic and chorea medications anytime during the study.

| Week 26 | | | Week 39 | | | Week 52 | | | Week 65 | | |
|---|---|---|---|---|---|---|---|---|---|---|---|
| | | Pri- | | | Pri- | | | Pri- | | | Pri- |
| | | dopidine | | | dopidine | | | dopidine | | | dopidine |
| | | 45 mg | | | 45 mg | | | 45 mg | | | 45 mg |
| | Placebo | bid | | Placebo | bid | | Placebo | bid | | Placebo | bid |
| n | 112 (0) | 97 (0) | n | 112 (0) | 97 (0) | n | 112 (0) | 97 (0) | n | 112 (0) | 97 (0) |
| Baseline | 30.3 | 31.4 | Baseline | 30.3 | 31.4 | Baseline | 30.3 | 31.4 | Baseline | 30.3 | 31.4 |
| mean | (9.64) | (11.16) | mean | (9.64) | (11.16) | mean | (9.64) | (11.16) | mean | (9.64) | (11.16) |
| (SD) | | | (SD) | | | (SD) | | | (SD) | | |
| n | 112 (0) | 96 (1) | n | 107 (5) | 92 (5) | n | 104 (8) | 92 (5) | n | 106 (6) | 91 (6) |
| (missing) | | | (missing) | | | (missing) | | | (missing) | | |
| Mean | 0.2 | −0.3 | Mean | 1.2 | 0.0 | Mean | 1.1 | 0.3 | Mean | 1.6 | 0.7 |
| (SD) | (5.57) | (5.86) | (SD) | (7.17) | (8.20) | (SD) | (8.03) | (7.24) | (SD) | (8.77) | (8.06) |
| Median | 0 | 0 | Median | 2 | 0 | Median | 1 | 0 | Median | 2 | 0 |
| Q1, Q3 | −3.5, | −3.0, 3.5 | Q1, Q3 | −4.0, | −5.0, 5.0 | Q1, Q3 | −3.0, | −5.0, 6.0 | Q1, Q3 | −5.0, | −5.0, |
| | 3.0 | | | 5.0 | | | 6.0 | | | 7.0 | 6.0 |
| Min, Max | −10, 15 | −17, 16 | Min, Max | −16, 17 | −40, 20 | Min, Max | −22, 20 | −25, 22 | Min, Max | −17, 26 | −23, 24 |
| LS Mean | −0.04 | −0.23 | LS Mean | 0.96 | 0.04 | LS Mean | 0.79 | 0.45 | LS Mean | 1.28 | 0.90 |
| (SE) | (0.533) | (0.571) | (SE) | (0.731) | (0.783) | (SE) | (0.721) | (0.767) | (SE) | (0.792) | (0.849) |
| 95% CI | −1.09, | −1.36, | 95% CI | −0.48, | −1.51, | 95% CI | −0.64, | −1.06, | 95% CI | −0.29, | −0.78, |
| | 1.02 | 0.89 | | 2.41 | 1.58 | | 2.21 | 1.96 | | 2.84 | 2.57 |
| LS Mean | | −0.20 | LS Mean | | −0.93 | LS Mean | | −0.34 | LS Mean | | −0.38 |
| Difference | | (0.781) | Difference | | (1.072) | Difference | | (1.053) | Difference | | (1.161) |
| (SE) vs | | | (SE) vs | | | (SE) vs | | | (SE) vs | | |
| Placebo | | | Placebo | | | Placebo | | | Placebo | | |
| 95% CI vs | | −1.74, | 95% CI vs | | −3.04, | 95% CI vs | | −2.41, | 95% CI vs | | −2.67, |
| Placebo | | 1.34 | Placebo | | 1.19 | Placebo | | 1.74 | Placebo | | 1.91 |
| p-value vs | | 0.7992 | p-value vs | | 0.3883 | p-value vs | | 0.7491 | p-value vs | | 0.7466 |
| Placebo | | | Placebo | | | Placebo | | | Placebo | | |

A strong beneficial effect of pridopidine on TMS was observed compared to placebo in HD patients without antidopaminergic drugs. Pridopidine showed less decline in TMS at week 26 (negative change indicates improvement) (change vs placebo −0.2), week 39 (change vs placebo −0.93), week 52 (change vs placebo −0.34) and week 65 (change vs placebo −0.38). From the results presented Pridopidine 45 mg bid provides

Less decline in motor function as measured by the widely used and accepted scale—the Total Motor Score (TMS) up to 65 weeks compared to placebo.

### Quantitative Q-Motor Measures:

Improvement in objective measures of motor functions were observed using the quantitative motor score test (Q-Motor). Tables 171-191 below provide data corresponding to FIG. 54-FIG. 74.

In the overall study population (mITT), pridopidine treatment showed improvements in all Q-Motor measures compared to placebo at all time points up to 65 weeks. The effect of pridopidine was stronger and most pronounced in HD subjects who were not taking neuroleptic medication and in patients not taking neuroleptics and/or chorea medications (negative scores indicate improvement).

The following Q-Motor measurements are presented:
1) Finger Tap Inter Onset Interval (101) mean (FIGS. 54-56 and Tables 171-173)
2) Finger tap 101 SD (FIGS. 57-59 and Tables 174-176)
3) Finger tap Inter Tap Interval (ITI) SD (FIG. 60 and Tables 177-179)
4) Pronation Supination 101 Mean (FIGS. 61-63 and Tables 180-182)
5) Pronation Supination 101 SD (FIGS. 64-66 and Tables 183-185)
6) Pronation Supination ITI Mean (FIGS. 67-69 and Tables 186-188)
7) Pronation Supination ITI SD (FIGS. 70-72 and Tables 189-191)

TABLE 171
Change from baseline to Weeks 26, 52 and 65 in Q-Motor Finger tap inter onset
interval (IOI)-Mean in all patients (mITT)

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 247 | 243 | n | 247 | 243 | n | 247 | 243 |
| Baseline | 353.3 | 353.2 | Baseline | 353.3 | 353.2 | Baseline | 353.3 | 353.2 |
| mean (SD) | (137.78) | (129.72) | mean (SD) | (137.78) | (129.72) | mean (SD) | (137.78) | (129.72) |
| n (missing) | 245 (2) | 239 (4) | n (missing) | 227 (20) | 218 (25) | n (missing) | 228 (19) | 219 (24) |
| Mean (SD) | 2.8 | −5.3 | Mean (SD) | 9.5 | 4.2 | Mean (SD) | 15.6 | 7.8 |
| | (84.08 | (89.92) | | (89.37 | (78.57) | | (91.13) | (81.26) |
| Median | 5.9 | −2.7 | Median | 11 | 7.5 | Median | 1. | 6.5 |
| Q1, Q3 | −16.2, | −36.7, | Q1, Q3 | −14.2, | −28.1, | Q1, Q3 | −15.5, | −27.6, |
| | 34.0 | 26.0 | | 44.4 | 37.7 | | 55.1 | 35.9 |
| Min, Max | −709, | −669, | Min, Max | −804, | −385, | Min, Max | −670, | −363, |
| | 226 | 455 | | 256 | 479 | | 290 | 373 |
| LS Mean | 1.64 | −6.39 | LS Mean | 7.63 | 4.91 | LS Mean | 14.56 | 8.00 |
| (SE) | (5.316) | (5.331) | (SE) | (5.493) | (5.534) | (SE) | (5.718) | (5.767) |
| 95% CI | −8.81, | −16.87, | 95% CI | −3.16, | −5.96, | 95% CI | 3.32, | 3.33, |
| | 12.08 | 4.08 | | 18.43 | 15.79 | | 25.80 | 19.33 |
| LS Mean | | −8.03 | LS Mean | | −2.72 | LS Mean | | −6.56 |
| Difference | | (7.135) | Difference | | (7.421) | Difference | | (7.756) |
| (SE) vs | | | (SE) vs | | | (SE) vs | | |
| Placebo | | | Placebo | | | Placebo | | |
| 95% CI | | −22.05, | 95% CI | | −17.31, | 95% CI | | −21.80, |
| vs Placebo | | 5.99 | vs Placebo | | 11.87 | vs Placebo | | 8.69 |
| p-value | | 0.2609 | p-value | | 0.7142 | p-value | | 0.3982 |
| vs Placebo | | | vs Placebo | | | vs Placebo | | |

TABLE 172
Change from baseline to Weeks 26, 52 and 65 in Q-Motor Finger tap inter onset interval
(IOI)-mean score in participants not taking neuroleptic medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| Baseline n | 141 | 136 | Baseline n | 141 | 136 | Baseline n | 141 | 136 |
| n (missing) | 141 | 134 | n (missing) | 131 | 125 | n (missing) | 131 | 125 |
| LS Mean | −0.2374 | −13.5095 | LS Mean | 5.4102 | −1.0423 | LS Mean | 17.3831 | 0.5041 |
| (SE) | (5.8711) | (6.0059) | (SE) | (6.2578) | (6.3914) | (SE) | (6.4555) | (6.5942) |
| 95% CI | −11.796, | −25.334, | 95% CI | −6.9115, | −13.627, | 95% CI | 4.6712, | −12.481, |
| | 11.3217 | −1.6851 | | 17.732 | −11.5426 | | 30.095 | 13.4895 |
| LS Mean | | −13.2721 | LS Mean | | −6.4525 | LS Mean | | −16.879 |
| Difference | | (8.4096) | Difference | | (8.9583) | Difference | | (9.243) |
| (SE) vs | | | (SE) vs | | | (SE) vs | | |
| Placebo | | | Placebo | | | Placebo | | |
| 95% CI vs | | −29.829, | 95% CI vs | | −24.091, | 95% CI vs | | −35.08, |
| Placebo | | 3.2844 | Placebo | | 11.1864 | Placebo | | 1.3217 |
| p-value vs | | 0.1157 | p-value vs | | 0.472 | p-value vs | | 0.069 |
| Placebo | | | Placebo | | | Placebo | | |

TABLE 173 A
Change from baseline to Weeks 26, 52 and 65 in Q-Motor Finger tap inter onset interval
(IOI)-mean score in participants not taking neuroleptic and chorea medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 112 (0) | 97 (0) | n | 112 (0) | 97 (0) | n | 112 (0) | 97 (0) |
| Baseline | 329.5 | 353.1 | Baseline | 329.5 | 353.1 | Baseline | 329.5 | 353.1 |
| mean (SD) | (95.29) | (111.84) | mean (SD) | (95.29) | (111.84) | mean (SD) | (95.29) | (111.84) |
| n | 112 (0) | 95 (2) | n | 104 (8) | 91 (6) | n | 104 (8) | 91 (6) |
| (missing) | | | (missing) | | | (missing) | | |
| Mean (SD) | 13.7 | −15.9 | Mean | 22.0 | −4.5 | Mean | 28.6 | −0.9 |
| | (49.61) | (58.07) | (SD) | (55.41) | (83.42) | (SD) | (60.42) | (78.18) |
| Median | 12.1 | −13.6 | Median | 13.5 | −3.8 | Median | 19.8 | 4.8 |
| Q1, Q3 | −7.9, 39.8 | −44.1, 19.1 | Q1, Q3 | −8.6, 52.5 | −42.2, 20.6 | Q1, Q3 | −1.9, 63.7 | −42.4, 31.8 |
| Min, Max | −166, 175 | −257, 117 | Min, Max | −145, 247 | −252, 479 | Min, Max | −143, 239 | −286, 276 |
| LS Mean | 11.17 | −14.66 | LS Mean | 17.45 | −2.11 | LS Mean | 24.77 | 2.58 |
| (SE) | (5.032) | (5.408) | (SE) | (6.688) | (7.127) | (SE) | (6.684) | (7.129) |
| 95% CI | 1.24, | −25.32, | 95% CI | 4.27, | −16.16, | 95% CI | 11.59, | −11.48, |
| | 21.09 | −3.99 | | 30.64 | 11.95 | | 37.96 | 16.64 |
| LS Mean | | −25.82 | LS Mean | | −19.56 | LS Mean | | −22.19 |
| Difference | | (7.411) | Difference | | (9.789) | Difference | | (9.789) |
| (SE) vs | | | (SE) vs | | | (SE) vs | | |
| Placebo | | | Placebo | | | Placebo | | |
| 95% CI vs | | −40.43, | 95% CI vs | | −38.87, | 95% CI vs | | −41.50, |
| Placebo | | −11.21 | Placebo | | −0.26 | Placebo | | −2.89 |
| p-value vs | | 0.0006 | p-value vs | | 0.0471 | p-value vs | | 0.0244 |
| Placebo | | | Placebo | | | Placebo | | |

TABLE 173B
Change from baseline to Weeks 26, 52, 65 and 78 in Q-Motor Finger tap inter
onset interval (IOI)-mean score Per protocol population in participants not taking
neuroleptic and chorea medications anytime during the study.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | 12.29 | 5.303 | 80 | −20.03 | 5.848 | −32.32 | 7.93 | <0.001 | 251.4% |
| 52 | 100 | 17.56 | 7.053 | 81 | −4.99 | 7.82 | −22.55 | 10.56 | 0.0341 | 126.4% |
| 65 | 102 | 25.26 | 6.944 | 81 | 1.79 | 7.667 | −23.47 | 10.372 | 0.0248 | 93.4% |
| 78 | 70 | 27.53 | 7.329 | 64 | 2.67 | 7.871 | −24.86 | 10.782 | 0.0223 | 90.9% |

A strong beneficial effect of pridopidine on Q-Motor was observed in patients not taking neuroleptics and/or chorea medications. Pridopidine treatment showed improvement in Q-Motor at week 26 (negative change indicates improvement) (change vs placebo −32.32, p<0.001) with improvement of 251.4% compared to placebo, week 52 (change vs placebo −22.55, p=0.0341) with improvement of 126.4% compared to placebo, in week 65 progression slowed by 93.4% (change vs placebo −23.47, p=0.0248) and in week 78 progression slowed by 90.9% (change vs placebo −24.86, p=0.0223). From the results presented Pridopidine 45 mg bid provides Improvement in motor function as measured by the widely used and accepted scale—the Q-Motor up to 78 weeks compared to placebo.

TABLE 174
Change from baseline to Weeks 26, 52 and 65 in Q-Motor Finger tap inter onset
interval (IOI)-SD score Full analysis set (mITT)

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 247 (0) | 243 (0) | n | 247 (0) | 243 (0) | n | 247 (0) | 243 (0) |
| Baseline | 121.6 | 129.5 | Baseline | 121.6 | 129.5 | Baseline | 121.6 | 129.5 |
| mean (SD) | (77.85) | (79.34) | mean (SD) | (77.85) | (79.34) | mean (SD) | (77.85) | (79.34) |
| n (missing) | 245 (2) | 239 (4) | n (missing) | 227 (20) | 218 (25) | n (missing) | 228 (19) | 219 (24) |
| Mean (SD) | 7.2 | −1.2 | Mean (SD) | 8.5 | 4.5 | Mean (SD) | 12.6 | 5.0 |
| | (44.54) | (59.58) | | (44.62) | (53.73) | | (54.97) | (62.27) |
| Median | 4.8 | −3.6 | Median | 7.7 | 1.2 | Median | 8.3 | 3.2 |
| Q1, Q3 | −12.7, | −23.0, | Q1, Q3 | −9.4, | −19.5, | Q1, Q3 | −11.4, | −20.6, 29.4 |
| | 26.7 | 17.9 | | 32.2 | 28.2 | | 34.7 | |
| Min, Max | −177, | −267, | Min, Max | −167, | −295, | Min, Max | −191, | −374, 368 |
| | 171 | 497 | | 135 | 173 | | 250 | |
| LS Mean | 6.61 | −0.15 | LS Mean | 8.35 | 8.33 | LS Mean | 12.74 | 8.26 |
| (SE) | (3.362) | (3.369) | (SE) | (3.405) | (3.427) | (SE) | (4.077) | (4.118) |
| 95% CI | 0.00, | −6.77, | 95% CI | 1.65, | 1.60, | 95% CI | 4.72, | 0.17, |
| | 13.21 | 6.47 | | 15.04 | 15.07 | | 20.75 | 16.35 |
| LS Mean | | −6.75 | LS Mean | | −0.02 | LS Mean | | −4.48 |
| Difference | | (4.509) | Difference | | (4.588) | Difference | | (5.591) |
| (SE) vs | | | (SE) vs | | | (SE) vs | | |
| Placebo | | | Placebo | | | Placebo | | |
| 95% CI vs | | −15.61, | 95% CI vs | | −9.04, | 95% CI vs | | −15.47, |
| Placebo | | 2.11 | Placebo | | 9.00 | Placebo | | 6.51 |
| p-value vs | | 0.1348 | p-value vs | | 0.9973 | p-value vs | | 0.4235 |
| Placebo | | | Placebo | | | Placebo | | |

TABLE 175
Change from baseline to Weeks 26, 52 and 65 in Q-Motor Finger tap inter onset interval
(IOI)-SD score in participants not taking neuroleptic medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 141 | 136 | n | 141 | 136 | n | 141 | 136 |
| n (missing) | 141 | 134 | n (missing) | 131 | 125 | n (missing) | 131 | 125 |
| LS | 7.3683 | −6.564 | LS | 7.8831 | 1.7014 | LS | 16.0972 | 0.6526 |
| Mean (SE) | (3.5546) | (3.6407) | Mean (SE) | (3.7028) | (3.7847) | Mean (SE) | (4.7178) | (4.8249) |
| 95% CI | 0.3703, | −13.732, | 95% CI | 0.5918, | −5.7513, | 95% CI | 6.8072, | −8.8484, |
| | 14.3663 | 0.6035 | | 15.1745 | 9.1541 | | 25.3873 | 10.1536 |
| LS Mean | | −13.9323 | LS Mean | | −6.1818 | LS Mean | | −15.4446 |
| Difference | | (5.0977) | Difference | | (5.3059) | Difference | | (6.7567) |
| (SE) vs | | | (SE) vs | | | (SE) vs | | |
| Placebo | | | Placebo | | | Placebo | | |
| 95% CI vs | | −23.968, | 95% CI vs | | −16.63, | 95% CI vs | | −28.749, |
| Placebo | | −3.8966 | Placebo | | 4.2663 | Placebo | | −2.1399 |
| p-value vs | | 0.0067 | p−value vs | | 0.2451 | p−value vs | | 0.0231 |
| Placebo | | | Placebo | | | Placebo | | |

TABLE 176 A
Change from baseline to Weeks 26, 52 and 65 in Q-Motor Finger tap inter onset interval
(IOI)-SD score in participants not taking neuroleptic and chorea medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 112 (0) | 97 (0) | n | 112 (0) | 97 (0) | n | 112 (0) | 97 (0) |
| Baseline | 109.0 | 127.9 | Baseline | 109.0 | 127.9 | Baseline | 109.0 | 127.9 |
| mean (SD) | (69.00) | (82.14) | mean (SD) | (69.00) | (82.14) | mean (SD) | (69.00) | (82.14) |
| n (missing) | 112 (0) | 95 (2) | n (missing) | 104 (8) | 91 (6) | n (missing) | 104 (8) | 91 (6) |
| Mean (SD) | 10.1 | −9.7 | Mean (SD) | 11.7 | −1.6 | Mean (SD) | 16.3 | −1.7 |
| | (38.89) | (46.09) | | (37.43) | (56.71) | | (44.56) | (62.06) |
| Median | 5.2 | −7.6 | Median | 9.9 | 1 | Median | 11 | 1.7 |
| Q1, Q3 | 10.2, | −27.4, 10.9 | Q1, Q3 | −4.8, | −25.8, 25.6 | Q1, Q3 | −5.4, | −27.8, 27.8 |
| | 26.0 | | | 33.3 | | | 38.2 | |
| Min, Max | −88, | −267, 131 | Min, Max | −102, | −295, 160 | Min, Max | −86, | −374, 187 |
| | 142 | | | 130 | | | 230 | |
| LS Mean | 8.15 | −7.95 | LS Mean | 9.53 | 0.78 | LS Mean | 14.26 | 0.93 |
| (SE) | (3.919) | (4.218) | (SE) | (4.318) | (4.604) | (SE) | (5.009) | (5.344) |
| 95% CI | 0.42, | −16.26, | 95% CI | 1.01, | −8.30, 9.85 | 95% CI | 4.38, | −9.61, |
| | 15.87 | 0.37 | | 18.04 | | | 24.14 | 11.47 |
| LS Mean | | −16.09 | LS Mean | | −8.75 | LS Mean | | −13.33 |
| Difference | | (5.777) | Difference | | (6.326) | Difference | | (7.338) |
| (SE )vs | | | (SE )vs | | | (SE )vs | | |
| Placebo | | | Placebo | | | Placebo | | |
| 95% CI vs | | −27.48, | 95% CI vs | | −21.23, | 95% CI vs | | −27.80, |
| Placebo | | −4.70 | Placebo | | 3.72 | Placebo | | 1.14 |
| p-value vs | | 0.0058 | p-value vs | | 0.168 | p-value vs | | 0.0707 |
| Placebo | | | Placebo | | | Placebo | | |

TABLE 176 B
Change from baseline to Weeks 26, 52, 65 and 78 in Q-Motor Finger tap inter
onset interval (IOI)-SD score in participants not taking neuroleptic and chorea medications
anytime during the study.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 100 | 13.65 | 5.198 | 81 | 0.69 | 5.737 | −12.96 | 7.756 | 0.0964 | 95.5% |
| 52 | 102 | 7.9 | 4.179 | 81 | −11.16 | 4.616 | −19.07 | 6.245 | 0.0026 | 223.7% |
| 65 | 100 | 9.23 | 4.611 | 80 | 0.38 | 5.099 | −8.84 | 6.892 | 0.201 | 96.3% |
| 78 | 70 | 16.93 | 5.492 | 64 | 5.08 | 5.918 | −11.86 | 8.09 | 0.1447 | 73.8% |

A pronounced advantageous impact of pridopidine on Q-Motor was observed in patients not taking neuroleptics and/or chorea medications. Pridopidine treatment showed improvement in Q-Motor at week 26 (negative change indicates improvement) (change vs placebo −12.96, v 0.0964) in week 26 progression slowed by 95.5% compared to placebo. week 52 (change vs placebo −19.07, v0.0026) with improvement of 223.7% compared to placebo, in week 65 progression slowed by 96.3% compared to placebo (change vs placebo −8.84, p=0.201) and in week 78 progression slowed by 73.8% compared to placebo (change vs placebo −11.86, p=0.1447). From the results presented Pridopidine 45 mg bid provides Improvement in motor function as measured by the widely used and accepted scale—the Q-Motor up to 78 weeks compared to placebo.

TABLE 177
Change from baseline to Weeks 26, 52 and 65 in Q-Motor Finger tap inter tap interval
(ITI)-SD score Full analysis set (mITT)

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 247 (0) | 242 (1) | n | 247 (0) | 242 (1) | n | 247 (0) | 242 (1) |
| Baseline | 124.9 | 138.5 | Baseline | 124.9 | 138.5 | Baseline | 124.9 | 138.5 |
| mean (SD) | (79.43) | (86.83) | mean (SD) | (79.43) | (86.83) | mean (SD) | (79.43) | (86.83) |
| n | 245 (2) | 238 (5) | n | 225 (22) | 218 (25) | n | 228 (19) | 219 (24) |
| (missing) | | | (missing) | | | (missing) | | |
| Mean | 17.2 | 9.1 | Mean | 26.6 | 14.3 | Mean | 26.6 | 15.7 |
| (SD) | (62.95) | (58.37) | (SD) | (61.38) | (60.70) | (SD) | (54.42) | (65.56) |
| Median | 8.9 | 3 | Median | 16.5 | 11.7 | Median | 19.6 | 9 |
| Q1, Q3 | −8.5, | −16.8, | Q1, Q3 | −3.6, | −10.7, | Q1, Q3 | −4.3, | −13.9, |
| | 33.6 | 28.8 | | 48.1 | 37.4 | | 49.7 | 39.8 |
| Min, Max | −121, 573 | −211, 364 | Min, Max | −238, 440 | −362, 310 | Min, Max | −115, 284 | −284, 290 |
| LS Mean | 15.23 | 7.74 | LS Mean | 24.45 | 14.13 | LS Mean | 25.01 | 16.05 |
| (SE) | (4.063) | (4.092) | (SE) | (4.246) | (4.284) | (SE) | (4.222) | (4.270) |
| 95% CI | 7.24, | −0.30, | 95% CI | 16.11, | 5.71, | 95% CI | 16.71, | 7.66, |
| | 23.21 | 15.78 | | 32.80 | 22.55 | | 33.30 | 24.44 |
| LS Mean | | −7.48 | LS Mean | | −10.32 | LS Mean | | −8.95 |
| Difference | | (5.496) | Difference | | (5.775) | Difference | | (5.747) |
| (SE) vs | | | (SE) vs | | | (SE) vs | | |
| Placebo | | | Placebo | | | Placebo | | |
| 95% CI vs | | −18.28, | 95% CI vs | | −21.67, | 95% CI vs | | −20.25, |
| Placebo | | 3.32 | Placebo | | 1.03 | Placebo | | 2.34 |
| p-value vs | | 0.174 | p-value vs | | 0.0746 | p-value vs | | 0.12 |
| Placebo | | | Placebo | | | Placebo | | |

TABLE 178
Change from baseline to Weeks 26, 52 and 65 in Q-Motor Finger tap inter tap interval
(ITI)-SD score in participants not taking neuroleptic medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 141 | 136 | n | 141 | 136 | n | 141 | 136 |
| n | 141 | 134 | n | 131 | 125 | n | 131 | 125 |
| (missing) | | | (missing) | | | (missing) | | |
| LS Mean | 2.6851 | −7.3255 | LS Mean | 6.7411 | 1.4397 | LS Mean | 12.9728 | 1.5786 |
| (SE) | (2.7744) | (2.8403) | (SE) | (3.1596) | (3.2295) | (SE) | (4.1022) | (4.1953) |
| 95% CI | −2.777, | −12.917, | 95% CI | 0.5192, | −4.92, | 95% CI | 4.8949, | −6.6826, |
| | 8.1472 | −1.7337 | | 12.9631 | 7.7994 | | 21.0506 | 9.8397 |
| LS Mean | | −10.0106 | LS Mean | | −5.3014 | LS Mean | | −11.3942 |
| Difference | | (3.9822) | Difference | | (4.5295) | Difference | | (5.8752) |
| (SE) vs | | | (SE) vs | | | (SE) vs | | |
| Placebo | | | Placebo | | | Placebo | | |
| 95% CI vs | | −17.85, | 95% CI vs | | −14.221, | 95% CI vs | | −22.963, |
| Placebo | | −2.1708 | Placebo | | 3.618 | Placebo | | 0.1746 |
| p-value vs | | 0.0125 | p-value vs | | 0.2429 | p-value vs | | 0.0535 |
| Placebo | | | Placebo | | | Placebo | | |

TABLE 179
Change from baseline to Weeks 26, 52 and 65 in Q-Motor Finger tap inter tap interval
(ITI)-SD score in participants not taking neuroleptic and chorea medications anytime during the study.

| Week 26 | | | Week 52 | | | Week 65 | | |
|---|---|---|---|---|---|---|---|---|
| | | Pridopidine | | | Pridopidine | | | Pridopidine |
| | | 45 mg | | | 45 mg | | | 45 mg |
| | Placebo | bid | | Placebo | bid | | Placebo | bid |
| n | 112 (0) | 97 (0) | n | 112 (0) | 97 (0) | n | 112 (0) | 97 (0) |
| Baseline | 110.5 | 122.7 | Baseline | 110.5 | 122.7 | Baseline | 110.5 | 122.7 |
| mean | (65.94) | (67.17) | mean | (65.94) | (67.17) | mean | (65.94) | (67.17) |
| (SD) | | | (SD) | | | (SD) | | |
| n | 112 (0) | 95 (2) | n | 103 (9) | 91 (6) | n | 104 (8) | 91 (6) |
| (missing) | | | (missing) | | | (missing) | | |
| Mean | 18.3 | 8.1 | Mean | 24.9 | 9.5 | Mean | 27.7 | 9.7 |
| (SD) | (70.19) | (49.93) | (SD) | (60.01) | (42.74) | (SD) | (51.58) | (45.69) |
| Median | 7.7 | −0.1 | Median | 15.6 | 9.5 | Median | 17.1 | 9.5 |
| Q1, Q3 | −6.9, | −18.1, | Q1, Q3 | −4.9, | −11.2, | Q1, Q3 | −4.3, | −16.0, |
| | 26.9 | 23.4 | | 39.1 | 29.7 | | 49.6 | 35.8 |
| Min, | −91, 573 | −85, 247 | Min, | −88, 440 | −143, 206 | Min, | −71, 247 | −153, 136 |
| Max | | | Max | | | Max | | |
| LS Mean | 17.86 | 7.18 | LS | 23.38 | 8.96 | LS | 27.22 | 10.81 |
| (SE) | (5.726) | (6.181) | Mean | (4.962) | (5.287) | Mean | (4.879) | (5.170) |
| | | | (SE) | | | (SE) | | |
| 95% CI | 6.57, | −5.00, | 95% CI | 13.60, | −1.46, | 95% CI | 17.60, | 0.61, |
| | 29.15 | 19.37 | | 33.17 | 19.39 | | 36.84 | 21.01 |
| LS Mean | | −10.67 | LS Mean | | −14.42 | LS Mean | | −16.41 |
| Difference | | (8.432) | Difference | | (7.256) | Difference | | (7.117) |
| (SE) vs | | | (SE) vs | | | (SE) vs | | |
| Placebo | | | Placebo | | | Placebo | | |
| 95% CI | | −27.30, | 95% CI | | −28.73, | 95% CI | | −30.45, |
| vs | | 5.95 | vs | | −0.11 | vs | | −2.37 |
| Placebo | | | Placebo | | | Placebo | | |
| p-value | | 0.207 | p-value | | 0.0482 | p-value | | 0.0222 |
| vs | | | vs | | | vs | | |
| Placebo | | | Placebo | | | Placebo | | |

TABLE 180
Change from baseline to Weeks 26, 52 and 65 in Q-Motor Pronation Supination inter
onset interval (IOI)-Mean Full analysis set (mITT)

| Week 26 | | | Week 52 | | | Week 65 | | |
|---|---|---|---|---|---|---|---|---|
| | | Pridopidine | | | Pridopidine | | | Pridopidine |
| | | 45 mg | | | 45 mg | | | 45 mg |
| | Placebo | bid | | Placebo | bid | | Placebo | bid |
| n | 247 (0) | 242 (1) | n | 247 (0) | 242 (1) | n | 247 (0) | 242 (1) |
| Baseline | 592.3 | 594.9 | Baseline | 592.3 | 594.9 | Baseline | 592.3 | 594.9 |
| mean | (192.91) | (169.31) | mean | (192.91) | (169.31) | mean | (192.91) | (169.31) |
| (SD) | | | (SD) | | | (SD) | | |
| n | 245 (2) | 238 (5) | n | 225 (22) | 218 (25) | n | 228 (19) | 219 (24) |
| (missing) | | | (missing) | | | (missing) | | |
| Mean | 9.5 | −3.0 | Mean | 19.6 | 13.5 | Mean | 18.1 | 18.2 |
| (SD) | (116.86) | (107.71) | (SD) | (128.74) | (107.16) | (SD) | (132.00) | (112.78) |
| Median | 9.1 | −2.2 | Median | 24.1 | 11.9 | Median | 22 | 16.4 |
| Q1, Q3 | −34.6, | −55.1, | Q1, Q3 | −42.1, | −43.2, | Q1, Q3 | −44.5, | −47.9, |
| | 48.2 | 47.5 | | 83.1 | 72.0 | | 94.6 | 72.3 |
| Min, | −537, 856 | −504, 541 | Min, | −527, 713 | −339, 496 | Min, | −663, 503 | −243, 439 |
| Max | | | Max | | | Max | | |
| LS | 8.39 | −3.25 | LS | 18.46 | 13.82 | LS | 17.46 | 19.73 |
| Mean | (7.467) | (7.503) | Mean | (7.902) | (7.959) | Mean | (8.255) | (8.333) |
| (SE) | | | (SE) | | | (SE) | | |
| 95% CI | −6.28, | −18.00, | 95% CI | 2.93, | −1.82, | 95% CI | 1.23, | 3.35, |
| | 23.06 | 11.49 | | 33.99 | 29.46 | | 33.68 | 36.10 |
| LS Mean | | −11.64 | LS Mean | | −4.64 | LS Mean | | 2.27 |
| Difference | | (10.005) | Difference | | (10.673) | Difference | | (11.208) |
| (SE) | | | (SE) | | | (SE) | | |
| vs | | | vs | | | vs | | |
| Placebo | | | Placebo | | | Placebo | | |
| 95% CI | | −31.30, | 95% CI | | −25.61, | 95% CI | | −19.75, |
| vs | | 8.02 | vs | | 16.33 | vs | | 24.30 |
| Placebo | | | Placebo | | | Placebo | | |
| p-value | | 0.2451 | p-value | | 0.6639 | p-value | | 0.8394 |
| vs | | | vs | | | vs | | |
| Placebo | | | Placebo | | | Placebo | | |

TABLE 181
Change from baseline to Weeks 26, 52 and 65 in Q-Motor Pronation Supination inter
onset interval (IOI)-Mean score in participants not taking neuroleptic medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 141 (0) | 136 (0) | n | 141 (0) | 136 (0) | n | 141 (0) | 136 (0) |
| Baseline | 563.1 | 593.1 | Baseline | 563.1 | 593.1 | Baseline | 563.1 | 593.1 |
| mean | (168.33) | (157.49) | mean | (168.33) | (157.49) | mean | (168.33) | (157.49) |
| (SD) | | | (SD) | | | (SD) | | |
| n | 141 (0) | 134 (2) | n | 130 (11) | 125 (11) | n | 131 (10) | 125 (11) |
| (missing) | | | (missing) | | | (missing) | | |
| Mean | 11.9 | −17.1 | Mean | 23.5 | 6.4 | Mean | 29.3 | 3.9 |
| (SD) | (103.90) | (81.50) | (SD) | (118.78) | (110.15) | (SD) | (114.04) | (102.77) |
| Median | 5.2 | −8.8 | Median | 21.3 | 5.2 | Median | 22 | 6.5 |
| Q1, Q3 | −34.6, | −61.8, 30.3 | Q1, Q3 | −41.6, | −54.3, 57.6 | Q1, Q3 | −42.8, | −66.6, 64.9 |
| | 37.4 | | | 69.9 | | | 97.3 | |
| Min, | −370, 856 | −226, 206 | Min, | −437, 713 | −339, 496 | Min, | −235, 503 | −230, 384 |
| Max | | | Max | | | Max | | |
| LS Mean | 9.46 | −16.84 | LS Mean | 20.02 | 7.25 | LS Mean | 25.90 | 7.19 |
| (SE) | (7.835) | (8.034) | (SE) | (9.616) | (9.828) | (SE) | (9.362) | (9.577) |
| 95% CI | −5.97, | −32.66, | 95% CI | 1.09, | −12.10, | 95% CI | 7.46, | −11.67, |
| | 24.88 | −1.02 | | 38.95 | 26.60 | | 44.33 | 26.05 |
| LS Mean | | −26.30 | LS Mean | | −12.77 | LS Mean | | −18.71 |
| Difference | | (11.241) | Difference | | (13.765) | Difference | | (13.412) |
| (SE) vs | | | (SE) vs | | | (SE) vs | | |
| Placebo | | | Placebo | | | Placebo | | |
| 95% CI vs | | −48.43, | 95% CI vs | | −39.87, | 95% CI vs | | −45.11, |
| Placebo | | −4.17 | Placebo | | 14.33 | Placebo | | 7.70 |
| p-value vs | | 0.02 | p-value vs | | 0.3544 | p-value vs | | 0.1643 |
| Placebo | | | Placebo | | | Placebo | | |

TABLE 182A
Change from baseline to Weeks 26, 52 and 65 in Q-Motor Pronation Supination inter onset interval (IOI)-
Mean score in participants not taking neuroleptic and chorea medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 112 (0) | 97 (0) | n | 112 (0) | 97 (0) | n | 112 (0) | 97 (0) |
| Baseline | 548.4 | 582.3 | Baseline | 548.4 | 582.3 | Baseline | 548.4 | 582.3 |
| mean (SD) | (151.15) | (157.61) | mean (SD) | (151.15) | (157.61) | mean (SD) | (151.15) | (157.61) |
| n | 112 (0) | 95 (2) | n | 103 (9) | 91 (6) | n | 104 (8) | 91 (6) |
| (missing) | | | (missing) | | | (missing) | | |
| Mean | 17.6 | −18.5 | Mean | 25.8 | 4.9 | Mean | 31.8 | 8.7 |
| (SD) | (103.74) | (81.13) | (SD) | (105.81) | (110.01) | (SD) | (101.62) | (111.16) |
| Median | 6.4 | −8.9 | Median | 23 | 2.7 | Median | 23 | 6.5 |
| Q1, Q3 | −33.5, | −61.2, | Q1, Q3 | −40.8, | −57.9, | Q1, Q3 | −38.3, | −66.6, |
| | 38.4 | 30.7 | | 69.9 | 58.1 | | 96.1 | 67.9 |
| Min, Max | −159, 856 | −226, 206 | Min, Max | −141, 713 | −339, 496 | Min, Max | −180, 492 | −230, 384 |
| LS Mean | 17.21 | −19.22 | LS Mean | 23.22 | 5.41 | LS Mean | 29.55 | 12.90 |
| (SE) | (8.928) | (9.601) | (SE) | (10.237) | (10.910) | (SE) | (10.432) | (11.113) |
| 95% CI | −0.39, | −38.15, | 95% CI | 3.04, | −16.11, | 95% CI | 8.98, | −9.02, |
| | 34.81 | −0.29 | | 43.41 | 26.92 | | 50.12 | 34.82 |
| LS Mean | | −36.43 | LS Mean | | −17.81 | LS Mean | | −16.65 |
| Difference | | (13.153) | Difference | | (14.993) | Difference | | (15.276) |
| (SE) vs | | | (SE) vs | | | (SE) vs | | |
| Placebo | | | Placebo | | | Placebo | | |
| 95% CI vs | | −62.37, | 95% CI vs | | −47.38, | 95% CI vs | | −46.77, |
| Placebo | | −10.50 | Placebo | | 11.75 | Placebo | | 13.48 |
| p-value vs | | 0.0061 | p-value vs | | 0.2362 | p-value vs | | 0.2772 |
| Placebo | | | Placebo | | | Placebo | | |

TABLE 182B
Change from baseline to Weeks 26, 52, 65 and 78 in Q-Motor Pronation
Supination inter onset interval (IOI)-Mean score in participants not taking neuroleptic and
chorea medications anytime during the study.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | 20.63 | 9.593 | 81 | −24.25 | 10.577 | −44.88 | 14.325 | 0.002 | 211.8% |
| 52 | 99 | 23.75 | 10.371 | 80 | −3.43 | 11.432 | −27.18 | 15.478 | 0.0807 | 113.7% |
| 65 | 100 | 31.15 | 10.75 | 81 | 9.08 | 11.833 | −22.07 | 16.028 | 0.1703 | 72.2% |
| 78 | 69 | 40.3 | 12.379 | 64 | 14.92 | 13.263 | −25.38 | 18.181 | 0.1646 | 64.7% |

Pridopidine demonstrated a significant and beneficial effect on Q-Motor was observed in patients not taking neuroleptics and/or chorea medications. Pridopidine treatment showed improvement in Q-Motor at week 26 with improvement of 211.8% compared to placebo (negative change indicates improvement) (change vs placebo −44.88, p=0.002), week 52 with improvement of 113.7% compared to placebo (change vs placebo −27.18, p=0.0807), in week 65 progression slowed by 72.2% compared to placebo (change vs placebo −22.07, p=0.1703) and in week 78 progression slowed by 64.7% compared to placebo (change vs placebo −25.38, p=0.1646). From the results presented Pridopidine 45 mg bid provides Improvement in motor function as measured by the widely used and accepted scale—the Q-Motor up to 78 weeks compared to placebo.

TABLE 183
Change from baseline to Weeks 26, 52 and 65 in Q-Motor Pronation Supination inter
onset interval (IOI)-SD-score Full analysis set (mITT)

| Week 26 | | | Week 52 | | | Week 65 | | |
|---|---|---|---|---|---|---|---|---|
| | | Pridopidine | | | Pridopidine | | | Pridopidine |
| | | 45 mg | | | 45 mg | | | 45 mg |
| | Placebo | bid | | Placebo | bid | | Placebo | bid |
| n | 247 (0) | 242 (1) | n | 247 (0) | 242 (1) | n | 247 (0) | 242 (1) |
| Baseline mean | 141.3 | 155.9 | Baseline mean | 141.3 | 155.9 | Baseline mean | 141.3 | 155.9 |
| (SD) | (94.18) | (97.76) | (SD) | (94.18) | (97.76) | (SD) | (94.18) | (97.76) |
| n (missing) | 245 (2) | 238 (5) | n (missing) | 225 (22) | 218 (25) | n (missing) | 228 (19) | 219 (24) |
| Mean (SD) | 20.5 | 13.1 | Mean (SD) | 31.7 | 18.8 | Mean (SD) | 31.1 | 21.2 |
| | (71.46) | (74.53) | | (70.47) | (71.06) | | (65.09) | (74.80) |
| Median | 10.6 | 7.2 | Median | 16.5 | 12.3 | Median | 20.9 | 12.5 |
| Q1, Q3 | −12.7, | −19.4, | Q1, Q3 | −7.9, | −13.5, | Q1, Q3 | −7.7, | −15.1, |
| | 37.6 | 35.6 | | 52.2 | 43.8 | | 62.7 | 49.5 |
| Min, Max | −118, 634 | −200, 574 | Min, Max | −127, 453 | −329, 347 | Min, Max | −123, 350 | −255, 319 |
| LS Mean (SE) | 18.56 | 11.37 | LS Mean (SE) | 29.63 | 19.09 | LS Mean (SE) | 29.66 | 22.01 |
| | (4.892) | (4.927) | | (4.984) | (5.026) | | (4.954) | (5.005) |
| 95% CI | 8.95, | 1.69, | 95% CI | 19.84, | 9.21, | 95% CI | 19.93, | 12.17, |
| | 28.17 | 21.05 | | 39.43 | 28.97 | | 39.40 | 31.84 |
| LS | | −7.19 | LS | | −10.55 | LS | | −7.66 |
| Mean | | (6.613) | Mean | | (6.758) | Mean | | (6.720) |
| Difference | | | Difference | | | Difference | | |
| (SE) | | | (SE) | | | (SE) | | |
| vs Placebo | | | vs Placebo | | | vs Placebo | | |
| 95% CI | | −20.18, | 95% CI | | −23.83, | 95% CI | | −20.87, |
| vs Placebo | | 5.81 | vs Placebo | | 2.74 | vs Placebo | | 5.55 |
| p-value | | 0.2776 | p-value | | 0.1194 | p-value | | 0.255 |
| vs Placebo | | | vs Placebo | | | vs Placebo | | |

TABLE 184
Change from baseline to Weeks 26, 52 and 65 in Q-Motor Pronation Supination inter
onset interval (IOI)-SD-score in participants not taking neuroleptic medications anytime during the study.

| Week 26 | | | Week 52 | | | Week 65 | | |
|---|---|---|---|---|---|---|---|---|
| | | Pridopidine | | | Pridopidine | | | Pridopidine |
| | | 45 mg | | | 45 mg | | | 45 mg |
| | Placebo | bid | | Placebo | bid | | Placebo | bid |
| n | 141 (0) | 136 (0) | n | 141 (0) | 136 (0) | n | 141 (0) | 136 (0) |
| Baseline mean | 130.9 | 147.1 | Baseline mean | 130.9 | 147.1 | Baseline mean | 130.9 | 147.1 |
| (SD) | (91.78) | (80.36) | (SD) | (91.78) | (80.36) | (SD) | (91.78) | (80.36) |
| n (missing) | 141 (0) | 134 (2) | n (missing) | 130 (11) | 125 (11) | n (missing) | 131 (10) | 125 (11) |
| Mean (SD) | 20.3 | 10.7 | Mean (SD) | 30.9 | 16.7 | Mean (SD) | 31.5 | 17.4 |
| | (75.62) | (59.27) | | (74.08) | (54.30) | | (65.53) | (56.30) |
| Median | 9.8 | 3.4 | Median | 15 | 13.7 | Median | 18.7 | 13.1 |
| Q1, Q3 | −11.9, | −19.1, | Q1, Q3 | −9.3, | −13.6, | Q1, Q3 | −8.3, | −9.1, 45.1 |
| | 29.7 | 33.7 | | 45.6 | 40.5 | | 53.4 | |
| Min, Max | −82, 634 | −114, 326 | Min, Max | −74, 453 | −168, 258 | Min, Max | −92, 350 | −150, 192 |
| LS Mean (SE) | 19.41 | 9.16 | LS Mean (SE) | 29.12 | 15.86 | LS Mean (SE) | 30.59 | 18.07 |
| | (5.614) | (5.757) | | (5.478) | (5.594) | | (5.378) | (5.497) |
| 95% CI | 8.35, | −2.17, | 95% CI | 18.33, | 4.85, | 95% CI | 20.00 | 7.25, |
| | 30.46 | 20.49 | | 39.91 | 26.88 | | 41.19 | 28.90 |
| LS | | −10.25 | LS | | −13.26 | LS | | −12.52 |
| Mean | | (8.048) | Mean | | (7.839) | Mean | | (7.705) |
| Difference | | | Difference | | | Difference | | |
| (SE) | | | (SE) | | | (SE) | | |
| vs Placebo | | | vs Placebo | | | vs Placebo | | |
| 95% CI | | −26.09, | 95% CI | | −28.69, | 95% CI | | −27.70, |
| vs Placebo | | 5.60 | vs Placebo | | 2.18 | vs Placebo | | 2.65 |
| p-value | | 0.204 | p-value | | 0.0919 | p-value | | 0.1053 |
| vs Placebo | | | vs Placebo | | | vs Placebo | | |

TABLE 185A
Change from baseline to Weeks 26, 52 and 65 in Q-Motor Pronation Supination inter onset
interval (IOI)-SD-score in participants not taking neuroleptic and chorea medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 112 (0) | 97 (0) | n | 112 (0) | 97 (0) | n | 112 (0) | 97 (0) |
| Baseline | 123.5 | 137.2 | Baseline | 123.5 | 137.2 | Baseline | 123.5 | 137.2 |
| mean (SD) | (71.38) | (79.32) | mean (SD) | (71.38) | (79.32) | mean (SD) | (71.38) | (79.32) |
| n | 112 (0) | 95 (2) | n | 103 (9) | 91 (6) | n | 104 (8) | 91 (6) |
| (missing) | | | (missing) | | | (missing) | | |
| Mean | 17.1 | 9.5 | Mean | 27.0 | 14.6 | Mean | 28.9 | 16.6 |
| (SD) | (75.52) | (56.80) | (SD) | (67.12) | (52.33) | (SD) | (58.91) | (54.76) |
| Median | 8 | 2.6 | Median | 14.6 | 11.1 | Median | 15.1 | 15.3 |
| Q1, Q3 | −12.7, | −19.4, 31.7 | Q1, Q3 | −9.3, | −12.7, 37.1 | Q1, Q3 | −8.0, | −9.1, 45.1 |
| | 25.8 | | | 44.3 | | | 51.5 | |
| Min, Max | −82, 634 | −100, 326 | Min, Max | −74, 453 | −168, 258 | Min, Max | −92, 251 | −150, 184 |
| LS Mean | 16.93 | 7.80 | LS Mean | 25.41 | 12.99 | LS Mean | 28.63 | 17.34 |
| (SE) | (6.207) | (6.706) | (SE) | (5.592) | (5.947) | (SE) | (5.585) | (5.920) |
| 95% CI | 4.69, | −5.42, | 95% CI | 14.38, | 1.26, | 95% CI | 17.61, | 5.66, |
| | 29.17 | 21.02 | | 36.44 | 24.72 | | 39.64 | 29.01 |
| LS Mean | | −9.13 | LS Mean | | −12.42 | LS Mean | | −11.29 |
| Difference | | (9.147) | Difference | | (8.170) | Difference | | (8.147) |
| (SE) vs | | | (SE) vs | | | (SE) vs | | |
| Placebo | | | Placebo | | | Placebo | | |
| 95% CI vs | | −27.17, | 95% CI vs | | −28.53, | 95% CI vs | | −27.36, |
| Placebo | | 8.90 | Placebo | | 3.69 | Placebo | | 4.78 |
| p-value vs | | 0.3191 | p-value vs | | 0.1301 | p-value vs | | 0.1674 |
| Placebo | | | Placebo | | | Placebo | | |

TABLE 185B
Change from baseline to Weeks 26, 52, 65 and 78 in Q-Motor Pronation
Supination inter onset interval (IOI)-SD-score in participants not taking neuroleptic and
chorea medications anytime during the study.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | 19.62 | 6.421 | 81 | 4.25 | 7.129 | −15.37 | 9.698 | 0.113 | 80.2% |
| 52 | 99 | 26.31 | 5.786 | 80 | 8.21 | 6.385 | −18.1 | 8.632 | 0.0375 | 71/6% |
| 65 | 100 | 30.38 | 5.739 | 81 | 16.36 | 6.3 | −14.02 | 8.539 | 0.1023 | 51% |
| 78 | 69 | 41.76 | 7.42 | 64 | 25.92 | 7.851 | −15.84 | 10.818 | 0.1452 | 43% |

By analyzing the 101-SD and other parameters related to pronation and supination movements, researchers and clinicians can gain insights into the motor deficits, coordination issues, or changes in motor performance specific to these actions. Pridopidine demonstrates a significant and beneficial effect on Q-Motor was observed in patients not taking neuroleptics and/or chorea medications. Pridopidine treatment showed improvement in Q-Motor. In week 26 progression slowed by 80.2% compared to placebo (negative change indicates improvement) (change vs placebo −15.37, p=0.113), In week 52 progression slowed by 71.6% compared to placebo (change vs placebo −18.1, p=0.0375), in week 65 progression slowed by 51% compared to placebo (change vs placebo −14.02, p=0.1023) and in week 78 progression slowed by 43% compared to placebo (change vs placebo −15.84, p=0.1452). From the results presented Pridopidine 45 mg bid provides Improvement in motor function as measured by the widely used and accepted scale—the Q-Motor up to 78 weeks compared to placebo.

TABLE 186
Change from baseline to Weeks 26, 52 and 65 in Q-Motor Pronation Supination inter
tap interval (ITI)-Mean-Full analysis set (mITT)

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 247 (0) | 242 (1) | n | 247 (0) | 242 (1) | n | 247 (0) | 242 (1) |
| Baseline | 403.9 | 413.2 | Baseline | 403.9 | 413.2 | Baseline | 403.9 | 413.2 |
| mean (SD) | (122.86) | (118.71) | mean (SD) | (122.86) | (118.71) | mean (SD) | (122.86) | (118.71) |
| n | 245 (2) | 238 (5) | n | 225 (22) | 218 (25) | n | 228 (19) | 219 (24) |
| (missing) | | | (missing) | | | (missing) | | |
| Mean | 10.7 | −7.5 | Mean | 15.5 | 7.2 (89.41) | Mean | 19.1 | 9.3 |
| (SD) | (91.14) | (74.91) | (SD) | (95.76) | | (SD) | (86.39) | (93.01) |
| Median | 5.7 | −6.8 | Median | 14.4 | 4.1 | Median | 16.1 | 8.2 |
| Q1, Q3 | −26.9, | −41.3, | Q1, Q3 | −27.4, | −31.7, | Q1, Q3 | −24.0, | −45.1, |
| | 44.3 | 28.1 | | 63.5 | 49.5 | | 69.9 | 44.9 |
| Min, Max | −398, 733 | −352, 250 | Min, Max | −551, 577 | −399, 347 | Min, Max | −426, 463 | −393, 384 |
| LS Mean | 8.95 | −7.36 | LS Mean | 13.04 | 6.92 | LS Mean | 17.28 | 9.87 |
| (SE) | (5.486) | (5.516) | (SE) | (6.100) | (6.151) | (SE) | (6.017) | (6.075) |
| 95% CI | 1.83, | −18.20, | 95% CI | 1.05, | −5.17, | 95% CI | 5.46, | −2.07, |
| | 19.73 | 3.48 | | 25.02 | 19.00 | | 29.11 | 21.81 |
| LS Mean | | −16.31 | LS Mean | | −6.12 | LS Mean | | −7.41 |
| Difference | | (7.360) | Difference | | (8.290) | Difference | | (8.171) |
| (SE) vs | | | (SE) vs | | | (SE) vs | | |
| Placebo | | | Placebo | | | Placebo | | |
| 95% CI vs | | −30.77, | 95% CI vs | | −22.41, | 95% CI vs | | −23.47, |
| Placebo | | −1.85 | Placebo | | 10.17 | Placebo | | 8.65 |
| p-value vs | | 0.0271 | p-value vs | | 0.4609 | p-value vs | | 0.3649 |
| Placebo | | | Placebo | | | Placebo | | |

TABLE 187
Change from baseline to Weeks 26, 52 and 65 in Q-Motor Pronation Supination inter
tap interval (ITI)-Mean-score in participants not taking neuroleptic medications anytime during the study.

| Baseline | | | Baseline | | | Baseline | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 141 | 136 | n | 141 | 136 | n | 141 | 136 |
| Week 26 | | | Week 52 | | | Week 65 | | |
| n | 141 | 134 | n | 130 | 125 | n | 131 | 125 |
| (missing) | | | (missing) | | | (missing) | | |
| LS Mean | 11.9314 | −15.8395 | LS Mean | 16.3222 | 2.3122 | LS Mean | 23.4674 | 0.6203 |
| (SE) | (6.4483) | (6.6141) | (SE) | (7.3589) | (7.519) | (SE) | (6.8576) | (7.0136) |
| 95% CI | −0.7635, | −28.86, | 95% CI | 1.833, | −12.493, | 95% CI | 9.964, | −13.191, |
| | 24.6264 | −2.8185 | | 30.8114 | 17.1172 | | 36.9707 | 14.4312 |
| LS Mean | | −27.7709 | LS Mean | | −14.01 | LS Mean | | −22.847 |
| Difference | | (9.2622) | Difference | | (10.5453) | Difference | | (9.8395) |
| (SE) vs | | | (SE) vs | | | (SE) vs | | |
| Placebo | | | Placebo | | | Placebo | | |
| 95% CI vs | | −46.005, | 95% CI vs | | −34.773, | 95% CI vs | | −42.222, |
| Placebo | | −9.5366 | Placebo | | 6.753 | Placebo | | −3.4723 |
| p-value vs | | 0.003 | p-value vs | | 0.1851 | p-value vs | | 0.021 |
| Placebo | | | Placebo | | | Placebo | | |

TABLE 188
Change from baseline to Weeks 26, 52 and 65 in Q-Motor Pronation Supination inter onset
interval (ITI)-Mean-score in participants not taking neuroleptic and chorea medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 112 (0) | 97 (0) | n | 112 (0) | 97 (0) | n | 112 (0) | 97 (0) |
| Baseline | 381.2 | 412.3 | Baseline | 381.2 | 412.3 | Baseline | 381.2 | 412.3 |
| mean (SD) | (111.74) | (107.75) | mean (SD) | (111.74) | (107.75) | mean (SD) | (111.74) | (107.75) |
| n | 112 (0) | 95 (2) | n | 103 (9) | 91 (6) | n | 104 (8) | 91 (6) |
| (missing) | | | (missing) | | | (missing) | | |
| Mean | 21.2 | −17.4 | Mean | 22.4 | −1.1 | Mean | 30.6 | 1.5 |
| (SD) | (88.72) | (63.77) | (SD) | (85.35) | (92.39) | (SD) | (78.52) | (81.37) |
| Median | 11.1 | −11.8 | Median | 13.7 | −5.1 | Median | 26.6 | 6.2 |
| Q1, Q3 | −20.4, | −51.5, 17.4 | Q1, Q3 | −24.8, | −51.4, 39.0 | Q1, Q3 | −15.6, | −52.1, 45.7 |
| | 40.6 | | | 47.7 | | | 69.2 | |
| Min, Max | −124, 733 | −156, 164 | Min, Max | −113, 577 | −224, 347 | Min, Max | −129, 463 | −184, 293 |
| LS Mean | 20.43 | −17.63 | LS Mean | 20.07 | −0.15 | LS Mean | 28.55 | 4.64 |
| (SE) | (7.448) | (8.029) | (SE) | (8.555) | (9.124) | (SE) | (7.867) | (8.380) |
| 95% CI | 5.74, | −33.46, | 95% CI | 3.20, | −18.14, | 95% CI | 13.04, | −11.89, |
| | 35.11 | −1.80 | | 36.94 | 17.85 | | 44.07 | 21.16 |
| LS Mean | | −38.06 | LS Mean | | −20.21 | LS Mean | | −23.92 |
| Difference | | (10.999) | Difference | | (12.547) | Difference | | (11.541) |
| (SE) vs | | | (SE) vs | | | (SE) vs | | |
| Placebo | | | Placebo | | | Placebo | | |
| 95% CI vs | | −59.74, | 95% CI vs | | −44.95, | 95% CI vs | | −46.68, |
| Placebo | | −16.37 | Placebo | | 4.53 | Placebo | | −1.16 |
| p-value vs | | 0.0007 | p-value vs | | 0.1087 | p-value vs | | 0.0395 |
| Placebo | | | Placebo | | | Placebo | | |

TABLE 189
Change from baseline to Weeks 26, 52 and 65 in Q-Motor Pronation Supination inter
tap interval (ITI)-SD-Full analysis set (mITT)

| Week 26 | | | Week 52 | | | Week 65 | | |
|---|---|---|---|---|---|---|---|---|
| | | Pridopidine | | | Pridopidine | | | Pridopidine |
| | | 45 mg | | | 45 mg | | | 45 mg |
| | Placebo | bid | | Placebo | bid | | Placebo | bid |
| n | 247 (0) | 242 (1) | n | 247 (0) | 242 (1) | n | 247 (0) | 242 (1) |
| Baseline mean | 124.9 | 138.5 | Baseline mean | 124.9 | 138.5 | Baseline mean | 124.9 | 138.5 |
| (SD) | (79.43) | (86.83) | (SD) | (79.43) | (86.83) | (SD) | (79.43) | (86.83) |
| n (missing) | 245 (2) | 238 (5) | n (missing) | 225 (22) | 218 (25) | n (missing) | 228 (19) | 219 (24) |
| Mean (SD) | 17.2 | 9.1 | Mean (SD) | 26.6 | 14.3 | Mean (SD) | 26.6 | 15.7 |
| | (62.95) | (58.37) | | (61.38) | (60.70) | | (54.42) | (65.56) |
| Median | 8.9 | 3 | Median | 16.5 | 11.7 | Median | 19.6 | 9 |
| Q1, Q3 | −8.5, | −16.8 | Q1, Q3 | −3.6, | −10.7, | Q1, Q3 | −4.3, | −13.9, |
| | 33.6 | 28.8 | | 48.1 | 37.4 | | 49.7 | 39.8 |
| Min, Max | −121, 573 | −211, 364 | Min, Max | −238, 440 | −362, 310 | Min, Max | −115, 284 | −284, 290 |
| LS Mean (SE) | 15.23 | 7.74 | LS Mean (SE) | 24.45 | 14.13 | LS Mean (SE) | 25.01 | 16.05 |
| | (4.063) | (4.092) | | (4.246) | (4.284) | | (4.222) | (4.270) |
| 95% CI | 7.24, | −0.30, | 95% CI | 16.11 | 5.71, | 95% CI | 16.71 | 7.66, |
| | 23.21 | 15.78 | | 32.80 | 22.55 | | −33.30 | 24.44 |
| LS | | −7.48 | LS | | −10.32 | LS | | −8.95 |
| Mean | | (5.496) | Mean | | (5.775) | Mean | | (5.747) |
| Difference | | | Difference | | | Difference | | |
| (SE) | | | (SE) | | | (SE) | | |
| vs Placebo | | | vs Placebo | | | vs Placebo | | |
| 95% CI | | −18.28, | 95% CI | | −21.67, | 95% CI | | −20.25, |
| vs Placebo | | 3.32 | vs Placebo | | 1.03 | vs Placebo | | 2.34 |
| p-value | | 0.174 | p-value | | 0.0746 | p-value | | 0.12 |
| vs Placebo | | | vs Placebo | | | vs Placebo | | |

TABLE 190
Change from baseline to Weeks 26, 52 and 65 in Q-Motor Pronation Supination inter
tap interval (ITI)-SD-score in participants not taking neuroleptic medications anytime during the study.

| Baseline | | | Baseline | | | Baseline | | |
|---|---|---|---|---|---|---|---|---|
| | | Pridopidine | | | Pridopidine | | | Pridopidine |
| | | 45 mg | | | 45 mg | | | 45 mg |
| | Placebo | bid | | Placebo | bid | | Placebo | bid |
| n | 141 | 136 | n | 141 | 136 | n | 141 | 136 |
| Week 26 | | | Week 52 | | | Week 65 | | |
| n | 141 | 134 | n | 130 | 125 | n | 131 | 125 |
| (missing) | | | (missing) | | | (missing) | | |
| LS | 18.3543 | 6.8138 | LS | 26.4817 | 11.2485 | LS | 28.1227 | 11.547 |
| Mean | (4.9779) | (5.1019) | Mean | (4.5891) | (4.6849) | Mean | (4.7567) | (4.8609) |
| (SE) | | | (SE) | | | (SE) | | |
| 95% CI | 8.5542, | −3.2302, | 95% CI | 17.4466, | 2.0244, | 95% CI | 18.7552, | 1.974, |
| | 28.1544 | 16.8577 | | 35.5168 | 20.4725 | | 37.4902 | 21.1199 |
| LS Mean | | −11.5405 | LS Mean | | −15.2332 | LS Mean | | −16.5757 |
| Difference | | (7.1358) | Difference | | (6.5694) | Difference | | (6.8173) |
| (SE) vs | | | (SE) vs | | | (SE) vs | | |
| Placebo | | | Placebo | | | Placebo | | |
| 95% CI vs | | −25.589, | 95% CI vs | | −28.167, | 95% CI vs | | −30.001, |
| Placebo | | 2.5075 | Placebo | | −2.2991 | Placebo | | −3.1506 |
| p-value | | 0.107 | p-value | | 0.0212 | p-value | | 0.0157 |
| vs | | | vs | | | vs | | |
| Placebo | | | Placebo | | | Placebo | | |

TABLE 191A
Change from baseline to Weeks 26, 52 and 65 in Q-Motor Pronation Supination inter tap
interval (ITI)-SD-score in participants not taking neuroleptic and chorea medications anytime during the study.

| Week 26 | | | Week 52 | | | Week 65 | | |
|---|---|---|---|---|---|---|---|---|
| | | Pridopidine | | | Pridopidine | | | Pridopidine |
| | Placebo | 45 mg bid | | Placebo | 45 mg bid | | Placebo | 45 mg bid |
| n | 112 (0) | 97 (0) | n | 112 (0) | 97 (0) | n | 112 (0) | 97 (0) |
| Baseline | 110.5 | 122.7 | Baseline | 110.5 | 122.7 | Baseline | 110.5 | 122.7 |
| mean (SD) | (65.94) | (67.17) | mean (SD) | (65.94) | (67.17) | mean (SD) | (65.94) | (67.17) |
| n | 112 (0) | 95 (2) | n | 103 (9) | 91 (6) | n | 104 (8) | 91 (6) |
| (missing) | | | (missing) | | | (missing) | | |
| Mean | 18.3 | 8.1 | Mean | 24.9 | 9.5 | Mean | 27.7 | 9.7 |
| (SD) | (70.19) | (49.93) | (SD) | (60.01) | (42.74) | (SD) | (51.58) | (45.69) |
| Median | 7.7 | −0.1 | Median | 15.6 | 9.5 | Median | 17.1 | 9.5 |
| Q1, Q3 | −6.9, | −18.1, | Q1, Q3 | −4.9, | −11.2, | Q1, Q3 | −4.3, | −16.0, |
| | 26.9 | 23.4 | | 39.1 | 29.7 | | 49.6 | 35.8 |
| Min, Max | −91, 573 | −85, 247 | Min, Max | −88, 440 | −143, 206 | Min, Max | −71, 247 | −153, 136 |
| LS Mean | 17.86 | 7.18 | LS Mean | 23.38 | 8.96 | LS Mean | 27.22 | 10.81 |
| (SE) | (5.726) | (6.181) | (SE) | (4.962) | (5.287) | (SE) | (4.879) | (5.170) |
| 95% CI | 6.57, | −5.00, | 95% CI | 13.60, | −1.46, | 95% CI | 17.60, | 0.61, |
| | 29.15 | 19.37 | | 33.17 | 19.39 | | 36.84 | 21.01 |
| LS Mean | | −10.67 | LS Mean | | −14.42 | LS Mean | | −16.41 |
| Difference | | (8.432) | Difference | | (7.256) | Difference | | (7.117) |
| (SE) vs | | | (SE) vs | | | (SE) vs | | |
| Placebo | | | Placebo | | | Placebo | | |
| 95% CI vs | | −27.30, | 95% CI vs | | −28.73, | 95% CI vs | | −30.45, |
| Placebo | | 5.95 | Placebo | | −0.11 | Placebo | | −2.37 |
| p-value vs | | 0.207 | p-value vs | | 0.0482 | p-value vs | | 0.0222 |
| Placebo | | | Placebo | | | Placebo | | |

TABLE 191B
Change from baseline to Weeks 26, 52 and 65 in Q-Motor Pronation Supination inter tap
interval (ITI)-SD-score in participants not taking neuroleptic and chorea medications anytime during the study.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | 20.11 | 6.004 | 81 | 3.54 | 6.674 | −16.58 | 8.99 | 0.0669 | 84.19907602 |
| 52 | 99 | 23.93 | 5.154 | 80 | 3.79 | 5.693 | −20.14 | 7.694 | 0.0096 | 85.78365994 |
| 65 | 100 | 28.82 | 5.005 | 81 | 9.01 | 5.491 | −19.8 | 7.447 | 0.0085 | 71.93777473 |
| 78 | 69 | 39.3 | 6.825 | 64 | 20.43 | 7.184 | −18.87 | 9.923 | 0.0591 | 53.58046974 |

A strong beneficial effect of pridopidine on Q-Motor was observed in patients excluding antidopaminergic drugs. Pridopidine showed less decline in Q-Motor at week 26 and slowed progression by 84.1% compared to the placebo (negative change indicates improvement) (change vs placebo −16.58, p=0.0669), in week 52 pridopidine treatment slowed progression by 85.7% compared to the placebo (change vs placebo −20.14, p=0.0096) in week 65 pridopidine treatment slowed progression by 71.9% compared to the placebo (change vs placebo −19.8, p=0.0085) and in week 78 pridopidine treatment slowed progression by 53.5% compared to the placebo (change vs placebo −18.87, p=0.0591). From the results presented Pridopidine 45 mg bid provides Improvement in motor function as measured by the widely used and accepted scale—the Q-Motor up to 78 weeks compared to placebo.

There was a strong and significant association between changes in Q-Motor assessment and changes in cUHDRS (p<0.0001 FIG. 73) and changes in TFC (P<0.0001, FIG. 74).

TABLE 192A
Change from baseline to Weeks 26, 52, 65 and 78 in QoL in participants not taking
neuroleptic and chorea medications anytime during the study. mITT group.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 112 | 5.28 | 2.428 | 96 | 0.44 | 2.576 | −4.83 | 3.541 | 0.1736 | 91.7% |
| 52 | 104 | 2.76 | 2.401 | 91 | −1.82 | 2.528 | −4.58 | 3.487 | 0.191 | 165.2% |
| 65 | 106 | 2.79 | 2.419 | 88 | −0.76 | 2.583 | −3.55 | 3.542 | 0.3172 | 126.9% |
| 78 | 72 | 4.5 | 2.958 | 69 | 0.6 | 3.083 | −3.9 | 4.273 | 0.3626 | 86.8% |

TABLE 192B
Change from baseline to Weeks 26, 52, 65 and 78 in QoL in participants not taking
neuroleptic and chorea medications anytime during the study. Per protocol group.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | 4.09 | 2.543 | 81 | −0.25 | 2.775 | −4.34 | 3.769 | 0.2507 | 106.2% |
| 52 | 100 | 1.51 | 2.471 | 81 | −2.35 | 2.683 | −3.86 | 3.654 | 0.2928 | 257.8% |
| 65 | 102 | 1.97 | 2.557 | 79 | −0.41 | 2.811 | −2.38 | 3.807 | 0.5323 | 121.1% |
| 78 | 70 | 3.84 | 3.11 | 63 | 0.68 | 3.35 | −3.16 | 4.576 | 0.4907 | 81.9% |

A strong beneficial effect of pridopidine on QoL was observed in patients excluding antidopaminergic drugs. Pridopidine showed less decline in QoL at week 26 and slowed progression by 91.7% compared to the placebo in the mITT all patients group and an improvement of 106.2% compared to the placebo was seen at 26 weeks in the Per protocol group. In week 52 an improvement of 165.2% in QoL was seen in the mITT all patients group compared to the placebo, and 257.8% improvement compared to placebo in the Per protocol group. In week 65 a 126.9% improvement in QoL compared to placebo was seen in the mITT group and a 121.1% improvement in the Per protocol group. In week 78 pridopidine treatment slowed progression by 86.8% in QoL compared to the placebo in the mITT all patients group and in the Per protocol group pridopidine treatment slowed progression by 81.9% compared to the placebo in QoL.

TABLE 193 A
Change from baseline to Weeks 26, 52 and 65 in TMS Gait and balance
subdomain in in participants not taking neuroleptic and chorea medications anytime during
the study. mITT group.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 112 | 0.06 | 0.113 | 96 | −0.13 | 0.122 | −0.19 | 0.166 | 0.2512 | 305.4% |
| 39 | 107 | 0.16 | 0.129 | 91 | −0.1 | 0.139 | −0.26 | 0.189 | 0.1727 | 159.2% |
| 52 | 104 | 0.25 | 0.121 | 91 | −0.06 | 0.129 | 0.32 | 0.177 | 0.074 | 122.7% |
| 65 | 105 | 0.45 | 0.129 | 91 | 0 | 0.138 | −0.45 | 0.189 | 0.0188 | 100% |

TABLE 193B
Change from baseline to Weeks 26, 52, 65 and 78 in TMS Gait and balance
subdomain in in participants not taking neuroleptic and chorea medications anytime during
the study. Per protocol group.

| | | | | | | | Pridopidine 45 mg bid vs | | | % Progression |
|---|---|---|---|---|---|---|---|---|---|---|
| | Placebo | | | Pridopidine 45 mg bid | | | Placebo | | | Slowed vs |
| Week | N | LS Mean | SE | N | LS Mean | SE | LS Mean | SE | P-Value | placebo |
| 26 | 102 | 0.06 | 0.118 | 81 | −0.18 | 0.13 | −0.23 | 0.176 | 0.1849 | 377.5% |
| 39 | 102 | 0.13 | 0.132 | 79 | −0.04 | 0.147 | −0.17 | 0.198 | 0.379 | 128.4% |
| 52 | 100 | 0.24 | 0.124 | 80 | −0.04 | 0.138 | −0.28 | 0.186 | 0.1375 | 115.4% |
| 65 | 101 | 0.42 | 0.131 | 81 | −0.05 | 0.145 | −0.48 | 0.196 | 0.0158 | 111% |
| 78 | 70 | 0.57 | 0.152 | 64 | 0.22 | 0.163 | −0.35 | 0.223 | 0.1148 | 64% |

### Summary:

Pridopidine treatment was associated with maintenance, no worsening or improvements in global progression as measured by cUHDRS and UHDRS-TFC, cognition as measured by SWR score and motor function as measured by TMS score, and Q-Motor. The benefit of pridopidine treatment was stronger, reaching statistical significance in HD patients not receiving neuroleptics and/or chorea medications.

HD patients not taking neuroleptics and or chorea meds, treated with 45 mg pridopidine twice daily showed less TFC decline than placebo up to week 52. These observations suggest that pridopidine at a dose of 45 mg bid is associated with maintenance/slower decline of functional capacity in HD.

Global progression in HD as measured by the cUHDRS includes function, motor and cognitive assessment and is the most sensitive scale to evaluate patients' overall function and disease state. Maintenance, less decline, or improvement in cUHDRS is perhaps the most pressing unmet therapeutic need in HD. Pridopidine shows beneficial effects in cUHDRS up to 65 weeks, most pronounced and statisticaly significant up to 52 weeks in patients not taking neuroleptics and chorea medications The study shows that pridopidine 45 mg bid provides improvement in a validated measure of cognitive function, the Stroop word reading (SWR) test up to 78 weeks, in HD patients. In patients not taking neuroleptics and chorea the effect was most pronounced and reached statistical significance at weeks 26, 39 and 53.

The study shows that pridopidine 45 mg bid provides improvement in motor function as measured by the widely used and accepted scale—the Total Motor Score (TMS) up to 65 compared to placebo, in all patients, the effect was strongest in patients not taking neuroleptics and chorea meds.

The study shows that pridopidine 45 mg bid provides an improvement in all the different measures Q-Motor: Finger Tap IOI-mean, Finger Tap IOI-SD, Finger Tap ITI-SD, Pronation Supination IOI mean, Pronation Supination IOI SD, Pronation Supination ITI mean and Pronation Supination ITI SD at 26, 52, 65 and 78 weeks. The beneficial effects of pridopidine in all Q-Motor measures were strongest and reached statistical significance in patients not taking neuroleptics and/or chorea meds.

These observations suggest that pridopidine at a dose of 45 mg bid is associated with improvement of motor function in HD.

The study shows that pridopidine 45 mg bid provides improvement in a validated measure of Quality of life up to 78 weeks, in HD patients. In patients not taking neuroleptics and chorea the effect was most pronounced.

The study shows that pridopidine 45 mg bid provides improvement in a validated measure of TMS gait and balance subdomain up to 78 weeks, in HD patients. In patients not taking neuroleptics and chorea the effect was most pronounced.

### REFERENCES CITED

Alexander G E, DeLong M R, Strick P L. Parallel organization of functionally segregated circuits linking basal ganglia and cortex. Annu Rev Neurosci. 1986; 9:357-81.
Bechtel, N. et al., Tapping linked to function and structure in premanifest and symptomatic Huntington disease. Neurology. 2010 Dec. 14; 75(24):2150-60.
Bezdicek O, Majerova V, Novak M, Nikolai T, Ruzicka E, Roth J. Validity of the Montreal Cognitive Assessment in the detection of cognitive dysfunction in Huntington's disease. Appl Neuropsychol Adult. 2013; 20(1):33-40.
Bowie C R, Harvey P D. Administration and interpretation of the Trail Making Test. Nat Protoc. 2006; 1(5): 2277-81.
Brown M, Sinacore D R, Binder E F, Kohrt W M. Physical and performance measures for the identification of mild to moderate frailty. J Gerontol A Biol Sci Med Sci. 2000 June; 55A(6):M350-5.
Byrne, L M et al., Neurofilament light protein in blood as a potential biomarker of neurodegeneration in Huntington's disease: a retrospective cohort analysis, Lancet Neurol published Jun. 7, 2017,
Carlsson A, Lindqvist M. Effect of chlorpromazine or haloperidol on formation of 3-methoxytyramine and normetanephrine in mouse brain. Acta Pharmacol Toxicol (Copenh). 1963; 20:140-4.
Cepeda C, Cummings D M, André VM, Holley S M, Levine M S. Genetic mouse models of Huntington's disease: focus on electrophysiological mechanisms. ASN Neuro. 2010 Apr. 7; 2(2):e00033.
Coenzyme Q10 in Huntington's Disease (HD) (2CARE), ClinicalTrials.gov Identifier: NCT00608881, clinicaltrials.gov/ct2/show/NCT00608881?term=2CARE%20+Huntington&rank=1, accessed Sep. 13, 2016.
Craufurd D, Thompson J C, Snowden J S. Behavioral changes in Huntington Disease. Neuropsychiatry Neuropsychol Behav Neurol. 2001 October-December; 14(4):219-26.
Dunlop B W, Nemeroff C B. The role of dopamine in the pathophysiology of depression. Arch Gen Psychiatry. 2007 March; 64(3):327-37.
Dyhring T, Nielsen E O, Sonesson C, Pettersson F, Karlsson J, Svensson P, Christophersen P, Waters N. The dopaminergic stabilizers pridopidine (ACR16) and (−)-OSU6162 display dopamine D(2) receptor antagonism and fast receptor dissociation properties. Eur J Pharmacol. 2010 Feb. 25; 628(1-3): 19-26.
Eddings, C. R., Arbez, N., Akimov, S., Geva, M., Hayden, M. R., & Ross, C. A. (2019). Pridopidine protects neurons from mutant-huntingtin toxicity via the sigma-1 receptor. Neurobiology of disease, 129, 118-129.
Exploratory Population Pharmacokinetic Modeling and Simulations With Pridopidine (Report Number: CP-13-013). Pharsight Consulting Services, 10 Jul. 2013.
Geva, M., Kusko, R., Soares, H., Fowler, K. D., Birnberg, T., Barash, S., . . . & Cha, Y. (2016). Pridopidine activates neuroprotective pathways impaired in Huntington Disease. Human molecular genetics, 25(18), 3975-3987.
Guy W. Clinical Global Impression: ECDEU assessment manual for psychopharmacology. Publication ADM-76-338, US Department of Health, Education, and Welfare Washington DC: US Government Printing Office. 1976: 217-22.
Hobart J C, Riazi A, Lamping D L, Fitzpatrick R, Thompson A J. Measuring the impact of MS on walking ability: the 12-Item MS Walking Scale (MSWS-12). Neurology. 2003 Jan. 14; 60(1):31-6.
Hocaoglu M B, Gaffan E A, Ho A K. The Huntington's Disease health-related Quality of Life questionnaire (HDQoL): a disease-specific measure of health-related quality of life. Clin Genet. 2012 February; 81(2):117-22.
Huntington Study Group TREND-HD Investigators. Randomized controlled trial of ethyleicosapentaenoic acid in Huntington disease: the TREND-HD study. Arch Neurol. 2008 December; 65(12):1582-9.
Huntington Study Group. Unified Huntington's Disease Rating Scale: Reliablility and Consistency, Movement Disorders, 1996, 11(2): 136-142.
Huntington Study Group. Dosage effects of riluzole in Huntington's disease: a multicenter placebo-controlled study. Neurology. 2003 Dec. 9; 61(11): 1551-6.
Huntington Study Group. Tetrabenazine as antichorea therapy in Huntington disease: a randomized controlled trial. Neurology. 2006 Feb. 14; 66(3):366-72.
Joffres C, Graham J, Rockwood K. Qualitative analysis of the clinician interview-based impression of change (Plus): methodological issues and implications for clinical research. Int Psychogeriatr. 2000 September; 12(3):403-13.
Johnson A C and Paulsen J S. Huntington's Disease: A Guide for Professionals. D. Lovecky and K. Tarapata eds. 2014. Huntington's Disease Society of Americas (HDSA)
Kieburtz K, Koroshetz W, McDermott M, et al. A randomized, placebo-controlled trial of coenzyme Q10 and remacemide in Huntington's disease. Neurol. 2001 Aug. 14; 57(3):397-404.
Kingma E M, van Duijn E, Timman R, van der Mast R C, Roos R A. Behavioural problems in Huntington's disease using the Problem Behaviours Assessment. Gen Hosp Psychiatry. 2008 March-April; 30(2): 155-6
Krishnamoorthy, A. and Craufurd, D. Treatment of Apathy in Huntington's Disease and Other Movement Disorders. Current Treatment Options in Neurology, 2011, 13(5):508-19
Landwehrmeyer B, Marder K, Biilmann Ronn B, Haglund Mon behalf of the MermaiHD and HART study investigators. Effects of the dopaminergic stabilizer pridopidine on motor symptoms in Huntington's disease: a meta-analysis. Presented at the World Congress on Huntington's Disease, 11-14 Sep. 2011, Melbourne (Australia).
Mahant N, McCusker E A, Byth K, Graham S; Huntington Study Group. Huntington's disease: clinical correlates of disability and progression. Neurology. 2003 Oct. 28; 61(8):1085-92.
Marder K, Zhao H, Myers R H, Cudkowicz M, Kayson E, Kieburtz K, Orme C, Paulsen J, Penney J B Jr, Siemers E, Shoulson I. Rate of functional decline in Huntington's disease. Huntington Study Group. Neurology 2000; 54:452-58
Marin R S, Biedrzycki R C, Firinciogullari S. Reliability and validity of the Apathy Evaluation Scale. Psychiatry Res 1991; 38(2):143-62.
Martinez-Horta S, Perez-Perez J, van Duijn E, Fernandez-Bobadilla R, Carceller M, Pagonabarraga J, et al. Neuropsychiatric symptoms are very common in premanifest and early stage Huntington's Disease. Parkinsonism Relat Disord 2016; 25:58-64.
Mestre T, Ferreira J, Coelho M M, Rosa M, Sampaio C. Therapeutic interventions for symptomatic treatment in Huntington's disease. Cochrane Database Syst Rev. 2009 Jul. 8; (3).
Myers R H, Sax D S, Koroshetz W J, Mastromauro C, Cupples L A, Kiely D K, Pettengill F K, Bird E D. Factors associated with slow progression in Huntington's disease. Arch Neurol. 1991 August 48(8): 800-4.
Natesan S, Svensson K A, Reckless G E, Nobrega J N, Barlow K B, Johansson A M, Kapur S. The dopamine stabilizers (S)-(−)-(3-methanesulfonyl-phenyl)-1-propyl-piperidine [(−)-OSU6162] and 4-(3-methanesulfonylphenyl)-1-propyl-piperidine (ACR16) show high in vivo D2 receptor occupancy, antipsychotic-like efficacy, and low potential for motor side effects in the rat. J Pharmacol Exp Ther. 2006 August; 318(2):810-8.
Open-label Extension Study of Pridopidine (ACR16) in the Symptomatic Treatment of Huntington Disease (OPEN-HART), ClinicalTrials.gov Identifier: NCT01306929, clinicaltrials.gov/ct2/show/NCT01306929, accessed Sep. 13, 2016.
Podsiadlo D, Richardson S. The timed “Up & Go”: a test of basic functional mobility for frail elderly persons. J Am Geriatr Soc. 1991 February; 39(2):142-8.
Ponten H, Kullingsjo J, Lagerkvist S, Martin P, Pettersson F, Sonesson C, Waters S, Waters N. In vivo pharmacology of the dopaminergic stabilizer pridopidine. Eur J Pharmacol. 2010 Oct. 10; 644(1-3):88-95.
Posner K, Brown G K, Stanley B, Brent D A, Yershova K V, Oquendo M A, Currier G W, Melvin G A, Greenhill L, Shen S, Mann J J. The Columbia-Suicide Severity Rating Scale: initial validity and internal consistency findings from three multisite studies with adolescents and adults. Am J Psychiatry. 2011 December; 168(12):1266-77.
Rao A K, Muratori L, Louis E D, Moskowitz C B, Marder K S. Clinical measurement of mobility and balance impairments in Huntington's disease: validity and responsiveness. Gait Posture. 2009 April; 29(3):433-6.
Reuben D B, Siu A L. An objective measure of physical function of elderly outpatients. The Physical Performance Test. J Am Geriatr Soc. 1990 October; 38(10):1105-12.
Schobel et al., (2017) Motor, cognitive, and functional declines contribute to a single progressive factor in early HD. Neurobiology 89:2495-2502.
Smith-Dijak, A. I., Nassrallah, W. B., Zhang, L. Y., Geva, M., Hayden, M. R., & Raymond, L. A. (2019). Impairment and restoration of homeostatic plasticity in cultured cortical neurons from a mouse model of huntington disease. Frontiers in Cellular Neuroscience, 13, 209.
Stout J C, Queller S, Baker K N, Cowlishaw S, Sampaio C, Fitzer-Attas C, Borowsky B; HD-CAB Investigators. HD-CAB: a cognitive assessment battery for clinical trials in Huntington's disease. Mov Disord 2014; 29(10): 1281-8.
Stroop J R. Studies of interference in serial verbal reactions. J Exp Psychol 1935; 18:643-62.
The EuroQol Group. EuroQol—a new facility for the measurement of health-related quality of life. Health Policy 1990; 16:199-208.
Waters S, Pettersson F, Dyhring T, Sonesson C, Tedroff J, Waters N et al. Pharmacology of the dopaminergic stabilizer pridopidine (ACR16). Clin Genet 2009; 76(S1):74 (Abstract D10).
Zhan L, Kerr J R, Lafuente M J, Maclean A, Chibalina M V, Liu B, Burke B, Bevan S, Nasir J. Altered expression and coregulation of dopamine signaling genes in schizophrenia and bipolar disorder. Neuropathol Appl Neurobiol. 2011 February; 37(2):206-19.

What is claimed is:

1. A method of maintaining, improving, or lessening the decline of motor function and functional capacity in a human patient afflicted with early stage Huntington disease (HD) [early HD, TFC is 7-13] wherein the method comprises orally administering to the patient with early HD, TFC 7-13, a pharmaceutical composition comprising pridopidine or a pharmaceutically acceptable salt thereof wherein assessment of said maintaining, improving, or lessening the decline of motor function and functional capacity comprises using a composite Unified Huntington Disease rating scale (cUHDRS), wherein said cUHDRS comprises measurement of the total functional capacity (TFC), total motor score (TMS), symbol digital modalities test (SDMT), and Stroop Word Reading Test (SWR) of the patient according to the following equation:
$$cUHDRS = [ ( \frac{T F C - 10.4}{1.9} ) - ( \frac{TMS - 29.7}{14.9} ) + ( \frac{S D M T - 28.4}{11.3} ) + ( \frac{SWR - 66.1}{20.1} ) ] + 10.$$

2. The method of claim 1, wherein the pharmaceutical composition comprises pridopidine or a pharmaceutically acceptable salt thereof and Compound 1, Compound 4, combination thereof or pharmaceutically acceptable salt thereof, wherein Compound 1 and Compound 4 are represented by the following structures:

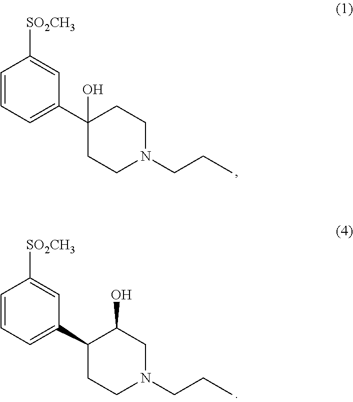 [SMILES: CCCN1CCC(O)(c2cccc(S(C)(=O)=O)c2)CC1 ; CCCN1CC[C@@H](c2cccc(S(C)(=O)=O)c2)[C@@H](O)C1]

3. The method of claim 1, wherein the pharmaceutical composition comprises pridopidine or a pharmaceutically acceptable salt thereof and Compound 1 or a pharmaceutically acceptable salt thereof.

4. The method of claim 1, wherein the pharmaceutical composition comprises pridopidine or a pharmaceutically acceptable salt thereof and Compound 4 or a pharmaceutically acceptable salt thereof.

5. The method of claim 1, wherein the pharmaceutical composition comprises pridopidine or a pharmaceutically acceptable salt thereof, Compound 1 or a pharmaceutically acceptable salt thereof and Compound 4 or a pharmaceutically acceptable salt thereof.

6. The method of claim 1, wherein said administration is for at least 26 weeks or at least 52 weeks.

7. The method of claim 6, wherein pridopidine or a pharmaceutically acceptable salt thereof is administered at a dose 45 mg bid for a period of at least 26 weeks or at least 52 weeks.

8. The method of claim 1, wherein the method comprises maintaining, improving, or lessening the decline of total functional capacity (TFC) of said patient.

9. The method of claim 1, wherein said method comprises maintaining, improving, or lessening the decline of motor function in said patient.

10. The method of claim 1, wherein said composite Unified Huntington Disease rating scale (cUHDRS) produces an improved longitudinal Signal to Noise (S/N) ratio compared with a longitudinal S/N ratio of at least one of the independent UHDRS clinical measures of TFC, TMS, SDMT, or SWR.

11. The method of claim 9, wherein the motor function is assessed by Q motor.

12. The method of claim 1, wherein the maintaining, improving, or lessening the decline of motor function and functional capacity does not include administering neuroleptics medications.

13. The method of claim 1, wherein the maintaining, improving, or lessening the decline of motor function and functional capacity does not include administering chorea medications.

14. The method of claim 1, wherein the pharmaceutical composition is administered twice per day.

15. The method of claim 1, wherein the pharmaceutical composition comprising pridopidine or a pharmaceutically acceptable salt thereof is administered at a dose of between 90-225 mg/day.

16. The method of claim 1, wherein the pharmaceutical composition comprising pridopidine or a pharmaceutically acceptable salt thereof is administered at a dose of 90 mg per day.

17. The method of claim 11, wherein the pharmaceutical composition comprising pridopidine or a pharmaceutically acceptable salt thereof is administered at a dose of 45 mg twice per day (b.i.d.).

18. The method of claim 1, wherein the pharmaceutically acceptable salt is selected from the group consisting from hydrochloride, hydrobromide, hydroiodide, nitrate, perchlorate, phosphate, acid-phosphate, sulphate, bisulfate, formate, gluconate, glucaronate, saccharate, isonicotinate, acetate, aconate, ascorbate, benzenesulphonate, benzoate, cinnamate, citrate, embonate, enantate, fumarate, glutamate, glycolate, lactate, maleate, gentisinate, malonate, mandelate, methanesulfonate, ethanesulfonate, naphthalene-2-sulphonate, phthalate, salicylate, sorbate, stearate, succinate, tartrate, pantothenate, bitartrate, and toluene-p-sulfonate, pamoate (i.e., 1,1′-methylene-bis-(2-hydroxy-3-naphthoate)) salt.

19. The method of claim 18, wherein the pharmaceutically acceptable salt is HCl salt.
